SEQUENCE LISTING (269 sequences)

SEQ ID NO: 1 - DNA, 813 bp, Sus scrofa
  source 1..813: mol_type = genomic DNA
atgccccgagacaacatggcctccctgatccaacggatcgcccgccaggcatgcctcaccttccggggcagcgggggcggccgcagctcttccgatcgcggcgcggcgccaggccctgaggcgcctgtgccgcagggcttcccggagaacctgagcaagctgaagagcctgctgacacaggtccgcgcagaggacctgaacatctccccgcgcaaggccacgctgcagccgttgccacccaacctgccgcccgtcacctatatgcacatctacgagactgacggcttcagcctcggcgtgttcttgcttaagagcggcacatccatcccgctccacgaccaccctggcatgcatggcatgctcaaggtgctctatggcaccgtgcgcatcagctgcatggacaagctggaggcaggcagcgggcaacggccgcgggccccgccaccagagcagcagttcgaaccgccgctgctggcccgggagcgggacgcggtgcggccgggagtgctgcgctcgcgggccgagtacactgaggccagcggtccctgcgtcctcacgccgcaccgggacaacctgcaccagatcgacgctgtggatgggcctgccgccttcttggatatcctggccccgccctacgacccggacgacggccgggactgtcactattaccgggtgctggagcctgtcagggccaaagaggcctccgactcggcctgtgacctgccccgagaggtgtggcttctggagaccccgcaggccgatgacttttggtgcgagggggagccctatccaggtcccagggtcttcccttga

SEQ ID NO: 2 - protein, 270 aa, Sus scrofa
MPRDNMASLIQRIARQACLTFRGSGGGRSSSDRGAAPGPEAPVPQGFPENLSKLKSLLTQVRAEDLNISPRKATLQPLPPNLPPVTYMHIYETDGFSLGVFLLKSGTSIPLHDHPGMHGMLKVLYGTVRISCMDKLEAGSGQRPRAPPPEQQFEPPLLARERDAVRPGVLRSRAEYTEASGPCVLTPHRDNLHQIDAVDGPAAFLDILAPPYDPDDGRDCHYYRVLEPVRAKEASDSACDLPREVWLLETPQADDFWCEGEPYPGPRVFP

SEQ ID NO: 3 - DNA, 1582 bp, Sus scrofa
  source 1..1582: mol_type = genomic DNA
atgatcacatctccgttgctggcttatgtggcgattctgtttttgtgtgtcttaaaagccagctccctggatacctttatcgccgctgtttatgaacatgcagtgatattgcctgatgcccccctcacaccagtatcccatgaagaggctttgatgttaatgaaccgcaatctggatcttttggagggcgcaatcatgtcagcagcaaagcagggtgcacatattattgtgactccagaagatggtgtttacggctttttcttcagcagggaatctatttactcatacttagaggacatcccagagcctcacgtgaactggatcccttgtaccaatcccaacagatttggccacactccagtacaaaaaagactcagctgcctggccagggacaactccatctacattgtggcaaatattggggacaagaagccatgcaatgccagtgaccccgactgtcctcatgacggccgttaccagtacaacactgatgtggtgtttgattctgagggcagactggtggcacgctatcataagcaaaatcttttcttgggtgaagatcaattcgatgcacccaaggaacccgagatcgtgactttcgacaccacctttggaagatttggcattttcacgtgctttgatattctcttccatgatcctgctgtgactctggtgaaagatttccaggtggacaccatactcttcccgacagcttggatgaatgttttgccacatttgacagctattgaattccactcagcttgggctatgggcatgagagtcaacttccttgcagccaatatacatttccccttaagaaaaatgacaggaagtggcatctatgcacctgattctccaagagcatttcattacgatatgaagacaaaggagggaaaactcctcctctcacaattggactcccacccacaccgcccagcagtgaattggacttcttatgccagtggtttaccagcccccttagtgggaaaccaggaatttaaaagcattgtcttttttgacgagttcaccttcttcgagctcaaaggagttgcaggaaattacacagtttgtcagaaagatctctgctgtcagctaagttacaggatgcttgagatgagagaagacgaagtttatgcactaggggcattcgatggactacatactgtggaagggagctattatttacagatttgtactttgttgaagtgtaaaacaatggacttacactcctgtggtgactcggtggaaactgcttccaccaggtttgaaatgttctccctcagtggcacttttggaacccagtatgtgttccctgaggtgttgctgagtgacatacagcttgcacctggagaatttcaggtgtcaagtgatggacgcttgttcagcctgaagccaccgtctggacctgtcttgactgtcactctgtttgggaggttatatgagagggatagtgcatcaggtgcttcagcagacctcgtggcacaaggactgagagtcatgctcggagttataataactattatgtactcattaagttggtagaatatttatacttttctctctttcacttcggatcctttaa

SEQ ID NO: 4 - protein, 513 aa, Sus scrofa
MITSPLLAYVAILFFCVLKASSLDTFIAAVYEHAAILPDAPLTPVSHEEALMLMNRNLDLLEGAVTSAAKQGAHIIVTPEDGVYGFFFSRESIYSYLEDIPDPHVNWIPCTNPSRFGHTPVQKRLSCLARDNSIYIVANIGDKKPCNASDPDCPHDGRYQYNTDVVFDSEGRLVARYHKQNLFLGEDQFDAPKEPEIVTFDTTFGRFGIFTCFGILFHDPAVTLVKDFQVDTILFPTAWMNVLPHLTAIEFHSAWAMGMRVNFLAANIHFPLRKMTGSGIYAPDSPRAFHYDMKTKEGKLLLSQLDSHPHRPAVNWTSYASGLPTPLVGNQEFKSTVFFDEFTFLELKGVAGNYTVCQKDLCCQLSYRMLEKREDEVYALGAFDGLHTVEGSYYLQICTLLKCKTMDLHSCGDSVETASTRFEMFSLSGTFGTQYVFPEVLLSDIQLAPGEFQVSSDGRLFSLKPPSGPVLTVTLFGRLYERDSASGASADLVAQGLRVMLGVIITIMYSLSW

SEQ ID NO: 5 - DNA, 1599 bp, Sus scrofa
  source 1..1599: mol_type = genomic DNA
atggccaagcgagttgcaattgtgggagctggggtcagtggcctggcctccatcaagtgctgcctggaggaagggctggagcccacctgctttgagaggagcgatgaccttggggggctgtggagattcactgaacatgttgaagaaggcagagccagcctctacaagtctgtggtttccaacagctgcaaggagatgtcttgttacccagactttccattcccagaagattacccaaactatgtgccaaattctcacttcctggaatatctcagaatgtatgcaaaccagttcaaccttctgaagtgcattcaattcaagactaaagtctgcagtgtaacaaaacatgaagattttaataccaccggtcaatgggatgtagtcactctgtgtgaaggaaagcaagagtcagccgtctttgatgctgtcatggtctgcactggttttctcactaacccatatttgccattggactccttcccaggcataaatacttttaaaggccagtatttccatagccgacagtataaacatccagatatatttaaggacaagagtgttcttgtggttggaatgggaaattcgggcacagacattgctgtggaggccagccacctggcaaaaaaggtgttcctcagcaccactggaggagcatgggtgatcagccgtgtctttgactcagggtacccatgggacatggtgttcatgacacgatttcagaacatgttcagaaattctctcccaactccaattgtgaattggttgatagcaaaaaagatgaacagctggttcaatcatgcaaattatggcttaatacccgaagacaggatacaactaagagagcctgtgctgaatgatgagctcccaggccgtatcatcactgggaaagtgctcatcaagccaagtataaaggaggtgaaggaaaactccgtggtatttaacagctccccagaggaagagcccattgatatcattgtctttgccactggatacacctttgctttccccttccttgatgagagtgtagtgaaggttgaagatggccaggcatcactatacaagtacatcttccctgcacatctgcagaagccaactctggctgttattggcctcatcaaacccttgggctccttactacccacaggggatacacaagctcggtgggctgttcgggtcctgaaaggtgtgaataagttaccaccatcaagtgtcatgatacaggaagttaatacaagaaaagaaaacaagcccagtgggtttggcttgtgttactgcaaagctttacaatccgattatattgcatacatagatgaactgctgacctatatcgatgcaaagcccaacatgttttctctgctcctgacggatccacacctggctttgaccatcttctttggcccatgcacaccataccagttccgcctgactggtccagggaaatgggaaggagccaggaatgctatcatgacccagtgggaccgaacattcaaggtcaccaaaactcgaattgtaaaagaatccccatctccctttgcaagcttacttaaactctttagctttctagccttgcttgtggccattttccagattttcctctaa

SEQ ID NO: 6 - protein, 532 aa, Sus scrofa
MAKRVAIVGAGVSGLASIKCCLEEGLEPTCFERSDDLGGLWRFTEHVEEGRASLYKSVVSNSCKEMSCYPDFPFPEDYPNYVPNSHFLEYLRMYANQFNLLKCIQFKTKVCSVTKHEDFNTTGQWDVVTLCEGKQESAVFDAVMVCTGFLTNPYLPLDSFPGINTFKGQYFHSRQYKHPDIFKDKSVLVVGMGNSGTDIAVEASHLAKKVFLSTTGGAWVISRVFDSGYPWDMVFMTRFQNMFRNSLPTPIVNWLIAKKMNSWFNHANYGLIPEDRIQLREPVLNDELPGRIITGKVLIKPSIKEVKENSVVFNSSPEEEPIDIIVFATGYTFAFPFLDESVVKVEDGQASLYKYIFPAHLQKPTLAVIGLIKPLGSLLPTGDTQARWAVRVLKGVNKLPPSSVMIQEVNTRKENKPSGFGLCYCKALQSDYIAYIDELLTYIDAKPNMFSLLLTDPHLALTIFFGPCTPYQFRLTGPGKWEGARNAIMTQWDRTFKVTKTRIVKESPSPFASLLKLFSFLALLVAIFQIFL

SEQ ID NO: 7 - DNA, 251 bp, Corynebacterium glutamicum
  source 1..251: mol_type = genomic DNA
cttgcagcgttttgctctgccatgaatccattgtgcaccttagctactccattagtgtgatcggggttattttttcacttcaatgggtggctaaaagacgtgggcacgtgagtaaactcatgcgcgcgaaacgatggaagtgaacccatacttttatatatgggtatcggcggtctatgcttgtgggcgtacctgtcccgcgagtgaggtcttacgcgcgggattcgtcttgtgaaaggttagctgacctg

SEQ ID NO: 8 - DNA, 250 bp, Corynebacterium glutamicum
  source 1..250: mol_type = genomic DNA
gccataacaggaatgttcctttcaaaaattgaggaagccttatgacctacaaccctactcagctgccgattattccgggtttgtgaccagctacccgataaataggtcggctgaaaaatttcgttgcaatatcaacaaaaaggcctatcattgggaagtgtcgcaccaagtacttttgcgaagcgccatctgacggattttcaaaagatgtatatgctcggtgcggaaacctacgaaaggatttttaccc

SEQ ID NO: 9 - DNA, 250 bp, Corynebacterium glutamicum
  source 1..250: mol_type = genomic DNA
gcgtctgaccgagctcgtagcttccaagctctaattagttcacatcgctaacgtgggcgatcgatgctcacggtgatgtgtcatcccaatagcccggggtgtgcctcggcgcaccccgggctattttgtgtctttaatcaatacaattgaataccggtgccagcgccacacaatgtgtggcaatctgggacagtgcatcacattgcaccagaagaattttttaaacaatcaaatctccaaggagtacggc

SEQ ID NO: 10 - DNA, 250 bp, Corynebacterium glutamicum
  source 1..250: mol_type = genomic DNA
tagatatcttgaaatcggctttcaacagcattgatttcgatgtatttagctggccgttaccctgcgaatgtccacagggtagctggtagtttgaaaatcaacgccgttgcccttaggattcagtaactggcacattttgtaatgcgctagatctgtgtgctcagtcttccaggctgctgatcacagtgaaagcaaaaccaattcgtggctgcgaaagtcgtagccaccacgaagtccaggaggacataca

SEQ ID NO: 11 - DNA, 250 bp, Corynebacterium glutamicum
  source 1..250: mol_type = genomic DNA
tagccccgatagtgtatgtgctgacctgcgtttatgcattttccttaggttgaaggaaatatcacacgacaaaagttgagtgatgcaggcataattggctatgggcaactgaagatgccacaatcaatgtttgatcaaacagaatcagacattgtgaatgtgggattatcggttcgcgcttcaccatgtttctgcatgatgaaattacatacatagttcagtgacagtcaccttttggaggagacacctt

SEQ ID NO: 12 - DNA, 250 bp, Corynebacterium glutamicum
  source 1..250: mol_type = genomic DNA
gcacttgattcagggtagttgactaaagagttgctcgcgaagtagcacctgtcacttttgccacaaataccaaattgcttatcagcatatgggctgtttgcttgccccagtcccagcagctgagacgcatccgctaaagccccaggaaccctgtacagaaagaaaacactcctctggctaggtagacacagtttataaaggtagagttgagcgggtaactgtcagcacgtagatcgaaaggtgcacaaag

SEQ ID NO: 13 - DNA, 250 bp, Corynebacterium glutamicum
  source 1..250: mol_type = genomic DNA
cacgtcacagctcccagacttaagattggtcacacctttgacacatttggaccacagttggttataaaatgggttcaacatcactatggttagaggtgttgacgggtcagattaagcaaagactactttcggggtagatcacctttgccaaatttgaaccaattaacctaagtcgtagatctgatcatcggatctaacgaaaacgaaccaaaactttggtcccggtttaacccaggaaggattgaccacc

SEQ ID NO: 14 - DNA, 250 bp, Corynebacterium glutamicum
  source 1..250: mol_type = genomic DNA
gcggatactgcagtcataggcgttgcttttcgacgtcgcaaagcgcaatttcctacctttaagatcctaatctgttgaggtcagccacaatttttcagaaaagttttgatagatcgacaggtaatgctttatactgacaacgtcgcaaggactacatttgcagccaagtctactacttgatcttcaaaggtcagcaattgtgaacaaagctacaaataaaccgttccgcccatgtcaatgaggagtcacc

SEQ ID NO: 15 - DNA, 250 bp, Corynebacterium glutamicum; identical to SEQ ID NO: 14
  source 1..250: mol_type = genomic DNA

SEQ ID NO: 16 - DNA, 250 bp, Corynebacterium glutamicum
  source 1..250: mol_type = genomic DNA
gagcgagcgaagtttcaaaatgccttcctgggggtgaaattagatcctgttaatgtactaaatttttcaaaagttttctcgttgaaaattaagaagctgggaaggtgaattgaattttgaggctttaaagcaaaaatgaacagcttggtctatagtggctaggtaccctttttgttttggacacctgtagggtggccgaagcaatgaaataggacaacaacgctcgaccgcgattattttttggagaatc

SEQ ID NO: 17 - DNA, 251 bp, Corynebacterium glutamicum
  source 1..251: mol_type = genomic DNA
ccgtaattggcgttgttgagtgcttcaagttcgtcagttgttaaagctctggtggcggcaagttctgcaagcgaaagcagatcttggggttgatcatcgcgggaagtcatattttattactctagtcggcctaaaatggttggattttcacctgctgtgacctggtaaaatggccactacccccaaatggtcacaccttttaggccgattttgctgacaccgggctatgccgtcaagtacgatcaataaca

SEQ ID NO: 18 - DNA, 250 bp, Corynebacterium glutamicum
  source 1..250: mol_type = genomic DNA
gttttcttgcgctgctgcagtgggcattgataccaaaaaggggctaagcgcagtcgaggcggcaagaactgctactaccctttttattgtcgaacggggcattacggctccaaggacgtttgttttctgggttagttaccccaaaaagcatatacagagaccaatgatttttcattaaaaaggcagggatttgttataagtatgggtcgtattctgtgcgacgggtgtacctcggctagaatttctcccc

SEQ ID NO: 19 - DNA, 250 bp, Corynebacterium glutamicum
  source 1..250: mol_type = genomic DNA
tcgtttagaaactcaaatccgctcgcagttggcgttttctggggcggttcagctagagttatgcgaaggatcccgtgcggcgtttatcttgtgaactcccccagggcaggaatgcagcaagggtcagcgagctctgacgggtgcgcgggatcccctaaaacgtctagagtagtggcttgaggtcactgctctttttttgtgcccttttttggtccgtctattttgccaccacatgcggaggtacgcagtt

SEQ ID NO: 20 - DNA, 250 bp, Corynebacterium glutamicum
  source 1..250: mol_type = genomic DNA
catccggataaaccaccatgaacggcagtggatcaaaagtcctgttggtgaagctgcgccccacagatcctgactgctgggagccatgaaaatagatcagcgcatccgtggtggaaccaaaaggctcaacaatacgaaacgttcgctttcggtcctgatgaaagagatgtccctgaatcatcatctaagtatgcatctcggtaagctcgaccaggacagtgccaccacaattttggaggattacaagaac

SEQ ID NO: 21 - DNA, 250 bp, Corynebacterium glutamicum
  source 1..250: mol_type = genomic DNA
aagaggcggaaaccacaagcgccaaggaactacctgcggaacgggctgtgaagggcaacttaagtctcatatttcaaacatagttccacctgtgtgattaatccctagaacggaacaaactgatgaacaatcgttaacaacacagaccaaaacggtcagttaggtatggatatcagcaccttctgaacgggtacgtctagactggtgggcgtttgaaaaactcttcgccccacgaaaatgaaggagcata

SEQ ID NO: 22 - DNA, 201 bp, Corynebacterium glutamicum
  source 1..201: mol_type = genomic DNA
cgcaaagctcacacccacgagctaaaaattcatatagttaagacaatatttttggctgtaaaagacagccgtaaaaacctcttgctcatgtcaattgttcttatcggaatgtggcttgggcgattgttatgcaaaagttgttaggttttttgcggggttgtttaacccccaaatgagggaagaaggtaaccttgaactcta

SEQ ID NO: 23 - DNA, 29 bp, Corynebacterium glutamicum
  source 1..29: mol_type = other DNA
ttgacaattaatcatcggctcgtataatg

SEQ ID NO: 24 - DNA, 812 bp, Balaenoptera musculus
  source 1..812: mol_type = genomic DNA
atgccccgagacaacatggcctccctgatccaacggatagcccgccaggcgtgcctcaccttccggggcagcgggggcagccgcagctcttccgaccgcggcgaggcgccaggccccgaggcgccgatgtcgcagggcttcccggagaacctgagcaagctgaagagcctgctgaccctggtccgcgcggaggacctgaacatctccccgcgcaaggccacgctgcagccgctgaggcccaacctgccgccagtcacctatatgcacatctgcgagacagacggcttcagcctcggcgtgttcctgctgaagagcggcacctccatcccgctgcacgaccacccgggcatgcacggcatcctcaaggtgctctacggcaccgtgcgcatcagctgcatggacaagctggaggtgggcagcgggcaacggccgcgggccccgccacccgagcagcagttcgagccggcgttgcagcccggggagcgggacgcggtaaggccgggtgtgctgcgctcgcgggcggagtacacggcggccagcggcccctgcgtcctcacgccgcaccgggacaacctgcaccagatcgacgctgtggaggggcccgccgctttcctggacatcctggccccgccctacgaccccgacgacggccgggactgccactattaccgggtgctggagcctgtcagggccagggaggcctccggctcggcctgtgacctgccccgagaggtgtggctcctggagaccccgcaggccgatgacttctggtgcgagggggagccctatccaggtcccaaggtcttcccttg

SEQ ID NO: 25 - DNA, 798 bp, Molossus molossus
  source 1..798: mol_type = genomic DNA
atggcctccctgatccaacggatcgcccgccaggcgtgcctcaccttccgaggcagcgggggcggccgcagtgcttccgaccgcggctcggtgccaggccccgaggcgccggggccgcagggcttcccggagaacctgagcaagctgaagagtctgctgacccaggtccgtgctgaggacctgaatatcgccccgcgcaaagcgacgctgcagccgctgccacccaacctgccacccgttacctacatgcacatctacgagacggacggcttcagcctcggcgtgttcctgctcaagagcggcacgtccatccctctgcacgaccacccgggcatgcacggcatgctcaaggtgctctacggcaccgtgcgcatcagctgcatggacaagctggaggcaggcggcgggcagcggcagcgagccccaccgccagaacagcagttcgagccgccgctgcagccccgggagcgggacgctgtccggcctggcgtgctccgctcgcgggccgagtacaccgaggccagcggtccctgcgtccttacgccgcaccgggacaacctgcaccaaatcgacgctgtggacgggcccgctgccttcctggacatcctggccccgccctacgaccccgacgacggccgggactgccactattaccgggtgctggaaccggtcagggccaaggaggcctccggatcgacctgtgacctgcctcgagaggtgtggcttctcgagactccgcaggccgatgacttctggtgcgagggggagccctatccaggtcccaaggtcgtcccttga

SEQ ID NO: 26 - DNA, 798 bp, Felis catus
  source 1..798: mol_type = genomic DNA
atggcctccctgatccaacggatcgcccgccaggcgtgtctcaccttccggggcagcgggggctgccgcagcgcttccgaccacggcgcggcgccgggctccgaggcccctatgcctcagggcttcccggagaacctgagcaagctgaagaacctgctgacccaagtccgcgcggaggacctgaacatcgccccgcgcaaggccacggtgcagccgttgccgcccaacctaccaccggtcacctatatgcacatttacgaaaccgacggcttcagcctcggcgtgttcctgctcaagagcggcacgtccattccgcttcatgaccacccgggcatgcacggcatgctcaaggtgctctacggcaccgtgcgcatcagctgcatggacaagctggaggcgggcggcgggcagcggccacgggccccgccgccagagcagcagttcgagccgccgctgaaagcccgggagcgggacgccgtgcggctgggcgtgctgcgctcgagggccgagtacaccgaggccagcggcccctgcgtcctcaccccacaccaggacaacctgcaccagatcgacgctgtggacgggcccgccgccttcctggacattctggccccgccctatgacccggacgatggccgggactgccactattaccgggtgctggagcctgtcagggacaaggagctttccggctcggcttgcgacctgcccagagaggtgtggctcctggagacccctcaagccgatgacttctggtgcgagggggagccctatccaggtcccaaggtcttcccttga

SEQ ID NO: 27 - DNA, 813 bp, Heterocephalus glaber
  source 1..813: mol_type = genomic DNA
atgccccgagacaacatggcttcccttatccaacggatagcccgccaggcttgcctcaccttccggggcaccgggggcgtccacggcgcttccgaccgcggcgcgacggctggccccgaggcgccgatgccccccggcttccccgagaacctgagcaagctgaagggcctgctgacccaggtccgcgccgaggacctcaacatcgccccgcgtaaggccacgctgcagccgctgcctcccaatctgccgccggtcacctacatgcacatctacgagacgcccggctttagcctcggcgtgttcctactcaagagcggcacgtctatcccgctgcacgaccaccctggcatgcatggtatcctcaaggtgctctacggcaccgtccgcatcagctgcatggacaagctggaagcgggcggtgggcagcgtccgagggcccagtcgctcgagcagcagttcgacccgccgctgcaggcccgggagcgggaggccatgcggccaggcatcctgcgctctcgggccgagtacaccgaagccagtggcccctgtgttctcacgccgcaccgagacaacctgcaccagatcgacgccgtggacggacccgctgccttcctggacatcttagccccgccctacgacccggatgacggccgggactgccactattaccgggtgctggagcctgtcaggcccaaagaggcctccagctcggcctgtgacctgccccgagaggtgtggctgctggagacgccgcaggccgatgacttctggtgcgaaggagaaccctatccaggacccaaggtcttcccttga

SEQ ID NO: 28 - DNA, 813 bp, Elephas maximus
  source 1..813: mol_type = genomic DNA
atgccccgagacaacatggcctccctgatccaacggatcgcccgccaggcgtgtctcaccttccggggtagcgggggcggccgcagcgcttccgaccgcggcgcggcgccaggccccgaggcgccgatgccgccgggcttcctagagaacctgagcaagctgaagagcctgctgacccaggtccgcgctgaggacctgaacatcgccccgcgcaaggccacgctgcagccactgccgcccagcctgccgcccgtcacctacatgcacatctacgagacggacggcttcagcctcggggtgtttctgctcaaaagcggcacgtccatcccactgcacgaccacccgggcatgcacggcatgctcaaggtgctctacggcaccgtgcgcatcagctgcatggacaagctggaggcgggcgacgggcaacggccgcgggcccctccgtccgagcagcagttcgagccgccgctgcaggcccgggagcgggacgccgtgcagctgggcgtgctgcgctcgcgggccgagtacaccgaggccagcggcctctgcatcctcacgccgcaccgggacaacctgcaccagatcgacgcagtggacgggccagccgccttcctagacatcctggccccgccctacgaccccgacgacggccgggactgccactattaccgggtgctggagcccgccgccaggcccaaggaggcctccggctcgacctgtgacctgccccgagaggtgcgtcagtcttggcatctcaggaaggactctgtcgtcactcatctgttgcagatcattctgaatcttgtactctag

SEQ ID NO: 29 - DNA, 903 bp, Mus caroli
  source 1..903: mol_type = genomic DNA
atggcctccctgatccagcgcatcgctcgccaggcgtgtctcaccttccgcggcggctcgacgggctccgaagggccggcgccgggcttcccggagaacctgagcctgctcaagagcctgctgacccaggtgcgcgccgaggacctcaacatcgcgccgcgcaaggcgctgccgcagccgctgccccgcaacctcccgccggtcacctacatgcacatctacgagacggagggcttcagcctgggcgtgttcctgctcaagagcggcacgtgcatcccgctgcacgaccacccgggcatgcacggtatgctcaaggtgctgtacggcacggtccgcatcagctgcatggacaagctggacacgggggccgggcatcggcggccgccgccagagcagcagttcgagcccccgctgcagcccttggagcgggaggccgtgcgaccgggcgtgctgcgttcccgggccgagtacaccgaggccagtgggccctgcgtgctcactccacaccgggacaacctgcaccaaattgatgctgtggacgggccagctgccttcctggacatcctggccccaccctacgacccggacgacggccgggactgccactattaccgtgtggtggagcccatcagacccaaggaggcttccggctctgcctgcgaccttccccgagaagtgtggctcctggagacaccacaggccaacaacttctggtgtgagggagagccctatccccaaggtcctaccttgaagctcctgccgcccggggaactgtgggccaaagatgtaccctgccctgctcaagaaaggcctggctgccctctaccttccataagggctcttctctctctgccccctcacctggaccatggatttactggagtgaccagcgccacatcttga

SEQ ID NO: 30 - protein, 270 aa, Capra hircus
MPRDNMASLIQRIARQACLTFRGSGGGRSSSDFGEAPGPEAPIPQGFPENLSKLKSLLTQVRAEDLNISPRKATLQPLRPNLPPVTYMHICETDGFSLGVFLLKSGTSIPLHDHPGMHGILKVLYGTVRISCMDKVEVGSGQRPRAPPPEQQFEPPLQPRERDAVQPGVLRSRAEYTEASGPCFLTPHRDNLHQIDAVDGPAAFLDILAPPYDPDDGRDCHYYRVLEPVRAKEASGSACDLPREVWLLETPQADDFWCEGEPYPGPKVFP

SEQ ID NO: 31 - protein, 270 aa, Homo sapiens
MPRDNMASLIQRIARQACLTFRGSWGGRGASDRDAASGAEAPMQPGFPENLSKLKSLLTQLRAEDLNIAPRKATLQPLPPNLPPVTYMHIYETDGFSLGVFLLKSGTSIPLHDHPGMHGMLKVLYGTVRISCMDKLDAGGGQRPRALPPEQQFEPPLQPREREAVRPGVLRSRAEYTEASGPCILTPHRDNLHQIDAVEGPAAFLDILAPPYDPDDGRDCHYYRVLEPVRPKEASSSACDLPREVWLLETPQADDFWCEGEPYPGPKVFP

SEQ ID NO: 32 - protein, 273 aa, Ursus maritimus
MPRDNMASLIQRIARQACLTFRGSGGCRSASDLGAAPGPEAPMPQGFPENLSKLKNLLTQVRAEDLNIAPRKATLQPLSPNLPPVTYMHIYETDGFSLGVFLLKSGTSIPLHDHPGMHGMLKVLYGTVRISCMDKLEAGGGPQPRAPPPEQQFEPPLQARERDAVRPGVLRSRAEYTEASGPCVLTPHQDNLHQIDAVDGPAAFLDILAPPYDPDDGRDGRDCHYYRVLEPVRDKEAPGSACDLPREVWLLETPQADDFWCEGEPYPGPRVFP

SEQ ID NO: 33 - protein, 270 aa, Nycticebus coucang
MPRDNMASLIQLIARQACLTFRGGGGGRRASDRGAASGPEAPMPPGFPENLSKLKSLLTQIRAEDLNIARRKATLQPLPPNLPPVTYMHIYETEGFSLGVFLLKSGMSIPLHDHPGMHGMLKVLYGTVRISCMDKLEAGGEQRPRAPPLEQQFEPPLQPRERDAMRPGVLRSRAEYTEASGPCVLTPHRDNLHQIDAVDGPAAFLDILAPPYDPDDGRDCHYYRVLEPVRSKEASGLACDLPREVWLLETPQADDFWCEGEPYPGPKVFP

SEQ ID NO: 34 - protein, 256 aa, Mus musculus
MPRDNMASLIQRIARQACLTFRGSSTGSEGPAPGFPENLSLLKSLLTQVRAEDLNIAPRKALPQPLPRNLPPVTYMHIYETEGFSLGVFLLKSGTCIPLHDHPGMHGMLKVLYGTVRISCMDKLDTGAGHRRPPPEQQFEPPLQPLEREAVRPGVLRSRAEYTEASGPCVLTPHRDNLHQIDAVDGPAAFLDILAPPYDPEDGRDCHYYRVVEPIRPKEASGSACDLPREVWLLETPQADDFWCEGEPYPGPKVLP

SEQ ID NO: 35 - protein, 250 aa, Salvelinus alpinus
MMPRDNMTSLVQKIGRQALVTFRNPSLVGDKAFLDNQGKLQTLMTEIRAADLKIAPRKVDCASTPLPHNPPVTYMHICETDQFSMGVFLLKSGASIPLHDHPGMYGMLKVMYGKVRITCFDRLDKSTNVASDTQFKPPLLPFQRDALRMSILRSVGEFTEESGPCILTPDRDNLHQIDAVDGPTAFLDILAPPYDPNDGRDCHYYKVLESDSEDKKAEAQGQKEVWLMEVPQPSDFWCGGEPYPGPEVNI

SEQ ID NO: 36 - protein, 278 aa, Phrynosoma platyrhinos
MPRDNMAASLIQRVARQARLTFRPPGTTTTSGGGGGGDGPRSAEALHRLRDLLGEVRAEDLRLSPRGPSAVPGRAQPPVSYMHICETESFSMGVFVLRAGACIPLHDHPGMNGLLKVLYGTLRIACLDTLPEDHDDDDDGGGGGGGAGASGPPLPHGRHHRHRHRGRHRALLRSHRLYTPSSAPCLLSPETDNLHQIDAVGGGPAAFLDILAPPYDPEHGRDCHYYRLLDGPGLLPPPPPSPEEDPPGLPKELWLQETPQAPDFWCGGEPYPGPQVSP

SEQ ID NO: 37 - protein, 284 aa, Vombatus ursinus
MPRDNMATLIQRIARQARLTFRRGGGGGGAPEAGFPENLQQLQQLLSQLRAEDLNISPRRAALPSPPPARPPHLPPVTYMHICETAEFSLGVFLLKSGTSIPLHDHPGMHGVLKVLYGTLRISCLDPLPGEGPGAPREGPQPQPHPPPPQQAPAAPPRFEPPLRPGQRAVVRRALLRSRAEYTEASAPCLLSPHRDNLHQIDAVDGPAAFLDILAPPYDPDDGRDCHYYRLLEAAAPAGPPDAGLGCGPAAPLPVPREVWLLETPQAADFWCEGEPYPGPKVTP

SEQ ID NO: 38 - protein, 247 aa, Notechis scutatus
MPRDEMAASLIQRVARQARATFGAGAGEAPAELQRLRELLGQVSAEDLGLLQPRGPSAVPGPSQPPVSYMHICETARFSMGVFVLRAGACIPLHDHPGMQGLLKVLYGTLRIACFDAADEEPEEQPDAASGAASSSPRPRRLRALLRSHRLYTAAAPPCLLSPQRDNLHQIAAEEAAGPAAFLDILAPPYDPARGRDCHYYRLADGQSLPPPPAQPVPPSALWLLETPQAPDFWCGGEPYPGPRVWP

SEQ ID NO: 39 - protein, 251 aa, Salmo salar
MPRDNKKSLIQKIANQANITFKDFNASAIGDNKVLLDNKGDLITLLTEIRAADLKMKPNFSHSEHNPPVTYMHICETESFSMGVFLLNNGASIPLHDHPGMNGMLKVLYGKVSISCFDLLDKPSDQVQSEIQFDPPLLPFQKDSLRRAVLRSTMWFTHDSSPCILTPQRENLHQIDTVDGPAAFLDILAPPYDPEDGRDCHYYKVLQTVPDADSKVEAQQQWEEETWLLEIGQPDGFWCGGEPYPGPKVSF

SEQ ID NO: 40 - protein, 242 aa, Adelges cooleyi
MASKIECVLKQSLITFSGSHTSKTFHDNLIKLKRYANELDANSITLDNRLLRCDTYELNSSFDKLSGSVLIRRVRAPVTYIEVYEDQNVSIGIFVLRNGAKIPLHDHPCMYGVLKVIHGKVRIQSYTPKKKDYSQLNECIISAVKMAPIIIGESDEPCVLCPVEGNIHQVDTVDGPAAFVDILAPPYRTDIPDVGKRYCRYFKELNMSKDIKLEVVPDPKDYWSDTAPYTGPQFNLNIDENS

SEQ ID NO: 41 - protein, 194 aa, Lipotes vexillifer
MPRDNMASLIQRIARQACLTFRGGSLLKSGTSIPLHDHPGMHGILKVLYGTVRISCMDKLEVGSGQRPRAPPPEQQFEPPLQPRERDALRPGVLRSRAEYTAASGPCVLTPHRDNLHQIDAVEGPAAFLDILAPPYDPDDGRDCHYYRVLEPVRAREASGSACDLPREVWLLETPQADDFWCEGEPYPGPKVFP

SEQ ID NO: 42 - protein, 245 aa, Mytilus edulis
MVSNIQKVAQTAYRLFSRICGNSDVLLSSQNVGLLTSMMNTIKDKDVNFDMSKISLESETPVSYIDIYEDPVMTMAIFIIRDGCKIPLHDHPGMYGFCKVIHGSARMKSFSDPQNFVGNPPENIASKIRPYLRQSIKPVVLEEESIFSSDDNCCVVTPNHGTYHELVAVNGPVAFLDILAPSYDHASGARICQYYEEIKGQNEEIKHLGKPLIWFTPISQPSDFWCDPEEYEGPSVVDALNEIVK

SEQ ID NO: 43 - protein, 228 aa, Pan troglodytes
MQPGFPENLSKLKSLLTQLRAEDLNIAPRKATLQPLPPNLPPVTYMHIYETDGFSLGVFLLKSGTSIPLHDHPGMHGMLKVLYGTVRISCMDKLDAGGGQRPRALPPEQQFEPPLQPREREAVRPGVLRSRAEYTEASGPCILTPHRDNLHQIDAVEGPAAFLDILAPPYDPDDGRDCHYYRVLEPVRPKEASSSACDLPREVWLLETPQADDFWCEGEPYPGPKVFP

SEQ ID NO: 44 - protein, 254 aa, Manis javanica
MPRDNMASLIQRIARQACLTFRGSGGGLSASDRCTAPGPEAPMPQGFPENLNKLKSLLTQVRAEDLNIAPRKATLQPLPPNLPPVTYMHICETDGFSLGVFLLKGGTCIPLHDHPGMHGMLKVLYGTVRISCIDKLEAGGGQRPRAPPPEKQFDPPLQARERDAVRPGVLRSRAEYTEASGPCVLTPHRDNLHQIDAVDGPAAFLDILAPPYDPDNGRDCHYYRVLEPIRAKEASGSACDLPREGSAHYYSTLT

SEQ ID NO: 45 - DNA, 1857 bp, Propithecus coquereli
  source 1..1857: mol_type = genomic DNA
atggtaaaatgctttcccttgaggaaaacagaacagcactctgaagtttgtccacacggagcctcctccggcaattcaccaacttcaatccaagcaatccttaagatacggtactggcagcacaggactcagccccagggaactttggacagagacacacacacgggacaacgctatctgaagagacacagggagaagacagccatttgcaagtcaaagagagaagcctggaacgtagccttccctcgccgccacaggaggaaacgaccctgctggggactcctaacctccagaatttcactggacttcagcatgatcacttctccgtcgctggcttatgtggcgattttggttttctgtgtctcaaaaaccagctctctggacacttttattgcagctgtttatgagcatgcggtgctattgcctaataccagcctcacaccagtgtctcgtgaggaggcgctggcgttaatgaatcggaatctggatcttttggaaggagcgatcgcgtcagcagcggagcagggcgcacatattatagtgaccccagaagatggtatttatggctggaacttcaacagggaatctctctacccgtacttggaggatatcccacaccctcaagtaaactggatcccctgtaatgatcctaacagatttggccacacccccgtacaagaaagactcagctgcctggccaaggagaactctatctatgttgtggcaaatattggggacaagaagccatgtaatgccagtgaccctcagtgtccccctgatggccgttaccagtacaacactgatgttgtgtttgattctcagggaaggctggtggcacgctaccataagcaaaatcttttcttgggtgaagatcaattcaatgcacccaaggagcctgaggttgtgactttcaacaccacctttggaaggtttggcatttttacgtgctttgatatactcttccaccatcctgctgttaccctggtgaaagatttccacgtggacaccatacttttcccaacagcttggatgaatgttttgccacatttgtcagctattgaattccactcagcgtgggctatgggcatgggggtcaatttccttgcatctaatatacattacccctcaatgaggatgacaggaagtggcatttatgcacccaattctgcaagagtatttcattatgatatgaagacaaaggagggaaaactcctcctctcacaactggattcccacccacaccaccctgtagtggtgaattggccatcttatgccagcagtgtggaagcactctcgactggaaaccaggaatttaagggcattatcttttttgatgaatacaccttcgtggagctcaaaggagtcggggggaattacacagtatgtcagaaagatctctgttgtcatctaagctacaagatgtcagagaagagatcagatgaagtttatgccctaggggcatttgatggactgcacaccgtggaaggacgctattatctacagatttgtactctggtgaagtgtaaaacgaccgacttaaacacttgtggtgactcagttgaaaccgcttctaccaggtttgaaatgttctccctcagtggcacttttggaacccagtatgtcttccctgaggtgttgttgagtgagattcagcttgcacctggagaatttcaggtgtcaagtgatggacgcttgtatagcctgaagccaacatctggacctatcttaacagtgactctgtttgggaggttgtatgagaaggacggagcatcaaacgcttcatcagacctcatagcgcaagcactaagaataatgctagcagttataatacctattgtatactcattaagttggtag

SEQ ID NO: 46 - DNA, 1545 bp, Equus asinus
  source 1..1545: mol_type = genomic DNA
atgatttcttctcagctgctcgcttatgtggcagttttggttttctgcaccttccaagcccgctccctggagacctttattgcagctgtttacgagcacgcagtgatattgcccaatgccaccctaacgccagtgtcccgggaagaggcattgctattaatgaaccgcaatctggatcttctggagggagcaatcgcctcagcagccaagcagggtgcgcacatcatcgtgactccagaagatggtgtttacggctggaacttcagcagggacacgctctatccgtacttggaggacatcccagaccccgaagtgaactggatcccttgcaacaatccccacagatttggccacactccagtccaagagaggctcagctgcctggccaaggacaactctatctacgtcgtggcaaatttcggggacaagaagccatgcaatgccagtgaccctgagtgcccccctgatggccgttaccagtacaacactgatgtggtgtttgatgctcagggaaaacttgtggcacgctaccataagcaaaaccttttcatgggtgaagatcaattcaatgcacccaaggagcctgaggtcgtgactttcaacaccacctttggaaagtttggcattttcacgtgcttcgatattctcttctacgaccctgctgttaccctggtgacagatttccaagtggacaccatcctcttcccgacagcctggatgaatgttttgccacatttgtcagctattgaatttcactcggcttgggctatgggcatgggggtcaatttccttgcatccaatatacattacccctcaatgaaaatgacaggaagtggcatctatgctcctgattctccaagagcatttcattatgatatgaagacggaggagggaaaactactcctctcacagctggactcccacccgcaccacccagcagtggtgaactggacttcttttgccagtggtgtgaaaacactctcaacgggaaaccaggaatttaacagcactgtcttttttgatgaattcaccttcctggagctcaccgaagtcacaggaaattacacagtttgtcagaaagctctctgctgtcacctaagctacaagatgtctgagaagagatcagatgaagtgtacgccctaggggcatttgatggcttacatactgttgaagggagttattatttacaggtttgcactctgttgaagtgtagaaccacggacttacacacttgtggtgactcggtggaaactgcttccactaggttcgatacgttctccctcagtggcacttttgggacccagcatgtcttccctgaggtgctgctgagtgaaattcagctcgcacctggagaattccaggtgtcaagtgatggacgcttgttcagtctgcagcccccctcgggacctgtcctgacagtgactctttatgggaggctgtacgagaaggaccaggtatccgacgcctcatcagacctcacagcacaagcactgagagtaatgctcgtagttctaatacctgttgggttctcaataagttggtag

SEQ ID NO: 47 - DNA, 1383 bp, Neomonachus schauinslandi
  source 1..1383: mol_type = genomic DNA
atgaaccgaaatctggatcttttggaggcagcaatcacttcagcagcaaaccagggtgcacatattattgtgactccagaagatggtatttatggctggaactttaccagggaaactatctacccatacctggaggatatcccagaccctggagtgaactggatcccttgtaataatcccaaaaggtttggctacaccccagtgcaagaaagactcagctgcctggccaaggacaactctatctacgttgtggcaaatattggggacaagaagccatgcaatgccagtgaccctcagtgtccccccgatggtcgttaccagtacaacaccgatgtggtgtttgattctcagggaaaactggtggcacgctatcataagcacaaccttttcatgggtgaaaatcaattcaatgtacccaagaagcctgaggttgtgacttttgacaccatctttggaagatttggcattttcacgtgcttcgatatactcttctatgatcctgctgttactctggtgaaagatttccatgtcgataccatagtcttcccaacagcatggatgaatgttttgccacatttgtcagctattcagttccactcagcttgggcaatgggcatgggggtcaacttccttgcatccaatatacatcacccctcaaagagaatgacaggaagtggcatctatgcacctgattctccaagagcatttcattatgatatgaagacaaaggagggaaaactccttctctcacaactggattctcactcacacagccctgtagtgaattgggcttcttatgccagtagtataaaggcattctcaacaggaaatcaggaatttaagggtactgccttttttgatgaattcaccttcttggagctcacaagagtcacaggaaattacacagtttgtcagaaaaaactctgctgtcatctaagctacaagatgtctgaaaagagaagagatgaagtttacgccctgggggcatttgatggactacatattgtcgaagggacgtattatttacagatttgtaccctgctgaaatgtaaaacaacaaacttaaacatttgtggtggtgcagttgaaacagcttctaccaggtttgaaatgttttccctcagtggcacttttggaactcagtatgtcttccctgaggtgttgctaagtgaaagtcagcttgcacctggagaatttcaggtgtcgagtgatggacgcttgttcagcgtgaagccgccatctggacctctcttgaccgtgactctatttgggagaatgtatgagaaggaccagacattaaaggcttcatcagacccccaggcacaaacactgggaataatgctcttagtcataatgccagttgtgtgctcattaagttggtag

SEQ ID NO: 48 - DNA, 1545 bp, Carlito syrichta
  source 1..1545: mol_type = genomic DNA
atggttgcttctcagttgctggcttatatggcaatcttggtgttctgtgtcttaaaagccagctccctggacacgtttcttgcagctgtttatgagcatgcagtgatactgcctgatgccaccctgacaccagtgtctcgtgaagaagcattggtgctaatgaataggaatttggatcttctggaaagagcggtcatatcagctgcacagaagggtgcacatattattgtgactccagaagatgccatttacggctggaacttcagcagggaatctgtctacccctacttggaggatatcccacaccctcaagtgaactggatcccttgtaataatccgaacagatttggccacactccagtccaagaaagactcagctgcctggccaagaacaactccatctatgtcgtggcaaatattggagacaagaagccatgcaatgccagtgaccctcagtgtccccctgatggccgttaccaatacaacactaatgtggtatttgattctcagggtacgctggtggcacgctaccataagcagcaccttttcataggtgaagatcacttcaatgcacccaagagccctgaggtcgtgactttcaacactacatttggaaggtttggcattttcacatgctttgatatactcttccatgatcctgcagttaccctggtgaaagatctccacgtgaacaccatactatttcccacagcttggatgaatgttctgccacatttgtcagcgattgaattccactcagcttgggctatgggcatgggagtcaacttccttgcctccaatatccattaccctttgatgaaaatgacaggaagtggcatctatgcacccaattctccaagggcatttcattatgatatgaagacagaagaaggaaaactcctcttctcgcaactggatgcccgcccataccaccccagagtggtgaattggacttcttacgccagtagtatagaggcagtctcatcagaaaaacagaaatttaagggagtcgtcttctttgatgaattcaccttcgtggagctcacagatgttatgggaaattacacagtttgtcagaaagatctctgctgtcatctgagctacaagatgtctgagaagagatcagatgaagtttacgcccttggggcatttgatgggctgcacacggtggaagggtgctattatctacaggtttgtactctgctgaagtgtaaaacaactgacttacacacgtgtggtgactcagtggaaacggcttctaccacgtttgagatgttctccctcagcggcacctttggaacccaatatgtcttccctgaggtgctgctgagtgaaatgcgacttgcacccggagaatttcaggtgtcagatgatggatgcttgtctagcctgaagccaacatctagacctgtcttaacagtgactctttttgggaggttgtatgagaaggattgtgcatcaaatgcttcatcagacctcacagcacaagcacgaagaaccacgctgatagtaatgatatgtattgtccatttattaagttggtag

SEQ ID NO: 49 - DNA, 1632 bp, Dipodomys ordii
  source 1..1632: mol_type = genomic DNA
atggtgattgctcggttgctggcttatttggcattttcagttttctgtgtcttaccagccaactgcctggacacttttactgcagctgtttatgaacatgccgtaatactgcctaacttcaccacaacaccagtgtctcgtgaagaggcactggtgttaatgaatcagaatctagacattttggaagaagcaatcacctcagcagcaaaacagggtgcacaaattattgtgactccagaagatggtgtttatggtgtgaatttcagcagggacaccatttacccatatttagaggacattccagacccggaagtcaattggatcccctgtaatcatcccaaaaggtttggtgatactcaaatacaagaaagactcagctgtctggccaagagcaactctatctatgtggtggcaaatataggagacaagaaaccatgcaatgccagtgaccctcgatgtccccctgatggtcgttaccaatacaacactaatgtggtgttcgattcacagggaaaactggtagcccgctaccacaagcaacatcttttcatgagtgaatcttttcacttcaatgcacccaaagatcctgaggtcgtgactttcaacactccctttggaaaatttggcatttttacatgctttgacatcctcttctacgaccctgccattaccctggtgaaagatttccatgtggacactatcctcttcccaacagcttggatggatgttctaccacacttgtcagccattgaattccactcagcttgggctatgggcatgggagtcaatttacttgcatccaatttacatcgcccccagaacagaatgacaggaagtggcatctatgcacctgattctccaagagcttttcattatgacaggaagacagaaaagggaaagctcgtcctctctcagctggattcccacccctaccgccccgaagtgaactggagttcttatgccagcaacataggagcactctcaactggaaaccaagaatttaagggcagtgccttttatgatgagtacactttcgtggagctcacaggaatcacaggaaattacactgtttgccagaaagatctctgctgtcatctaaactaccagatgtctgagaagagatccaatgaaatttatgctctaggggcatttgatggactgcacacagttgaagggtgttaccatctacaggtctgcactatgttaaagtgtaaaaccactaacagacatacctgtggtgcttcagttgacacggcttctactaggtttgaaatgttctccctcagtggcacttttgggacccagtatgtctttcctgaggtgttgttgagtgaagttcggctcgcacctggagaatttaaggtaacaacctttgcttcatttccccagttaattgcatgtgaaaggaaatactggtttatttcaaaagctactgaaatcatacatatttccattgggaaaagggaaagattatctttgaggatcaggaaaggcaccgcaagcagcctctgccataaagtcttaccaccagcatgttctgccttactatggacttaccaacacacccaaggatcactggccataggaatgaaaaatctgagtgacacagagaaagcagctttatttatatga

SEQ ID NO: 50 - DNA, 1257 bp, Ictidomys tridecemlineatus
  source 1..1257: mol_type = genomic DNA
atgttttggaaggagcgatcacgttagctgcaaatcagatttggctacaacccagtacaagaaagactcagctgcctggccaagaataactctatctatgtggtggcaaatataggggacaagaagtcatgcaaagccagtgagcctcagtgtcctcctgatggccgttaccaatacaatactaacgtggtgtttgattctcagggaaaactagtggcccgctaccataagcaaaaccttttcatgggggaagaccaattcaatgcacctaaagagcctgagatggtgacatttaacacaacctttgggaagtttggcattttcacttgctttgatattctcttccatgatcctgctgttgccctggtgaaagacctccacgtggacaccgtactcttcccaacagcttggatgaatgtcctgccacatttgtcagcggttgagttccactcagcttgggctatgggcatgggagtcaatttccttgcatctaatttacataacccctcaaatagaatgacaggaagtggtatttatgcacctgattttccaagagcatttcattatgatatgaagacaccagagggaaaacttatcatctcaacactggattctcacccatcccattccatagtgaattggtcttcttacgccagcagtatacatgcattcccaacaggaaaccaagaattcaagggcactgtcttttttgatgaattcacctttgtggagctcacagaaattgcaggaaattacacagtttgtcagaaagagctctgctgtcatttaagctatgaaatgtctgaaaagagatcagatgaagtttatgccttaggagcatttgatggactgcacgttgttgaagggcgctattacctacagatttgtactctgctgaaatgtgaaaccactgacttgcacacctgtggtggttcagttgacacagcttccactaggtttgaatcgttctccctcagtggcactttcggaacccaatatgtcttccctgaagtactgctgagtgacgttcagcttgcacctaaagaatttcaggtgtcaagtgatggacgtttgtttagcttgaagccaacatctggacctctgttaacggtcactctctttgggagggtgtacgagaaggatatggcatcaaatgggtcacctgacctctcaacatattcagtaaagataatgttagtagttatgatacctattgtgttctcgttatgttggtagaatatttacattttctctctt

SEQ ID NO: 51 - DNA, 1539 bp, Rattus norvegicus
  source 1..1539: mol_type = genomic DNA
atgagcctgtctcggtggctggcgtgggctgcagcgttttccgtcttctgtgtcttaaaagtcagctctctggacactttcctcgcagctgtttacgagcatgctgtgatcctgcctaaagtcaccctgttacctgtgtctcacagtgaggcactggcattaatgaaccagaatctggaccttctggaaggagcgatcttatcagcagctaagcagggtgcgcacattattgtgactccagaagatggtatatacggtgtgcagttcaccagggataccatctacccatacctggaggatatcccagaccctcaagtaaactggataccctgtgataatccggaaagatttggctctaccccggtgcaggagagactcagctgcttggccaagaacaactcaatctatgtcgtggcgaacatgggagacaagaagccatgcaacaccagcgactctcactgtccacctgacggccgattccaatacaacactgatgtggtgtttgattcccggggtaaattggttgccagataccataagcaaaaccttttcatgggagaagagcaattcaatgcccccccggagcctgaggtcgtgacttttgacaccccctttggaaagtttggcatcttcacctgtttcgatattctcttccatgatcctgctgtcaccctagtgacagagttccaggtggacaccatactgttcccaactgcctggatggacgtgcttcctcatttggcagccattgaattccactcagcttgggctataggcatgggagtcaatttcctagcagctaatctacatattcccttgaggagaatgacaggaagtggtatctatgcacccgattctccacgggcctttcattatgacaggaagacacaggaaggaaagctcctccttgcacagctggactctcacccgagtcactccccagtgaactggacttcctatgccagcagtgtagaagcacccccaactgaaaaggaggaatttcggagttttgttttctttgatgagtttacctttgtggagctcaaagggatcacaggaaattacactgtttgccagaacgacctctgctgtcatctaagctaccagatgtctgagaagcgagctgatgaagtttatgctcttggggcttttgacgggttgcacaccgtggaagggcagtatcacctgcagatttgcattctgctaaagtgtaaaactaccaatttacgcacctgtggtagttcagtggacacagcttctaccaggtttgaaatgttctccctcagtggcacttttggaacgcggtatgtcttccctgaggtgttgctgagtgaagtcaagcttgctcctggggagtttcaggtgttgagtgacgggcgcctggttagcctgaagccaacgtctggacctgtgttaacagtcggtctctttgggaggttgtatgagaaggactgggcatcaaatgcttcctcagacctcatagcacactcactgataataatgctgatcctagctcctattatacattacttgtgttaa

SEQ ID NO: 52 - DNA, 1542 bp, Homo sapiens
  source 1..1542: mol_type = genomic DNA
atgactactcagttgccagcttacgtggcaattttgcttttctatgtctcaagagccagctgccaggacactttcactgcagctgtttatgagcatgcagcgatattgcccaatgccaccctaacaccagtgtctcgtgaggaggctttggcattaatgaatcggaatctggacattttggaaggagcgatcacatcagcagcagatcagggtgcgcatattattgtgactccagaagatgctatttatggctggaacttcaacagggactctctctacccatatttggaggacatcccagaccctgaagtaaactggatcccctgtaataatcgtaacagatttggccagaccccagtacaagaaagactcagctgcctggccaagaacagctctatctatgttgtggcaaatattggggacaagaagccatgcgataccagtgatcctcagtgtccccctgatggccgttaccaatacaacactgatgtggtatttgattctcaaggaaaactggtggcacgctaccataagcaaaaccttttcatgggtgaaaatcaattcaatgtacccaaggagcctgagattgtgactttcaataccacctttggaagttttggcattttcacatgctttgatatactcttccatgatcctgctgttaccttggtgaaagatttccacgtggacaccatagtattcccaacagcttggatgaatgttttgccacatttgtcagctgttgaattccactcagcttgggctatgggcatgagggtcaatttccttgcatccaacatacattacccctcaaagaaaatgacaggaagtggcatctatgcacccaattcttcaagagcatttcattatgatatgaagacagaagagggaaaactcctcctctcgcaactggattcccacccatcccattctgcagtggtgaactggacttcctatgccagcagtatagaagcgctctcatcaggaaacaaggaatttaaaggcactgtctttttcgatgaattcacttttgtgaagctcacaggagttgcaggaaattatacagtttgtcagaaagatctctgctgtcatttaagctacaaaatgtctgagaacataccaaatgaagtgtacgctctaggggcatttgacggactgcacactgtggaagggcgctattatctacagatttgtaccctgttgaaatgtaaaacgactaatttaaacacttgcggtgactcagctgaaacagcttctaccaggtttgaaatgttctccctcagtggcactttcggaacccagtatgtctttcctgaggtgttgctgagtgaaaatcagcttgcacctggagaatttcaggtgtcaactgacggacgcttgtttagtctgaagccaacatccggacctgtcttaacagtaactctgtttgggaggttgtatgagaaggactgggcatcaaatgcttcatcaggcctcacagcacaagcaagaataataatgctaatagttatagcacctattgtatgctcattaagttggtag

SEQ ID NO: 53 - DNA, 1539 bp, Mus musculus
  source 1..1539: mol_type = genomic DNA
atgggcatgtcttggtggctggcgtgtgctgcagcgttttctgccctctgtgtcttaaaagccagttcgctggatactttcctcgcggctgtttacgagcatgctgtgatcctgcctaaggacaccctgttgccagtgtctcacagtgaggctctggcattaatgaaccagaatctggaccttctggaaggagcgatcgtatctgcagcgaacgagggtgcgcacattattgtgactccagaagatggcatatacggtgtgcgtttcaccagggatacgatctacccatacctggaggagatcccagaccctcaagtaaactggataccctgtgataaccctaaaagatttggctctaccccggtgcaggagagactcagctgcttggccaagaacaactccatctatgttgtggcgaacatgggagacaagaagccgtgtaacaccagcgactctcactgtccacctgacggcagattccagtacaacactgatgtggtgtttgattcccagggtaaactggttgcgagataccataagcaaaacattttcatgggagaagatcagttcaatgtccccatggagcctgagtttgtgactttcgacaccccctttggaaagtttggcgtcttcacctgtttcgatattctcttccatgatcccgctgtcaccctggtgacagaattccaggtggacaccatactgttcccaaccgcctggatggacgtccttcctcatttggcagccattgaattccactcagcttgggctatgggcatgggggtcaatttcctagcagctaatctacataatccctcgaggagaatgacaggaagtggtatctatgcacccgattctccaagggtctttcactacgacaggaagacccaagaaggaaaactcctcttcgctcagctgaaatcccacccaattcactccccggtgaactggacttcctatgctagcagtgtagaatcaaccccaaccaaaacccaggaatttcagagtattgtcttttttgatgagtttacctttgtggagctcaaagggatcaaaggaaattacactgtttgccagaatgacctctgctgtcacctaagctaccagatgtctgagaagcgagcagatgaggtttatgcctttggagcctttgatgggctgcacaccgtggaagggcagtactacctacagatctgcatcctgctaaaatgtaaaactaccaatttacgcacctgtggtagttcagtggacacggcttttaccaggtttgaaatgttctcgctcagcggcacttttggaacccggtatgtcttccctgaagtgttgctgagtgaggtcaagctcgcacctggggagtttcaggtgtcaagtgatgggcgcctggttagcctgaagccaacctcgggacctgtgttaaccatcgggctctttgggaggttgtatgggaaggactgggcatccaatgcttcctcagacttcatagcacactcgctgataataatgctgattgtgacgcctattatacattacttgtgctga

SEQ ID NO: 54 - DNA, 1311 bp, Mesocricetus auratus
  source 1..1311: mol_type = genomic DNA
atgctgtgatcctgccaaatgacaccctgacacccgtgtctcacagtgaggcgctggcactaatgaatcagaatctgaatcttctggaaggagcgatcgtgtctgcagcaaagctgatttggctacaacccagtacaggagagactcagctgcttggccaagaataattcaatctatgttgtggcgaacatgggagacaagaaaccatgcaacactagtgaccctcagtgtcctcctgatgggcgtttccagtacaacactaatgtggtgtttgattccctgggcaaactggttgcacgataccataagcaaaaccttttcatgggtgaagaacaattcaacgcacccatggagcccgagttggtgactttcaacaccccgtttggaaggtttggcatcttcacatgcttcgatatcctcttccacgatcctgctgtcaccctggtgaccaagtttcaggtggacaccatactgttcccaaccgcttggatggacgttctgcctcatttggcagccattgaattccactcagcttgggctatgggcatgggggtcaatttacttgcagccaatatacatagtccctcgaggagaatgacaggaagtggtatctatgccccagactctccaaaggcctttcactatgacaggaaaaccaatgagggaaaacttgttctagcgcagctggattcccacccaagtcgctccactgtgaactggacttcttatgccagcagtgtagaagcacccgcaacaggaaaccaggaatttcagagcatcattttctttgatgagtttacctttgtggagctcaaaggaatcacaggaaattacactgtttgccagaatgatctctgctgtcatctaagctaccagatgtctgagaagcgagctgatgaagtttatgcccttggggcatttgatggcttgcacactgtggaagggcagtatcatctgcagatctgtactctgctgaaatgtaaaaccaccagtttacacacctgtggtgattcagtggacacagcttctaccaggtttgaaatgttctccctcagtggcacatttggaactcagtatgtcttccccgaggtgttgctgagtgaagtcaagcttgcacctggggaatttcaggtgtcaagtgacggacgtttgtttagcctgaagccaacatctggacctgtgttaacagtcactctgtttgggaggttgtatgagaaggactcggcatctagtgcttcctcagacctcataacacactcactaatgataataacggtgattgtcctacctattgtacattacttatgttaa

SEQ ID NO: 55 - protein, 512 aa, Physeter catodon
MSQLLDYVAIFFFCVLKASSLDTFTAAVYEHAVILPNATLIPVSHEEALLLMNQNLDLLEAAITSASKQGAHIIVTPEDGIYGFNFNRESIYPYLEDIPDPQVNWIPCNNPDRFGHTPVQERLSCLAKDNSIYIVANIGDKKPCNTSDPHCPPDGRYQYNTDVVFDSEGKLVARYHKQNLFLGEDQFNAPKKPEVVTFNTTFGKFGIFTCFDILFHDPAMTLVKDFHVDTILFPTAWMNVLPHLSAIEFHSAWAMGMGVNFLASNLHYPSMKMTGSGIYAPDSPRAFHYDMKTEKGKLLLSQLASHPHPTAVVNWTSYASGIEAPSMGNQEFKGIIFFDEFTFLELKEVTGNYTVCQKDLCCHLSYKMSEKRSDEVYVLGAFDGLHTVEGSYYLQICTLLKCKTMDFHTCGDSVETASTRFETFSLSGTFETQYVFPEVLLSEIQLAPGEFQVSSDGRLFSQKPLSGPILTVTLFGRLYKKDSAPNAFSDLMAQALRVMLIVVIPIVYSLGW

SEQ ID NO: 56 - protein, 514 aa, Myotis myotis
MITSQLLTCVAISIFCALKASSLDTFIAAVYEHAVILPNATLAPVSRELALGLMNRNLDLLEGAITSAAKQGAHIIVTPEDGLYGWNFNRESIYPYLEDIPDPQVNWIPCNHPNRFGHTPVQERLSCLAKENSIYVVANIGDKKPCNASDPQCPSDGRYQYNTDVVFDSQGKLVARYHKQNLFLGEDQFNVPKEPEVVTFDTTFGRFGIFTCFDILFHDPAVALVKDFHVDTILFPTAWMNVLPHLSAIEFHSAWAMGMKVNFLASNIHHPSKKMTGSGIYAPDSPRAFHYDMKTEKGKLLLSRLDSHPKHPVMVNWTSYASGIEAFSMRNKEFKGTVFFDEYTFLELRGVTGNYTVCQKDLCCHLSYKMSEKRADEVYALGAFDGLHTVEGNYHLQICTLLKCKTTSLHTCGHAVETASTRFEMFSLSGTFGTQYVFPEVLLSEIQLAPGEFQVLSDGRLLSLKPPPGPVLTVTLFGRVYEKDHTSNASSGFMAYTLTIMLIVITSILYSLSR

SEQ ID NO: 57 - protein, 512 aa, Mus musculus
MGTSWWLACAAAFSALCVLKASSLDTFLAAVYEHAVILPKDTLLPVSHSEALALMNQNLDLLEGAIVSAAKQGAHIIVTPEDGIYGVRFTRDTIYPYLEEIPDPQVNWIPCDNPKRFGSTPVQERLSCLAKNNSIYVVANMGDKKPCNTSDSHCPPDGRFQYNTDVVFDSQGKLVARYHKQNIFMGEDQFNVPMEPEFVTFDTPFGKFGVFTCFDILFHDPAVTLVTEFQVDTILFPTAWMDVLPHLAAIEFHSAWAMGMGVNFLAANLHNPSRRMTGSGIYAPDSPRVFHYDRKTQEGKLLFAQLKSHPIHSPVNWTSYASSVESTPTKTQEFQSIVFFDEFTFVELKGIKGNYTVCQNDLCCHLSYQMSEKRADEVYAFGAFDGLHTVEGQYYLQICILLKCKTTNLRTCGSSVDTAFTRFEMFSLSGTFGTRYVFPEVLLSEVKLAPGEFQVSSDGRLVSLKPTSGPVLTIGLFGRLYGKDWASNASSDFIAHSLIIMLIVTPIIHYLC

SEQ ID NO: 58 - protein, 513 aa, Homo sapiens [sequence omitted]

SEQ ID NO: 59 - protein, 512 aa, Monodelphis domestica
MIPFQLQSYIAFFALWAFKTNCLDTFIAAVYEHAVILPPDNIKLDSREEALALMNENMDILEEAVISAAKQGAHIIVTPEHGIYGFIFNRVNLYPYLEDIPHPKVNWTPCTDDSRFGSTPVQKRLSCMARNNSIYMVANIGDRKSCDPTESKCPSNGYYQYNTNVVYDSEGKLVARYYKKYMLMNEAQFDTPTEPQLVTFDTTFGKFGILTGFDILFHDPVVPLVEKLKVDTILLPVAWMNTLPHLYPTAFHAAWAMGMRVNLLVANIHSISNQMTGSGIYAPDSVKAYYFDKKSDDGKLLVSELESHPIVRPPVNWTLYASSIKPFSSEQEEFKALVLFDMFTFKELINDTGNYTVCQNTLCCHLSYKMSEKRTDEVYVVGAFDGLHIAEGEYYLQICTLLKCLTTLIETCGQPVENASTWFDMFSLSGTFDSPHVFPELLLSGLQIAPTEFEILSDGRIINQNGTSAPVLMLTLFGRQYEKTKPPNASVDLETTSVMMITMFFVLYSTIW

SEQ ID NO: 60 - protein, 512 aa, Mus pahari
MNMSWWLACTAAFSVLCVLKASSLDTFLAAVYEHAVILPKDTLLPVSHSEALALMNQNLDLLEGAIVSAAKQGANIIVTPEDGIYGVRFTRDTIYPYLEDIPDPQVNWIPCDNPKRFGSTPVQERLSCLAKNNSIYVVANMGDKKPCNTSDSHCPPDGRFQYNTDVVFDSQGKLVARYHKQNIFMGEDQFNVPMEPEFVTFDTPFGKFGIFTCFDILFHDPAVTLVTEFQVDTILFPTAWMDVLPHLAAIEFHSAWAMGMGVNFLAANLHNPSRRMTGSGIYAPDSPRVFHYDRKTQEGKLLLAQLDSHPSHSPVNWTSYASSVESTPTKNQEFQSIVFFDEFTFVELKGIKGNHTVCQNDLCCHLSYQMSEKRADEVYAFGAFDGLHTVEGQYYLQICILLKCKTTNLRTCGSSVDTASTRFEMFSLSGTFGTRYVFPEVLLSEVKLAPGEFQVSSDGRLVSLKPTSGPVLTVGLFGRLYGKDWSSNASSDFIAHSLIIMLIMTPIIHYLC

SEQ ID NO: 61 - protein, 517 aa, Nematostella vectensis
MFRWFVAKFSLLALVIISHIKSVPESEFIAAVYEHVPPLNRPKEITRANALGIMMRNIDTYEEQMVIARDKNSSIIVFPEYGLTGWNQTRSVFKHFLENIPDPKISSNPCLDPGINKTTPILYRLSCLARKYAMYLVVNMGDIKPCQKASDPHCPGDGRYQYNTNVVFSDNGTLVARYHKQHPFMNEMKVVNRPRVPELVTFQTPFGKFGTFVCFDVLFQAPAVQLVTSVGIDHVVFPTAWFDVLPLFPAIGFHSSWARGIGVNFLAANTHVPSFANTGSGIYSSSGAREYYRSFSASGKLLIASLPKSPKKSPSNMTYVKTPGNTNDDVFYSDLFGDRFLFKELDKAEAIIRVCYPGSKSCCTLHYKMAAKLPDELYALGVFDGLHTLEGQYYLRVCTLIKCFGMSRSSCGSQVSSASTIFDSFALRAELNASHVFPAVVAHGVTLLRDEWDHDVPVKQVESKRRLSKGLLSATLFGRVYSLDGGKTSSTGRATCGPTFILTLRIVCIAMISVNLT

SEQ ID NO: 62 - protein, 510 aa, Bos taurus
MIMSQLLNYVAVLFFCVSRASSLDTFIAAVYEHAVILPNATLVPVSPEEALAVMNRNLDLLEGAVTSASKQGAHIIVTPEDGIYGFNFTRESIYPYLEDIPDPQVNWIPCNNPDRFGHTPVQQRLSCLAKDNSIYIVANIGDKKSCNASDPQCPPDGRYQYNTDVVFDTKGKLVARYHKQNLFLNEDQFNAPKEPEVVTFNTTFGKFGIFTCFDILFHDPAVTLVRDSRVDTILFPTAWMNVLPHLSAIEFHSAWAMGMRVNFLASNLHYPLKKMTGSGIYAPDSPRAFHYDMKTEEGKLLLAQLDSHPHPTPVVNWTSYASGVEAHSVGNQEFTGIIFFDEFTFLELKEIGGNYTVCQRDLCCHLSYKMSEKRSDEVYALGAFDGLHTVEGSYYLQICTLLKCKTTDLHTCGDSVETASTRFEMFSLSGTFGTQYVFPEVLLSEIQLAPGEFQVSNDGRLFSLKPTSGPVLTVTLFGRLYEKDSAPNTLSDLTTQALRLNPKTDAWKSK

SEQ ID NO: 63 - protein, 586 aa, Bulinus truncatus
MSLVLWVPGFSTVEEEKKSSRDIMTSSIVAVLHVTALLLMCVGGVFTSRSKFKAAVYGHAVILPEDPTKVVTRAEAVLNMNKNLDVYREQARKAREQDAEIIVFPEDGLYGFRFKRESIYPYLEYIPDPRVVSWSPCDDPRRHHDTEVQMSLSCMAKENSIYVVANMGDKQACDVLTDPKCPSDGRYQFNTNVAFGPDGTLLARYHKYHLFYELQFDTPELELIHFDTPFGRFGLMVCFDVLFYDPGIPLILNLNISNIVFPTAWMDALPLLSALGFHSSFARGLGVNFLSANIHLPDYRFHGSGIYGPDGAEISYYGAKSTSKPKLLVAEVNSLGNPKPNLNLSASFPFSNRLGELRDSTETCDSESDEFNLKRYFKMPNINDTFESVLFGDVFTFVSLDKPSGHLAVCQKNVCCDLQYSIDEQSFGRELFAFGAFDGLHTYEGQYYFQICTLVRCAAADNRTTCGSVTSTSETNFLHLSMSAEMQTDFIYPQIVLSNNDGELQLAEPQSWDFSDGYLSTKKGFNKTLLSAALFGRYYSRDGCISQEQTDSVEIKVKLGNDGSSSILSFPWILCISILECVLIFK

SEQ ID NO: 64 - protein, 547 aa, Caerostris darwini
MARSRALLFLILFVHSSCLFAKKDFYRAAVLELSQFTNISYPAADILRINLNIYDLAASTAAKHEADIIVFPESGLLPFEKPNREWLLDFIEDIPDPKKMLTNPCAEPDLFSDRPIITKLSCLARHNNIYVVANTADFKKCETDESCDKDNSNCTVCPKDGHFFYNTNVVFNREGTLIAKYYKRHLYFEPEMNTPHKPEDAYFDTDFGNFSTFICFDLMFKEPVKAIGNLSVLNVAYPTYWFDHTPLIFFATPYQQAWAMANKVNLLAANGHHPQTGSLGSGIYSSSKGALIYTHNPDGRSKLLISNVPISPRDDTIDTNNLNPMRFFIENGTIVAAKGEEKPIFKKECLNTVLGDGKPGDYRCGPTNVDQYEFKKLQGNKGKITTCSNKLCCWLDYDAHSMNEDYYFGVSGKDLNFYGEFSFGTEACFIARCESYEGKACTNFKLLSHTIFRKVEIRGNFTTKYILPFAVDSEMRLTNRDNWSLDESRIMYDNVNKKSSLLFFGVYGRLFEKDKDLYNQPDGAVRISCTGNWLIFGLLVSLFLFYF

SEQ ID NO: 65 - protein, 496 aa, Chrysochloris asiatica
MIASQLLAFVAVPVFCILKVSAMDTFIAAVYEHAVVLGNTSLTPVSREEALALMNQNLDLLEGAITSAAMQGAHIIVTPEDGIYGWHFTRDALYPYLEDIPDPQVNWIPCNDPNRFGHTPVQERLSCLAKNNSIYVVANIGDKKLCHTSDPQCPSDGRYQYNTNVVFDSEGKLVARYHKQNLFMGEVQFNSPKMAEVVTFNTTFGRFGIFTCFDILFHDPAVTLVKDFHVDTILFPTAWMNVLPHLSAVEFHSAWAMGMGVNFLAANIHHPMMKMTGSGIYAPDSPPAFHYDMKTKEGKLLLSSLNSHPYSSRVNWSSHASRMEAHSKGNQEFKNTVFFDEFTFVELMGIKGNYTVCQKDLCCHLSYKMTEKRSDEVYALGAFDGLHTVEGTYYLQICTMLKCKTTNIKTCGNSVETASTRFEMFSLSGTFGTQYVFPEVLLSEVQLAPGDFQVSSDGQLSSLKPTSGPVLTVTLFGRWYEKDQMANASSTTEHNH

SEQ ID NO: 66 - protein, 488 aa, Ptilonorhynchus violaceus
MLPFQTLLPTVVFAVTALQALASDTFIAAVYEHAVILPHPTGEPVSPDQALALMNQNMNVLEGAIKEAARQGAHIIVTPEDGIYGWRFTRESIYPYLEDIPDPVVNWIPCTDPSRFGPAPVQERLSCMARNNSIYVVANIGDKKPCNSSDPDCPRDGRYQYNTDVVFDPEGKLVARYHKYNLFMVETQFNYPKEPEAVTFETPFGKFGIFTCFDILFYEPAVVLVNKMQVDTVLFPTAWMNVLPFLTAVEFHSAWAMGMRVNLLAANTHKTILAMTGSGIYAPAGARTYSYNMETEDGHLLIAELDAHPRLSPASPPAVSWNSYALSVERFSQNDHEFTGIIFHDRFTFTELTKPEGNLTVCQKDLCCHLSYKMAGKRDDEAYVLGAFDGLHVVEGQYYLQICTLLKCQSTDLSTCGQPVETAQTKFEMFSLSGTFGTSYVFPEVLYSGVQLAPGEFEVLNDGRLISKAKPTKPVVTVTLFGRWYEKD

SEQ ID NO: 67 - protein, 544 aa, Nephila pilipes
MERDKVTVGNETSLLTGDEDDCLSDNVPQHPNENLETMIAALLSLTAFLAGELSSVKSDGDFYTAAVYEHARYDSATDSAQVIIQRNLANYTIATEIAKSKGADCIVFPEYGVFSPGERSQLKRFLEDIPDPKASRVNPCEESETYSDRPILYALSCLAKNNSIFVVANTGDIKKCEGEPDCPSDGDFHFNTNVVFDRTGTLLVRYHKEHLFYEFGMDLARETQQDTTFETDFGTFATYICFDIDYADMSRVARQPDVDGIMFPTMWVEQPPQMKSIQIWEAWAIANNSTILASNIQLPGYNALGSGIFHGSGPLTYTFNPDGISKLLVAKVPKSGSEPVSPDASITAITETRTWDWKDDGKDVGYECSSRVLGQATDIRTEYRCVEENVTDYSYTELEGPSGHFEICNNGLCCSLDYTTTGLFNRFFLGAFNGLYNHFGRYSWYEEDCILVRCDARGDRPCATFPMKSETVFTQVLLKGNFSSEIVFPSVLENDMRLTPKSDWRFERPNEQEAFIQFNSETGRKVITVGMKGRSYDKDPPYQR

SEQ ID NO: 68 - protein, 467 aa, Sylvietta virens
MLPSQALLPAVLLALAALQALASDTFIAAVYEHAVILPDATEEPVPPSQALALMNKNMDVLEGAVEEAARQGAHIIVTPEDGIYGWRFTRESIYPYLEDIPDPAVNWIPCTDPSRFGPAPVQERLSCMARNHSIYVVANIGDKKPCDSSDPDCPEDGHYQYNTDVVFDTQGKLVARYHKYNLFFESQFNYPKEPEAVTFETPFGKFGIFTCFDILFYEPAVVLVSKMQVDTVLFPTAWMNFLPFLTAVQLHSAWAMGMRVNVLAANTHNTSMEMTGSGIYAPAGAKTYFYNTKTEDGHLLIAELDAHPRLSPASPPAVSWNSYALSVERFSQNDHEFTGVIFEDPFTFMELTKPGGNVTVCQKDFCCHLRYKMAEKRDDEVYVLGAFDGLHVVEGQYYMQICTLLKCPSSNLSTCGGEHVETAQTKFEMFSLSGTFGTSYVFPEVLYSGVQLAPGEFEVTGHPSLLS

SEQ ID NO: 69 - protein, 444 aa, Poecile atricapillus
MLPSQVLLPAVLFALAALQALASDTFMAAVYEHAVILPGPTEEPVSPDSALTLMNKNMDVLEGAIKEAAQQGAHIIVTPEDGIYGWRFTRESIYPYLEDIPDPVVNWIPCTDPSRFGPAPVQERLSCMARNNSIYVVANIGDKKPCDSSDPDCPRDGRYQYNTDVVFDTQGKLVARYHKYNLFQGESQFNYPKEPEAVIFETPFGKFGIFTCFDILFYEPAVVLVSKMQVDTVLFPSAWMNVLPFLTAIEFHSAWAMGMRVNVLAANTHNTSMAMTGSGIYAPAGARTYSYNMKTEDGHLLIAELDAHPRLSPASPPAVSWNSYALNVGRFSQNDHEFTGIIFEDPFTFTELTKPGGNLTVCQKDLCCHLTYKMAEKRDDEVYVLGAFDGLHVIEGQYYLQICSLLKCLSTNLSTCGQPVEMAQTKFEMFSLSGTFGTSYVFPE

SEQ ID NO: 70 - protein, 441 aa, Araneus ventricosus
MSYAEYIPDPKVQKFNPCVENTKDRTILYTVSCLARENNIYIVANLVGSEPCETTCDAAEVENCDAKCPDDGVFFYNTDVAFDREGNLIARYRKVHPYYREIVNVPVETEVGIFETEFGKFGMIVCFDLVFKEAVLLKDEYDIDTLLFPTYWYDDIVPLNAVEFQQAWAIGNGVNVLSANTHAPGTGSMGSGIFSKDDGALVYTYEPDGKSKLLLANVGIKGDSEIPNISSLIEITPDSAVPITETGEKFPVHCYDKVAGPALNGEIEYRCLKLKLENYSLTKLEKPSGEVQACNNELCCTLSYSTQNLQDEYYLAVYNGLNDAFDYFHWWEEICVLTRCDPFGRNPCALQPSISKTVFQKVELKGNFKSEKVYPTVSKTNNRLAPKSEWNFEQGETKAVLSFQSTLNLPLLKVGLLGRSYDRDPEFIPYFPKEFKKQQIN

SEQ ID NO: 71 - DNA, 1599 bp, Phacochoerus africanus
  source 1..1599: mol_type = genomic DNA
atggccaagcgagttgcaattgtgggagctggggtcagtggcctggcctccatcaagtgctgcctggaggaagggctggagcccacctgctttgagaggagcgatgaccttggggggctgtggagattcactgaacatgttgaagaaggcagggcaagcctctacaagtctgtggtttccaacagctgcaaggagatgtcttgttacccagactttccattcccagaagattatccaaactatgtgccaaattctcagttcctggaatatctcagaatgtatgcaaaccagttcaaccttctgaagtgcattcaattcaagactaaagtctgcagtgtaacaaaacgtgaagattttaataccactggccaatgggatgtagtcactctgtgtgaaggaaagcaagagtcagccgtctttgatgctgtcatggtctgcactggttttcttactaacccatatttgccattggactccttcccaggtataaatacttttaaaggccagtacttccatagccgacagtataaacatccagatatatttaaggacaagagtgttcttgtggttggaatgggaaattctggcacagacattgctgtggaggccagccacctggcaaaaaaggtgttcctcagcaccactggaggagcatgggtgatcagccgtgtctttgactcagggtacccatgggacatggtgttcacgacacgatttcagaacttgttcagaaattctctcccaactccaattgtgaattggttgatagcaaaaaagatgaacagctggttcaatcatgcaaattatggcttaatacccgaagacaggatacaactaagagagcctgtgctgaatgatgagctcccaggccgtatcatcactgggaaagtgctcatcaagccaagtataaaggaggtgaaggaaaactctgtggtatttaacagctccccagaggaagagcccattgatatcattgtctttgccactggatacacctttgctttccccttccttgatgagagtgtagtgaaggttgaagatggccaggcatcactatacaagtacatcttccctgcacatctgcagaagccaactctggctgttattggcctcatcaaaccctttggctccatactacccacaggggaaacacaagctcggtgggctgttcgggtcctgaaaggtgtgaataagttaccaccatcaagtgtcatgatacaggaagttaatacaagaaaagaaaacaagcccagtgggtttggcttgtgttactgcaaagctttacaatctgattatatcacatacatagatgaactgctgacctatatcgatgcaaagcccaacatgttttctctgctcctgaaggatccacacctggctttgaccatcttctttggcccatgcacaccataccagttccgcctgactggtccagggaaatgggaaggagccaggaacgctatcatgacccagtgggaccgaacattcaaggccaccaaaactcgaattgtaaaagaatccccatctccctttgcaagcttacttaaactctttagctttctagtcttgcttgtggccattttccagattttcctctaa

SEQ ID NO: 72 - DNA, 1611 bp, Lagenorhynchus obliquidens
  source 1..1611: mol_type = genomic DNA
atggccaagagagttgcaactgtgggagctggggtcagtggcctggcctccatcaagcgctgtccggaagaaggactagagcccacctgcttcgagaggagcgacgaccttggggggctgtggagattcaccgaacatgttgaagaaggcagagccagcctctacaagtctgtggtttccaacagctgcaaggagatgtcttgttacccagactttccattcccagaagattatccaaactatgtgccaaattctgtattcctggaatatctcaaaatgtatgcaaaccagttcaaccttctgaaatgcattcaattcaagactaaagtcttcagtgtaacaaaacgcccagatttttctgtgaccggccaatgggaggtagtcactctgcatgaagagaagcaagagtcagccatctttgacgctgtcatggtctgcactagttttcttactaacccatatttgccactggactcctttccaggtataaatactgttaaaggccagtattttcatagctgagagtataaacatccagatatatttagagacaagagagttcttgtgattggaatggggaattctggcacagacattgctatggaggccagccacgtggcaaagaaggtatcattcttgatagttaccgaacacccctgggcgtgagtgatcagccaggtctttgactcaggctacccatgggacatggtgctcgtgatgcgatttcagaccatgttcagaaattctctcccaactccagttgtgaattggttgatagcaagaaggatgaagagctggttcaatcgtgaaaattatggcttaataccagaagacaggacacaactaagagagtctgtgctaagtaatgagctcccaggccgtatcatcactgggaaagtgctcatcaagccaagcgtaaaggaggtgaaggaaaactctgttgtatttaacaacaccccaaaggaaaagcccattgatatcattgtctttgccactggatacacctttgctttccccttcctccatgagactgtggtgaaagttgaagatggccaagcatcactgtacaagtacatcttccctgcacatctgcaaaagccaaccctggctgttattggcctcatcaaacccttgggctccatgataaccacaggggaaatgcaagcttgatgggctgttcaggtcctgaaaggtgttaataaattaccaccatcaagtgtcatgatagaggaagttaatgcaggaaaaaaaaaaaggccagtgggtaagtttggcttgtgttactgcaaagctttacaatcccattatatcccatgcatagatgaactcctgacctatattgatgcaaagcccaacctgttctctctgctcctgacagatccacgcctggctttgaccatcttcttcggcccatgcacaccataccagttccgtttgacccgcccagggaaatgggaaggagccagaaatgctatcatgaccctgtgggaccaaacattccaggtcacaaaaactagagttgtacaagaatccccttctccctttgcaagcttacttaaactcttcagctttctagctttgcttgtggccattttcctgattttcctataa

SEQ ID NO: 73 - DNA, 1609 bp, Meriones unguiculatus
  source 1..1609: mol_type = genomic DNA
atggtgaagcgagtcgcgattgtgggagccggggtcagcggcctggcctccatcaagtgctgcctggaggagggactggagcccgtctgcttcgagaggagcggggacctggggggactttggagattcactgaagatgttgaagaaggaagagccagtctttacaagtctgtggtttctaacagcagcaaggagatgtcctgttacccagattttccttttccagaagattacccaaactttgtgccaaattctttattcctggaatatctcaaactgtatgcaaacaagttcaaccttctacgatgcattcatttcaataccaaagtgtgcagtataagcaaatgcccagattttgctgtctccggacagtgggaagtggtcactgcccgtggacaagaacagacgcagagctcagccatctttgatgccgtcatggtctgcactggttttctaactaaccctcacttgcccctggattcctttccaggtatacaaacttttaaagggcagtacttccacagccgacagtataagcatccggatgtatttaaggataagcgagtccttgtggttggaatgggaaattctggcacggatattgccgtggaggccagccacttagcaaaaaaggtgttcctgagcaccaccggaggggcatgggtgatcagcagagtctttgattcggggtacccgtgggacatggtattcatgacacggtttcagaacatgttcagaaactctctcccaacgcccattgtgagttggttgatatcaaataagatgaacagctggttcaaccatgtgaattacggtgtggctccagaagacaggattcagctgagagaacctgtcctaaatgatgagctcccaggccgcatcatcactgggaaagtgtttatcaagccaagcattaaggaggtgaaggaaaactctgtcatattcaacagcagcccaaaggaagagcctattgatgtcatcgtttttgccactggatacaccttcgctttccccttcctcgatgaatctattgtcaaagttgaggatggccaggcatcactatacaagtacatcttccctgcgcatctgccaaaaccaactctggctgtgattggcctcatcaaacccctgggctccatgttacccacaggcgacacacaagcccgatgggttgttcaggtcttgaaaggtgcggttacattaccaccaccgagtgtcatgatgcaagaagttgatgaaaggaagaaaaacaagcacagcgggtttggcttgtgctactgcaaggctctgcaaacagattacataacatacatagatgacctcctgacctctatcaacgcaaaaccgaatctgtggtccatgctcctgactgacccgcgcctggctctgagcatcttcttcggcccctgctcaccttaccatttccgcctgactggtccagggaaatgggaaggagccagaaaggccatcttgacccagtgggaccgaaccctgaaagtcaccagaactcgaattgtacaagaatccccatattcctttgaaactttgtttaaactctttagtgttctggctttgcttgtagctattttcttgattttcctgtaagtga

SEQ ID NO: 74 - DNA, 1599 bp, Dipodomys ordii
  source 1..1599: mol_type = genomic DNA
atggtgaagcgggtggccatcgtgggagccggggtcagcggcctggcctccaccaagtgctgcctggaggaaggtctggagcccacctgcttcgagaggagcgaggacctgggcggcctctggaggtacaccgaatatgtggaagagggcagagccagtctctacaactctgtggtctccaacagcagcaaggaaatgtcctgttattcagattttccattcccagaagattacccaaactttgtgccaaattctctcttcctggaatatctcaaaatgtatgcaaaccgatttgatcttctgaaatgcattcaattcaagaccaaagtgtgcagtgttaccaagcgcccagatttcgccacttctggccagtgggaagtggtcactctgcacgaagggaagcagagctcagccaccttcgatgcagtcatggtctgcaccgggtttcttactaacccttctgtgccgctggattccttcccaggcatcaaaacctttaaaggccagtacttccatagccggcactataaacatccaaatatatttaaagacaagcgagtccttgtggttggaatggggaattccggcaccgacattgctgtggaggccagccacctggccaaaaaggtgttcctcagcaccactggaggggcgtgggtgatcagccgtgtctttgactcggggtacccctgggacatggtgttcatgacacgattccagaatatgttcagaaattttcttccaactccaattgtgtcttggttgatagccaaaaagatgaacagctggttcaatcatgaaaattacggtgtaataccagaagacagggttcagatgagagagcctgtgctgaatgatgagctgcctggccgcatcatcaccgggaaagtgtcgatcaagcctagcattaaggaagtgaaggagaactcggtcatattcaacaacaccccgaaggaagagcccattgatatcatcatctttgccacgggctacaccttcgatttccccttcctcgaggaatcggtagtgaaagttgaagatggccaggcatcactgtacaagtatatctttccagtgcatctgccgaagccaacgctggccgtgattaacctcatcaaacctctgggctccatgctgcccacaggagaaacacaagctcgatgggtggtccaagtattcaaaggtaccactctactaccaccagcgagcgtcatgatgaaggaagtcaatgagagaaaagagaacaaacccagtgggtttggcttgtgctactgcaaggctctacagtcggattacatcccatacatcgatgatctcttgacggccatcaacgcgaagcccagcttgtggtccatgttcctgaccgacccgcggctggcctgcgccgtcttctttggcccctgcacaccataccagttccgcctggcgggcacaggaaagtgggaaggagccagacaagccatcctgacgcagtgggaccgagccctgaaagtcaccaaaactcgcactgtgcaaaaacccccctctgcctttgacatctcgcttaaactctttggcttgctggctttgctggtagctgtgttcttggttttcctctga

SEQ ID NO: 75 - DNA, 1410 bp, Bos mutus
  source 1..1410: mol_type = genomic DNA
atggccaaacgagttgcgattgtgggcgctggggccactggcctggcttccatcaagtgctgtctggaggaagggctaaagcccacctgcttcgagaggagtgatgaccttggggggctgtggagattcaccacaaaagtctgcagtgtaagaaaacgcccagattttactgccacaggccagtgggaggtagtcactctgtgtgaagggaagcaagagtcagccatctttgatgctgtcatggtctgtactggtttttttactaatccgaatctaccactggactcctttccaggtataaataattttaaaggccagtatttccacagccgagagtataaacatccagatatatttagggacaagagcgttcttgttattggaatgggaaattctggcacagacattgctgtggaggccagccacctggcaaagaaggtgttcctcagcaccaccggaggggcatgggtgatcagccgtgtctttgactcagggtacccatgggacatggttttcatgacacgatttcagaacacattcagaaattctcttccaactccaattgtgaactggttgatagcgaaaaggatgaacagctggttcaatcatgcaaattatggcttaataccagaagacagggtacagctaagagagcctgtgctaaatgacgagctcccaggccgtatcatcactgggaaagtactcatcaagccaagagttaaggaagtgaaggaaaactctgtcgtatttagcaacaccccaaaggaagagcccatcgatatcattgtttttgccactggatacacttttgctttccccttccttgatgagactgtagtgaaagtcgaagatggccaggcatcgctatacaagtacatctttcctgcacatctgccaaagccaaccctggctgtcattggtctcatcaaacccttgggctccttactacccacaggagatacgcaagctcgatgggctgttcgggtcctgaaaggtgtgattaagttaccaccatcaagcacaatgattgaggaagttaatgaaaggaaaaaaaacaagcccagtgggtttggcttgtattactgcaaagctttggaaaccgattatatcacatatatagatgaactcctgaccgctatcaacgcaaagcccaacctgttctctctgctcctgacggatccacgcctggctttggccatcttctttggcccatgcacaccataccagttccgtttgactggtccagggaaatgggaaggagcccgaaatgctatcatgacccagtgggaccgaacattcaaggccaccaaaactcgaattgtacaagaatccccgtctctctttgcaagcttacttaaagtcttcagctttctagctttgcttgtggccattttcctgattttcctataa

SEQ ID NO: 76 - DNA, 1170 bp, Moschus berezoyskii
  source 1..1170: mol_type = genomic DNA
atggtctgcactggttttcttactgatccatatttaccactggactgctttccaggtataaatacttttaaaggccagtatttccacagccgagagtataaacatccagatatatttagggacaagagagttcttgttattggaatgggaaattccggcacagacattgctgtggaggccagccacctggcaaaaaaggtgttcctcagcaccaccggaggggcatgggtgatcagccgtgtctttgactcagggtacccatgggacatggttttcatgacacgatttcagaacacattcagaaattctcttccaactccaattgtgaattggttgatagcaagaaggatgaacagctggttcaatcatgcaaattacggcttaataccagaagacagggtacagctaagagagcctgtgctaaatgacgagctcccaggccgtatcatcactgggaaagtactcatcaagccaagcgttaaggaagtgaaggaaaactctgttgtatttagcaacaccccaaaggaagagcccatcgatatcattgtttttgccactggatacacttttgctttccccttccttgacgagactgtagtgaaagtcgaagatggccaggcatcgctatacaagtacatctttcctgcacatctaccaaagccaacgctggctatcattggtatcatcaaacccttgggctccttactacccacaggggatatacaagctcgatgggctattcaggtcctgaaaggtatgattaagctaccaccatcaagcataatgatcgaggaagttaatgaaaggaaaaaaaacaagcccagtgggtttggcttgccttactgcagagctttgcaaaccgattacatcacatatgtagatgaacttctgaccgctatcaatgcaaagcccaacctgttctctctgcttctgacggatccacgcctggctctggccatcttctttggtccatgcacaccataccagttccgtctgaccggtccagggaaatgggaaggagcccgaaatgccatcatgacccagtgggaccgaacattcaaggccaccaaaacacgaattgtacaagaacccccgtctctctttgcaagcttacttaaactcttcagctttctagctttgcttgtggccattttcctgattttcctataa

SEQ ID NO: 77 - DNA, 1410 bp, Chrysochloris asiatica
  source 1..1410: mol_type = genomic DNA
atggccaagagagttgcaatagtgggagccggggtcagtggcctggcctgcatcaagtgctgtctggaggaaggattggagcccatctgctttgagaggagtgacgacttgggagggctgtggagattcactaccaaagtctgcagtataacaaagcgcccagatttttctgtctctggccaatgggatgtgatcactctacatgaagggaagcaagagtccgctgtctttgatgctgtcatggtctgcactggttttcttactaatccaagtttgcccctggactcctttccaggtataaaagcttttaaaggccaatactttcatagccgagaatataagcatccagatatatttaaggacaagtgtgtccttgtgattggaatgggtaactctggtacagacattgctgtggaagccagccacctggcaaaaaaggtgttcctgagcacaactgcaggggcatgggtgataagccgagtgtatgactcagggtacccatgggacatggtgttcatgacacgatttcagaacatgttcagaaatgctctcccaactccaattgtgtcttggttgcttgcaagaaagatgaacagttggttcaatcacacaaattacggcgtagttccagaagacagggtacagctgagagagcctgtgctaaatgatgagcttccaggtcgtatcatcacgggcaaagtgctcatcaagccgaatgtaaaggaggtgaaggaacattcagtcatatttaaaaactccccaaaagaagaacccattgatatcattgtctttgccactggatacacctttgcttttcccttccttgatgagtctgtagtgaaagttgaagatggccaggcatcactgtacaagtatatcttccccacgcacctgcaaaagtcaactctggctttcatcggtctcatcaaacccttgggctcgatgatacccacaggagaaatgcaagctcgatgggctgttcgagtactgaaaggtgtgaatacattaccaccatcaagtgttatggcaaaggaggttaacgaaaggaaagaaaacaagcctagtgggtttggcttgtgttactgcaaggctttacaatcagattatatcccatacatggatgaacttctaacctatattgatgcaaaacccaacctgttctccatgctcctgacggatccacgcctggctttgaccatcttctttggtccatgttcaccataccagttccgcttgactggaccagggaaatgggaaggagccagaaacgccatcctgacccagtgggaccgaacattcaaggtcaccaaaactcgagttttactagactcctcatctgtgtttgaaaacttacttaaactctttagctttctggctttgcttatggccattttcctgattttcttgtaa

SEQ ID NO: 78 - DNA, 1599 bp, Erinaceus europaeus
  source 1..1599: mol_type = genomic DNA
atggctagacaagtggcaatcgtgggtgctggggtcagtggcttggcctctatcaagtgctgcctggaggagggattggaacccacctgctttgagaggagcgatgatctcgggggactatggagattcaccgaacatgtggaagaaggcagagccagcctctacaagtctgtggtttccaacagctgcaaggagatgtcctgttactcagactttccattcccagaacattacccaaactacgtgccaaattcccagttcctggaatatctcaaaatgtatgcacatcgatttgatcttctgaaatgcattcaatttaagactaaagtctgcagtgtaacaaaatgcccaaactttgatgtcactggccaatgggaagtggtcactctgcatgaagggaaacaagagtcaagtgtctttgatgcagtcatggtctgcactggttttcttactaacccccacttgccactggactcctttccaggtataaaaacctttaaaggccagtacttccacagccgacagtataaacatccagatatatttaaggacaagagagttctcgtggttggcatgggaaattctggcactgacatcgctgtggaggctagtcatgtggcggagaaggtatacctcagcaccacaggaggagcctgggtaatgagccgagtctttgattcagggtacccatgggacatggtattcatgacacgcttccagaacatgtttagaaatttgctccccactccagttgtgtccaggttgatagcaagaaagatgaacagctggttcgatcacacaaattatggtcttgtaccagaagacagaattcagctgaaggagcttgtgctgaatgatgagcttccaggccggatcatcaccgggaaagtgttcatcaggccaagcataaaggaggtgaaggcgcactcagtcatgttcaacaacagtccaaaagaagagcccattgacatcatagtctttgccacaggctacacttttgccttccccttccttgatgagagtgtaataaaagttgaagctggccaggcatcactctataagtatatcttccctgcacatctgaagaagtcaaccctggcagttattggcctcatcaaacccttgggctccatgatacccactggagaaacacaagctcgctgggctgtccgagtcctgaaaggtgtgaataagttaccacctccaagtgtcatgatagaagaagttaatgcaagaaaagaaaacaaacccactgggtttggcttgtgctactgcaaagctttacaatcagattacattctatacatcgatgaactcctgacttccatcaatgcaaaacccaacctgttctccttgctcctgacagacccacacctggcttgggccatcttctttggcccctgctcaccataccagtttcgcctaactggaccagggaagtgggagggggctcgaaatgccatcctgacccagtgggaccgaacattcaaggccaccagaactcgaattgtccaagaaccgccatctccttttgtaagcttccttaagttcttcagttttctggctttgctggtggctgttttcctgatccttctttaa

SEQ ID NO: 79 - DNA, 1407 bp, Artibeus jamaicensis
  source 1..1407: mol_type = genomic DNA
atgtcctgttactcagactttccattcccggaagattatccaaactatgtgcccaatgcccagttcctggaatatctcaaaatgtatgcaaaccacttcaaccttctgaaatgcattcaattcaagactaaagtgtgcagtgtaactaaatgcccaaatttcactgtcactggccaatgggacgtggtcactctgcacgaagggaagcaagagtccactgtctttgatgctgtcatggtctgcactggtttgcttacttacccaaatttgccactggactcctttccaggtataaatacattcaaaggccaatactttcatagccgacagtataaacatccagacatatttaaggacaagagagttcttgtgatcggagcaggaaattctggtgcagacatagctgtggaggccagccacgtggcaaaaaaggtgttcctcagcaccacaggaggcgcgtgggtgatcagccgcctctgcaactcagggttgccctgggacatggtgttcacgactcgatttcagagcatgttcagaaattctctcccaaccccaattgtgacttggtggatggcaagaaagataaacagctggttcaatcatgcaaattacggcttagtcccaaaagacaggacacagctgaaagagtatgtgataaatgatgagctcccaggccgcatcatcactggaagagtgttcatcaagccaagcgtaaaggaagtgaaggaagactcagtcatatttaacgaccccccagaggaacagcccattgacatcattgtcttcgccactggatacacctttgctttccccttccttgatgaaaccgtggtgaaagtcgaaaatgaccaggcatcgctgtacaagtacatcttcccggcacatctggaaaagccaaccctggctgtcattggcctcatcaactccttaggctccatgatacccacaggagaaacacaagctcgatgggctgttcgagtcctgaaaggtgtgaataacttaccaccttgcagtgtcatgagagaagaagttaataggaggaaagagaacaagcccagtgggtttggcttccgctactgcaaagttttacaatcagattatatcccatacatagatgagcttctgacttatatcaatgctaaacccaacctgttctctctgctcctgacggacccacgcctggcgttgaccatcttcttcggcccatgcacaccataccagttccgcctgaccggcccaggaaaatggaaaggagccagaaaagccatcttgacgcagtgggaccgaaccatcaaggtcaccaaaacccgcactgtacatgaatccccatttccctttgcaagcttactcactgcctttctggttttgcttgtggccattttcctgacttgcctaaagatgagctaa

SEQ ID NO: 80 - DNA, 1407 bp, Gracilinanus agilis
  source 1..1407: mol_type = genomic DNA
atggtcaaacgagttgcaattattggagctggtgtgagtggcctcacctccatcaaatcctgcttagaagaggggctggagcccacctgctttgaaaggagtggtgacattggagggttatggcaatttacaactattgtctgcaatgtgaaaaaaagaccagatttcgctacttctggccagtgggaggtaaccactaagcatgaagggaagcaagaatcagctatatttgatgctgtcatggtctgcactggttatctcactaacccatttttgccactagactcttttccaggtatcaaaacctttaagggtcagtacttccatagcagagagtacaagcatccagacttgttcaaggataagagagtccttgtggttggcatgggcaactctggaacagacattgctgtggaggccagtcatgtggctaaaaaggttttgctcagcaccactggaggtgcatggttgataagtcgagtttttgaccatgggtatccttgggacatggtgttcactactcgatttcaaagtgtattaagaaattgcctcccaactccagttgtaaattggctaattggcaaaaggatgaatggctggttcgatcatgcaaactatggtttggttccagaaaacaggactttcttaagagaactaatattgaatgatgagctcccaggacgcataattaccgggaaggtatcgattaaacccagtgtggaggcagtgaaggaaaattctgttttgtttaagaataccccaaaagaagagccaattgatataattgtctttgccacaggatatacttttgattttcctttccttgatgaatctatagtgaaagttgaaaataatcaggcttctctatataagtacatattccctgttcatctggaaaagccaactttggctgtcattggcctcatcaaacctttggtatctttagtctgcacaacagaagtacaagcccgatgggtcacgcgtgtcctaaagggcataaataagttaccaccaccagaggtcatgagagaagatgttaacaaattaaaattaaatagagccactgggtttggcttacacttcaatatggttttgcaaacagatactatcagatacttagatgagctcttgtcctccatcgatgcaaaacccaatatcttgtctctgttcctggaggacccgctcctggctttcaagattttctttggcccacttacctcataccagtaccgtctgactggcccaggaaaatgggatggagccaggaatgccattctaacccagtgggaccggtcattcaagccaatgaaaacgcgagttgtgcaagaggttccacatccctttttcaacctgctcaaaatctttggttttttagttctccttgtagctatatttcttacattctaa

SEQ ID NO: 81 - DNA, 1494 bp, Cercocebus atys
  source 1..1494: mol_type = genomic DNA
atggccaagcgagttgccattgtgggagctggggtcagcggcctggcctccatcaagtgctgtctggaagaaggactggagcccacctgctttgagaggagcgacgaccttggagggctgtggagattcaccgaacatgttgaagaaggcagagccagtctctacaagtctgtggtttccaacagctgcaaggagatgtcttgttactccgactttccattcccagaagattatccaaactatgtgccaaattctcaattcctggaatatctcaaaatgtatgcaaaccacttcaaccttctgaaacacattcaattcaagaccaaagtctgcagtgtaacaaaatgttcagattttactgtctctggccagtgggaggtggtcactatgcgtgatgagaagcaagagtcagccatctttgatgctgtcatggtctgcactggctttcttactaacccttatttgccactggactcctttccaggtattaatgcctttaaaggccagtactttcatagccggcaatataagcatccagatatatttaaggacaagagagtccttgtgattggaatgggaaattctggcacagacattgctgtggaggccagccacctggcggaacaggtgttcctcagcaccaccggagggggatgggtgatcagccgaatctttgactcaggctacccatgggacatggtgttcatgacacgatttcagaacatgttgagaaattctctcccaactccaattgtgacttggttgatgatgcgaaagataaacaactggctcaatcatgcaaattacggcttaataccagaagagagaactcagctgaaagagtttgtactaaatgatgagctcccaggccgcatcatcactgggaaagtgttcatcaggccaagcataaaagagtctgtagtgaaagttgaagatggccaggcctcactgtacaagtatatcttccctgcacatctgcaaaagccaaccctggccattattggcctcatcaaacccttgggctccatgatacctacaggagaaacacaagctcgttgggctgttcgagtcctgaaaggtgtaaataagttaccaccaccgagtgtcatgatagaggaaattaatgcaaggagagaaaacaagcccagttggtttggcttgtgctactgcaaggctttacaatcagattatatcacatacatagatgaactcctgacctatatcaatgcaaaacccaagctgttctctatgctcctaacggatccacacctggctctgaccgtcttctttggcccatgctcaccataccagttccgcttgactggcccaggaaaatgggaaggagccagaaatgccatcatgacccagtgggaccgaacattcaaggtcaccaaaggtcgagttgtacaagagtctccatctccctttgaaagttttcttaagctctttagctttctggctttgcttgtggctatttttctgattttgctataa

SEQ ID NO: 82 - protein, 532 aa, Phacochoerus africanus
MAKRVAIVGAGVSGLASIKCCLEEGLEPTCFERSDDLGGLWRFTEHVEEGRASLYKSVVSNSCKEMSCYPDFPFPEDYPNYVPNSQFLEYLRMYANQFNLLKCIQFKTKVCSVTKREDFNTTGQWDVVTLCEGKQESAVFDAVMVCTGFLTNPYLPLDSFPGINTFKGQYFHSRQYKHPDIFKDKSVLVVGMGNSGTDIAVEASHLAKKVFLSTTGGAWVISRVFDSGYPWDMVFTTRFQNLFRNSLPTPIVNWLIAKKMNSWFNHANYGLIPEDRIQLREPVLNDELPGRIITGKVLIKPSIKEVKENSVVFNSSPEEEPIDIIVFATGYTFAFPFLDESVVKVEDGQASLYKYIFPAHLQKPTLAVIGLIKPFGSILPTGETQARWAVRVLKGVNKLPPSSVMIQEVNTRKENKPSGFGLCYCKALQSDYITYIDELLTYIDAKPNMFSLLLKDPHLALTIFFGPCTPYQFRLTGPGKWEGARNAIMTQWDRTFKATKTRIVKESPSPFASLLKLFSFLVLLVAIFQIFL

SEQ ID NO: 83 - protein, 532 aa, Bos taurus
MAKRVAIVGAGATGLASIKCCLEEGLKPTCFERSDDLGGLWRFTEHVEEGRASLYKSVVSNSCKEMSCYPDFPFPEDYPNYVPNSQFLDYLKMYANRFNLLECIQFKTKVCSVRKRPDFTATGQWEVVTLCEGKQESAIFDAVMVCTGFLTNPNLPLDSFPGINNFKGQYFHSREYKHPDIFRDKSVLVIGMGNSGTDIAVEASHLAKKVFLSTTGGAWVISRVFDSGYPWDMVFMTRFQNTFRNSLPTPIVNWLIAKRMNSWFNHANYGLIPEDRVQLREPVLNDELPGRIITGKVLIKPRVKEVKENSVVFSNTPKEEPIDIIVFATGYTFAFPFLDETVVKVEDGQASLYKYIFPAHLPKPTLAVIGLIKPLGSLLPTGDTQARWAVRVLKGVIKLPPSSIMIEEVNERKKNKPSGFGLCYCKALETDYITYIDELLTAINAKPNLFSLLLTDPRLALAIFFGPCTPYQFRLTGPGKWEGARNAIMTQWDRTFKATKTRIVQESPSLFASLLKVFSFLALLVAIFLIFL

SEQ ID NO: 84 - protein, 532 aa, Bos taurus
MGKRVAIVGAGVSGLASIKCCLEEGLEPTCFERSDDLGGLWRFTEHVEEGRASLYESVVSNSCKEMSCYSDFPFPEDYPNYVPNSQFLEYLKMYANHFNLLKHIQFKTKVCSVTKCSDFTVSGQWEVVTLHKEKQESAIFDAVMVCTGFLTNPYLPLDSFPGINAFKGQYFHSRQYKHPDIFKDKRVLVIGMGNSGTDIAVEASHLAKKVFLSTTGGAWVISRIFDSGYPWDMVFMTRFQNMLRNSLPTPIVTWLMARKINNWLNHANYGLMPDDRTQLKEFVLNDELPGRIITGKVFIRPSIKEVKENSVIFNNTSKEEPIDIIVFATGYTFAFPFLDESVVKVEDGQASLYKYIFPANLQKSTLAIIGLIKPLGSMIPTGETQARWAVRVLKGVNKLPPPNVMIEEVNSRKENKPGWFGLCYCKALQADYITYIDELLTYINAKPNLFSMLLMDPHLALTVFFGPCSPYQFRLTGPGKWEGARNAIMTQWDRTFKVTKVRVVQKSPSPFESFLKLFSFLALLVAIFLIFL

SEQ ID NO: 85 - protein, 532 aa, Suncus etruscus
MARAVAIVGAGVAGLASIKCCLEEGLEPTCFERSDDLGGLWRFTEHVEEGRASLYKSVVSNSCKEMSCYSDFPFPEHYPNYVPNSYFLDYLRMYADHFDLLRCIRFKTKVCSVTKSPDFSVTGKWEVITQQEGKQESATFDAVMVCTGFLTIPHLPLDSFPGIKNFKGEYFHSRRYKHPDIFKDKRVLVIGMGNSGTDIAVEASHVAKKVYLSTTGGAWVMSRVFDSGYPWDMVFTTRFQNLLRNSLPTSLVTRLIARKMNSWFNHANYGVIPKDRTQLREPVLNDELPGRIITGKVLIRPSIREVRAHSVLFCDMPEEEPVDIIVLATGYTFAFPFLDETVVKVEEGQASLYKYIFPAHLPKPTLAVIGLIKPLGSMVPTMETQARWAVRVLKGVNKLPPSAVMMEEVRVRKQNKHTGFGLHFCQALQSDYIPYIDELLDAIGAKPNLLALLLTDPRLAWALFFGPFTPYQFRLMGPGKWAGARHAILSQWDRTVKVTKTRVVQESTSSVISFLKLIGFLVVLMAVFLIFL

SEQ ID NO: 86 - protein, 469 aa, Pan paniscus
MAKRVAIVGAGVSGLASIKCCLEEGLEPTCFERSDDLGGLWRFTSKVCSVTKCSDFAVSGQWEVVTMHEEKQESAIFDAVMVCTGFLTNPYLPLDSFPGINAFKGQYFHSRQYKHPDIFKDKRVLVIGMGNSGTDIAVEASHLAEKVFLSTTGGGWVISRIFDSGYPWDMVFMTRFQNMLRNSLPTPIVTWLMERKINNWLNHANYGLIPEDRTQLKEFVLNDELPGRIITGKVFIRPSIKEVKENSVIFNNTSKEEPIDIIVFATGYTFAFPFLDESVVKVEDGQASLYKYIFPAHLQKPTLAIIGLIKPLGSMIPTGETQARWAVQVLKGVNKLPPPSVMIEEINARKENKPSWFGLCYCKALQSDYITYIDELLTYINAKPNLFSMLLTDPHLALTVFFGPCSPYQFRLTGPGKWEGARNAIMTQWDRTFKVIKARVVQESPSPFESFLKVFSFLALLVAIFLIFL

SEQ ID NO: 87 - protein, 469 aa, Homo sapiens
MAKRVAIVGAGVSGLASIKCCLEEGLEPTCFERSDDLGGLWRFTTKVCSVTKCSDSAVSGQWEVVTMHEEKQESAIFDAVMVCTGFLTNPYLPLDSFPGINAFKGQYFHSRQYKHPDIFKDKRVLVIGMGNSGTDIAVEASHLAEKVFLSTTGGGWVISRIFDSGYPWDMVFMTRFQNMLRNSLPTPIVTWLMERKINNWLNHANYGLIPEDRTQLKEFVLNDELPGRIITGKVFIRPSIKEVKENSVIFNNTSKEEPIDIIVFATGYTFAFPFLDESVVKVEDGQASLYKYIFPAHLQKPTLAIIGLIKPLGSMIPTGETQARWAVRVLKGVNKLPPPSVMIEEINARKENKPSWFGLCYCKALQSDYITYIDELLTYINAKPNLFSMLLTDPHLALTVFFGPCSPYQFRLTGPGKWEGARNAIMTQWDRTFKVIKARVVQESPSPFESFLKVFSFLALLVAIFLIFL

SEQ ID NO: 88 - protein, 530 aa, Desmodus rotundus
MAKRVAIVGAGVSGLASIKCCLEEGLEPTCFERSDDLGGLWRFTEHVEEGRASLYKSVVSNSCKEMSCYSDFPFPEDYPNYVPNAQFLEYLKMYANHFNFLKCIQFKTKVCSVTKRPDFTVTGQWDVVTLREGKQESTVFDAVMVCTGLLTDPNLPLDSFPGINTFKGQYFHSRQYKYPDIFKDKRVLVIGAGNSGADIAVEASHVAKKVFLSTTGGAWVISRVCDSGLPWDMLFTTRFQNVFRNSLPTPVVTWWMARKINSWFNHANYGLVPEDRTQLKEYVINDELPGRIITGRVLIKPSVKEVKEDSVIFNGSPEEQPIDIIVFATGYTFAFPFLDETVVKVENGQASLYKYIFPAHLEKPTLAVIGLINSLGSMIPTGETQARWAVRVLKGVNNLPPQSVMREEVNARKENKPSGFGLCYCKALQSDYIPYIDELLTYIDAKPNLFSLLLTDPRLALTIFFGPCTPYQFRLTGPGKWKGARNAILTQWDRTIKVTKTRTVHESPFPFASFLTVFLVLLVAIFLTCL

SEQ ID NO: 89 - protein, 532 aa, Gopherus flavomarginatus
MVKRVAIIGAGVSGLTSIKCCLDEGLEPSCFERSDDIGGLWRFTEDVEDGRPSLYKSMVSNTCKEMSAFTDFPYPEDFPVFVSNSQVLEYLRMYAKHFGLLKHIQFKTTVISVRKCPDFSATGQWAIVTESNGKQESSVFDAVMVCIGYLSDPSLPLESFPGIETFQGEYFHSRYYKHPDVFEGKRVLVIGMGNSGVDIAVEASHVAKKVMISTGRGAWVISRVFDNGYPWDMVFLTRFMNVIRNSLPGPITTWLVAHRTSQWFDHANYGLIPKDRDVIREPVLNDDLPGCIITGKITIKPSLKEIKENSVIFQNSPTEEPVDIIVFATGYKFSFPFLEESVVKVEKKQASLYKYIFPPHLEKPTLAVIGLIKPFGAVMPVVEIQARWVTRVFKGLCQLPPVSTMVEEINETKKNKIRWFGLSYDEILKTDCLVYIDELASLIGTKPNVLALLLKDPVLAMKIFFGPCTAYQYCLTGPGKWNGARHAILTQWDRTVKPTRTRVVEEPPNFCFNLLKLISLFALFAAIFLGFK

SEQ ID NO: 90 - protein, 519 aa, Dromaius novaehollandiae
MRVAVVGAGVSGLTALKCCLAEGLAPTCFEQSRDVGGLWRYTEHIEDGRPSLYTSVVSNTSKEMSAFSDFPFPEDFPVFLPHARLLEYLQRYAERFALRQHIHFGTTVVSVRKRPDFAATGQWDVVTEADGTRESHVFDAVMVCSGNFSDPSLPLHCFPGIERFQGQYFHSRQYKHPDVFQGKRVLVVGMGNSGVDIAVEASRVAAKVTVCTGRGAWVFSRVFDHGYPWDMVFNTRFMNLIRNSLPGALTRGLISYRVNQWFNHENYGLQPEKSCLVREPVLNDDLPSYIITGKITIKPGVKEFKENSAIFYNCPEEEPIDIVVFCTGYNISFPFLEEADVKVENNHASLYKYVFPAHLQRPTLAVLGLIKPLGAIMPVTEMQARWVTRVFKGRRSLPHLLFSDSLRFGLSFDEVLKIDCLVYSDEIASFIGVKPNVLALLFRDPKLALSVFFGPCTPYQFRLQGPGCWDGARQAILTQWDRTLKPTRTRVTSSSSGIWPSFLTLFGFLLLMAAVFFGF

SEQ ID NO: 91 - protein, 535 aa, Agelaius phoeniceus
MRVAVVGAGVSGLAATKCCLDEGLEPTCFEQSQDIGGLWRYTEHIEAGRPSLYPSVISNTSKEMSAFSDFPFPEHFPVFLPNALLLDYLRRYTEHFSLRQHIRFGSTVVSIRKHPDFATTGQWDVVTEAGGKQTSHVFDAVMVCSGNFSEPSLPLQCFPGIERFRGQHFHSRQYKHPDVFQGKRVLVVGMGNSGVDIAVEASRVAAKVTISTTRGAWLLSRVFEHGYPWDMILNTRLMSLIKTNLPGPLSWWLINYKVNQWFNHENYGLQPENRYSWCWLVREPVLNDELPSYILTGRITIKPGVKEFKDNSVLFHNCPEEEPIDIVVFCTGYTISFPFLEESIVRVENKHASLYKYVFPPQLQKPTLAVLGLIKPFGAIMPVAEMQARWVSRVFKGRRANVTLLCPKMQLRQNWTWFVPSPREIPELRILKTEWLVYLDTLASFIGAKPSVLRLLCTDPWLALTIFFGPCSPYQYRLGGPGRWQGARQAILTQWDRVLKPTRTRVPAGSSSSSLSLLTVVGFLLLLAAVIFGFL

SEQ ID NO: 92 - protein, 521 aa, Crypturellus undulatus
MRVAVVGAGVSGLTAIKCCLDEGLKVTCFERSQDIGGLWRYTENIEDGRPSLYTSVVSNTSKEMSAFSDFPYPEDFPVFLPHARLLEYLRRYAERFALRQRIQLETTVLSVRKRPDFATTGQWDVVTEAGGKRTSHVFDAVMVCSGNFWEPSLPLHAFPGIEKFQGKYFHSRQYKHPDVFQGKRVLVVGMGNSGVDIAVEASRVAAKVTLSTGQGAWVISRVFDHGYPWDMVFNTRFMSLLRNNLPGPLARGLISHRVNQWFNHENYGLQPKKRYFSEPVLNDDLPSYIITGKITIKPGVKEFKENSVVFYSCPEEEPIDIVVFCTGYNVSFPFLEEADVKVENKHASLYKYVFPTHLQRPTLAVLGLIKPLGAIMPVIEMQARWVTRVFKGRRLMPVSLHPIFSNSLRFGLLFDEVLKTDCLVYSDELASFIGAKPNVLALLFSDPKLAFTIFFGPCTPYQFRLRGPGRWDGARQAIFTQWDRMLKPMRTRVTSRSSSVWPSLLTVFGFLLLVAAVVFGF

SEQ ID NO: 93 - protein, 524 aa, Regulus satrapa
MRVAVVGAGVSGLTATKCCLDEGLEPTCFEQSQDLGGLWRYTEHIEAGRPSLYPSVISNTSKEMSAFSDFPFPEHFPVFLPNALLLDYLRRYAERFSLREHIKFGTTVVSIHKHPDFATTGQWNVVTEAGGKQTLDVFDAVMVCSGNFSEPSLPLQCFPGTRFQGQYFHSRQYKHPDVFQGKRVLVVGMGNSGVDIAVEASRVAAKVTISTTRGAWLLSRVFEHGYPWDMILNTRLMSLIKTNLPGPLSWWLINYKVNQWFNHENYGLQPEKRYLPVLNDDLPSYILTGRITIKPGVKEFKDNSVLFHNCSEEEPIDIVVFCTGYIVSFPFLEESIVRVENKHASLYKYVFPTHLQKPTLAVLGLIKPFGAIMPVAEMQARWVSRVFKGRETNVSLPVPQNAAQTGQVLSLQFGLTFDEVLKTEWLVYLDTLASFIGAKPSVLGLLCTDPRLALTIFFGPCSPYQYRLEGPGRWEGARQAILTQWDRVLKPTRTRVPAGSSSSFPSLLTVVGLLLLLAAVIFGF

SEQ ID NO: 94 - protein, 495 aa, Mirounga angustirostris
MPCESGKEHVEEGRASLCKSVVSNSCKEMSCYSDFPFPEDYPNYVPNSQFLEYLKMYANWFSLLECIQFKTKVCKVTKCPDFTVTSQWEVVTQHEGKQESAIFDAVMVCSGFLTNPYLPLDSFPGINTFKGQYFHSQQYKHPDIFKDKRVLVIGMGNSGTDIAVEASHLAKQVFLSTTGGAWVMSRVFDSGYPWDMVFMTXFQNMFRNSLPTPFVTWLMARKMNSWFNHANYGLVPEDRTQLREPVLNDELPGCIITGKLLIRPSIKEVNENSVVFNNTPKEEPIDIIVFATGYTFAFPFLDETVVKVENGQASLYKYIFPAHLPQPTLAVINLIKPLGSMIPIGETQARWVVRVLKGINKLPPRSVMIDEVNARKEDTPSGFGLCYCKALQSDYITYIDELLTSINAKPNLFSLLLMDPRLALTIFFGPCTPYQFRLTGPGKWKGARNAILNQWDRTFKVTKTRIVQESPSPFASLLKLLSLLALLMAIFLIFL

SEQ ID NO: 95 - protein, 468 aa, Artibeus jamaicensis
MSCYSDFPFPEDYPNYVPNAQFLEYLKMYANHFNLLKCIQFKTKVCSVTKCPNFTVTGQWDVVTLHEGKQESTVFDAVMVCTGLLTYPNLPLDSFPGINTFKGQYFHSRQYKHPDIFKDKRVLVIGAGNSGADIAVEASHVAKKVFLSTTGGAWVISRLCNSGLPWDMVFTTRFQSMFRNSLPTPIVTWWMARKINSWFNHANYGLVPKDRTQLKEYVINDELPGRIITGRVFIKPSVKEVKEDSVIFNDPPEEQPIDIIVFATGYTFAFPFLDETVVKVENDQASLYKYIFPAHLEKPTLAVIGLINSLGSMIPTGETQARWAVRVLKGVNNLPPCSVMREEVNRRKENKPSGFGFRYCKVLQSDYIPYIDELLTYINAKPNLFSLLLTDPRLALTIFFGPCTPYQFRLTGPGKWKGARKAILTQWDRTIKVTKTRTVHESPFPFASLLTAFLVLLVAIFLTCLKMS

SEQ ID NO: 96 - protein, 483 aa, Bubalus carabanensis
MNKWFCFIVPTQDNMAKRVAIVGAGVTGLASIKCCLEEGLKPTCFERSDDLGGLWRFTTKVCSVRKRPDFTATGQWEVVTLCEGKQESAIFDAVMVCTGFLTNPYLPLDSFPGINTFKGQYFHSREYKHPDIFRDKSVLVIGMGNSGTDIAVEASHLAKKVFLSTTRGAWVISRVFDSGYPWDMIFMTRFQNKFRNSLPTPIVNWLIARRMNSWFNHANYGLVPEDRVQLREPVLNDELPGRIITGKVLIKPSVKEVKENSVIFSNTPKEEPIDIIVFATGYTFAFPFLDETVVKVEDGQASLYKYIFPAHLPKPTLAVIGLIKPLGSLLPTGDTQARWAVRVLKGVIKLPPSSIMIEEVNERKKNKPSGFGLCYCKALETDYITYIDELLTAINAKPNLFSLLLTDPRLALAIFFGPCTPYQFRLTGPGKWEGARNAIMTQWDRTFKATKTRIVQESPSLFASLLKVFSFLALLVAIFLIFL

SEQ ID NO: 97 - protein, 389 aa, Moschus berezovskii
MVCTGFLTDPYLPLDCFPGINTFKGQYFHSREYKHPDIFRDKRVLVIGMGNSGTDIAVEASHLAKKVFLSTTGGAWVISRVFDSGYPWDMVFMTRFQNTFRNSLPTPIVNWLIARRMNSWFNHANYGLIPEDRVQLREPVLNDELPGRIITGKVLIKPSVKEVKENSVVFSNTPKEEPIDIIVFATGYTFAFPFLDETVVKVEDGQASLYKYIFPAHLPKPTLAIIGIIKPLGSLLPTGDIQARWAIQVLKGMIKLPPSSIMIEEVNERKKNKPSGFGLPYCRALQTDYITYVDELLTAINAKPNLFSLLLTDPRLALAIFFGPCTPYQFRLTGPGKWEGARNAIMTQWDRTFKATKTRIVQEPPSLFASLLKLFSFLALLVAIFLIFL

SEQ ID NO: 98 - DNA, 1542 bp, Synthetic construct
  source 1..1542: mol_type = other DNA
atgatcacctctccgttgctggcttatgttgcgattctgttcttgtgtgtcctgaaagccagctccctggatacctttatcgccgctgtttatgaacacgcggtgattcttcctgatgcccccttgacaccagtatcccacgaagaggctctcatgctgatgaaccgcaacctggatcttttggaaggcgcaatcatgtcagcagcaaagcaaggtgcgcacatcattgtgactccagaagatggtgtttacggcttcttcttcagccgcgaatctatctactcctacctcgaggacatcccagagccgcacgtgaactggatcccttgcaccaatcccaaccgctttggccacaccccagtacaaaagcgactcagctgcctggcccgggacaactccatctacattgtggccaatatcggagacaagaagccatgcaacgcctcggaccccgactgtcctcatgacggccgttaccagtacaacaccgatgtggtctttgattccgagggccgactggtcgcacgctaccacaagcaaaatcttttcttgggtgaagatcaattcgatgcacccaaggagcccgagatcgtgacgttcgacaccacctttggccgttttggcatcttcacgtgctttgatattctcttccatgatcctgctgtgaccctggtcaaagatttccaggtagacaccatcctcttcccgaccgcgtggatgaacgttctcccacatttgaccgctattgaattccactcggcttgggctatgggcatgcgtgtcaacttccttgcggccaatatccacttcccgctgcgaaaaatgacaggatcgggcatctatgcacctgattctccacgcgcctttcattacgatatgaagaccaaggaaggaaaactcctcttgtcacaattggattcccacccgcaccgcccagcagtgaattggacgtcttatgcctcaggtttgccagcccccttggtgggtaaccaggaattcaaatccattgtcttttttgacgagttcaccttcttcgagcttaaaggtgttgcgggaaactacacagtttgccagaaggatctctgctgccagctttcttaccggatgcttgagatgcgtgaagacgaagtttatgcgctcggcgcattcgatggacttcatactgtggaaggtagctattacttgcagatttgcacattgctgaagtgtaaaaccatggacctgcactcctgtggtgactcggtcgaaactgcttccacccgctttgaaatgttctccctcagcggcacgtttggcacccagtatgtgttccctgaggtgttgctgtcggacattcagcttgcgcctggagaatttcaggtttcatccgatggccgcttgttcagcctgaagccaccgtctggaccggtccttactgtcactctgtttggccgcctctacgagcgtgattctgcatcaggtgcttcagcagacctcgtggcacaaggactgcgagtcatgctcggtgttatcattactatcatgtactccttgtcctggtaa

SEQ ID NO: 99 - DNA, 1599 bp, Synthetic construct
  source 1..1599: mol_type = other DNA
atggccaagcgcgttgcaattgttggagctggtgtctcaggcctggcctcgatcaagtgctgcctggaagagggcctggagcccacctgcttcgagcgttccgatgaccttggcggcctgtggcgattcaccgaacacgttgaagaaggtcgcgcctctctctacaagtctgtggtttccaactcctgcaaggagatgtcttgttacccagacttcccattcccagaagattaccctaactacgtgccaaattctcacttcctggaatatctccgaatgtatgcaaaccagttcaaccttctgaagtgcattcaattcaagaccaaagtctgctcggtaaccaaacatgaggactttaataccaccggtcaatgggatgtagtcactctgtgtgagggaaagcaagagtcagccgtcttcgatgcggtcatggtctgtactggttttctcacgaacccatatttgcctttggactccttcccgggcatcaatacttttaaaggccagtatttccacagccgtcagtacaagcacccggatatttttaaggacaagtctgttcttgtggttggtatgggtaactcgggcacagacattgctgtggaagcgagccacctggcaaaaaaggtgttcctcagcaccacgggcggagcatgggtgatcagccgtgtctttgactcaggctacccgtgggacatggtgttcatgacacgctttcagaacatgttccgcaattcgctccctaccccaattgtgaattggttgattgcaaaaaagatgaacagctggttcaatcatgcgaattatggcctgatccccgaagaccgcatccaactgcgtgagcctgtgctgaacgatgagctcccaggtcgtatcatcactggaaaagtgcttatcaagccatccatcaaggaagtgaaggaaaactccgtggttttcaacagctccccagaggaagagcccattgatatcattgtctttgccactggatacacctttgctttcccgttccttgatgagtccgtagtgaaggttgaagatggccaggcatcactttacaagtacatcttccctgcacatctgcagaagccaactctggctgttattggcctcatcaaacccttgggctccttgttgcccaccggcgacacccaagctcggtgggcggttcgggtccttaaaggtgtgaacaagttgccgccttcctcggtcatgatccaggaagttaatacacgcaaagaaaacaagccctcaggatttggtttgtgttactgcaaagctctccaatccgattatatcgcatacatcgatgaactgctgacctacatcgatgcgaagcctaacatgttttctctgctcctgacggatccacacctggcgttgaccatcttctttggcccgtgcacaccttaccagttccgcctgaccggtccgggaaaatgggaaggtgcccgaaacgctatcatgacccagtgggaccgcaccttcaaggtcaccaaaacgcgcattgtaaaagagtccccatctccctttgcatccctcttgaaactctttagctttcttgcgttgcttgtggccattttccagattttcctctaa

SEQ ID NO: 100 - DNA, 1563 bp, Bos taurus
  source 1..1563: mol_type = genomic DNA
atgatcacttcctctttacaaacctctgtggcagtttttgccctagtaattctacatgtcagcactctggatacttttaaagctgcagtgtatgaacatgctgtcatactgccaaatgacacagaaacacctgtttctccggatgaggccttgttcctcatgaacaagaacatagatattctggagaaggcaattaagcaggcagctgagcagggtgctcagattatcgtgaccccagaagatgcactttatggatggaaatttaccagggaaaccattttcccttatctggagaatatcccagatcctcaggtgaactggattccatgtcaagaaccccacagatttggtcatacaccagtacaagcaagactcagctgcctggccaagaacaactctatctatgtcgtggcaaatatgggtgacaaaaagccatgtaattcccataataccacttgtccttctaatggccattatcaatacaacaccaacgtggtgtatgatgcaaagggaaagctggtggcgcgttaccataagtaccacctctaccgtgagattcagtttgatgtccccgaaaagccagagttggtgactttcaacaccacctttggaaagtttggcattttcacgtgctttgatattctcttccatgatcctgctgtgaccctggtgaaagattcccacgtggacaccatattgtttcccacagcttggatgaacgttttgccctttatgacagctattgaatttcattcggcttgggcaatgggaatgagagttaatcttcttgcagcaaacacacataatgtcagcctaaaaatgacaggcagtggtatctatgcaccacactctcccaaagtctaccatcatgatatggaaacagagtcaggaaagatcctctttgcagaggtggattcccatccccgaaactcctcgacctacccaccagccatgaattggagtgcctacgccacctccatcacaccctttccaggacctaaaaattccttcaggggatttatttcccgtgatgagttcaactttacaaaactctctgaaagtgcaggaaaccttacagtctgtcaaaaagagctctgctgtcatttaagctacagaatgttagcaaaggagggggatgaagtgtatgttctaggagcttttgcaggacttcatgggagaaggagaagagaatattggcaggtatgcacaatgctgaaatgcagatctactgatgtgacaacttgtggacagccagtagaaactgctttgacgagatttgaaatgttctctctaagtggcacatttggaacagagtatgtttttcctgaagtgctacttactaaaatcaagctggcacctgaaaaatttgaggtactagaagatgggcgtttggtaaacaagaatggattatctgagcctgtcctaacagtgtcgctctttgggagatggtacaccaaggacgcacattccatctcaggcgggaccagcaactcagcaacagcgtacctgatgatacccacattattaatgatcataaccttgcgggatgctgtgatggtgtag

SEQ ID NO: 101 - DNA, 1563 bp, Ovis aries
  source 1..1563: mol_type = genomic DNA
atgatcacttcctctttacaaacctctgtggcagtttttgccctaataattctacatgtcagtgctctggatacctttaaagctgcagtgtatgaacatgctgtcatactgccaaacgacacaaaaacacctgtttctccggatgaggctttgttcctcatgaacaagaacatagatatcctggagaaggcaattaagcaggcagctgagcagggtgctcagatcattgtgaccccagaagatgcactttatggatggaaatttaccagggaaaccattttcccttatctggagaatatcccagatcctcaggtgaactggattccatgtcaagacccccacagatttggtcatacaccagtacaagcaagactcagctgcctggccaagaacaactctatctatgtcgtggcaaatatgggtgacaaaaagccatgtagttcccgtaataccacgtgtccttctaatggccattatcagtacaacaccaacgtggtatatgatgcaaagggaaagctggtggcgcgttaccataagtaccacctctaccatgagcttcaatttgatgtccccgaaaagccagagctggtgactttcaacaccgcctttggaaagtttggcatcttcacgtgctttgatattctcttccatgatcctgctgtgaccctggtgaaagatttccacgtggacaccatattgtttcccacagcttggatgaacgttttgccctttttgacagctattgaatttcattcggcatgggcaatgggaatgagagttaatcttcttgcagcaaacacacataatgtcagcctgaaaatgacaggcagtggtatctatgcaccacactctcccaaagtctaccatcatgatatggaaacagagtcaggaaagatcctctttgcagaggtggattcccatccccgaaactcctcgacctccccaccagccgtgaactggagtgcctacgccacctccatcacaccctttccaggacctaaaaattccttcaggggatttatttcccgcgatgagttcaacttcacaaagctctctgaaagtgcaggaaaccttacagtctgtcaaaaagagctctgctgtcatttaagctacagaatgttaggaaaggaggaggatgaagtgtatgttctaggagcttttgcaggacttcatgggagaaggagaagagaatattggcaggtatgcacaatgctgaaatgcagatctactaatttgacaacttgtggacagtcagtagaaactgctctgacgagatttgaaatgttctctctaagcggcacatttggaacagagtatgtttttcctgaagtgctacttactaaaatcaagctggcacctgaaaaatttgaggtactagaagatgggcgtttggtaaacaagaatggattatctgaacctgtcctaacagtgtcgctctttgggaggtggtacaccaaggacatacattccatctcaggcgggaccagcaactcagcaacaacgtacctgatgatacccacattattaatgatcatagtcttgcagtatgctgtgatggtgtag

SEQ ID NO: 102 - DNA, 1563 bp, Balaenoptera sp.
  source 1..1563: mol_type = genomic DNA
atgatcacttcctcttttcaaacttccgtggcagttttggccctaattattctgcatgtcagcgctttggatactttcaaagctgcagtatatgaacatgcggtcatattgccgaatcacacagaaacacgtgtttctcaggatgatgccttgctcctcatgaacaagaatatagatattctggagatagcaattaagcaggccgctgaacagggtgctcagatcattgtgactccagaagatgcaatttatggatggaaatttaccagggaagccattttcccttatctggaggatattccagatcctctggtgaattggattccatgtcaagacccacacagatttggtcgtacaccagtacaagcaagactcagctgcctggccaagaacaactctatctatgtcttggcaaatattggggacaaaaagccatgtaattcctgtgactccacgtgtcctcctaatggccattatcaatacaacaccaacgtggtgtatgatgcaaagggaaagctggtggcacgttaccataagtacaacctctactctgagcttcagtttgatgtccccaaaaagccggagttggtgactttcaacacctcctttggaaagtttggcattttcacatgctttgatattctcttccatgatcctgctgtgaccctggtgaaagatttccacgtggacaccatattgtttcccacagcttggatgaatgttttgccctttttgacagctattgaatttcattcagcttgggcaatgggaatgagagttaatcttcttgtggccaacacacataatgtcagcctaaagatgacaggcagtggtatctatgcaccacactcgcccaaagtatatcattatgatatggaaacagagctgggaaagatcctctttgcagaggtggattcgcatccccgaaactctctgacctacccaccagctgtgaattggagtgcctacgccaccaccatcaaaccattcccaggacagaaaaactcctttaggggctttatttcccgggatgagttcaacttcacagaactctttgaaaatgcaggaaaccttacagtctgtcaaaaagagctgtgctgtcatttaagctacagaatgttaggaaaagaggagaatgaagtgtatgttctaggagcttttgctggacttcatggccgaaggagaagagagtattggcaggtatgcacaatggtgagatgtaaatctactaatctgacaacttgtggacagccagtagacactgctctgacaagatttgaaatgttctccctaagtggcacatttagaacggagtatgttttccctgaagtgctacttactaaaatcaatctggcacctggaaaatttgaggtactaaaagatgggcgtttggtaaacaagaatggatcatctgagcctatcctaacagtgtcactgtttgggagatggtacactgaagacgtacgttccatctcaggcaggaccagcaactcagcgacaacttacctgataatatccacattattaatgatcatagctttgcaaaatattgtgatggtgtag

SEQ ID NO: 103 - DNA, 1566 bp, Sus scrofa
  source 1..1566: mol_type = genomic DNA
atgatcgcttcctcttttcaaactacgttagcagtttttgccctaattatcctgcatgtccgtgcactggatacttttaaggctgctgtatatgaacatgctgtcatcctgccaaatgacacaaaaacacctgtttcttgggatgatgctttgctcctcatgaacaagaatatggatattctggagagagcaattaagcaggcagctgagcagggttctcaaatcattgtgactccagaagatgcactttatggatggaaatttaccagggaaaccatttttccctatctggaggatgtcccagatcctcagaagaactggattccatgtcaagacccccgcagatttggtcacacaccagtgcaagtaagactcagctgcctggccaagaataactctatctatgtcttggcgaatattggagacaaaaagccctgtaattcccatgattccaaatgtcctcctaatggctattatcaatacaacaccaacgtggtatatgattcagagggaaagctggtggcacgttaccataagtaccacctctaccatgagcttcagtttgatgtccctgaaaagccggaggtggtgactttcaacaccccttttggaaagttcggcattttcacgtgcttcgatattctcttccgtgatcctgctgtgacgctggtgcaagatttccacgtggacaccatattgtttccggcagcttggatgaatgtgttgccccttttgacagctattgaattccattcggcttgggcaatgggcatgagagttaatcttcttgcagccaacgtacatcatgtccgcctaaatatgacaggcagtggtatctatgctccacattctcccaaagtataccattatgacatggaaacggactcgggaaaaatcctccttgcggaggtggatgcgcatccccgcacctccccgacctacccaacagccgtgaactggggtgcctacaccctaagcatccagccgttcccaatactgcaaaactccttcaggggattcatttcgcgggatgagttccacttcacagaactctctgaaagtgcaggaaaccttacggtctgtcataaagagctctgctgtcatttaagctacagaatgctagcgaaggaggagaatgaagtgtatgttctaggagcttttacaggacttcatggccgaaggaganaggagtattggcaggtatgcacaatgttgaaatgcaaatctactaatctgacaacttgcggacagcctgtagaaactgctctgacgagatttgagttgttctctctaagtggtacatttggaacagagtatgtttttcccgaagtgctccttactaaaatcaatctggcacctggaaaatttgaggtactgaaagatgggcgtttggtaaacaagaacggaacatctgagcctgtcctaggagtgtcactgtttgggagatggtacattagggatgtaccactttccatctcgggaggaccagcacctcagcaaaaaatgtacctgataatttccgcattattaatgatcatagctttgcaaaatattgtcatggtatag

SEQ ID NO: 104 - DNA, 1563 bp, Echinops telfairi
  source 1..1563: mol_type = genomic DNA
atgactgctgcttctttccaaacctccgcagcagtgtttgccctacttaccctgcatgtgggtgccctgaatactttcacagccgcggtgtacgaacatgccgtcatactgccaaataaaacactgacaccggtttctcagcaagatgccttggccctgatgaacaagaatatagatatcctggaggaagttattaagcaggcagctgagcagggcgctcaaatcattgtgactcctgaagatgctctctatggatgggaatttggatgggaaactgtcttcccttttttggaggatgtcccagcccctgaggtggactggattccatgccgagaccctcacagatttggtcacacaccagtacaaacaagactcagttgcatggccaagaacaactctatctatgtcgtggcaaatattggggacaaaaagaagtgtaactcccaggactccacctgcccttccactggctattttcagtacaataccaatgtggtgtacaacgcggaggggaaactggtggctcgttatcataagcaccacctctacggtgagtttcaatttaatgccactagaaagccagaagtggtgacattcgacaccacctttggaaagtttggtattttcacctgctttgatatactcttccgtgagcctgctgtgcgactggtggaagagtcccaagtcgacaccatattgttcccaacagcttggatgaatagcttaccctttctgacagctattgaatttcattcagcttgggcaatgcaaatgagagtgaatcttcttgctgctaacctacatcataccagtaaaaggatgacagggagtggcatctatggcccgaagggccccagcgtgtatcactatgatatggaaacggcatcgggacagctcctcctctcccaggtggactcccacccccggcgctcccctgactaccccgcagctgtcgactgggacgcctatgccaggagcatcaaaccattcccaacacagaagcatgcctttgggggcaacgttcacgctgaccagttcaacttcaccgagctgtatgaacgtgcaggaaaccttactgtctgccagaaagagctctgctgtcatttaagctacagaatgttaggaaaagaagagagtgaagtctatgttctaggagctttcacgggactccacgggaaaagggcgaaagagtactggcaggtctgcacgttgctgaaatgcaaaacgactatcttgacatcttgtggagaggtggtagaaactgcttctacaagatttgaaatgttctctctaagtggcaccttcgaaaccgagtatgtttatcccgaagtgttacttagtaaaattcagctgtcacctggaaaatttgaggtactaaaagatggacgtttggtagacaagaatgggacatctggacctattgtaaccgtgacactgtttggaaggtggtacgaaagggacagacttcagaattcagccaagaccagcaattcagcaatacacttcctactgatcgccacatcattcatgatcacagctttgcaaaatattgccatgacatag

SEQ ID NO: 105 - DNA, 1563 bp, Oryctolagus cuniculus
  source 1..1563: mol_type = genomic DNA
atgatcacttcctcttttccaacccgcgtggcagtttttgccctgatcagcctgcgtgtcggtgctctggacacttttgtagctgcagtgtatgaacatgccgtcgttttgccaaagaaaacagaaacgcctgtttctcaagacgatgccttgcttctcatgaacaagaatatagatattctggagagtgcgattaagcaggcagcagggaagaatgctcgaatcattgtgactccagaagatgcactttatgggtggaaatttaccagagaaacaattttcccttatctggaggatatcccagatcctcaggtgaactggatcccatgtcaagacccccacagatttggtcacacaccagtacaagcaagactcagctgcctggccaaaaacaattctatctatgtcttggcaaatattggagacaagaagccttgtaattcccatgactccacatgtcctcataatggctattatcaatacaataccaatgtggtgtacgattcagagggaaaactggtggcacgttatcataagtaccacctatactctgaacctcagtttgatgttcctgagaaaccagagctggtaactttcaacaccacatttggaaagtttggcattttcacatgttttgatatactcttccatgatcctgctgttaccttggtgaaagatttccatgtggacaccatattgctccctacagcttggatgaatgttctgcccctcttgacagctattgaattccattcagcttgggccatgggaatgagagttaatcttcttgtggccaatacacatcatgtcagcttaaatatgacagggagtggcatctatgtaccacattctcccaaagtgtaccactatgacatggaaacagacttgggaaagctcctcctttcagaggtggattcacatccccaaacctcttcttcctacccaacagctgtgaattggagtgcctatgccatggccatcagaccattctcagtacagaaagacactttcagaggatttatttctcgggacgagttcaattttacagaactttttgaaaatgcaggaaatattacaatttgtcaaaaagagctctgctgtcatttaagctacagagtgctagaaaaagaagcagatgaaatttatgtcctaggagcctttacaggatttcatggccgaaggaggagagaatactggcaggtatgcacaatgttgaaatgtaaaactactaatctgaccacatgtggacggccagtagagactgcttctacacgatttgaaatgttctccctcagtggcacatttggaacagagtatgtttttcctgaagttctactcactggaattcatctatcacctggaaaatttgaagtgctaaaagatgggcgtttggtaaataagaatggattatctgggcctatcctaacagtgtcactgtttgggaggtggtacacaaaggacacactttacaaatcatctgggaccaacaattcagcaataccttacctgctaatcttcattgtcctaatgactgcagattttcaaaatattgtattgctgtaa

SEQ ID NO: 106 - DNA, 1563 bp, Lepus europaeus
  source 1..1563: mol_type = genomic DNA
atgatcacttcctcttttccaacccatgtggcagtttttgccctgatcagcctgcgcgtcggtgctctggacacttttgtagctgcagtgtatgaacatgccgtcattttgccaaagaaaacagaaacgcctgtttctcaagacgatgccttgctcctcatgaacaagaatatagatattctggagagtgcgattaagcaggcagcagggaagaatgctcgaatcattgtgactccagaagatgcactttatgggtggaaatttaccagagaaacgattttcccatatctggaggatatcccagaccctcaggtgaactggatcccatgtcaagacccccacagatttggtcacacaccagtacaagcaagactcagctgcctggccaagaacaactctatctatgtcttggcaaatattggagacaagaagccttgtaattcccatgactccacatgtcctcataatggctattatcaatacaataccaatgtggtgtacgattcagagggaaaactggtggcacgttatcataagtaccacctatactctgagcctcagtttgatgttcctgagaaaccggagttggtaactttcaacaccacatttggaaagtttggcattttcacatgttttgatatactcttccatgatcctgctgttaccttggtgaaagatttccatgtggacaccatattgttccccacagcatggatgaatgttctgcccctcttgacagctattgaattccattcagcttgggccatgggaatgagagttaatcttcttgtggccaatacacatcatgtcagcctaaatatgacagggagtggcatctatgcaccacattctcccaaagtgtaccactatgacatggaaacagacttgggaaagctcctcctttcagaggtggattcacatcccaaaacctcttcttcctacccaacggctgtgaattggagtgcctatgccatggccatcagaccattctcagtacagaaagacactttcagaggatttatttctcgggacgagttcaattttacagaactttttgaaaatgcaggaaatattacaatttgtcaaaaagagctctgctgtcatctaagctacagaatactagaaaaagaagcagatgaaatttatgtcctaggagcctttacaggatttcatggccgaaggaggagagaatactggcaggtatgtactatgctgaagtgtaaaactactaatctgaccacatgtggacagccggtcgagactgcttctacacgatttgaaatgttctccctcagtggcacatttggaacagagtatgtttttcctgaaattctactcactgaaattcatctatcacctggaaaatttgaagtgctaaaagatgggcgtttggtaaataagaatggattatctgggcctatcctaacagtgtcactgttcggaaggtggtacacaaaggacacactttacaaatcatctgggaccagcaattcagcaataccttacctgctaatcttcattgtcctaatgattgcagatttgcaaaatattgtattgctgtaa

SEQ ID NO: 107 - DNA, 1548 bp, Dasypus novemcinctus
  source 1..1548: mol_type = genomic DNA
atgatcacgtcaccttttctaccttcggcagtagttttggccctaatcattctgaatgtcggtgcactgggcacttttaaagctgccgtgtacgagcatgctgtgatattgccaaaccaaacactcagagttgtttctcaggaagatgcgctgctcttgatgaacaagaatctagatatcctggaggcagagataaagcaggcagctcagcagggtgctcaaatcattgtgactccagaagatgccctttatggttggactttcaccagggagactgttttcccttatctggaggacatcccagacccccaggagaactggattccatgtcagaacccccagagatttggtcacacaccaatacaaaaaagactcagctgcctggccaaggacaactctatatatatcctggcaaatattggggacaaaaagcagtgcgattcccggaactccgcctgtcctcgcaatggttattatcaatacaataccaacgttgtgtatgataaagagggaaaactggtggcgcgttaccataagtaccatctctacaatgagcctcagtttaacgcccctaaaaggccggaattggtgaccttcaacactgattttggaaagtttggcattttcacgtgctttgatatactctttaaagagcctgcagtgaccctggtggaagaattacaagtggacaccatactgtttgccacagcttggatgaatgtcttgccctttttgacagctattgaattccactcggcttgggcaatgggaatgagagtcaatgttcttgcagccaacatacacaatcccagcctaaatatgacagggagtggtatctactcaccacattcccccaaagcataccattatgatatgaaaacaaagtttggaaggctccttctttcagagttggattcacatccccgaggttctgtatcttacccaccagctgtgagctggacaacctacgccaagtccatcaaaccattcccagtacagaaacccgccttcctgagaagtatttccagagacgagttcaacttcacggaactttttgaaagtgcaggaaatcttacagtctgtcaaaaagagctctgctgtcatttaagctacagaatgttagaaaaagacacaaaagaatcctacgttctcggagcatttacaggacttcacggcagaaggcaacaagagtactgggaggtatgtacaatactgaaatgtgaagctaataatttcatgacttgtggacggacaggggaaactgcttctacaagatttgaaatgttctccctaagtggcacatttaaaacagagtatgtctttcctgaagtgttacttgataaaattcacctgtcacccggaaaatttgaggtactaaaggatggccgtttggtaaacaagaatgggacatctgggcagatcctaacagtgtcactgtttggcaggtggtatgccaaggatgagctttccaactcaggcaagacccacagttcagcagtgccaacgctggcattagtaatgatagtagctttgatgtatgatgtgtga

SEQ ID NO: 108 - DNA, 1404 bp, Camelus dromedarius
  source 1..1404: mol_type = genomic DNA
atgaacaagaatatggatattctggagaaagcaattaagcaggcagctgagcagggtgctcaaattattgtgactcctgaagatgccatttatggctggaaatttacccgggaaaccattttcccttatcttgagtatatcccagaccctcaggtgaactggattccatgtcaacaaccccacagatttggtcacacaccagtacaagtaagactcagctgcctggccaagaacaactctatctatgtcttggcgaatattggggacaaaaagccatgtaattccaggggctccacgtgccctcctagtggctattaccaatacaacaccaacgtagtgtatgatacacagggaaagctggtggcacgttaccataagtaccacctctactctgagcctcagtttgatgtccctgaaaagccagaggtggtgactttcaacaccaccttcggaaggtttggcattttcacgtgcttcgatatactcttccatgatcctgctgtgaccctggtgaaagatttccaggtggacaccgtattgtttcccacagcgtggatgaatgttttgccccttttgacagctattgaattccattcagcctgggctatgggcatgggagttaatcttttggtggccaacacacatcacgtcagcctaagaatgacaggcagcggtatctatgcaccacactctcccaaggtctattattatgataaggaaacagagttgggaaagcttctccttgcagaggtggactcgcatccccgaatctctccgacctacccaacagccgtgaattggagcgcctacgccacaaccatcaaaccattcccagtacagaagcacactttcgggggatttatttcccgggatgagttcaacttcacagagctctttgaaaatgcaggaaacctgacagtctgtcaaaaagagctctgctgtcatttaagctacagaatgctgggaaaagaggagaatgaagtgtatgtgctaggagcttttacaggacttcatggccgaaggagaagtgaatattggcaggtatgcaccctggtgaaatgcaaatctactaacttgacgacttgtggacagccagtagaaactgctctgacaagatttgaaatgttctccctaagtggcacatttggaacagagtatgtttttcctgaagtgctacttactaatatccatctggcacctggaaaatttgaggttctaaatgatgggcgtttggtaaacaagaatggattatctgagcctatcctaacagtgtcactgtttgggaggtggtacactaaagacgcactctccatctcaggtgggaccagcaacgcagcaacaacttacctgttaatattcacatcattaatgatcatagctttacaaaatactgtgatggtataa

SEQ ID NO: 109 - DNA, 1380 bp, Homo sapiens
  source 1..1380: mol_type = genomic DNA
atggtcacttcctcttttccaatctctgtggcagtttttgccctaataaccctgcaggttggtactcaggacagttttatagctgcagtgtatgaacatgctgtcattttgccaaataaaacagaaacaccagtttctcaggaggatgccttgaatctcatgaacgagaatatagacattctggagacagcgatcaagcaggcagctgagcagggtgctcgaatcattgtgactccagaagatgcactttatggatggaaatttaccagggaaactgttttcccttatctggaggatatcccagaccctcaggtgaactggattccgtgtcaagacccccacagatttggtcacacaccagtacaagcaagactcagctgcctggccaaggacaactctatctatgtcttggcaaatttgggggacaaaaagccatgtaattcccgtgactccacatgtcctcctaatggctactttcaatacaataccaatgtggtgtataatacagaaggaaaactcgtggcacgttaccataagtaccacctgtactctgagcctcagtttaatgtccctgaaaagccggagttggtgactttcaacaccgcatttggaaggtttggcattttcacgtgctttgatatattcttctatgatcctggtgttaccctggtgaaagatttccatgtggacaccatactgtttcccacagcttggatgaacgttttgccccttttgacagctattgaattccattcagcttgggcaatgggaatgggagttaatcttcttgtggccaacacacatcatgtcagcctaaatatgacaggaagtggtatttatgcaccaaatggtcccaaagtgtatcattatgacatgaagacagagttgggaaaacttctcctttcagaggtggattcacatcccctatcctcgcttgcctacccaacagctgttaattggaatgcctacgccaccaccatcaaaccatttccagtacagaaaaacactttcaggggatttatttccagggatgggttcaacttcacagaactttttgaaaatgcaggaaaccttacagtctgtcaaaaggagctttgctgtcatttaagctacagaatgttacaaaaagaagagaatgaagtatacgttctaggagcttttacaggattacatggccgaaggagaagagagtactggcaggtctgcacaatgctgaagtgcaaaactactaatttgacaacttgtggacggccagtagaaactgcttctacaagatttgaaatgttctccctcagtggcacatttggaacagagtatgtttttcctgaagtgctacttaccgaaattcatctgtcacctggaaaatttgagcaaacctga

SEQ ID NO: 110 - DNA, 1560 bp, Globicephala melas
  source 1..1560: mol_type = genomic DNA
atgatcatttcctcttttcaaacctccgtggcagtttttgcgctaattattctgcatgtcagtgctttggctactttcaaagctgcagtatatgaacatgctgtcatattgccaaatgacacagaaacacccgtttctcaggattatgccttgctcctcatgaacaggaatatagatattctggagagagcaattaagcaggctgctgaacagggtgctcagatcattgtgactccggaagatgcaatttacggatggaaatttaccagtgaagccattttcccttatctggaggatattccagatcctctggtgaattggattccatgtcaagacccacacagatttggtcatacaccagtacaagcaagactcagctgcctggccaaggacaactctatctatgtcgtggcaaatatcggggacaaaaagccatgtcattcccgtgactccgcgtgtcctcctaatggccattatcaatacaacaccaacgtggtgtatgatgcaaatggaaagctggtggcacgttaccataagtacaacctctactctgagcttcagtttgatgtccccgaaaagccagagctggtgactttcaacacctcctttgggaagtttggcattttcacatgctttgatatcctcttccatgatcctgctgtgaccctggtgaaagatttccacgtggacactatattgtttcccacagcttggatgaatgttttgccctttttgacagctattgagtttcattcagcttgggcaatgggaatgagagttaatcttcttgtggccaacacacataatgtccgcctaaaaatgacaggcagtggtatctatgcaccacactctcccaaagcgtaccattatgatatggaaacagagttgggaaagatcctctttgcagaggtggattcgtatccccgaaactctccaacctacccaccagctgtgaattggagtgcctacgccaccaccatcaaaccattcccaggacagaaaaattcctttaggggctttatttcctgggatgagttcaacttcacagaactctttgaaaatgcaggaaaccttacagtctgtcaaaaagagctgtgctgtcatttaagctacagaatattaggaaaagagaatgaagtgtatgttctaggagcttttgctggacttcatggcagaaggagaagagagtattggcaggtatgcacaatggtgaaatgtaaatctactaatctggcaacttgtggacagccagtagacactgctctgacaagatttgaaatgttctccctaagtggcacatttagaacggagtatgtttttcctgaagtgctacttactaaaatcaatctggcacctggaaaatttgaggtactaaaagatgggcgtttggtaaacaagaatggatcatctgagcctatcctaacagtgtcactgtttggcagatggtacactaaagacgtacgttccatctcaggcgggaccagcaactcagcaacgacttacctgataatatccacattattaatgatcatagctttgcaaaatattgtgatggtgtag

SEQ ID NO: 111 - DNA, 1542 bp, Pteronotus mesoamericanus
  source 1..1542: mol_type = genomic DNA
atgcaatttcatttctgggagagaggccaggctaggacgtccttcaggctgtggcagagcccaaggctcctcctcaccatcctgagttcctcggtgtctttgtcatgtctccattgcgtggttgacgtgaatctgctgtgtccttctcggcaagatccaggagttggtgcccaggagaaccttgtgaaggatataaagcagggtgctcaaattattgtgactccagaagacgcaatttatggatggaattttaccagggaaaccattttcccttacctggaggatatcccagaccctcaggtggactggattccatgtcaagacccccacagatttggtcacacgccagtacaagaaagactcagctgcctggccaagaacaactctatctatgttttggcaaatattggagacaaaaagccatgtaattctaatgacttcacatgtcctcctaatggctattatcaatacaatactaatgtggtatatgataaaaatggacacctggtggcacgttaccacaagtaccacctctattctgaacctcagtttgatgtgcctgaaaagccggagctggtgactttcaacaccacctttggaaggttgggcattttcacctgcttcgatatactcttccacgatcctgctgtgaccctggtgaaggatctccacgtggacaccatactgtttcccacagcttggatgaatgtcttgccccttttaacagctattgaattccattcagcttgggcaatgggaatgagagtcaatcttcttgtggccaatacacatcatgtccgcctaaatatgacaggcagtggcatttatgcaccacagtctcccaaagtataccactacgatatggacacggagctgggaaagatccttctttcagaagtggattcgcatcctcgaagctcacctgcctacccagcggctgtgaactggagtgactatgccacgaccatcaaaccattcccagtacagaaaaacactttcaggggatttatttcccgggatgcgttcaacttcacagaactttctgcaattgcaggaaaccttacagtctgtcaaaaagatctctgctgtcatttaagctatggaatgttaagaaaagaagagaatgaagtatacgctctaggagctttctcaggacttcatggccgaaggagaagagagtattggcaggtatgcacaatgctgaaatgcaaaacaactaatttgacaacttgtggacaaccagtagaaactgctgtgacaagatttaatatattctccctaagtggcacatttggaacagagtatgtttttcctgaagtgctacttactaaaattcatctgtcacctggaaaatttgaggtactaaaagatgggcgcttgataaacaagaatggaacatctgagcctatcctaacagtgacactctttgggaggtggtacgctaaggactcacattccgtctcaggagggatcagcaatttggaaacaacttacctgttaatatccactttatcaatgatcatagctttacaaatattgtga

SEQ ID NO: 112 - DNA, 1554 bp, Equus caballus
  source 1..1554: mol_type = genomic DNA
atgattgcttcctcttttcgaacctctgcggctgtttttgccgtaattatcctgcatgttggggccctggatactttcatagctgcagtttatgaacacgccgtcatactgccaaaaacaacagtgacacctgtctctcaggacgatgccttgctcctcatgaacaagaatatagatattctggagagagcgattaagcaggcagcaaagcagggtgctcaaatcattgtgactccagaagatgccctttatggttggacatttaccagggaaaccattttcccttatctggaggacgtcccggaccctcaggcgaactggattccgtgtcaggactccaacagatttggttacacaccagtacaagtaagactcagctgcctggccaagaacaactcaatctatgtcttggcaaatatgggggacaagaagccatgtaatcgccataactccatgtgtcctcccaatggctattatcaatacaacaccaatgtggtatatgatgcggaggggaaactggtagcacgctaccataagtaccacctctactctgagcctcagtttgatgtccctgaaaagcccgagttggtgactttcaacaccacctttggaaggtttggcattttcacgtgctttgatattttcttccacgatcctgctgtcaccctggtgaaagatttccatgtggataccatcctgtttcccacagcttggatgaatgtcttgccccttctgacagctattgaattccactcggcttgggctgtgggcatgagagttaatcttcttgcggccaacacccacaatatccacatgaatatgacaggcagcggtatctatgcaccacagtctcccaaagtataccaccatgacatggagacagagtcgggaaagctcctccttgcggaagtggattcgcatccccgagactctcctgcctacccaacagctgtgaactggagtgcccatgccacaaccatcagaccattcccaggaaagaaaaacaccttccgggcatttatttcccgggacgctttcaacttcacagaactttttgaaaatgcaggaaaccttacagtctgtcaaaaagatctctgctgtcatttaagctacaggatgttaggaaaggaagaggatgaagtgtatgctctgggagctttttcaggacttcacggccgaaggagaaaagagtactggcaggtgtgcacaatgctgaaatgcaaaactactaatttgacaacttgtgggcggccggtagaaactgcttctacaagatttgaaatgttctccttaagtggcacatttggaacagagtatgtcattccagaagtgctacttactaaaattcacctgtcacctggaaaatttgaggtactacaagatgggcgtttggtgaacaagaacggaccatccgagcctatcctgacagtgtctctctttgggagatggtatgctaaggattcatcttccatcccaggcggaaccagcaattcagcaacaacttacctgctgatacccattttattgatgatcatagcttggcagatactgtga

SEQ ID NO: 113 - DNA, 1332 bp, Jaculus jaculus
  source 1..1332: mol_type = genomic DNA
atgggcgctcagatcattgtgactccagaggatgcactttatggatggaagtttactagggaaagcattttcccttacttggaagatgtccccgaccctcaggtggactggattccatgtcgagatccccacagatttggtcacacacccatacaagaaagactcagctgcctggccaagaacaactctatctatgtcgtagcaaatattggagacagaaagccatgcaagtcccatggatgccctcataatggctactatcagtacaatactgatgtggtatacgataaagagggaaaactcgtggcacgctatcataagtaccacctctactcagagattcagtttgatatccccgaaaaaccagagttggtgactttcaacaccacatttggaaggtttggcattttcacgtgctttgatatcctcttccatgaccccgccgtgaccctagtgagagatttccacgtggacaccatactgtttcccacagcatggatgaatgttttgccccttttgacagctattgaattccattcagcatgggcaatgggcatgggagttaatcttcttgcagctaacctacatcatgtcaggctaaatatgacagggagtggtatctatgcaccacactctccaaaagtgtaccactatgacatggaaacagacttgggaaagatgctcttggcagaactcgattcccatccccggtcttctcctagctacccagtagcagtgaattggagtacctacgccgtggctcaagagccattcccgctacagagaaaagcttttggcggatttatttcccgggatgagttcaatttcacagaactttttgaagatgcaggaaatcttacagtctgccaaaaagaattatgttgtcacttaagctacagaatgttaggaaaagaagagcatgaagtgtatgttgtgggagctttcacgggacttcatggccgaaggagaagagagtactggcaggtatgcacaatgctgaagtgtaaatcggctagtctcaagacttgtggacaaccggtagaaaccgcttccacgagatttgatatgttttccctcagtggtacattcggaacagagtatgtttttcctgaagtgctcctttctaaagtgcatctgtcccctggaaatttcgaggtgctggaagatgggcgtttgacaaacaagaatgggacacctggaccgatcttaaccgtgtctctgtttgggaggtggtacacgaaggattccctttacagctcatgtatgaccaacaatcccatgttaatttacctgctcatttccacactattaatgatgtttaaaatattgtaa

SEQ ID NO: 114 - protein, 520 aa, Bos taurus
MITSSLQTSVAVFALVILHVSTLDTFKAAVYEHAVILPNDTETPVSPDEALFLMNKNIDILEKAIKQAAEQGAQIIVTPEDALYGWKFTRETIFPYLENIPDPQVNWIPCQEPHRFGHTPVQARLSCLAKNNSIYVVANMGDKKPCNSHNTTCPSNGHYQYNTNVVYDAKGKLVARYHKYHLYREIQFDVPEKPELVTFNTTFGKFGIFTCFDILFHDPAVTLVKDSHVDTILFPTAWMNVLPFMTAIEFHSAWAMGMRVNLLAANTHNVSLKMTGSGIYAPHSPKVYHHDMETESGKILFAEVDSHPRNSSTYPPAMNWSAYATSITPFPGPKNSFRGFISRDEFNFTKLSESAGNLTVCQKELCCHLSYRMLAKEGDEVYVLGAFAGLHGRRRREYWQVCTMLKCRSTDVTTCGQPVETALTRFEMFSLSGTFGTEYVFPEVLLTKIKLAPEKFEVLEDGRLVNKNGLSEPVLTVSLFGRWYTKDAHSISGGTSNSATAYLMIPTLLMIITLRDAVMV

SEQ ID NO: 115 - protein, 520 aa, Bos javanicus
MITSSLQTSGAVFALIILHVSTLDTFKAAVYEHAVILPNDTETPVSPDEALFLMNKNIDILEKAIKQAAEQGAQIIVTPEDALYGWKFTRETIFPYLENIPDPQVNWIPCQEPHRFGHTPVQARLSCLAKNNSIYVVANMGDKKPCNSRNTTCPSNGHYQYNTNVVYDAKGKLVARYHKYHLYREIQFDVPEKPELVTFNTTFGKFGIFTCFDILFHDPAVTLVKDSHVDTILFPTAWMNVLPFMTAIEFHSAWAMGMRVNLLAANTHNVSLKMTGSGIYAPHSPKVYHHDMETESGKILFAEVDSHPRNSSTYPPAMNWSAYATSITPFPGPKNSFRGFISRDEFNFTKLSESAANLTVCQKELCCHLSYRMLAKEGDEVYVLGAFAGLHGRRRREYWQVCTMLKCRSTDVTTCGQPVETALTRFEMFSLSGTFGTEYVFPEVLLTKIKLAPEKFEVLEDGRLVNKNGLSEPVLTVSLFGRWYTKDAHSISGGTSNSATAYLMIPTLLMIITLRDAVMV

SEQ ID NO: 116 - protein, 520 aa, Moschus berezovskii
MITSSLQTSVAVFALIILHVSTLDTFKAAVYEHAVILPNDTKTPVSPDEALFLMNKNIDILEKAIKQAAEQGAQIIVTPEDALYGWKFTRETIFPYLENIPDPQVNWIPCQDPHRFGQTPVQARLSCLAKNNSIYVVANMGDKKPCSSRNTTCPSNGHYQYNTNVVYDAKGKLVARYHKYHLYSELQFDVPEKPELVTFNTTFGKFGIFTCFDILFHDPAVTLVKDFHVDTILFPTAWMNVLPLLTAIEFHSAWAMGMRVNLLAANTHNVSLKMTGSGIYAPHSPKVYHHDMETESGKILFAEVDSHPRNSSTYPPAVNWSAYATSITPFPGAKNSFRGFISRDEFNFTKLSESAGNLTVCQKEICCHLSYRMLGKEGNEVYVLGAFAGLHGRRRREYWQVCTMLKCRSTNLTTCGQPVETALTRFEMFSLSGTFGTEYVFPEVLLTEIKLAPEKFEVLEDGRLVNKNGSSEPVLTVSLFGRWYTKDTHSISGGTSNSATMHLMIPTLLMIITLQYTVMV

SEQ ID NO: 117 - protein, 538 aa, Orcinus orca
MIISSFQTSVAVFALIILHVSALATFKAAVYEHAVILPNDTETPVSQDYALLLMNRNIDILERAIKQAAEQGAQIIVTPEDAIYGWKFTREAIFPYLEDIPDPLVNWIPCQDPHRFGHTPVQARLSCLAKDNSIYVVANIGDKKPCHSRDSTCPPNGHYQYNTNVVYDANGKLVARYHKYNLYSELQFDVPEKPELVTFNTSFGKFGIFTCFDILFHDPAVTLVKDFHVDTVLFPTAWMNVLPFLTAIEFHSAWAMGMRVNLLVANTHNVCLKMTGSGIYAPHSPKAYHYDMETELGKILFAEVDSYPRNSPTYPPAVNWSAYATTIKPFPGQKNSFRGFISRDEFNFTELFENAGNLTVCQKELCCHLSYRMLGKENEVYVLGAFAGLHGRRRREYWQVCTMVKCKSTNLATCGQPVDTALTRFEMFSLSGTFRTEYVFPEVLLTKINLAPGKFEVLKDGRLVNKNGSSEPILTVSLFGRWYTKDVRSISGSANRGRWRETEDWTRSGETSVCSPISVVPSSVPYLTAVVCSSSTWF

SEQ ID NO: 118 - protein, 520 aa, Mesoplodon densirostris
MIISSFQTSVAVFALIILHVSALDTFKAAVYEHAVILPNHTETHVSQDDALHLMNKNIDILERAIKQAAEQGAQIIVTPEDAIYGWKFTRETIFPYLEDIPDPLVNWIPCQDPHRFGHTPVQRRLSCLAKNNSIYVLANIGDKKPCNSYDSACPPNGHYQYNTNVVYDAKGKLVARYHKYHLYSELQFDVPEKPELVTFNTSFGKLGIFTCFDILFHDPAVTLVKDFHVDTILFPTAWMNVLPFLTAIEFHSAWAMGMRVNLLVANTHNVSLKMTGSGIYAPHSPKAYHYDMETELGKILFAEVDSYPRNSPTYPPAVNWSAYATTVKPFPGQKNSFRGFISRDEFNFTELFENAGNLTVCRNELCCHLSYRMLGKEENEVYVLGAFAGLHGRRRREYWQVCTMVKCKSTNLTTCGQPVDTALTRYEMFSLSGTFRTEYVFPEVLLTKINLAPGKFEVLKDGRLVNKNGSSEPILTVSLFGRWYTKDVRSISGGTSNSATTYLIIYTLLMIIALQNTVMV

SEQ ID NO: 119 - protein, 519 aa, Globicephala melas
MIISSFQTSVAVFALIILHVSALATFKAAVYEHAVILPNDTETPVSQDYALLLMNRNIDILERAIKQAAEQGAQIIVTPEDAIYGWKFTSEAIFPYLEDIPDPLVNWIPCQDPHRFGHTPVQARLSCLAKDNSIYVVANIGDKKPCHSRDSACPPNGHYQYNTNVVYDANGKLVARYHKYNLYSELQFDVPEKPELVTFNTSFGKFGIFTCFDILFHDPAVTLVKDFHVDTILFPTAWMNVLPFLTAIEFHSAWAMGMRVNLLVANTHNVRLKMTGSGIYAPHSPKAYHYDMETELGKILFAEVDSYPRNSPTYPPAVNWSAYATTIKPFPGQKNSFRGFISWDEFNFTELFENAGNLTVCQKELCCHLSYRILGKENEVYVLGAFAGLHGRRRREYWQVCTMVKCKSTNLATCGQPVDTALTRFEMFSLSGTFRTEYVFPEVLLTKINLAPGKFEVLKDGRLVNKNGSSEPILTVSLFGRWYTKDVRSISGGTSNSATTYLIISTLLMIIALQNIVMV

SEQ ID NO: 120 - protein, 520 aa, Sus scrofa
MIASSFQTTLAVFALIILHVRALDTFKAAVYEHAVILPNDTKTPVSWDDALLLMNKNMDILERAIKQAAEQGSQIIVTPEDALYGWKFTRETIFPYLEDVPDPQKNWIPCQDPRRFGHTPVQVRLSCLAKNNSIYVLANIGDKKPCNSHDSKCPPNGYYQYNTNVVYDSEGKLVARYHKYHLYHELQFDVPEKPEVVTFNTPFGKFGIFTCFDILFRDPAVTLVQDFHVDTILFPAAWMNVLPLLTAIEFHSAWAMGMRVNLLAANVHHVRLNMTGSGIYAPHSPKVYHYDMETDSGKILLAEVDAHPRTSPTYPTAVNWGAYTLSIQPFPILQNSFRGFISRDEFHFTELSESAGNLTVCHKELCCHLSYRMLAKEENEVYVLGAFTGLHGRRRXEYWQVCTMLKCKSTNLTTCGQPVETALTRFELFSLSGTFGTEYVFPEVLLTKINLAPGKFEVLKDGRLVNKNGTSEPVLGVSLFGRWYIRDVPLISGGPAPQQKMYLIISALLMIIALQNIVMV

SEQ ID NO: 121 - protein, 504 aa, Phyllostomus discolor
MPFHFQEGGQTRTSFRLWRSQRLLLTILSSSVSLSCRHHVADVHLLCPAWQDPGVGAQGAEIIVTPEDALYGWSFTRETIFPYLEDIPDPRVNWIPCQDPHKFGHTPVQVRLSCLAKDNSIYVLANIGDKKPCKSNDPTCPSNGYYQYNTNVVYDKKGQLVARYHKYHLYSEPQFDVPEKPELVTFNTNFGKFGIFTCFDILFHDPAVKLVKDFRVDTILLPTAWMNVLPLLTAIEFHSAWAMGMRVNLLVANTHHVRLNMTGSGIYAPHSPKAYHYDMDTELGKILFSKVDSHPRSSPDFPAAVNWADYATTIKPFPVQKGTFKGFISRDAFNFTELSASAGDLTVCQKDLCCHLSYEMSRKEENEVYALGAFSGLHGRRGREYWQVCTMLKCRTTDVTTCGQPVETALTRFNVFSLSGTFGTEYVFPEVLLTKIRLSPGKFEVLKDGRLVNKNGMSEPILTVSLFGRWYAKDAHSASGGIGNLETTNLLISTLLMITILQIL

SEQ ID NO: 122 - protein, 520 aa, Homo sapiens
MVTSSFPISVAVFALITLQVGTQDSFIAAVYEHAVILPNKTETPVSQEDALNLMNENIDILETAIKQAAEQGARIIVTPEDALYGWKFTRETVFPYLEDIPDPQVNWIPCQDPHRFGHTPVQARLSCLAKDNSIYVLANLGDKKPCNSRDSTCPPNGYFQYNTNVVYNTEGKLVARYHKYHLYSEPQFNVPEKPELVTFNTAFGRFGIFTCFDIFFYDPGVTLVKDFHVDTILFPTAWMNVLPLLTAIEFHSAWAMGMGVNLLVANTHHVSLNMTGSGIYAPNGPKVYHYDMKTELGKLLLSEVDSHPLSSLAYPTAVNWNAYATTIKPFPVQKNTFRGFISRDGFNFTELFENAGNLTVCQKELCCHLSYRMLQKEENEVYVLGAFTGLHGRRRREYWQVCTMLKCKTTNLTTCGRPVETASTRFEMFSLSGTFGTEYVFPEVLLTEIHLSPGKFEVLKDGRLVNKNGSSGPILTVSLFGRWYTKDSLYSSCGTSNSAITYLLIFILLMIIALQNIVML

SEQ ID NO: 123 - protein, 520 aa, Ochotona princeps
MITSSFLTCVAIFALITLHVGALDTFTAAVYEHAVILPKETETPISQDDALLLMNKNMNVLESAVKEAAKMNAQIIVTPEDALYGWRFTREIIFPYLEEIPDPQVNWIPCQDPHRFGHTPVQTRLSCLAKNNSIYVLANIGDKKPCNSQSSTCPRNGYYQYNTNVVYDSEGKLVARYHKYHLYSEPQFDVLEKPELITFNTTFGRFSIFTCFDILSHDPAVTLVKEFHVDTVLFPSAWMNVLPPLTAVGFHSAWAMGMRVNLLVANIHHVSQNMTGSGIYTPDSPKAYYYDTKRESGHLLVSELDSHPRNSPSYPTAVNWSAYAVTIAPFPVQKNTFIGFISQDGFNFTELLENDGNITVCHKELCCHLSYRMLEKEKDXVYVLGAFTGLHGRRRREYCQVCTMLKCQTTNLTTCGKPVETASTRFEMFSLSGTFAAEYVFPEVLYSEIRLSPGKFEVLKDGRLVNKNGVSEPILTGSLFGRWYAKDLPHKSCGNNNSTIFYMPVFTILMIAYLQNIVMV

SEQ ID NO: 124 - protein, 497 aa, Alligator mississippiensis
MMSLQPYLYAMISTLSVLQVFASDTYTAAVYEHAVILPDVTDKPVSLDEALVLMNRNIDVLEVAIKEAAQQGAHIIVTPEDSVYGWNFTREALYPYLEDIPDPQVNWIPCTDPQRFAPAPVQERLSCLARNNSIYVVANMGDKKPCNCSDPKCPRDGHYQYNTNVVFDSQGKLVARYHKYNLFRSETQFDYPKEPEMVTFNTSFGNFGIFTCFDILFHDPAVVLVKKLHVDTVLFPTAWMNVLPFLSAIEIHSAWAMGIGVNLLASNTHNTLMAMTGSGIYAPDGPRAYHYDMETENGHLLTTQLSSHPHLSPTYPAAVNWSSYATSIKQFHVDKNTFMGAVRSDNFTFTELITIAGNHTVCQKDLCCHLIYRMVEKREDEVYVLGAFDGLHGSVIKYYWQICTLLKCKTMDLKTCGQPVTTTQTRFEAFSLSGTFGTKYVFPEVLLSGVRLAPGEFQVLNDGCLKNNNGPAKLILTVTLFGRYYEKDPPHPLRNSP

SEQ ID NO: 125 - protein, 497 aa, Ceuthmochares aereus
MLPSQNLLHTAVLALTVLQALASDTFIAAVYEHSVILPNATDKPVSPNDALMLMNKNMDVLERAIKEAALQGAHIIVTPEDGIYGWRFTRETIYPYLEDIPDPAVNWIPCINPTRFSPAPVQERLSCIARNNSIYVVANTGDKKPCNSSNHDCPSDGRYQYNTDVVFDPEGKLVARYHKYNLFRGETQFNYPKEPEIVTVETPFGKFGIITCFDILFREPATVLVSKLQVDTVLFPTAWMNVLPFLTAVEFHSAWAMGMGVNLLSANTHNINASMTGDGLFTPEGPAAYRYDSKTEEGRLLLAELSSRPRLSPTYPPSVNWSLYATSIKKFPEEKDTFAKAVQRDIFTFSELGYTAGNRTVCQGDLCCHLVYQMSGKSKDEVYVLGAFDGLGGSLIKYHWQICTLLKCKSTDLNTCGQPVEMAQTKFEMFSLSGTFGTNYVFPEVLYSEVQLAPGEFEVLHDGRLKSKHGTSKPLLTATLFGRLYEKDPPHPLRTSV

SEQ ID NO: 126 - protein, 518 aa, Felis catus
MITPRLLAYVAISVSCVLKVSSLDTFIAAVYEHAVILPNTTLIPVSREEALALMNRNLDLLEGAITSAAKQGAHIIVTPEDGIYGWNFNRETIYPYLEDIPDPRVNWIPCNYPNRFGHTPVQERLSCLAKHNSIYIVANIGDKKPCNASDPQCPPDGRYQYNTDVVFDSQGKLVARYHKYNLFMGEDQFNVPKKPEVVTFDTIFGRFGIFTCFDILFHDPAVTLVKDFHVDTIVFPTAWMNVLPHLSAIEFHSAWAVGMGVNFLAANIHHPHKRMTGSGIYAPDSPRAFHYDMKTKEGKLLISRLNSSSYDPGVVNWASYASSIKAFSTGNQEFKSTAFFDEFTFLELTRVTGNYTVCQKELCCHLSYKMSEKKPDEVYALGAFDGLHIAEGSYYLQICTLLKCKTPNLRTCGDAVESASTRFEMFSLSGTFGTQYVFPEVLLSDSQLAPGEFQVSSDGRLFSVKPPSGPVLTVTLFGRLYEKDWILKASSDLRAQAPGVMLVVIALSVCSFSWSPIG

SEQ ID NO: 127 - protein, 513 aa, Phodopus roborovskii
MSVSRWLACAVLSVFYVLQVSSLDTFVAAVYEHAVIMPSDTPSPLSHSEALAIMNRNLDLMEGAIVSAAKQGAHIIVTPEDGIYGVHFTRDTIYPYLEDIPDPQVNWIPCDNANRFGYTPVQERLSCLAKNNSIYVVANMGDKKPCNTSDPQCPPDGRFQYNTNVVFDSLGKLVARYHKQNLFMGEEQFNAPTEPELVTFNTPFGRFGIFTCFDILFHDPAVTLVTKFQVDTILFPTAWMDVLPHLAAIEFHSAWAMGMGVNFLAANIHSPLRRMTGSGIYAPDSPRAFHYDRKTNEGKLLLAQLDSHPSSSSVNWTSYASSVETPSTGDQEFQSVVFFDEFTFVELKGITGNYTVCQNDLCCHLSYQMSEKRADEVYALGAFDGLHTVEGQYHLQICTLLKCKTTNLRTCGSSVDTAATRFEMFSLSGTFGTQYVFPEVLLSQVKLAPGEFQVSSDGRLFSLKPTSGPVLTVTLFGRLYEKDWVSSTSSDLLTHSLMIIIMIIITPIIHYLC

SEQ ID NO: 128 - protein, 553 aa, Moschus berezovskii
MIMSQLLNYVAFLFFCVSRADSLDTFIAAVYEHPVILPNATLVPVSPEEALAVMNRNLDLLEGAVTSASKQGAHIIVTPEDGIYGFNFTRESIYPYLEDIPDPQENWIPCNNPNRFGHTPVQQRLSCLAKDNSIYIVANIGDKKSCNASDPQCPPDGRYQYNTDVVFDSKGKLVARYHKQNLFLSEDQFNAPKEPEVVTFNTTFGKFGIFTCFDILFHDPAVTLVRDSHVDTILFPTAWMNVLPHLSAIEFHSAWAMGMRVNFLAANLHYPLKKMTGSGIYAPDSPRAFHYDMKTEEGKILLAQLDSHPHPTPVVNWTSYASGVEVHSMGNQEFTDTIFFDEFTFLELKGIGGNYTVCQRDLCCHLSYKMSEKRADEVYALGAFDGLHTVEGSYYLQICTLLKCKTTDLHTCGDSVETASTRFEMFSLSGTFGTQYVFPEVLLSEIQLAPGEFQVSSDGRLFSLKPTSGPVLTVTLFGRLYEKDSAPNALSDLTTQALRIRRLVSIHNYRSECCIIPWRRKWQPTPVFLPGEFPWTEEPGGLQYMGSRRVGHD

SEQ ID NO: 129 - protein, 491 aa, Amazona guildingii
MLPSQPLLHTAVFALTVLQALASNTFIAAVYEHAVILPKAAIKPVSPDDALALMNSNMDILEGAIKEAAQQGAHIIVTPEDGIYGWRFTRETIYPYLEDIPDPVVNWIPCTDPTSIYLELKEVPFRNPAAYTVCCITQGWSEWCQQRTSRGLKFVHSPPLPXVVYDPEGKLVARYHKYNLFKGETQFNYPKEPEATTFETPFGKFGIFTCFDILFHEPAVVLVSELQVDTVLFPTAWMNVLPFLTAVEFHSAWAIGMGVNLLSANTHNINFAMTGSGLFTPEGPVAYHYDSETDEGRLLVAELSAQPRLSPNYPPAINWSLYATSIKKFPEEDTFLGAVRKDIFTFSELRHKAGNYTVCQRDLCCHLIYQMSNKSKDEVYVLGAFNGLHGSFIKYYWQICTLLKCKSTDPETCGHPVETAQTKFEMFSLSGTFGTNYVFPEVLYSGVQLAPGEFEVLSDGRLKSKHGTSKPLITATLFGRLYEKDLPHPLR

SEQ ID NO: 130 - protein, 520 aa, Patella sp.
MKKNLDIYAEQTRIASSQNVDIIVFPEDGIYGLNFNRDTIKPYLEDIPDPTEITWNPCNQSEYTNGVEVQITLSCMARNNGIYIVANMGDKKVCDADSICPDDGQRQYNTDVVFDPQGNLVARYHKQHLYFEAQFDTPSTVERVTFDTPFGKFGIITCFDILFKSPAVDLVEDLGVKNVIFPTAWMDSLPLLSAIQFHSAFAKLFKVNLLAANLHFPLFNFHGSGIYSPRRTEAYYYNDGLFSPGKLLIADVKSSPTHYRRHQVVPDTHGFRGGNSLGASFVHGQDTAEVNTQSRRNGGHYNTASRNGIISHQLADQASGVHLNNDLGPLTHRSIRNRIHNHAHLHRVNRHMVENGWATIMADNDNYQYLPLTEPKAKVKICQNEVCCYLDYQKQNTSNDELYALGVFDGLHQIEGLSWYTQVCVLLKCEFTDIASCGQPTKIAAGHFEYLKLWGTFTTEYVFPEVLVSDDGKLRLEHEHWKYLHNRIEFVNRGKESPLLSATLYGRHYDRDNSTVSNSV

SEQ ID NO: 131 - protein, 657 aa, Dreissena polymorpha
MFLELRLNLSIDRKNNIMEQNCLTKYFKTLLVCSLFCLTQCLKTDIKKFFTAAVYEHAVYQPPVTPLPLPRAQALEIMMTNIRVYQEQTKIATSKGAHIIVFPEYGVYGLSYKTRELIFPFMENIPEPERNWNPCEHPDVYPETEIQFALSCMARENNIYVVANIGDKQPCNVSLDPSCPRTGYYQYNTNVAYDAMGTLVAKYHKYNLFYENWFDKPSKVDISVFNTPFGRFGLIICFDILFEHPPIDLICRNNISNIAFPTAWMDAHPFYTSIQFHSAFAVGLGVNFLAANIHYPSLRFHGSGIYTQQGAAGYCFNTSNESGGQLIVQKIPVVDQSRLDACRGLNISLSDLNDQMLELDKTVNNGRETLQHTKRKGWGKGREKGRGNGQGQGKMHGHGQRNGQGQGKRKETRQEKGRGKRKGQGNGQGKGKGPRKLDLLSQPKFHKDMFPYDLYKNPFNLVPLRPEGRKTIVCHTDLCCHLSFSFEPTDRRSAEKPTRKSHAPDDFQASQHSTDLRNFTENRKDQDDIRAHLADNQRSSVEDFYAIGAYDGRYKNRSTGYYIQVCIVLRCRKQQNVVNCSASMAEDFRIRLSHFRLVGNFSATFQYPSILLQGESDFRLTEHSRRWSFSNGQLVANERFSSPLAVATIFARDYGRD

SEQ ID NO: 132 - protein, 533 aa, Patella vulgata
MLPVILVVAGILLNSMSTNAYNRKTFTAAVYEHSLIFPPNRSTVASRHNALADVMVNLRIYDQQAKFAASQDVDILVFPEDGLYGYTFNRDSIKHYLEEIPDPKSEVWNPCTQPNRYSKTEIQVYLSCMALNNSMYIVANMGNKQRCTRRTDSNCPSDGQYQYNTNVVYAPNGRLIARYHKQNLFYEYQFDRPRSVNYAFFETPFGRFGTFTCFDILFEEPAITLIERYNVSNIVFPTAWMDALPLLSAIQFHSAFAQGMGVNFLAANIHVPNKRFHGSGIYTPDGPKKFYYNDVGYGGGKLIISQLNVIENRHQHVAYNKVQRPSSEQKTFHSYVFYDKFTFVELQRSSGVLSVCNNRLCCHLEYAINKDNSDMYAFGAFDGLHTHEGQYYLQICTLLRCANKTRSSCGQPTKQAYSEFNDIKIYGTFQTNYVFPEVLKSRKRDGQLNLATNGWKYNNGVLRATRKTTHPVLSVSLFGRIYSRDKLPPGYHFTGSGSKSLSSSLYVSLLTPALLLAVYGSSIACGLFTRMNN

SEQ ID NO: 133 - protein, 517 aa, Mizuhopecten yessoensis
MIIKFIYVAVLTSSVGGFLFQNETRTTFRAAVYEHAVHLFPSSSSTTRQTALANMMMNLEEYKRQADIAGGQDVDILVFPEMGIYGLGLSREGIAPYLENIIDPEKEVWSPCDYPHRYPNSEVQHFLSCLAKNNSLYIVANFGDRQPCSQSLDPNCQAQYQYNTDLVYDPRGILVAKYHKVNLYFEKSFDFPPVNKAVTFDTPFGVFALAICFDVLFYDPIIDLIINHGVRNVAFPTAWMDRLPFLSSIEYHSAFAAGMGVNMLAANINYPRMRFHGSGIYTPNGASRFIYNDQTASGKLLVSDLSVINKHGSVTLPPRTPTLKTPHVTGSIFKSKLHYDSYNLTLISGISGHATVCHNTICCQLNYSRQNTNETFAFGAFDGLHGRNGSYFMYLQVCTLLKCTRTGNTSCDDRNMQSQTMFNNVHITGYNFTSYIYPEVLLSQNGSLGLSIGRWTYSYGVLHAPTGYEAPLVSTSLYGRLYSTDDSNGRESCSIRLINRKSLITDLVVTLTCRISE

SEQ ID NO: 134 - protein, 520 aa, Octopus sp.
MNFVYTIIFFLQLPVLLSAAEKTFTAAVFEPTIPRRSSMGLVSRDNAIEQMKHVLKGYAEVAARASKKGVDILVFPENGLIAHLDGVTRLGIRNFLDLIPEPNKEKPWIPCDEPYLYPRTEIQQVISCIAKKNKIYLVVNVGDIQPCNRTDDANCPYDGRYQYNTNVVYSSEGALVSRYHKKHLFYEPYYNSPKIAEYGVFDTPFGRFASIVCFDIYFNDSLNELVMKHQVSNILFPNLWFDTPPFFFSSLFHNSIAKAFNINLISANIKINNFVRGGSGIYTPSINRYVYNPLNPAPSDEYIQLQVKNLEKSKRTFPCPPIRKVPESDNPDVKTATMTNLFPVSAVMLHKYSGVVSVCKYNACCSLQYEFMSNRTSVFNINYILAITNEESVDLTNPEGVDMQSCILCPIKDFKMSCLMSLPAGIPDGEVSFRKLKMTAHNFNTSYFFPHIATFNSSTYDINTSWKHDRGEIEITEPVKKVFNAVIFGRLYPNSATATFAIFPAILLILLNLISKELLV

SEQ ID NO: 135 - protein, 554 aa, Formica exsecta
MRTRILDLLLIYSSLNFCCYGNLEDCTDGPDEDYCNENFISAPAQISTPDSPTYIAAVVEYHPEYTTDSNETLKKNSDAYVEHIKTARVHNADIIVFPEDGLTTVKFPKREEMQNWTTIIPSASFNYTPCTDSTIEVSETLKKISCAARNNTIYVVINIAEKLPCIEDVCPKDKMFYYNSNVVFDRTGKIIARYRKTNLFGEQQFNVTSKPEIVIFDTDFGVKFGTFICFDILFHEPALQLTRVHQVTDIVYPTAWFSEAPFLTAVQTQAGWSFSEDVNFLVSGYNEPASGNTGSGIYLGREGIGKAIMSKTTRDEILVFEVPKKKKVFNKDHHDHSNDYDHKVYNHKKENIHEELWRKQFSNTMADNDTIFLIHDNINVFETFSLEGNVTRNVCKNNFCCDFKVEVVTIDPSKKYRLIAFSGIRLYATVEAGVRACGIVQCLNDSVSSCGSVHDSKTVFSRIEIDTTFYDYKNILIMPSTLRSDLLPLSENWTYNEHTHDDHVHISMLLNNNTNNLTTFGIYSRYFNKNNASRIVFDIYYFIALLVSLCLSRL

SEQ ID NO: 136 - protein, 516 aa, Drosophila simulans
MAKNYWGFSLFCLALGLMLNLSQQASLFSRDFYTAGVVEFKPSNDLSDNLAGYLEIIHSQNATSTDIIVFPESTLNSAGSTTFVPNPEDQIIPCLSDPNATYYEEFLVTLSCAARNASKYIVINLTEKQKCEDVPEDTRPCASNGLNVFNTNVVFDRQGVVVSRYRKVHLYGEPKNSTFLPELSTFETDFGVTFGHFICFDILFYTPAHQLVDQGVTDFVYPAMWFSQLPFLTAVQIQLGWSYANNVNLLAAGASDPIVGNSGSGIYHGRSGSLRSVMRQESGERSIYVARVPKYRAKRRMKRDLNRQVATSSSFNIKRDYLENFTSEELKIDDGKTGNLSQNLCHGGFCCHFDLAWRSLRKPARNTSHYSYRVGIYEGWRNEKRLDVNYIRNCGLFTCSGDSIDDCGQLLPDLHRPLVTFTHLEIRVTYPQSRKFLLFPDTLLDNLLPLEPKQFEWSQKRISEESQQVRLALRKSLEVKHLLTFGIYGNYYNNECTYGVGTTEEQLECGYKNPKI

SEQ ID NO: 137 - protein, 535 aa, Trichonephila clavipes
MKRILDFQLFFTTVLSLLALELAAGPDHYRAAVAETVRFSDISCPPKEVVKRNLDIYEAAVKASAKNDVDIIVFPECGIFPCEKVNRSVLRTFVEDIPNPKRESINPCKQSDKFHNHSILSKLSCLAKKHNLYIVANTFDFKKCKPVETCDADGFDTCTSEESNCPSDGNFFYNTNIVFDRKGSLVSRYYKKHLYFERTLNKPDFQQDATFHTDFGNFTTVICFDLIFKESVEDVKKPGIINLAYPTYWYDHVPQVFFATAYQQAWAMTNKVNLLAANGHMPKTGTLGSGIYSAHKGALIYTHNPDFRTKLLISNVPRSLNKEKANSIELNEKKFYLESGAAIEVVGEEEEERNFRSDCTTTVLGEANDTMDSYRCFDSTLNDYQYFKLEGNEGTHEICSNEFCCSLSYQAHSMDEDYFLAVSGKPLTYYDTFSYGIQSCFLARCRSSDGHPCKHFLLKSRTIFQNVHIKANFTTRHVYPFALDSDVRLTNRKNWSFDGKSQITYENLNKKSSVTFFGLHGRLYDVDKILKKLLK

SEQ ID NO: 138 - protein, 509 aa, Parasteatoda tepidariorum
MTTSWFLLLSMFFVACTDIDATAYTAAAYDHARVGRLNDPPNLVVEMNLKKYIKAGKIAKSKGADIIVYPEYGIFYPGNRTQLKHFLEDVPDPKKTRTAPCDEVELYMKRPILYTLSCVAKHNAMYVVANMGDIKDCSGVSECPEDGVFHFNANIVFDRNGRLILRYYKEHLFFELGMDLAREEQDPVFTTDFGTFATYICFDILFKRMSQVAKWPQIDGIMFTSMWAENPPQGLSVAFWQGWALGNNATIVVSNIQSPSRYALGSGIFHKTHGSLGYTYNPDRVSKVVVAKVPVSHQPVLTSRPSLTAFTDTKVWDMKSEEEDVPKVCSNLLIDATDDSTEKLYCSDEKTTNYTLVKLPKSGGHVKACNNGMCCSVSYSAETIEEDYYLGVYNGTINAYDLNRYCWCEENCFLARCDAIKGRPCSTFPRKTNTVFYHVELKSNFSTPFVYPSVVSSKSRLVPMTEWEFDVGHSSDSQSSIHFHSKYGEQLLSVGFRARCFDRDPPYVR

SEQ ID NO: 139 - protein, 487 aa, Rhipicephalus sanguineus
MQGMWLLLTAVLCSSTAHAFKAAVYEHVQSKATPTETAVAENLEAYLWATGNASSEKADIIVFPEDGILYRLKDREEVAKWAEDIPDLGVMACGSQNMSLLSNLSCMAQSNSIYLVANLIDRKPCANETLTTAVTATRSSISTTNGPSDRNGPLSQVILHMLLIETFSISRCILPTTRYHKNHLFVEPLMNPADPYEFAVFKTDFGAQVGLFICFDVLFAESSLLVEKHNVTLGVMSSWWFDELPGWYSVGVQQAWSIHNGIPLLASGIQRLEMGSLGSGIYAGLRGPLNYTYSPDGKSKLLMADLSNATSLNPRYPGDELHPKQRFLNQGDVSDHAAQVLGRPKGYSRVCHGDFCCSLSYETQELNDNFVLLARNGLRNIGTYMQLGIQQCILAVCESRNNTVCKNFSTRSTTKFTELQLTAQFATNAVFPVVASDELALTPKDKWQFETTSTNASTLTLKENGKDEDILHIVLYARLYDADRFTK

SEQ ID NO: 140 - protein, 524 aa, Caerostris extrusa
MMASGFKLLCLAVALHMSSIGLCADEKYYRAAVHEIRQYNDNSSSGSELISKNMELYNQAADIASFENCDILVYAEKGLFPSKKSNSSWFIYYAEDVPSGKERANPCEQEEYSDRPILKNLSCIAQEHSLTVVATLIDIKKCIVSEDCERTTINYCVSNADDCPEDGIYRFNTQVVFNKRGTLIARYYKQHPFFEEGITTPKYAANVYYKTNYGTFVSYIGFDFFFYDSVKGVEKPWINAVTYANWWFDHTPFNYFSVSNQQAWSMNNGVAVLSSDVHEPNLGSLGSGIYIPGKGALIYSYNPDGKSKLLISNVPKSNSLSDNESLNTKFYYIDENGTGTVLNGEEPRNFKSQCSIDVLGEETSSFTDYRCHKTEVHQYTFTKLNDTSGQIDTCSNGFCCSLSYTAESMNETFYFGVSGHRLKFYDTYWFGVQACFLARCEDVDGKQCRNFLLTSDTIFHDVEITGSFETKHIYPQAMDTELRLTDRNEWKFDGQSMSYESVNQNPLLFMGLYGRLYRRDGLVK

SEQ ID NO: 141 - DNA, 1503 bp, Mus musculus
  source 1..1503: mol_type = genomic DNA
atggcttcattacattttcctcaatgggcagtgagttttgtcttctttgcccaggctgtgggttcaatggacacttttattgctgctgtgtatgaacatgctgttatactgccaaacaaaactgaaagtcctgtttccactgaagaggctttgctcctgataaacaagaacatagacattttggagagtgcaatcaagctggcagccagacagggtgcacatatcattgtgacgccagaagatggaatctatggttggatcttcaccagggagaccatttacccctacctagaggatataccagaccctgaagtgaactggattccctgtagagaccctaggaggtttggctacacaccagtacaggagagactgagctgccttgccaaggagaactctatctatattatggcaaatattggggacaagaagccatgcaatgctactgatcctcattgtcccccggatggccgttaccaatataataccaatgtggtcttcgattctaagggtaggctaacagcccgctaccataagtacaatctttttgaaccagagattcagtttgatttccccaaagattcagagctggtgacctttgacaccccgtttgggaagtttggcatcttcacttgctttgacattttctcttatgacccagctgtggtggttgtgaaggacacccaggtcgacagtgttctcttacccacggcgtggtacaacaccctgcccctgctttcagcagttccattccattcggtgtgggccagagccatgggggtcaacgtgcttgctgcaaacacccacaacaccagcatgcatatgacagggagtggaatctacagcccggaagctgtccgagtgtaccactatgacatggagacagagagtggccaactgctgctttcagagctgaggtctcggcctcgccagcacgccacccctgcagaggttaactggagcgcttatgccaggactgtgaagccgttctcatcggggcaggcagacttcccaggaaagatttattttgacgaatttagcttcaccaagcttacaggaagtgctggcaattacacagtttgccaaaaggacctgtgctgtcacctgacttacaagatgtctgaaagccgaatggacgaggtgtatgttctgggtgcctttgatggactccatacaggggaaggccagtattacctacagatatgtacattgctgaagtgtcaaaccaccaactcgagaacttgtggggaacccgtggggtcagcttttacaaagtttgaagaattctctctcagtggcacctttcggacaaaatatgttttcccacagatcgtgctaagtgggagtcaacttgccctggaaagatattatgaagtctcaagagatggacgtctgaggagtcgaggtggagcccctttgcctatcttagtgatggccctgtatggaagagtgtttgagagagaccctccgcgcttagggcagggacctgggaagctgcagtga

SEQ ID NO: 142 - DNA, 1503 bp, Mus caroli
  source 1..1503: mol_type = genomic DNA
atggcttcattacattttcctcaatgggcagtgagttttgtcctctttgcccaggctgtggggtcaatggacacttttattgctgctgtgtatgaacatgctgttatactgccaaacaaaactgaaagtcttgtttccactgaagaggctttgctcctgataaacaagaacatagacattttggagagtgcaatcaagctggcagccagacagggtgcacatatcattgtgacgccagaagacggaatctatggttggatcttcaccagggagaccatttacccctacctagaggatataccagaccctgaagtgaactggattccctgtagagaccctaggaggtttggctacacaccagtacaggagagactgagctgccttgccaaggagaactctatctatattgtggcaaatattggggacaagaagccatgcaatgctactgatcctcattgtcccccggatggccgttaccaatataataccaatgtggtcttcgattctaagggtaggctaacagcccgctaccataagtacaatctttttgaaccagagattcagtttgatttccccaaagattcagagctggtgacctttgacaccccgtttgggaagtttggcgtcttcacttgctttgacattttctcttatgacccagctgtggtggttgtgaaggacgcccaggtcgacagtgttctcttacccacggcgtggtacaacaccctgcccctgctttcagcagttccattccattcagcgtgggccagagccatgggggtcaacgtgcttgctgcaaacacccacaacaccggaatgcacatgacagggagtggaatctacagcccagaagccgtccgagtgtaccactacgacatggagacagagagtggccaactgctgctttcagagctgaggtctcggccccgccagcatgctacccctgcagaggttaactggagcgcttatgccaggactgtgaagccgttctcatcggggcaggcagacttcccaggaaagatttattttgatgaattcagcttcaccaagcttacgggaagtgctggcaattacacagtttgccaaaaagacctgtgctgtcatctgacttacaagatgtctgagagccgaatggacgaggtgtatgtcctgggtgcctttgacggactccatacagtggaaggccagtattacctacagatatgtacactgctgaagtgtcaaaccaccaactctagaacttgtggggaacccgtggggtcagcttttacaaagtttgaagaattctctctcagtggcacctttcggacaaaatatgttttcccacagattgtcctaagtgggagtcaacttgccctggaaagatattatgaggtctcaagagacggacgtctgaggagtcgaggtggagcccctttgcctatcttagtgatggccctgtatggaagagtgtttgagagagaccctccacgcttagggcagggacctgggaagctgcagtga

SEQ ID NO: 143 - DNA, 1503 bp, Mus pahari
  source 1..1503: mol_type = genomic DNA
atggtctcattacattttcctcaatgggcagtgagttttgtcctctttgcccagactgtgggttcaatggacacttttattgctgccgtatatgaacatgctgttatactgccaaacgaaacagaaactcctgttcccaccgaagaggctctgctcctgataaacaagaacatagacattttggagagcgcaatcaagctggcagccagacagggtgcacatatcattgtgacgccagaagacgggatctacggttgggtcttcaccagggagaccatttacccctacctagaggatataccagaccctgaagtgaactggattccctgtagagaccctaagaggtttggctacacaccagtacaggagagacttagctgcctcgccaaggagagctctatctatattgtggcaaatattggggacaagaagccctgcaatactactgatcctcactgtccccgggatggccgttaccagtataataccaatgtggtcttcgattctaagggtaggctaacagcccgctaccataagtacaatctttttgaaccagagattcagtttgatttccccaaagattcagagctggtgacctttgacaccccgtttgggaagtttggcatcttcacttgctttgacattttctcttatgacccagccgtggtggttgtaaaggacgcccaggttgacagtgttctcttacccacggcgtggtacaacaccctgcccctgctatcagcagctccattccattcagcgtgggccagagccatgggggtcaacgtgcttgctgcaaacacccacaacaccaggatgcacatgacagggagtggcatctacagcccggaagccgtccaagtgtaccactacgacatggagacagagagtggccaactgctgctttcagagctgaggtctcggccccgccagcatgccacccctgcagaggttgactggagcgcttatgccaggactgtgaagccgttctcatcggggcaggcagacttcccgggaatgatttattttgatgaattcagcttcaccaagcttatgggaagtgctggcaattacacagtttgccaaaaggacctgtgttgtcacctgacttataagatgtctgagagccgaatggacgaggtgtatgtcctcggtgcctttgatggactccatacagtggaaggccagtattacctacagatatgtacattgctgaagtgtcaaaccaccaacttgagaacttgtggggaacccgtggggtcagcttttacaaagtttgaagaattctctctcagtggcacctttcggacaaaatatgtcttcccacagatcgtcctaagtgggagtcaacttgccctggaaagatattatgaagtctcaagagacggtcgtctgaggagccgaggtggagccgctttgcctgtcttagtgatggccctctacggaagagtgtttgagagggaccctccacgcttagggcagggacctgggaagctgcaatga

SEQ ID NO: 144 - DNA, 1503 bp, Arvicanthis niloticus
  source 1..1503: mol_type = genomic DNA
atggtttcattatattttttacaatgtgtagtgagtcttgtcctctttgctcaggctgcgggttcaatggacacctttattgctgcggtatatgagcatgctgttatactgccaaacaaaacagaaagtcctgtttccaccgaagaggctttgctcctgatgaacaagaacatagacattctggagaatgcgatcaagctggcagccagacagggtgcacagatcattgtgaccccagaagatggaatctatggctggatcttcaccagggagactatttacccctacctagaggatataccagaccctgatgtgaactggattccctgtagagaccctaaaaggtttggctacacaccagtacaagagagactcagctgcctcgccaaggagaactctatttacatcgtggcaaatattggggacaagaagccatgcaacgctactgatcctcagtgccccagggatggccgttaccaatataataccaatgtggtcttcagttctgagggtaggctagtagcccgctaccataagtacaatctttttgaaccagagattcagtttgattttcccaaagattcagagttggtgacctttgacaccccctttgggaagtttggcgtcttcacttgctttgacattttttcttacgacccagctgtgatggttgtgaaagactttcaggttgacagtgttctcttacccacggcgtggtacaacactctgcccctgctttcagccgttccattccattcagcatgggccagaggcatgggggtcaacctgcttgctgcaaacacccacaacaccagaatgcacatgacagggagtgggatctacagtccggaggctgtcagggtttaccactacgacatggagacagagagcggccagctactgctttcagagctgaggtctcggccccgccagcaggcctcccctgcagacgttgactggagtgcctatgccaggactgtcaagccattctcatcggagcaggccgacttcccagggatgatttattttgatgaattcagcttcaccaagcttacaggaagtgctggcaattacacagtttgtcaaaaagacctgtgctgtcacctgacttacaagatgtctgagagccgaatggatgaggcttatgccctgggtgcctttgatggactccataccgtggaaggccagtattacttacagatatgtacattgctaaagtgtcaaaccactaacttgagaacttgtggggaacccgttgggtcagcttttacgaagtttgaggaattctctctcagcggcacctttgggacgaaatatgttttcccacagatcgtcttaagtgggagtcaactggccctggaaagatattatgaagtctcaagagatggacgtctgaggagccaaagtggggcctctttgcctgtcttagtgatggccctgtacggaagagtgtttgagaaagattctccacgcttaggtcagggacctgggaagttgcaatga

SEQ ID NO: 145 - DNA, 1506 bp, Homo sapiens
  source 1..1506: mol_type = genomic DNA
atgattatatcacattttccaaaatgtgtggcagtttttgccctccttgctctgagtgttggtgcactggacacttttattgctgcagtatatgagcatgcggtgatattaccaaacagaacagaaacacctgtttcaaaagaagaagctttgctcctgatgaacaagaacatagatgttttggagaaagcagttaagctggcagcgaagcagggtgcacatatcattgtgaccccagaagatggaatctatggttggatcttcaccagggagagcatttacccctatctagaggatataccagaccctggagtgaactggattccatgtagagacccctggagattcggcaacacaccagtgcaacaaagactcagctgcctggccaaggacaactctatctatgtcgtggctaatattggggacaagaagccatgcaatgctagtgactctcagtgtccccctgatggccgttaccaatacaacactgatgtggtgttcgattctcagggaaaactgttggcacgctaccataagtacaatctttttgcacctgaaattcagtttgatttccccaaggattcagaacttgtgacttttgacactccctttgggaagtttggcatttttacttgctttgacattttttctcatgacccagctgcggtggtggtggatgaagtttcaattgacagcattctctaccccacagcatggtacaacacgctgcccctcctctcggctgttcccttccattcagcatgggccaaggccatgggagtcaatctacttgctgcaaatacccacaacaccagcatgcacatgacagggagtggaatctacgccccagaagcagtcaaggtgtaccactatgacatggaaacagagagtggtcagctgttgctatcagaactgaagtctcggccccgccgtgagcccacctaccctgcagctgttgactggcatgcgtatgccagcagtgtcaagccattttcctctgaacagtcagattttctggggatgatttattttgatgagtttaccttcaccaagcttaagagaaatacaggaaattacacagcttgccagaaagatctgtgttgtcacttaacttacaagatgtctgagaagcgaacagacgagatctatgccctaggtgcttttgatggactgcacacagtagaaggccaatattacttacagatatgtgcattactgaagtgtcaaaccactgacctggaaacgtgtggagaacctgtggggtcagcttttaccaagtttgaagacttctccctcagtggcacatttggaacgcgttatgttttcccacagatcattctaagtgggagtcagcttgcccctgaaagacattatgagatttcaagagatggacgcttgaggagccgaagtggagcccctttgcctgtcttagttatggccctgtatggaagagtgtttgagaaggaccctccacgcttagggcagggatctgggaaattccagtga

SEQ ID NO: 146 - DNA, 1506 bp, Cynocephalus volans
  source 1..1506: mol_type = genomic DNA
atgattatatcacattttccaaaatgtgtggccatttctgccctctttgtcctgagtgttggtgcactggatacttttattgctgcagtgtatgagcatgctgttatcttaccaaacagaacagaaacacctgtttcaaaagaagaagctttattcctgatgaacaagaatatagatgttttggagaaagcagtcaagctggcagccaagcagggtgcacatatcattgtgacaccagaagatggaatctatggttgggtattcaccagggagagcatttacccttatctggaggatataccagactctgaagtgaactggattccatgtagagatcctcaaaggtttggctacacaccagtgcaagaaagactcagctgcctggccaaggacaactccatctatgttgtggcaaatattggggacaagaagccatgcaatgccagtgaccctcaatgtcctcccgatggtcgttaccaatacaacacggatgtggtgtttgattctgagggtaggctggtggcacgctaccataagtacaatctttttgcacctgaaattcaatttgatttccccaaaacatcagaagttgagacttttgacactccctttgggaagtttggaattttcacttgctttgacattttttctcatgacccagctgtggtggtggtggacaagtttcaagtcgacagtgttatctaccccacagcgtggtacaacacgctgccccttctctcggctgttccattccattctgcatgggccagagccatgggagtcaatctgcttgctgcaaatacccacaacaccagcatgcacatgacagggagtggtatctatgccccagaagcagtcaaggtatatcactatgacatggaaacagagagtggtcagctgatgctctcagaattgaagtctcggcctcgccgtgaacccacctatcctgcagttgttgactggggtgcatatgctgaaagtatgaagccattctcgtctgagcagtcacattttccagggctgatttattttgatgagttcaccttcaccgagcttaagagaaacacaggaaattacacagtgtgccagaaagacctgtgttgtcacttgacttacaagatgtctgagaagcgaacagatgaggtctatgcactaggtgcttttgatggactgcacacagtagaaggccaatattacttacaggtgtgcacattactgaagtgtcaaaccactgacctgagaacttgtgggcagccagtggggtcagtttttaccaagtttgaagaattctccctcagtggcacatttggaaccagttatgttttcccgcagctcattctaagtgggagtcagcttgcccctgaaagacattatgaggtttcaagagatggacgtctaaggggccgaagtggagcccctttgcctgtcttagttatggctctgtatggaagagtgtttgagaaggaccctccacgcttagggcagggacctgggaaattgcaatga

SEQ ID NO: 147 - DNA, 1506 bp, Sapajus apella
  source 1..1506: mol_type = genomic DNA
atgagtagatcatattttccaaaatgtgtggcactttctgccctccttgtcctgagtgttggtgcactggacacatttattgctgcagtatatgagcatgcggtgatattaccaaacagaacagaaacacctgtttcgaaagaagcagctttgctcctgatgaacaagaacatagatgttttggagaaagcagttaagctggcagccaagcagggtgcacatatcattgtcaccccagaagatggaatctatggttgggtcttcaccagggagagcatttacccctacctggaggatataccagaccctgaagtgaactggattccatgtagagaccctgggagattcggcaacacaccagtgcaagaaagactcagctgcctggccaaggacaactctatctatgtcgtggctaatattggggacaagaagccatgcaatgccagtgaccctcagtgtccccccgatggccgttaccaatacaacactgatgtggtgtttgattctcagggaaaattggtggcgcgctaccataagtacaatctttttgcacctgaaattcaatatgatttcccgaaggattcagaatttgtgacttttgacactccctttgggaagtttggcatttttacttgctttgacattttttcttatgacccagctgtggtggtggtggatgagtttcaagttgacagcattgtctaccccacagcatggtacaatacgctgcccctcctctcggctgttcccttccattcagcatgggccaaggccatgggagtcaatctgcttgccgcaaatacccacaacaccagcatgcacatgacagggagtggactttacgccccagacgcagtcaaggggtaccactacgacatggaaacagagagtggtcagctgatgctatcagaattgaagtctcggcctcgccgtgagcccacctaccctgtagctgttgactggcatgcttatgccagcagtgtcaagcctttctcctctgagcagtcagattttccagggatgatttattttgatgagtttaccttcactgagcttcaaagaaatacaggaaattacacagtttgccagaaagacctgtgctgtcacttaacttacaagatgtctgagaagcgaacagatgaagtctatgccctaggtgcttttgatggactgcacacggtagaaggccaatattacttacagatatgcacattactgaagtgtcaaaccaccgacctcgaaacatgtggagagcctgtgggttcagcttttaccaagtttgaagaattctccctcagtggcacattcagaacgcgttatgttttcccacagatcactctaagtgtaaatcagcttgcccctgaaagacattatgagatttcaagagatggacgcctgaggagccgaagaggagcccctttgcctgtcttagtcatggctctgtatggaagagtctttgagaaggaccctccacgcttagggcagggacctgggaagttacagtga

SEQ ID NO: 148 - DNA, 1506 bp, Panthera leo
  source 1..1506: mol_type = genomic DNA
atgattacatcatattttccaaaatatgtggcactatttgccatctgtgtcctatgtgtcggtgcactggacactttcattgctgcagtgtatgaacacgctgtcatattaccaaacagaacagagacaccagtttccaaagaagaagctttgctcctgatgaacaagaacatagatgttttggagaacgcagttaagctggcagccaggcagggtgcacatatcattgttaccccagaagatggaatttatggctgggttttcacaagagagaccatttacccctatctggaagatatcccagacccagaggtgaactggattccatgtacagatccccagaggtttggctacaccccagtgcaacaaaggctcagctgcctggccaaagacaactccatctacgttgtggcaaatattggggacaagaagccatgcaatgccagcgaccctcagtgtccccccgatggccgttaccagtacaacactgatgtggtgtttgattctgagggtaggctggtggcacgctaccataagtacaatctttttgcgcctgaaattcaatttgatttccccaaggattcagaatctgtgacttttgagactccttttgggaagtttggcattttcacttgctttgacattttttctcacaccccagctgtggtggtggtggatgagtttcaagtggacagcgttctctacccggtggcgtggtacaacacactgccccttctgtcggctgttcccttccactctgcgtgggccagagccatgagagtcaacttgcttgctgcgaatacccacaacaccagcatgcacatgacagggagtggaatctacgcaccggaagcggtcaaagtgtatcactatgacatggaaacagagagtggccagctgatgctgtccgaactgaagtcccggccgcgaagtgaacccacctatcctgcagctgttgactggagtgcttatgccagaagtgtcaagccattctcgtctgaacagtcgaattttccggggatgatttattttgatgagctcaccttcattgagcttaagagaaacacaggaaattacacagtttgccagaaagacctgtgctgtcacttaacttacaagatgtcggagaaacgaacagacgaggtttacgcactaggtgcttttgatggactgcacacagtagaaggtcaatattacctacagatatgcacattactgaagtgtcaaatcagtgacctgcgaacttgtggggagcccgtggggtcagctttcaccaagttggaagaattctccctcagcggcacatttgcaacacgttatgttttcccacagatcattctcagtggaagtcagcttgctcctgaaaggtattatgaggtcttgagagacggacgtctgaggagccgaagcagaacccctctgcctgtcttagttttggccctgtacggaagagtgtttgacaaagaccctccgcgcttggggcaaggaccccggagattagaatga

SEQ ID NO: 149 - DNA, 1506 bp, Lynx canadensis
  source 1..1506: mol_type = genomic DNA
atgattacatcatattttccaaaatatgtggcgctatttgccatctgtgtcctatgtgtgggtgcattggacactttcgttgctgcagtgtatgagcacgctgtcatattgccaaacagaacagagacaccagtttccaaagaagaagctttgctcctgatgaacaagaacatagatgttttggagaacgcagttaaactggcagccaggcagggtgcacatatcattgttaccccagaagatggaatttatggctgggttttcacaagagagaccatttacccctatctggaagatatcccagacccagaggtgaactggattccatgtacagatccccagaggtttggctacaccccagtgcaacaaaggctcagctgcctggccaaggacaactccatctacgttgtggcaaatattggggacaagaagccatgcaatgccagtgaccctcagtgtccccccgatggccgttaccagtacaacactgatgtggtgtttgattctgagggtaggctggtggcacgctaccataagtacaatctttttgcgcctgaaattcaatttgatttccccaaggattcagaatctgtgacttttgagactccttttgggaagtttggcattttcacttgctttgacattttttctcacaccccagctgtggtggtggtggatgagtttcaagtggacagcgtcctctacccggtggcgtggtacaacacgctgccccttctgtcggctgttcccttccactctgcgtgggccagagccatgagagtcaacttgcttgctgcgaatacccacaacaccagcatgcacatgacagggagtggaatctacgcaccggaagcggtcaaagtgtatcactatgacatggaaacagagagtggccagctgatgctgtccgaactgaagtcccggccgcgaagtgaacccacctatcctgtagctgttgactggagtgcttatgccagaagtgtcaagccattcttgtctgaacagtcaaattttccggggatgatttattttgatgagctcaccttcattgagcttaagagaaacacaggaaattacacagtttgccagaaagacctgtgctgtcacttaacttacaagatgtcggagaaacgaacagatgaggtttacgcactaggtgcttttgatggactgcacacagtagaaggtcaatattacctacagatatgcacattactgaagtgtcaaatcagtgacctgagaacttgtggggagcccgtggggtcagctttcaccaagttggaagaattctccctcagcggcacatttgcaacacgttatgttttcccacagatcattctcagtggaagtcagcttgctcctgaaaggcattatgaggtcttgagagacggacgtctgaggagccgaagcagaacccctctgcctgtcttagttttggccctgtacggaagagtgtttgacaaagaccctccgcgcttggggcaaggaccccggagattagaatga

SEQ ID NO: 150 - DNA, 1506 bp, Panthera tigris
  source 1..1506: mol_type = genomic DNA
atgattacatcatattttccaagatatgtggcactatttgccatctgtgtcctatgtgtcagtgcactggacactttcattgcttcagtgtatgaacacgctgtcatattaccaaacagaacagagacaccagtttccagagaagaagctttgctcctgatgaacaagaacatagatgttttggagaacgcagttaagctggcagccaggcagggtgcacatatcattgttaccccagaagatggaatttatggctgggttttcacaagagagaccatttacccctatctggaagatatcccagacccagaggtgaactggattccatgtacagatccccagaggtttggctacaccccagtgcaacaaaggctcagctgcctggccaaggacaactccatctacgttgtggcaaatattggggacaagaagccatgcaatgccagcgaccctcagtgtccccccgatggccgttaccagtacaacactgatgtggtgtttgattctgagggtaggctggtggcacgctaccataagtacaatctttttgcgcctgaaattcaatttgatttccccaaggattcagaatctgtgacttttgagactccttttgggaagtttggcattttcacttgctttgacattttttctcacaccccagctgtggtggtggtggatgagtttcaagtggacagcgttctctacccggtggcgtggtacaacacactgccccttctgtcggctgttcccttccactctgcgtgggccagagccatgagagtcaacttgcttgctgcgaatacccacaacaccagcatgcacatgacagggagtggaatctacacaccggaagcggtcaaagtgtatcactatgacatggaaacagagagtggccagctgatgctgtccgaactgaagtcccggccgcgaagtgaacccacctatcctgcagctgttgactggagtgcttatgccagaagtgtcaagccattcttgtctgaacagtcaaattttccggggatgatttattttgatgagctcaccttcattgagcttaagagaaacacaggaaattacacagtttgccagaaagacctgtgctgtcacttaacttacaagatgtcggagaaacgaacagatgaggtttacgcactaggtgcttttgatggactgcacacagtagaaggtcaatattacctacagatatgcacattactgaagtgtcaaatcagtgacctgcgaacttgtggggagcccgtggggtcagctttcaccaagttggaagaattctccctcagcggcacatttgcaacacgttatgttttcccacagatcattctcagtggaagtcagcttgctcctgaaaggcattatgaggtcttgagagacggacgtctgaggagccgaagcagaacccctctgcctgtcttagttttggccctgtacggaagagtgtttgacaaagaccctccgcgcttggggcaaggaccccggagattagaatga

SEQ ID NO: 151 - DNA, 1449 bp, Hippopotamus amphibius
  source 1..1449: mol_type = genomic DNA
atgattatatcatattttccaaaatatgcggccactttggccttctttgtcctgagtgccggtgctctggacactttcattgctgcagtgtatgagcacgctgttgtattaccaaacaggacagaaacacctgtgccgaaagaagcagctttgctcctgatgaacaagaatatagatgttttagagaaagcagttaaactggcagccaggcggggcgcgcatatcattgtgaccccagaagatggaatttatggctgggtcttcacaaggggcaccgtttatccctatctggaggatatcccagaccctgaagtgaactggattccatgtagagattcccagaggtttggccgcaccccagtgcaagaaaggctcagctatttggccagggacagctctatctacttcgtggcaaatattggggacaagaagccatgcaataccagtgaccctcagtgtcccccgnacggtcattaccaatacaacactgatgtggtgtttgattctgagggtaggctggtggcctgctaccacaaggacaatctttttgcacctgaaattcaatttgatttccccaaggattcagaatttgtgacttttgacacaccctttgggaagtttggcattttcacttgctttgacattttttctcatgagccagctatggtggtggtggaggagttacaggttgatagtgttctccaccccatggcctggtacaacacactgcccctgctctcggcggtccccttccattctgcgtgggcccgagccctgcgagtcaatctgctagccgccaatacccacaccaccagcatgcacgtgacaggtacttcatgccgctcctgccccaatgggcagccgtgggtgtgctatgacacggaaacagagagtggccagctgatgctctcagaattgaagttgtggcctcgaagagaacgcagttaccctgcagctgttgactggagtgcttatgccagaggtgtcaaaccattcccatctaaactgttggattttccagggatgctttattttgatgagttcaccttcaccaagcttgagagaagcacaagaaattacacagcttgtcagaaagacctgtgttgtcacnnaacttacaagatgtctgagaaacgaacccatgaggtatatgtactaggtccctttgatggactgcacacggtagaaggtcaatattacttacaggtatgcacattactgaagtgtcaaaccactgacctgagaacttgtggagagcctgtggggtcagcttttaccaaatttgaagatctctccctcaggggcacatttggaacgcgttatgttttcccacagatcattcttagtgggagtcagcttgcccctgaaagacattatgaggtttcaagagatggacgtctgcggagccaaggtggaggcccttttcctgtcttggtcctggccctgtag

SEQ ID NO: 152 - DNA, 1500 bp, Gracilinanus agilis
  source 1..1500: mol_type = genomic DNA
atggctactgcctggtttcccacttgtgtgatagttttggcgatctgtatcctcgatggcggtgcctcagacacatttaaggctgctgtatatgagcatgctgttatattgccaaataggacagaaatacctgtttctcagaaagatgccctgatgttgatgaataagaacttagatgttttggagaaggcaatcaaattggcggctaagcagggtgcagacattattgtgactcctgaagatggagtttatggctgggatttcacaagggaaaccatttacccctacttagaagatatcccagaccctcaagaaaactggattccatgtactgaagcccacagatttggccgtactccagtgcaaaaaagacttagctgcttggccagagagaattctatctacgtggtggcaaatattggagacaagaaagaatgtggtgctaatgaccccacatgcccaccagatggtcggtaccaatacaacactaatgtggtgtttgattcagatgggaaactggtggcccgctaccataagtacaatctttttgcaactgaagttcagttcaacttccctaaagattcaccatatgtgacctttgatacaccctttggaaagtttggcattttcacttgctttgacatattttcttatgaacctgctgcagctgtggtggataaattccaagtcgacagcatactgtaccccacagcctggtacaatactctgcccctcttgtctgctgttccattccattcagcctgggccagggccatgagagtcaatctcctttcagcgaacactcacaacaccaggatgcacatgacagggagtggaatctatgcaccagaggcagttaaagtttatcactatgacatggaaacaaaaagtggccaactattgctctcagaactgaagacacggccacgactttcttctgactaccctgcagtggtagattggagttcctatgccaaaagtgccaagccattcatttctgatcagccaaagttccatggaagtatctattttgatgaatttaccttcattgaacttactaaagaaacaggaaattacaccatttgccagaaagaactctgctgtcacttgagctacaagatgtctgagaaaagaactgatgaagtctatgcactaggggcttttgatggccttcacattgttgaaggacaatattatttacagatatgcacattattgaagtgccaaacaactgacttaaaaacctgtggtgagccagttggatcagctttcaccagatttgaagaattctccctcagtggcacattcaacaccagatatgtcttcccccagttagtactaagtgggactcaacttgcacatgaacaacactatgaggtatcaaatgatggtcgtctgaagagcagaagtggtgtctcattgcctgttttagtgatggcgctatatgggagagtatttgagaaggacccacatcgtttaggacagggtgatcatccttga

SEQ ID NO: 153 - DNA, 1347 bp, Puma yagouaroundi
  source 1..1347: mol_type = genomic DNA
atgaacaagaacatagatgttttggagaacgcagttaaactggcagccaggcagggcacacatatcattgttaccccagaagatggaatttatggctgggttttcacaagagagaccatttacccctatctggaagagatcccagacccagaggtgaactggattccatgtacagatccccagaggtttggctacaccccagtgcaacaaaggctcagctgcctggccaaggacaactccatctacgttgtggcaaatattggggacaagaagccatgcaatgccagtgaccctcagtgtccccctgatggccgttaccagtacaacactgatgtggtgtttgattctgagggtaggctggtggcacgctaccataagtacaatctttttgcgcctgaaattcaatttgatttccccaaggattcagaatctgtgacttttgagactccttttgggaagtttggcattttcacttgctttgacattttttctcacaccccagctgtggtggtggtggatgagtttcaagtggacagcgtcctctacccggtggcgtggtacaacacgctgccccttctgtcggctgttcccttccactctgcgtgggccagaaccatgagagtcaacttgcttgctgcgaatacccacaacaccagcatgcacatgacagggagtggaatctacgcaccggaagcggtcaaagtgtatcactatgacatggaaacagagagtggccagctgatgctgtccgaactgaagtcccggccgcgaagtgaacccacctatcctgcagctgttgactggagtgcttatgccagaagtgtcaagccattcttgtctgaacagtcaaattttccggggatgatttattttgatgagctcaccttcattgagcttaagagaaacacaggaaattacacagtttgccagaaagacctgtgctgtcacttaacttacaagatgtcggagaaacgaacagatgaggtttacgcactaggtgcttttgacggactgcacacggtagaaggtcaatattacctacagatatgcacattactgaagtgtcaaatcagtgacctgagaacttgtggggagcccatggggtcagctttcaccaagttggaagatttctccctcagcggcacatttgcaacacgtcatgttttcccacagatcattctcagtggaagtcaacttgcgcctgaaaggcattatgaggtcttgagagacggacgtctgaggagccgaagcagaacccctctgcctgtcttagttttggccctgtacggaagagtgtttgacaaagaccctccgcgcttggggcaaggaccccggagattagaataa

SEQ ID NO: 154 - DNA, 1545 bp, Microcebus murinus
  source 1..1545: mol_type = genomic DNA
atgatcacttctccgttcctggcttatgtggcaatttgggttttctgtgtcttgagaaccagctctctggacacttttattgcagctgtttacgagcatgcggtgctattgcctaatgccagcctcacaccagtatctcgtgaggaggcgctgaaatggatggatcagaatctggatattttggaaggagccatcacgtcagcagcagagcagggtgcacatattatcgtgactccagaagatggtatttatggctggcacttcagcagggaatctgtctacccgtacttggaggatatcccagaccctcaagtaaactggatcccctgtgataatcctaacagatttggccacaccccagtacaagaaagactcagctgcctggccaaggacaactctatctatgttgtggcaaatattggggacaagaagccatgcaatgccagtgaccctcagtgtccccctgatggccgttaccaatacaacactgatgtggtatttgattctcagggtaggctggtggcacgctaccataagcaaaatcttttcatggttgaaaatcaattcaatgcacccaaggagcctgagattgtgactttcaacaccacctttggaaggtttggcatttttacatgcttcgatatactcttccatgatcctgctgttaccctggtgaaagacttctacgtggataccatactcttcccaacggcttggatgaatgttttgccacatttgtcagccattgaattccactcagcgtgggctatgggcatgggggtcaatttccttgcatctaatacacattacccctcaatgaggatgacaggaagtggcatctatgcccccaattctgcaagagcatttcattatgatatgaagacaaaggagggaaaactcctcctctcacaactggattcccacccacgccaccctgtagtggtgaattggacatcttatgccagcagtgtggaagcactctcaactggaaaccaggaatttaagggcattatcttttttgatgaatacaccttcgtggagctcaaaggagttatggggaattacacagtttgtcagaaagatctctgttgtcatctgaactacaagatgtcagagaaaagatcagatgaagtttatgccctaggggcatttgatggactgcacacggtggaaggacgctattatctacagatttgtactttggtgaagtgtaaaacgactgacttaaacacttgtggtgactcagttgaaacggcttctacccggtttgaaatgttctccctcagtggcactttcggaactcagtatgtcttccctgaggtattgttgagtgagatacagcttgcacctggagaatttcaggtgtcaagtgatggacgcttgtttagcctgaagccaatatctggacctgtcttaacagtgactctgtttgggaggttgtatgagaaggaccgagcatcaaacgcttcatccaacctcatagagcaagcactaagaataatgctagcagttataatatctattgtatactcattacgttggtag

SEQ ID NO: 155 - DNA, 1257 bp, Balaenoptera musculus
  source 1..1257: mol_type = genomic DNA
atggccttcctactgaaggcgcccagcagggaatctatctacccatacttggaggatatcccagaccctcaagtgaactggatcccttgtaataatcccaacagatttggccgcaccccagtgcaagaaaggctcagctgtctggccaaggacaactccatctacatcgtggcaaatattggggacaagaagccatgcaataccagtgaccctcactgtccccctgatggtcgttaccaatacaacactgatgtggtgtttgattctgaggggaaactggtggcccgctaccataagcaaaatcttttcttgggtgaagatgagttcaatgctcccaaggaacctgaggtcgtgactttcaacaccacctttggaaagtttggcattttcacgtgctttgatattctcttccatgatcctgctgtgactctggtgaaagatttccacgtggacaccatactcttcccgacagcttggatgaatgttttgccacatttgtcagctattgaattccactcagcttgggctatgggtatgggagtcaacttccttgcatccaatctacattacccctcaaagaaaatgacaggaagtggcatctacgcacctgattctccaagagcatttcattatgatatgaagacagaggagggaaaactcctcctctcacaactggcctcccacccacaccccacagcagtagtgaattggacttcctatgccagtggtatagaagccccctcaatgggaaaccaggaatttaagggcaccatcttttttgacgagttcaccttcttggagctcaaaggagtcgcaggaaattacacagtttgtcagaaagatctctgctgtcatctaagttacaggatgtctgagaagagatcagatgaagtttatgtactaggggcatttgatggactacatactgtggaagggagctattatttacagatttgtactctgttgaagtgtaaaacaacggacttacacacttgtggtgattcagttgaaactgcttccaccaggtttgaaatgttctccctcagtggcacttttgaaacccagtatgtcttccctgaggtgctgctgagtgaaattcagcttgcacctggagaatttcaggtgtcaagtgacggacgcttgtgcagccagaagccactatctggacctatcttgacagtaactctgtttggaaggttatatgagaaggatagttcaccaaatgctttctcagaccccatggcacagatcatttaa

SEQ ID NO: 156 - DNA, 1617 bp, Rhinopithecus roxellana
  source 1..1617: mol_type = genomic DNA
atgattggatcatattttccaaaatgtgtggcagttgttgccctccttgccttgagtgttggtgcattggacacttttattgctgcagtatatgagcatacagtgatattaccaaacagaacagaaacccctgtttcaaaagaagaaggtttgctcccgatgaacaagaacatagatgttttggagaaagcagttaagctggcagcgaagcgggtattatccttttatacttgtaaaggagacttgcagtttgataaaagagtatttggaattcatgagaaacttttttgccccacttgttttgagcagggtgcacatatcattgtgaccccagaagatggaatctatggttgggtcttcaccagggagagcatttacccttatttggaggatataccagaccctggagtgaactggattccgtgtagagacccctggagatttggcaacacaccagggcaacaaagactcagctgcctggccaaggacaactctatctatgttgtggctaatactggggacaagaagccatgcaatgccagtgaccctcagtgtccccctgatggccgttaccaatacactgatgtggtgtttgattctcagggaaaactggtggcacgctaccgtaagtacaatctttttgcacctgaaattcaattcgatttctccaaggattcagaacttgtgacttttgacactccctttgggaagtttggcatttttacttgctttgacattttttctcatgacccagctgttgtggtggtggatgagtttcaagttggcagcattctctaccccacagcatggtacaacacgctgcccctcctctcggctgttcccttccattcagcatgggccaaggccatgggagtcaatctacttgctgcaaatacccacaacaccagcatgcatatgacagggagtggaatctacaccccagaagcagtcaagatgtaccactatgacatagaaacagagagtggtcagctgttgctatcagaactgaagtctcagccttgccgtgagcccacctaccctgcagctgttgactggcatgcgtatgccagcagtgtcaaaccgttttcctctgagcagtcagattttccggggatgatttattttgatgagtttaccttcactgagcttaagagaaatataggaaattacacagtttgccagaaaaatctgtgttgtcacttaacttacaagatgtctaagaagcaagcagatgaggtctatgctctaggtgcttttgatggactgcacacagtagaaggccaatattacttacagctagaaatgcttaaatatatgtgcacattactgaagtgtcaaaccactgacctggaaacgtgtggagaagctgtggggtcagcttttaccaagtttgaggacttctctctcagtggcacattcggaacgcattatgttttcccacagctcattctaagtgggagtcagcttgcccctgaaagacattatgagatttcaagagatggacacttgaggagccgaagtagagcccctttgcctgtcttagttatggccctgtatggaagagtgtttgagaaggaccctccacgcttagggcagggacccgggaagttccagtga

SEQ ID NO: 157 - DNA, 1284 bp, Felis catus
  source 1..1284: mol_type = genomic DNA
atgactgtgactccagaagatgcgctttatggatggaaattttccagagaaaccatttctctttatctgaaggatatcccggcccctcaggtgaattggattacatgtcaaaacacccagaggtttggctacaccccagtgcaacaaaggctcagctgcctggccaaggacaactccatctacgttgtggcaaatattggggacaagaagccatgcaatgccagtgaccctcagtgtccccccgatggccgttaccagtacaacactgatgtggtgtttgattctgagggtaggctggtggcacgctaccataagtacaatctttttgcgcctgaaattcaatttgatttccccaaggattcagaatctgtgacttttgagactccttttgggaagtttggcattttcacttgctttgacattttttctcacaccccagctgtggtggtggtggatgagtttcaagtggacagtgttctctacccggtggcgtggtacaacacgctgccccttctgtcggctgttcccttccactctgcatgggccagagccatgagagtcaacttgcttgctgcgaatacccacaacaccagcatgcacatgacagggagtggaatctatgcaccggaagcagtcaaagtgtatcactatgacatggaaacagagagtggccagctgatgctgtccgaactgaagtcccggccgcgaagtgaacccacctatcctgcagctgttgactggagtgcttacgccagaagtgtcaagccattcttgtctgaacagtcaaattttccggggatgatttattttgatgagctcaccttcattgagcttaagagaaacacaggaaattacaccgtttgccagaaagacctgtgctgtcacttaacttacaagatgtcggagaaacgaacagatgaggtttacgcactaggtgcttttgatggactgcacacagtagaaggtcaatattacctacagatatgcacattactgaagtgtcaaatcagtgacctgagaacttgtggggagcccgtggggtcagctttcaccaagttggaagaattctccctcagcggcacatttgcaacacgttatgttttcccacagatcattctcagtggaagtcagcttgctcctgaaaggcattatgaggtcttgagagacggacgtctgaggagccgaagcagaacccctctgcctgtcttagttttggccctgtacggaagagtgtttgacaaagaccctccgcgcttggggcaaggaccccggagatttgaatga

SEQ ID NO: 158 - protein, 500 aa, Mus musculus
MASLHFPQWAVSFVFFAQAVGSMDTFIAAVYEHAVILPNKTESPVSTEEALLLINKNIDILESAIKLAARQGAHIIVTPEDGIYGWIFTRETIYPYLEDIPDPEVNWIPCRDPRRFGYTPVQERLSCLAKENSIYIMANIGDKKPCNATDPHCPPDGRYQYNTNVVFDSKGRLTARYHKYNLFEPEIQFDFPKDSELVTFDTPFGKFGIFTCFDIFSYDPAVVVVKDTQVDSVLLPTAWYNTLPLLSAVPFHSVWARAMGVNVLAANTHNTSMHMTGSGIYSPEAVRVYHYDMETESGQLLLSELRSRPRQHATPAEVNWSAYARTVKPFSSGQADFPGKIYFDEFSFTKLTGSAGNYTVCQKDLCCHLTYKMSESRMDEVYVLGAFDGLHTGEGQYYLQICTLLKCQTTNSRTCGEPVGSAFTKFEEFSLSGTFRTKYVFPQIVLSGSQLALERYYEVSRDGRLRSRGGAPLPILVMALYGRVFERDPPRLGQGPGKLQ

SEQ ID NO: 159 - protein, 500 aa, Mus caroli
MASLHFPQWAVSFVLFAQAVGSMDTFIAAVYEHAVILPNKTESLVSTEEALLLINKNIDILESAIKLAARQGAHIIVTPEDGIYGWIFTRETIYPYLEDIPDPEVNWIPCRDPRRFGYTPVQERLSCLAKENSIYIVANIGDKKPCNATDPHCPPDGRYQYNTNVVFDSKGRLTARYHKYNLFEPEIQFDFPKDSELVTFDTPFGKFGVFTCFDIFSYDPAVVVVKDAQVDSVLLPTAWYNTLPLLSAVPFHSAWARAMGVNVLAANTHNTGMHMTGSGIYSPEAVRVYHYDMETESGQLLLSELRSRPRQHATPAEVNWSAYARTVKPFSSGQADFPGKIYFDEFSFTKLTGSAGNYTVCQKDLCCHLTYKMSESRMDEVYVLGAFDGLHTVEGQYYLQICTLLKCQTTNSRTCGEPVGSAFTKFEEFSLSGTFRTKYVFPQIVLSGSQLALERYYEVSRDGRLRSRGGAPLPILVMALYGRVFERDPPRLGQGPGKLQ

SEQ ID NO: 160 - protein, 500 aa, Rattus rattus
MVSLYFPQCVVSFVLFAQAMGAMDTFIAAVYEHAVILPNKTESPVSNEEALLLINKNIDILENAIKLAARQGAHIIVTPEDGIYGWIFTRETIYPYLEDIPDPEVNWIPCRDPKRFGLTPVQERLSCLAKENSIYIVVNIGDKKPCNATDPQCPRDGRYQYNTNVVFDSEGRLTARYHKFNLFEPEIQFDFPKDSELVTFDTPFGKFGVFTCFDIFSYDPAVMVVKDSRVDSVLLPTAWYNTLPLLSAVPFHSAWARAMGVNVLAANTHNTRMHMTGSGIYSPDAVRVYHYDMETESGQLLLSELRSRPHQHGSPAEVDWSAYARTVKPFSSEQADFQGTIYFDEFSFTKLTGSAGNYTVCQKDLCCHLTYKMSESRMDEAYVLGAFDGLHTVEGQYYLQICTLLKCQTTNLRTCGEPVGSAFTKFEEFSLSGTFGTKYVFPQIVLSGSQLALERYYEVSRDGRLRSRGGAPLPVLVMALYGRVFERDPSRLGQGPGKVQ

SEQ ID NO: 161 - protein, 510 aa, Peromyscus californicus
MVPLYFPKCVVSFALFAQAVGALDTFIAAVYEHAVILPNKTETPVSAEEALLLMSKNIDVLEKAIKLAAEQGAHIIVTPEDGIYGWVFTRETIYPYLEDIPDPEVNWIPCRDPKRFGYTPVQERLSCLAKDNSIYVVANIGDKKPCNATDPQCPLDGRYQYNTNVVFDSEGRLTARYHKYNLFEPEIQFDFPKDSELVTFDTPFGRFGIFTCFDIFSYDPAVVTVKDFQVDSVLYPAAWYNTLPLLSAVPFHSAWARAMGVNLLAANTHNTTMHMTGSGIYSPEAVGVYHYDMETDSGQLLLSELKSRPRQSTPAAEVDWSAYARSIKPFSSEQADFPGMLYFDEFSFTKLSGSTGNVTVCQKDLCCYLTYKMSEKRMDEAYVLGAFDGLHTVEGQYYLQICTLLKCQTTNLRTCGEPVGSAFTKFEEFSLSGTFGTNYVFPQLVLSGSQLALERYYEVSRDGRLRSRGGVPLPVLVMALYGRMFEKDPPRLGQGPRKLQRNPFTGDCQD

SEQ ID NO: 162 - protein, 501 aa, Molossus molossus
MIASYFPKYGAVLALFVLSVGALDTFTAAVYEHAVILPNRTETPVSKEEALLLMNKNIDVLEKAVKLAARQGAHIIVTPEDGVYGWVFTRETIYPYLEDIPHPEVNWIPCKDPQRFGYAPVQERLSCLAKDNSIYVVANIGDKKPCNASDPQCPPDGRYQYNTDVVFDSEGRLVARYHKYNLFAPEIQFDFPKDSELVTFDTPFGKFGIFTCFDIFSHDPAVVVVTEFQVDSVLYPTAWYNTLPLLSAVPFHSAWARAMGVNLLAANTHSTRMHMTGSGIYAPEAVKVYHYDMETESGQLMLSELRSRPRREHAYPAPVDWSAHAKGVKPFSSEQSDFPGMIYFDEFTFTELKRKAGNYTVCQKDLCCHLTYRMSEKRTDEVYALGAFDGLHTVEGQYYLQICTLLKCQTTDLGTCGEPVGSAFTKFEEFALSGTFGTSYVFPQLILSGSQLAPERHYEVSRDGRLRSRSGAPLPVLVMALYGRVFEKDPPRLGQGPGKCQ

SEQ ID NO: 163 - protein, 501 aa, Homo sapiens
MIISHFPKCVAVFALLALSVGALDTFIAAVYEHAVILPNRTETPVSKEEALLLMNKNIDVLEKAVKLAAKQGAHIIVTPEDGIYGWIFTRESIYPYLEDIPDPGVNWIPCRDPWRFGNTPVQQRLSCLAKDNSIYVVANIGDKKPCNASDSQCPPDGRYQYNTDVVFDSQGKLLARYHKYNLFAPEIQFDFPKDSELVTFDTPFGKFGIFTCFDIFSHDPAAVVVDEVSIDSILYPTAWYNTLPLLSAVPFHSAWAKAMGVNLLAANTHNTSMHMTGSGIYAPEAVKVYHYDMETESGQLLLSELKSRPRREPTYPAAVDWHAYASSVKPFSSEQSDFLGMIYFDEFTFTKLKRNTGNYTACQKDLCCHLTYKMSEKRTDEIYALGAFDGLHTVEGQYYLQICALLKCQTTDLETCGEPVGSAFTKFEDFSLSGTFGTRYVFPQIILSGSQLAPERHYEISRDGRLRSRSGAPLPVLVMALYGRVFEKDPPRLGQGSGKFQ

SEQ ID NO: 164 - protein, 501 aa, Felis catus
MITSYFPKCVALLAICVLCVGALDTFIAAVYEHAVLLPNRTETPVSKEEALLLMNKNIDVLENAVQLAARQGAHIIVTPEDGIYGWVFTRETIYPYLEDIPDPEVNWIPCTDPQRFGYTPVQQRLSCLAKDNSIYVVANIGDKKPCNASDPQCPPDGRYQYNTDVVFDSEGRLVARYHKYNLFAPEIQFDFPKDSESVTFETPFGKFGIFTCFDIFSHTPAVVVVDEFQVDSVLYPVAWYNTLPLLSAVPFHSAWARAMRVNLLAANTHNTSMHMTGSGIYAPEAVKVYHYDMETESGQLMLSELKSRPRSEPTYPAAVDWSAYARSVKPFLSEQSNFPGMIYFDELTFIELKRNTGNYTVCQKDLCCHLTYKMSEKRTDEVYALGAFDGLHTVEGQYYLQICTLLKCQISDLRTCGEPVGSAFTKLEEFSLSGTFATRYVFPQIILSGSQLAPERHYEVLRDGRLRSRSRTPLPVLVLALYGRVFDKDPPRLGQGPRRFE

SEQ ID NO: 165 - protein, 503 aa, Tachyglossus aculeatus
MIFTAFPVYAVFFALWAPGARTEDSFIAAVYEHAVILPNGTQTPVTQDDALMLMDKNLDVLEKAIKQAASEGAHIIVTPEDGIYGWVFTRDTLYPYLEDIPDPQVNWIPCTDPNRFGRAPVQKRLSCLARDNSIYVVANMGDKKPCNTTDSQCPRDGRYQYNTNVVFGADGKLVARYHKYNLFEPEVQFDFPKDSQDVTFETPFGKFGIFTCFDIFSHDPAVTVVEKYQVDSVLYPTAWDNTLPLLSAAPFHSAWARSMRVNLLAANTHNTSMHMTGSGIYAPDGIRAFHYDMETESGQLLLSELKSRPRLWPSYPVAVDWSAYAKHVKPPSREYKKFGGKIYFDEFTFTDLIQAGENYTVCENDLCCHLSYKMTEKRADEVFVLGVFNGLHIVEGQYYLQVCTLLKCQTTDLRTCGEPIGSAFTKFEEFSLSGTFGTNYVFPQVILSGSQLATEQQYEVTSDGRLRNRGSVPLPILVMALYGRVYEKDPPRLGQGVRPLRPL

SEQ ID NO: 166 - protein, 483 aa, Myotis lucifugus
MITSSFQASVAVLALVTLHVSALDTFIAAVYEHAVLRPKGTETPVSRDDALLLMNKNIDILEKAIKQAAEQGAQIIVTPEDALYGWKFTRETIFPYLEDIPDPQVDWIPCQDPQRFGHTPIQTRLSCLAKNNSIYVLANIGDKKRCNSQDSTCPPNGYYQYNTNVVYDAKGQLVARYHKYHLYSEPQFDVPEKPELVTFNTTFGRFGIFTCFDILFHDPAVSLVKDFHVDTILYPTAWMNVLPLLSAIEFHSAWAMGMRVNLLAANIHHVQQNMTGSGIYAPHSPRVYHYDMETDSGRLLLSEVDSRPRTSPAYPTAVDWSAYATTIKPFPVQKNTFSGFISRDAFNFTALFENAGNLTVCHKDLCCHLSYRMLRKEENEVYVLGAFTGLHGRRRREYWQVCTMLKCKTTDLTTCGQPVETALTRFDIFSLSGTFRTEYVFPEVLLTKVHLSPGKFEVGGLEEYFYFICSLKPSKIGYGNCIT

SEQ ID NO: 167 - protein, 514 aa, Equus asinus
MISSQLLAYVAVLVFCTFQARSLETFIAAVYEHAVILPNATLTPVSREEALLLMNRNLDLLEGAIASAAKQGAHIIVTPEDGVYGWNFSRDTLYPYLEDIPDPEVNWIPCNNPHRFGHTPVQERLSCLAKDNSIYVVANFGDKKPCNASDPECPPDGRYQYNTDVVFDAQGKLVARYHKQNLFMGEDQFNAPKEPEVVTFNTTFGKFGIFTCFDILFYDPAVTLVTDFQVDTILFPTAWMNVLPHLSAIEFHSAWAMGMGVNFLASNIHYPSMKMTGSGIYAPDSPRAFHYDMKTEEGKLLLSQLDSHPHHPAVVNWTSFASGVKTLSTGNQEFNSTVFFDEFTFLELTEVTGNYTVCQKALCCHLSYKMSEKRSDEVYALGAFDGLHTVEGSYYLQVCTLLKCRTTDLHTCGDSVETASTRFDTFSLSGTFGTQHVFPEVLLSEIQLAPGEFQVSSDGRLFSLQPPSGPVLTVTLYGRLYEKDQVSDASSDLTAQALRVMLVVLIPVGFSISW

SEQ ID NO: 168 - protein, 514 aa, Egretta garzetta
MLHKKHSRSCEAAAMLPSQSLLHAAVFALTVLQALASDTFIAAVYEHAVILPDATDEPVSPDDALALMNKNMDVLEGAVKEAAQQGARIIVTPEDGIYGWRFTRETIYPYLEDIPDPAVNWIPCTDPTRFAPAPVQERLSCMARSNSIYVVANIGDKKPCNSSDAGCPRDGRYQYNTNVVFDSEGKLVARYHKYNLFMSETQFNYPKEPEAVTFETPFGKFGIFTCFDILFREPAVVLVSELQVDTVLFPTAWMNVLPFLTAVEFHSAWAMGMGVNFLAANTHYTNISMTGSGIYAPNGARTYYYNMKTEDGHLLIAELDSHPRLSPASPPAVSWSSYASSVESFSPNDHDFGGLIFHDWFTFTELTKPEGNLTVCQKDLCCHLSYKMAGKQKDEVYVLGAFDGLHVVEGQYYLQICTLLKCKNTDSNTCGQPVESAQTRFERFSLSGTFGTSYVFPEVLYSGVQLAPGEFEVLNDGRLISKKRTTKPVVTVTLFGRWYEKDHLKQDPQSPAFP

SEQ ID NO: 169 - protein, 520 aa, Bos javanicus; identical to SEQ ID NO: 115

SEQ ID NO: 170 - protein, 521 aa, Sus scrofa
MIASSFQTTLAVFALIILHVRALDTFKAAVYEHAVILPNDTKTPVSWDDALLLMNKNMDILERAIKQAAEQGSQIIVTPEDALYGWKFTRETIFPYLEDVPDPQKNWIPCQDPRRFGHTPVQVRLSCLAKNNSIYVLANIGDKKPCNSHDSKCPPNGYYQYNTNVVYDSEGKLVARYHKYHLYHELQFDVPEKPEVVTFNTPFGKFGIFTCFDILFRDPAVTLVQDFHVDTILFPAAWMNVLPLLTAIEFHSAWAMGMRVNLLAANVHHVRLNMTGSGIYAPHSPKVYHYDMETDSGKILLAEVDAHPRTSPTYPTAVNWGAYTLSIQPFPILQNSFRGFISRDEFHFTELSESAGNLTVCHKELCCHLSYRMLAKEENEVYVLGAFTGLHGRRRXEYWQVCTMLKCKSTNLTTCGQPVETALTRFELFSLSGTFGTEYVFPEVLLTKINLAPGKFEVLKDGRLVNKNGTSEPVLGVSLFGRWYIRDVPLSISGGPAPQQKMYLIISALLMIIALQNIVMV

SEQ ID NO: 171 - protein, 546 aa, Bos indicus
MLHKCSFLYKYYMNVTFTCRTQDTLAMITSSLQTSVAVFALXILHVSTLDTFKAAVYEHAVILPNDTETPVSPDEALFLMNKNIDILEKAIKQAAEQGAQIIVTPEDALYGWKFTRETIFPYLENIPDPQVNWIPCQEPHRFGHTPVQARLRSLAKNNSIYVVANMGDKKPCNSHNPXCPSNGHYQYXTNVVYDAKGKLVARYHKYHLYREIQFDVPEKPELVTFNTTFGKFGIFTCFDILFHDPAVTLVKDSHVDTILFPTAWMNVLPFMTAIEFHSAWAMGMRVNLLAANTHNVSLKMTGSGIYAPHSPKVYHHDMETESGKILFAEVDSHPRNSSTYPPAMNWSAYATSITPFPGPKNSFRGFISRDEFNFTKLSESAGNLTVCQKELCCHLSYRMLAKEGDEVYVLGAFAGLHGRRRREYWQVCTMLKCRSTDVTTCGQPVETALTRFEMFSLSGTFGTEYVFPEVLLTKIKLAPEKFEVLEDGRLVNKNGLSEPVLTVSLFGRWYXKDAHSISGGTSNSATAYLMIPTLLMIITLRDAVMX

SEQ ID NO: 172 - protein, 520 aa, Patella sp.; identical to SEQ ID NO: 130

SEQ ID NO: 173 - protein, 561 aa, Biomphalaria sp.
MGKEVRLLLLLLGTLSSVLANRSKFKGAVYGHAVLLPKDSLKVISRAEALDNMNKNLDVYREQVTKAKSQGAEIIVFPEDGLYGFQFTRQSIYPYLEYIPDPRTELWSPCDEPGRYNDTEVQRSLSCLAKTNSLYLVANMGDKQPCDAATDRTCPPDGRYQYNTNVVYGPDGRLLARYHKYNLFYELQFNAPELELSYFDTPFGRFGLMICFDVLFHDPGLPLILSLNISNIIFTSAWKDALPLLSALGYHSSFARGLGINMLGANIHIPDYRFHGSGIYGPDGTIISFYGSTNASKPKLLVADVNVLDDTQPNKIVSSIVQQNLKQARMNEADDKRVTKDTTKNSKKTFESLLFYDMFTFAALDKPSGHIKVNQKHVHCELRYSIPEELFGRELYAFGAFDGLHTYEGQYYFQICALVKCADARNRSTCGSVTYDAITPFVRLSISGEMQTPFVYPQIILSNGIGELSLAEPDSWTYTGKSLKTDKEFYKPVLSAALFGRDYFRDTDGQNKGDNLYKSQEDATDKEDKSEIYLGLVSGQNIFIFLLGFVCLLLLVNMAIF

SEQ ID NO: 174 - protein, 501 aa, Mya arenaria
MTVGSPGMLLTLLLVLGGRILAGLETPLADSYRAAVYEHNVSFPRHRDVVPRLEALQLMEENLGKFDEQARQAAQQKAKIIVFPEYGVYGLGWTRQKLAPFLEYIPDPAGNPWNPCDNPNGFVSAVQTLLSCIAKNNSIYVVANIGSQQPCISTDKSCPSDHVYQFNTNVVYDPRGIFIARYFKYNLDSVEMQYFDRPPVVNHTIFDTPFGRFATVCSNDILYKEPTLTLVKTMNITNLAMPAAWKEELPLMSAIEFHSAFAEGLLINVLAANLHVVDLGYYGSGLYWPTGTSINASYFYDDDPYSEGSLVVETLTPSIIPSDVWDPPQFTENKRFAPRRLVNNAEQSSLTHKIKINGDIYTAKVFKFGEAYSANVCQGSLCCDADFEGGASGDMFAFGAFEGMHSGNNSYYLQSCALIKCAGRTVESCGQPTKHTSTGIDKMSMSGNFSYPYQFPEVFTVGDHPEIVTQIWYYQGSVIIDVGLYGDLLTVAIQAIDYSRQ

SEQ ID NO: 175 - protein, 537 aa, Dissostichus mawsoni
MKHLYSRTQMLFMSLREGTMFLSVIVFSSLSVSTALGRTEPVVDSSYIAAVYEHNIILNPDPRVPLSRPAALQHMQRNLDIYEQQAARAAQQGVQILVFPEDGIQGFNFSRSSIFNYLETIPDPQQESWNPCTEPGKYNNTEVLQRLSCMARRHNLYLVANMADLQPCPLKTDPSSSCPSDGRWQFNTNVVFRSDGLLVGRYHKNNLYFEAAFDTPPQPEIITFDTPFAGRFGLIICFDILFYEPKVHLVTKGVRQLIFPTAWMNQLPLLDAIQFQHAFSLGANVTLLAANIRNDQMIMTGSGIYTPFSVKSHHAQKGDPDEGRLLVARVPVLDPLWAGQSVTDGSTAKDSGFCHKESCLDFPAPETSSSVPPSSSASSTSTFISSMMYDPFTFVLLEATEGKVNVCNGTFCCHLQYRLIPQGDSKELYALGAFGGTHTVNGRYAVQVCALVRCAGLDTSSCGQEVEEAESKMDFQLEGRFASRYVYPSVLASKFVLEQPKHFETAPDGTVTMKHSNMTGGLVTACLYGRMYHLENE

SEQ ID NO: 176 - protein, 620 aa, Priapulus caudatus
MAPIRLQCLRVGSPHRRALVVAAVLLATLQYPSATATARRTSSDDSSCTYTAAVLQYYLQTPGPGSDPSEVMRSNLLAYEEYLMQAAAQGASIIVFPETGITGFLQKDREIVLKWLQEIPEPNTEAALDGQRSSDSPDSCDSGCVATSGDDFVLESLKCMAACHKITIVANLGEVQYCQPAASSTVPTQVQSSAAAEDAVPGCPPDGRFQFNTNVVVDDNGALVVRYRKQHLYNEAAFDDPPVPEAGVFDTPFGRFTTFVCFDINFYNTSADVVLHDGVDSVAMSAAWPIALPTSSPQQTHSGWALGTGVNLLTANVRGVTDGLTFGGSGIYSGVSGNLTYTREDAPMQPELVIADVPCYRKRSWESYAHRPYSVEIYDAPVAAIARPAGGGGSGNKRRMKNKKNSRKRNEHRRRPGNEPNSENSETGDNSPRTGGEREQHTSAPPPKYFKNYVIGVLFNFTALEADIGRASVCYGDLCCHLDYAMSARGRRSDELYALAAFNDYYTAVTDQFWWQVCMVVKCRTSSYKTCGESVTRAKTKFKALTLSGDFWVEFISPSVRVDGDYLLAPLDQWSFDNDGEVMFSTKGIADPLITAEIQAFEPEKHDQSKRGVDVTCTCT

SEQ ID NO: 177 - protein, 526 aa, Ruditapes philippinarum
MDYYLWNMEAKKNVVCCFLLSIGISMITSAKVKVAVYEHVVISPDDPEALFTRQEAFQWMSKNLNVFVEQARKASEQGAKIIVFPEYGITGFDHSRNSIVPFLEDIPDSSTDWNPCMEPDRFADTLVLRNLSCTAQMYKIYIVANMGDMKTCDKMTDETCPHDGRYQFNTNVIFDNNGQLVARYHKLHMYDETPLFDPAPNAEHVYFDTPFGRFGTIVCFDIMQNEPTQVLLNKYSITNLLVTSAWNVFLPFIIPQQMYSGLAKKSGINVIASNIRNSLYQMAGSGIFGNDIQSLSDVNSNGAEGILLVSEIETEEAKIYAPNTIMSINSSGKVKQFINKTYEGTYRFGLNMSYAIMEGQSGIVTSCKDDVCCVVTYQFKSRDENETIILSVVDELLYKPGIIHMQYCAVFKCSPHTIYSCDYDDIFVINSIFADIKIQGYFDNGVTFPLVTTIDNNELTMDMDKYTFDDKNAILHSNQFENPLLAAVVYNTLHLDVIKTGASSNVAQGTTLSLSTFLLIYIYKAS

SEQ ID NO: 178 - protein, 511 aa, Paramuricea clavata
MAKQTLFFMLFVLSISSRILAKEYKVATFEHHPFFILAPEPLSREKALQMMRKNTAVYSEQARAARKQGAQIIVFPEDGIYGFFHSRETIKPYLEPIPTVSNTTSIIPCTDPKPNTEILQQLSCIARNNSIAVVANMGDIQYCSQRDPNCPKDGRYQYNTDVIFDKDGRLLAKYHKQHLYFEKAFNTPTKCEFVTFVTSFQVRFGVFTCFDMLFKDPAIDLVQTYGVQNIVVPTAWYKGFPALVSIEYHQAWSRVNCVNLVTANVYYPNRGLNFTGTGIYDCGEAKTYTYGEETIEKRLLFATLSGNREAQNDKTDMLYRWRRDIEMVKEFGMDRKIPQDIDNENRMLDDPYDSIKTFKAKIMGNVFTLTKLTSPASELSVCVKGLCCNLKYGLPEGKQFTETFAMGTFIGNNPDGFYWEVCLLLKCASSEESSCGTTVMDSGTVFQSFTMSGNFSSTALVFPMALGSGVSLFSTDKVKFANKSISGKALQKPLLTASLLGRVYSKDKPHN

SEQ ID NO: 179 - protein, 559 aa, Nilaparvata lugens
MGDIQYSIFKSEIHNHNNAVERKKMISTVRSFQRLFLLFLVACTLFATSIIVNGYLNSGRDKYVAAVVEYAPLEWDGFEKPKDLFLRNANRFVTIIERAAKENADIIVFPECGLTTLNIPVKREGITEYTTFIPNPVEKRTPCTDNDTGLAEHLRVLSCAAKNNKMYVAVNMDEKITCESDMQGCPKDHLIFFNTNIVFDRNGTIIAKYRKFNLFKEPEFNSTIFPIYAVFKTDFGATFGMFICFDILFAQPAEVIVSKYKVTDILYSTAWFSELPFLTAVQTQAGYSYSMNVNLLASGYNRPDLYSGGSGIYAGEDGPVVTTMPAFRTTRLLVGELTIYGTGRKRPVCEYADSCPLSKGKVIDIHFNDTHDKEGMLFLKDDLSKYSTEFIEETTSYEHAMIQHLGDDKETNLEYSRNGFNCSITVTWFRDDTSSNNKGPVYRLVAHSGIRSFAGVRSAYIEVCALVACADRTIESCGHRPSFENVSSRTQVKNVNIHTESLNITTPFVSTLDSNIYPLMGNELSFIESIDKFRGYIQKTYLFLEKPKDDLITFGVYKL

SEQ ID NO: 180 - protein, 512 aa, Ischnura sp.
MGLLHGILFVLLIPLCAAHRKTYRAAVVEYHPLGDRHDNASHILNLNVAEYETLIEEAKRKGADIVVFPEYGLTQLAASSSRNLAIQFAQSVPTPPAPGTPSSAAQVPCLLPNSSMAIRRLSCSARENQIYVVVNLLEIAPCSITDHACPEDGFFIYNTNVAFDRNGGVVSKYHKYNLFKEPSVNTPKHSMYSTFDTDFGVTFGMFICFDILFEDPGYTLVKRMNVNNMIYSTAWISELPFLTATQTQQSWSLGLGVNLLASGFNNPMGGNGGSGIYAGGSGRLNLLMPSTAGTTLLLADVPVVSSNRLRGDSLSEPELSLDLQTGNQATSGHMFVLHDDLSNYKSSEVLKNGLNELNMCQNNLCCNFKADVQLSSNGSQAPKYRFVITDSIIKYARTYPAHVQVCGLIYCKGDDVSKCVYFPDLPETLDNVSFRSLGISGNFSSPRMVLPSTLTTSLEPLKISSGNLMEKDEAITLEYNSDTRGLLTFALYGRIFQQNMIRNEEIMDNDIY

SEQ ID NO: 181 - protein, 533 aa, Argiope bruennichi
MKLPGIQFLCFFVMLSMGSAQMRSRRPYYRAAVHEHKQYDDKTSSGREIINKNLRKYEVAARLAARNYVDLIVFPEKGLFPMKMDNPTWFLDYAENIPRGDEMTNPCYQEKFSSSPILTNLSCIARKYSLFVVATLIDVKDCKVRRHCENRKNKNSCVSDGSDCPDNGHFNFNTLVVFDRMGTLIVRYYKRHPFIPLEKGISTPQYPESRYFDTEFGSFATDIGFDFLINDSFIDIAKRPETTGVTYGNWWFDHTPFFYFSVPNQQAWCLTNKVTVLSSDVHSPNLASLGSGIYIPGKGAVIYSYNPDGKSKLLISNIPTSFFYDFQSTLPPLNKRFFYINDDDSISELNGEEPRNFKDKCGENVLGMNPSSLTDYRCKQSEVHQYTFVKLNKTEDYINVCSNNFCCSLEYQAESMDETFYFAVSGNRLKFYDIYWFGVQSCFLSRCEPVDSKPCRNFLLKSNTKFNSVKITGSFDTNHIYPHVLDSELRLTDRDEWKFDGTSQLSYQNLKGKNLLHADLYGRLYREDGLVNN

SEQ ID NO: 182 - protein, 573 aa, Patella sp.
MMYKIMLLRIVATYTCLSLVEGYRPSYRAAVYEHALFQPTHFRTVSRNQALRDMKKNLDIYAEQTRIASSQNVDIIVFPEDGIYGLNFNRDTIKPYLEDIPDPTEITWNPCNQSEYTNGVEVQITLSCMARNNGIYIVANMGDKKVCDADSICPDDGQRQYNTDVVFDPQGNLVARYHKQHLYFEAQFDTPSTVERVTFDTPFGKFGIITCFDILFKSPAVDLVEDLGVKNVIFPTAWMDSLPLLSAIQFHSAFAKLFKVNLLAANLHFPLFNFHGSGIYSPRRTEAYYYNDGLFSPGKLLIADVKSSPTHYRRHQVVPDTHGFRGGNSLGASFVHGQDTAEVNTQSRRNGGHYNTASRNGIISHQLADQASGVHLNNDLGPLTHRSIRNRIHNHAHLHRVNRHMVENGWATIMADNDNYQYLPLTEPKAKVKICQNEVCCYLDYQKQNTSNDELYALGVFDGLHQIEGLSWYTQVCVLLKCEFTDIASCGQPTKIAAGHFEYLKLWGTFTTEYVFPEVLVSDDGKLRLEHEHWKYLHNRIEFVNRGKERPLLSATLYGRHYDRDNSTVSNSV

SEQ ID NO: 183 - protein, 568 aa, Lonchura striata
MLYHDPAVVMASRFQINTILFLTAWVNTLPLLSAAQFYLVQALGMGVNFLSANTHNSTLDMTGSGIYAPDSPRAYYYNTEMDNGHLLVTELSSHPHLSSDYPVAVNWTLSAIFPCSTCGQPVETVQTKFEMFSLSGIFGISYIFPEVLYSGVQLAPGEFKGAHIIVTPEDGIYGWRFTRESIYPYLENIPDPVVNWIPCTDPSRFGPAPVQERLSCMARNNSIYVVANIGDKKPCDSSDPSCPRDGRYQYNTDVVFDAQGKLVARYHKYNLFLGEDQFNYPKEPEAVTFETPFGKFGIFTCFDILFYEPAVVLVSKMQVDTVLFPTAWMNVLPFLTAIEFHSAWAMGMRVNVLAANTHNTSMEMTGSGIYAPAGARTYSYNKKTEDGHLLIAELDAHPRLSPVSPPAVSWNSYALSVERFSQNDHEFTGIIFEDLFTFTELTKPGENLTVCQKNLCCHLSYKMAEKRDDEVYVLGAFDGLHVIEGQYYLQICTLLKCPSTNLSTCGQPVETAQTKFEMFSLSGTFGTSYVFPEVLYSGVQLAPGEFKVMGHPCDLSDISGQKPTTRAT

SEQ ID NO: 184 - DNA, 642 bp, Corynebacterium glutamicum
  source 1..642: mol_type = genomic DNA
gtggctgctagcgcttcaggcaagagtaaaacaagtgccggggcaaaccgtcgtcgcaatcgaccaagcccccgacagcgtctcctcgatagcgcaaccaaccttttcaccacagaaggtattcgcgtcatcggtattgatcgtatcctccgtgaagctgacgtggcgaaggcgagcctctattcccttttcggatcgaaggacgccttggttattgcatacctggagaacctcgatcagctgtggcgtgaagcgtggcgtgagcgcaccgtcggtatgaaggatccggaagataaaatcatcgcgttctttgatcagtgcattgaggaagaaccagaaaaagatttccgcggctcgcactttcagaatgcggctagtgagtaccctcgccccgaaactgatagcgaaaagggcattgttgcagcagtgttagagcaccgcgagtggtgtcataagactctgactgatttgctcactgagaagaacggctacccaggcaccacccaggcgaatcagctgttggtgttccttgatggtggacttgctggatctcgattggtccacaacatcagtcctcttgagacggctcgcgatttggctcggcagttgttgtcggctccacctgcggactactcaatttag

SEQ ID NO: 185 - protein, 213 aa, Corynebacterium glutamicum
MAASASGKSKTSAGANRRRNRPSPRQRLLDSATNLFTTEGIRVIGIDRILREADVAKASLYSLFGSKDALVIAYLENLDQLWREAWRERTVGMKDPEDKIIAFFDQCIEEEPEKDFRGSHFQNAASEYPRPETDSEKGIVAAVLEHREWCHKTLTDLLTEKNGYPGTTQANQLLVFLDGGLAGSRLVHNISPLETARDLARQLLSAPPADYSI

SEQ ID NO: 186 - DNA, 669 bp, Corynebacterium glutamicum
  source 1..669: mol_type = genomic DNA
atggcaggagcagtgggacgcccccggagatcagctccgcgacgggcaggcaagaatcctcgcgaggagattcttgacgcctctgctgagcttttcacccgtcaaggcttcgcaacaacctccacgcatcaaatcgctgatgccgtgggaatccgccaagcctcgctgtattatcacttcccgtccaagacggaaatcttcctcaccctgctgaaatctactgtcgagccgtccactgtgctcgccgaagacttaagcaccctggacgccggacctgagatgcgcctctgggcaatcgttgcctccgaagtgcgtctgctgctgtccaccaagtggaacgtcggtcgcctgtaccaactccccatcgttggttctgaagagttcgccgagtaccacagccagcgcgaagccctcaccaacgtcttccgcgacctcgccaccgaaatcgtcggtgacgacccccgcgcagaactccccttccacatcaccatgtcggtgatcgaaatgcgtcgcaacgacggcaagattccaagcccgctttccgcagacagcctcccggagaccgcaattatgcttgccgacgcctccctcgccgtcctcggcgcgccgctgcccgccgaccgggtcgaaaaaacgcttgaactaatcaagcaggctgacgcgaaataa

SEQ ID NO: 187 - protein, 222 aa, Corynebacterium glutamicum
MAGAVGRPRRSAPRRAGKNPREEILDASAELFTRQGFATTSTHQIADAVGIRQASLYYHFPSKTEIFLTLLKSTVEPSTVLAEDLSTLDAGPEMRLWAIVASEVRLLLSTKWNVGRLYQLPIVGSEEFAEYHSQREALTNVFRDLATEIVGDDPRAELPFHITMSVIEMRRNDGKIPSPLSADSLPETAIMLADASLAVLGAPLPADRVEKTLELIKQADAK

SEQ ID NO: 188 - DNA, 1371 bp, Corynebacterium glutamicum
  source 1..1371: mol_type = genomic DNA
ttagaacaacgccccagcgaaggtcagctcgacagcttgggctgcagcttccaccgcgtccagcagtgctaccaattcgtcatcagtggtggtcagcggcggcgcgatgtggaatcggttgccggagatcatcggccacacgccgcgttccttgaattctgcagcaccggcagccatggcagtggcgtctgcattgaactccactgcccagaagaatccgatgccccgcacgtcagcgatcgctacgttttcttccgctagttcacgaaggcgaggttcgatcagttcagcgccaagtcgagctacgcgtggaatgatctctccttccgcgtaaatctccagcgctgccttggcgggtgctactgcaagtgggtgtccggagtaggtgagtccgccggagtatgcctctgatccgaaggtatcgcggattgattgggtcatcacgatgccaccgagtggggcgtaacctgcgttaacacccttggcgaaggtgatcatgtctggctggaaatcgtcgccagcatgctcgtaagcaaacagttttccggtgcgtccgaatccgaccatgacttcgtcggcgatgaagaggatgccgtgcttgttgcaaagttcgcgcacgccatttaagtaacctgctggtggcaggatgattcctgatgatcccaccactggctccaggacgatcgctgcgatcatgccagcaccttcaaacgcgatgacatcttccaagtgcttgagtgcgcgttcgcattcttcttcttgggtggtggcaaagaatgaggagtggtgcaggaatggtgcccagaagtggtagatatctgggtcggtggtggggttgcccaggcggcggtgttcgccggtgagcatcatcgcggatccggttgcgccgtggtagctgcggtatgcggacagaattttgttgcgtccggtgtgcaggcgagccatgcggatggagtgctcgatggcgtcggcgccgccgttggtgaaaaacacgtgggagaattcgccacgggccatcgacacgatctttgcagcaacatcagagcgcacatcattgccgaaggccgggttgatgttggtcaaccgggctgcttggcgctggatcgcctcaactaatcgagggttgttgtggcctaagtttgccgagacaagttgggaacccatgtcgatgaaggcgttgccgtcgaagtcgtacagcgtggaaccttcggcagctgcccatactttggggctgattttgtcctgcgcggaccaggagtggaaaacgtgcgcgcgatcgttgtcgaatgcccgtttttcctcttcttttgcttgcacagatccgaattcgatgaattcaccgtcaaagttggtgtaacccttcaatgccaa

SEQ ID NO: 189 - protein, 456 aa, Corynebacterium glutamicum
MALKGYTNFDGEFIEFGSVQAKEEEKRAFDNDRAHVFHSWSAQDKISPKVWAAAEGSTLYDFDGNAFIDMGSQLVSANLGHNNPRLVEAIQRQAARLTNINPAFGNDVRSDVAAKIVSMARGEFSHVFFTNGGADAIEHSIRMARLHTGRNKILSAYRSYHGATGSAMMLTGEHRRLGNPTTDPDIYHFWAPFLHHSSFFATTQEEECERALKHLEDVIAFEGAGMIAAIVLEPVVGSSGIILPPAGYLNGVRELCNKHGILFIADEVMVGFGRTGKLFAYEHAGDDFQPDMITFAKGVNAGYAPLGGIVMTQSIRDTFGSEAYSGGLTYSGHPLAVAPAKAALEIYAEGEIIPRVARLGAELIEPRLRELAEENVAIADVRGIGFFWAVEFNADATAMAAGAAEFKERGVWPMISGNRFHIAPPLTTTDDELVALLDAVEAAAQAVELTFAGALF

SEQ ID NO: 190 - DNA, 492 bp, Corynebacterium glutamicum
  source 1..492: mol_type = genomic DNA
atgacttcagcaatcaccaccgcaactgatcttcgctccgtactgcgaaacgtaccaaccccaattagcttcattgcaacccacaccgatcagcctctgggcatgatcgttggttcattcgtcagcattagcgccgaaccaccattggtaggcatcttcttgcagaagagctcttcttcatggccagctatcgagcaggcattagttaccggccaagagctaggcatttctatcctcggcggggcacacgcagaccatgtgcgtaagctttctggcccatccgaccagcgctttgaaaaccttgggtgggcatccaccgaaaacggtgcgattcaccttgaaggcgctgatgcacaactaaccacgaaacttcatgatctccaggaaatcggcgatcacttctttgcagttctagaagttattgacgcttccgctgaccaagacttcagctcagcgctggtgtaccaccgctcacaggtgtcctcgctgtag

SEQ ID NO: 191 - protein, 163 aa, Corynebacterium glutamicum
MTSAITTATDLRSVLRNVPTPISFIATHTDQPLGMIVGSFVSISAEPPLVGIFLQKSSSSWPAIEQALVTGQELGISILGGAHADHVRKLSGPSDQRFENLGWASTENGAIHLEGADAQLTTKLHDLQEIGDHFFAVLEVIDASADQDFSSALVYHRSQVSSL

SEQ ID NO: 192 - DNA, 1146 bp, Corynebacterium glutamicum
  source 1..1146: mol_type = genomic DNA
atgacattaactttccattggttcctatccacttcaggcgattcccgcggcatcatcggcggcggtcacggtgcagaaaaatccggcacctcccgcgaattgagccacagctacctcaagcagttggcgctagctgccgagaccaacggttttgaatctgtcctgacaccaacgggcacgtggtgcgaagatgcgtggattactgacgcttctttgattgaggcgacaaaacgcttgaagttcctcgttgcgcttcgccctgggcagattggacctacgctgtctgctcaaatggcttctactttccagcgtctgtctggcaaccgtttgctgatcaatgtggtcaccggtggggaagatgcggagcagcgtgcgtttggtgatttcttgaacaaggaggagcgctacgcccgtaccggagaattcttggatatcgtgagccgcttgtggcgaggcgaaaccgtcacgcaccacggtgaacacctgcaggtggagcaagctagccttgcgcatccgccagagattattccggagattctttttggtggatcgtcgccagctgcaggtgaggtggctgcacgttatgcggacacctatctcacgtggggtgaaactcccgatcaggtggcgcagaaaatcaactggatcaacgagctagcagcacagcgcggccgggaactgcgccatggaatccgcttccatgtgatcacccgcgatacgtctgaagaagcatgggtggtggcagagaagttgattagcggggtcactccagaacaggtcgctaaggctcaagccgggtttgcaacgtctaagtcggaggggcagcgccggatggctgagctgcacagcaagggtcgtgcctttactagtggctcaactgctcgtgatctggaggtgtatcccaatgtgtgggcaggcgtcggtttgcttcgcggaggtgcaggaacagcccttgtgggctcgcatgaagaggtcgccgatcgcatcgaagaatacgcagcactcggcttggatcagtttgtactgtcgggttatccaaacttggaggaggccttccacttcggtgagggtgtgattccggagctgctgcgccgcggtgtggatatcaaaaatcaagaatcacgagttttggaacctgttgggtaa

SEQ ID NO: 193 - protein, 381 aa, Corynebacterium glutamicum
MTLTFHWFLSTSGDSRGIIGGGHGAEKSGTSRELSHSYLKQLALAAETNGFESVLTPTGTWCEDAWITDASLIEATKRLKFLVALRPGQIGPTLSAQMASTFQRLSGNRLLINVVTGGEDAEQRAFGDFLNKEERYARTGEFLDIVSRLWRGETVTHHGEHLQVEQASLAHPPEIIPEILFGGSSPAAGEVAARYADTYLTWGETPDQVAQKINWINELAAQRGRELRHGIRFHVITRDTSEEAWVVAEKLISGVTPEQVAKAQAGFATSKSEGQRRMAELHSKGRAFTSGSTARDLEVYPNVWAGVGLLRGGAGTALVGSHEEVADRIEEYAALGLDQFVLSGYPNLEEAFHFGEGVIPELLRRGVDIKNQESRVLEPVG

SEQ ID NO: 194 - DNA, 1179 bp, Corynebacterium glutamicum
  source 1..1179: mol_type = genomic DNA
atgagtgaaaacaatcccactaccttgcactggttcctacccacctatggcgattctcgcggaatcacagccggcgggcatggcttcggcttccactccggaagccggacagcagacctcgattacctctcccaaattgccctggccgctgaacgaaacggttttgaatccgtcctgactcccactggattgtggtgcgaagatgcgtggatcaccaccgcagcgctgctgtctaggacatcaaaactgaaattcctcgttgctattcgaccaggccaagttagccccaccatcatcgcgcagcagggtgctgccttccagaaattctcaaataaccgcctgctcatcaacgtcgtggtgggtggcgaagaccatgaacagcgcgctttcgctgattattcttccaaagaggagcgctaccacaaggctgatgaaaccttagagatcatcgatcacctatggaacagcgcagaacctctaaatttccagggtgaattcctcagtgtggaaaacgcggtattgaaggaacagcccgaggtttccccaccgatttactttggcggatcctcacaactcggcatcgaaatcgcagcccaacattccgatgtttatctcacctggggtgaacctgcggaaaaggtagaggagaagcttgcccgggtgcgcgccgaagcagataagcgaaaccgcgaactagactatggcatccgcctgcatgtcattgctcgaccaactgaggatgaagcctggtcagtggctcaaaatcttcttgaccaacttgatcaggaagaggttgcccgcattcaggaagggcttgcgcgttctcaatcggaaggtcagcgtcgcatgacggaacttcatggacaaggggcagcattcacagcaggagcagatgctcgctcccttgaaattgcaccgaatctctgggcaggtgttgggctagtccgcggtggcgccggcacagcgttggtgggttcctatgagcaagtcgcgcaagcaattttgcgataccgcgatattggtctgagccacttcattttctccggctatccacatttggaggaaacctatcacgtgggcgaaggagtggtacctgagctcctcaaattgggtgttccggtgaacaaccatgaagaacaacgcaacgacgtggtagcgactccgtttatttccagatag

SEQ ID NO: 195 - protein, 392 aa, Corynebacterium glutamicum
MSENNPTTLHWFLPTYGDSRGITAGGHGFGFHSGSRTADLDYLSQIALAAERNGFESVLTPTGLWCEDAWITTAALLSRTSKLKFLVAIRPGQVSPTIIAQQGAAFQKFSNNRLLINVVVGGEDHEQRAFADYSSKEERYHKADETLEIIDHLWNSAEPLNFQGEFLSVENAVLKEQPEVSPPIYFGGSSQLGIEIAAQHSDVYLTWGEPAEKVEEKLARVRAEADKRNRELDYGIRLHVIARPTEDEAWSVAQNLLDQLDQEEVARIQEGLARSQSEGQRRMTELHGQGAAFTAGADARSLEIAPNLWAGVGLVRGGAGTALVGSYEQVAQAILRYRDIGLSHFIFSGYPHLEETYHVGEGVVPELLKLGVPVNNHEEQRNDVVATPFISR

SEQ ID NO: 196 - DNA, 1068 bp, Corynebacterium glutamicum
  source 1..1068: mol_type = genomic DNA
atgcttgcacagcgaacactccccattcacatcaccgccccccacctacccgtcgcgcgcgtatttcatcaaattcgcgccacagacgccgatcgcacctctctgcaacgcgatcttgaactctcccaagctggcatcacgcggcatgtgtcagcgcttattgatgcaggtctcgtggaggaaacccgagtggattccggggcgcgctcggggcgaccgcgcacaaaattaggcatcgacggccgccatctcaccgcctggggagtgcacattggcctgcgcagcacggattttgcggtgtgcgatttagccggccgagtgattaggtatgagcgcgtggaccatgaagtttcacactccacgccgtcggaaacgctgaattttgtcgcacataggttacaaacattgagcgccggcttgcccgagccccgcaatgtgggcgtggcattatctgcccacttaagcgccaacggaaccgtcacttccgaagattatggctggtcagaggtggaaattggggcacacctccccttccccgccaccatcggatcaggtgttgcggcgatggccggttcggaaattatcaacgcgccactgacccaatccacgcagtccacgctgtatttctacgcccgcgaaatggtctcccacgcctggattttcaacggcgctgtccaccgccccaacagcggccgcacgccgacggcgttcggaaatacaaataccttaaaagatgcttttcgacgtggactcacaccaacaactttctccgatttagtccaactctcccacaccaacccgcttgcacgacagatcctcaacgagcgcgcccacaaacttgccgacgccgtaaccaccgccgttgatgttgtcgaccccgaagccgtcgtcttcgccggcgaagccttcaccctggatccggaaactcttcgcattgtggtgacccagctccgagcaaacaccggcagccaactgagaatccaacgcgcagacgcctacattctccgcaccgcggccatccaggtggcgctgcatccgatccgtcaagatccgttggcatttgtgtaa

SEQ ID NO: 197 - protein, 355 aa, Corynebacterium glutamicum
MLAQRTLPIHITAPHLPVARVFHQIRATDADRTSLQRDLELSQAGITRHVSALIDAGLVEETRVDSGARSGRPRTKLGIDGRHLTAWGVHIGLRSTDFAVCDLAGRVIRYERVDHEVSHSTPSETLNFVAHRLQTLSAGLPEPRNVGVALSAHLSANGTVTSEDYGWSEVEIGAHLPFPATIGSGVAAMAGSEIINAPLTQSTQSTLYFYAREMVSHAWIFNGAVHRPNSGRTPTAFGNTNTLKDAFRRGLTPTTFSDLVQLSHTNPLARQILNERAHKLADAVTTAVDVVDPEAVVFAGEAFTLDPETLRIVVTQLRANTGSQLRIQRADAYILRTAAIQVALHPIRQDPLAFV

SEQ ID NO: 198 - DNA, 1065 bp, Corynebacterium glutamicum
  source 1..1065: mol_type = genomic DNA
gtgcgttatattgctttagcgaaagtaacttccatgcatcaacgttcgatgagcttcactttcattcgtacttttttcgtactttttggcatcacgttgttagtgtcatgcgttccagagcctcctgactcctacactaaagaatccactgtgctgcggtatcaggtctctgatttcaatctaaacttcgtggaattagcagttgcgcttgggtatttgaacaacattgagctccaagtagtcggatctgtacaaggcggcgttgagtccattgaatcgctcaaaaaggatgacattgacttcgcggcagtccccttcattggccttgttgcaggagagatagccaccggtgcgcccatcaaagcagtggccgcaagttacggaatttcccacgattcttcttctgcacttctagtccttaaagacagtgagatacacgaagtgcacgatctcattggcaaaacagttggcataaacaccctcggtgctctgggatctgcgatggttgagcgtcatctattcgacgccggtctcaccgaacctgagatcgtgagcgtcactcaacgtgcattacccggtgagtacttagaacaacgcctctaccaggggcaagttgatgcaatttgggtcaccgatagcgctaaacaccaagcgcttgaaactggagattttcggatcttggcagaggattcagaccttgtgcaggaactcaacactggctgcatggtggtgtcgcaaaaactcatcgacgagcaccccgcagtggttggagaattagtggatggagtagctcaggcaatcgagtttgaacgatcccactcccctgaagaagtgcgcgaagtttatttcaactacctcgaagcccatggtcagagtgatagaatatccagctttagatattgggagcattcgggcatcgcaacccgaggtggagtgctcagtgatagggagttcagcatgtggtcccactggattgaccgccaatacgacgtccccgatatcaatccagcaagtatttacaccaaccaattcaacccataccgaaaagtaaacccctcgccataa

SEQ ID NO: 199 - protein, 354 aa, Corynebacterium glutamicum
MRYIALAKVTSMHQRSMSFTFIRTFFVLFGITLLVSCVPEPPDSYTKESTVLRYQVSDFNLNFVELAVALGYLNNIELQVVGSVQGGVESIESLKKDDIDFAAVPFIGLVAGEIATGAPIKAVAASYGISHDSSSALLVLKDSEIHEVHDLIGKTVGINTLGALGSAMVERHLFDAGLTEPEIVSVTQRALPGEYLEQRLYQGQVDAIWVTDSAKHQALETGDFRILAEDSDLVQELNTGCMVVSQKLIDEHPAVVGELVDGVAQAIEFERSHSPEEVREVYFNYLEAHGQSDRISSFRYWEHSGIATRGGVLSDREFSMWSHWIDRQYDVPDINPASIYTNQFNPYRKVNPSP

SEQ ID NO: 200 - DNA, 732 bp, Corynebacterium glutamicum
  source 1..732: mol_type = genomic DNA
ctaggcaggtgtggtgatttcgagccactcaaggagttgagcggtgtaggaggcaaaggaggggtgggtgcggcgatcgccggggatatctacagtcaaactgtgtgtgatggcgccgtcgtcaagaagaaggacgtggtcggccagggcgatggcctcggaaacatcatgggtgaccagcagaactcccaagtttcgggtgttcacggttttgagcagcaggtcttgggctgtcagtcttgtcagagcatcgagggcgccgaagggttcgtcgagaagcaaaagctctggctcggagatgagcgctcgcgcaagggagacgcgctgggcttggccgccggagagcgtgaggggccaggcggcgtcggagtcgggaagtttgacttctgcaagcagtgccgaggcgcgttcttgtgcttctgaccaggaaatatcagtgcggttgaggccaaaggtcacattatcgagcaccgttttccacggcaacaggcgaggctcttggaaggcaacggccgggtttccggaaatttctacagagccggaatgctctttagataggcccgccaacacgcgcaggatggtggattttcctgaacctgagcgtccgatcagcgcggttacttcgccgcagttgatggtgaggtcgattccttggaggacttctttagttccgtatgatttgcgcaatccacggacagtggctgcgggtttgagtgacaatgtggcagtcat

SEQ ID NO: 201 - protein, 243 aa, Corynebacterium glutamicum
MTATLSLKPAATVRGLRKSYGTKEVLQGIDLTINCGEVTALIGRSGSGKSTILRVLAGLSKEHSGSVEISGNPAVAFQEPRLLPWKTVLDNVTFGLNRTDISWSEAQERASALLAEVKLPDSDAAWPLTLSGGQAQRVSLARALISEPELLLLDEPFGALDALTRLTAQDLLLKTVNTRNLGVLLVTHDVSEAIALADHVLLLDDGAITHSLTVDIPGDRRTHPSFASYTAQLLEWLEITTPA

SEQ ID NO: 202 - DNA, 771 bp, Corynebacterium glutamicum
  source 1..771: mol_type = genomic DNA
ttatgcgtttcggtagcggaaggtgtgacgttcccaagctctgatcagcgcttcagacagcagaccgaggctggcgtagacaatgaggccgaaaataacgaggtcggtgcggtaaaaatcgcgcgcattgttgatgaggaagccgagtccttcgcgggcgttgacctgttccgccacgatcagtgagagccaggcggccgcacttgcttggcgcaggccgacaaaaagttgcggcgcggcagaaggaatgatgatggtccgcaacctctggaaaaatccgaagcccatcacgtggccggcttccagaagctttggatcaatttgcctgaacccgctggcggtgttgaggtacagcggatacagcacgccgaggctaataatcagtactttcggcagctcaccgataccgaaccagatgataaacagcggcacaagacccaggtgaggcagcgcgcgggcagcttgaatgagcggatcaacggcggtgtctgcaaatctggacatgcctgtcaatacacctagggaaatgcctaggacagcaccgagggcgaagcctagaaggacgcgttggcttgagacgaggagggcgtcgagaagcgtgccatttgtggcgacctcccagctggcggccaagatcgtggttggtgccggcagaatccgctccgggatggcgcccaggctcgagccgagctgccagaaaacaagcaccgcaagcggtgaatagatgcgcgcgggcagcgcgattttggggcgagtcaaggttgtagtcat

SEQ ID NO: 203 - protein, 256 aa, Corynebacterium glutamicum
MTTTLTRPKIALPARIYSPLAVLVFWQLGSSLGAIPERILPAPTTILAASWEVATNGTLLDALLVSSQRVLLGFALGAVLGISLGVLTGMSRFADTAVDPLIQAARALPHLGLVPLFIIWFGIGELPKVLIISLGVLYPLYLNTASGFRQIDPKLLEAGHVMGFGFFQRLRTIIIPSAAPQLFVGLRQASAAAWLSLIVAEQVNAREGLGFLINNARDFYRTDLVIFGLIVYASLGLLSEALIRAWERHTFRYRNA

SEQ ID NO: 204 - DNA, 810 bp, Corynebacterium glutamicum
  source 1..810: mol_type = genomic DNA
ttaaccgatcgtcataacgatgaccgtggtgattccgacgatgaccacaaatgctctaaaaacactggggcgaagtcggcgggcgtaccgtccgccgatgtatccacctaaagctgagccaagcgcgattaaggcaacggtcggccaggagatcaactcaggggagatgattataaaaacactggctgcgatgagattaactgccgccactgtgaggtttttgatggcgttgagttcctgcatctgcagcgttgatgtgatgccaagcaatgcgatgagaagaatgccttgagctgcgctgaagtaccctccatacatgcccagcaacagggcaccacatactgagacgatgaaggtggttcggctcgggaaaggcaattgcctaaacccagcagagatgccacctgaagtatgagcgccaacatgcttcttaattgatggacctgcgataaccaacagcgtgccgaatccaatcagccagggaattactgctgtaaaaacatctgcggagaaatgcagcaagagcgaggcgccggtgatccctccgaggattgatgctggcagcagaaatctgatggtttttacatgggcgtgtagttcacgtctataagcaaccacacccgaaatacttccaggaacgatgccgatggtgttggcgatggtggcagtcaacggtggaaggcccaggaacacgagaataggaaacgacactaatgttcctgctccgacaattacattgatgcttcctgcgaggaaacctgcaccaaagatcgctcctaattcaacaatttgtaaccactcgattgacat

SEQ ID NO: 205 - protein, 269 aa, Corynebacterium glutamicum
MSIEWLQIVELGAIFGAGFLAGSINVIVGAGTLVSFPILVFLGLPPLTATIANTIGIVPGSISGVVAYRRELHAHVKTIRFLLPASILGGITGASLLLHFSADVFTAVIPWLIGFGTLLVIAGPSIKKHVGAHTSGGISAGFRQLPFPSRTTFIVSVCGALLLGMYGGYFSAAQGILLIALLGITSTLQMQELNAIKNLTVAAVNLIAASVFIIISPELISWPTVALIALGSALGGYIGGRYARRLRPSVFRAFVVIVGITTVIVMTIG

SEQ ID NO: 206 - DNA, 1455 bp, Corynebacterium glutamicum
  source 1..1455: mol_type = genomic DNA
atggtgtggggcatggaacacacttcagcattgacgctcatagactcggttttggaccctgacagcttcatttcttggaatgaaactccccaatatgacaacctcaatcaaggctatgcagagaccttggagcgggctcgaagcaaggccaaatgcgatgaatcggtaattactggagaaggcaccgtggagggcattccggtagccgttattttgtccgatttttccttcctcggcggttctttgggcacggtcgcgtcggtgcgcatcatgaaggcgattcaccgcgccacagagctgaaactcccactgctggtctcccctgcttccggtggtgcgcgcatgcaggaagacaatcgagcttttgtcatgatggtgtccataaccgcggctgtgcagcgtcaccgcgaggcgcatttgccgttcctggtgtatttgcgcaatcccacgatgggtggcgccatggcctcgtggggttcatctgggcatctcacttttgcggaacccggcgcgcagataggtttcctgggtcctcgcgtggtggagttaaccactgggcatgcgcttccagacggtgtgcagcaggcggagaatttggtgaaaactggtgtgattgatggaattgtgtcgccactccaattgcgtgcagcggtggcaaaaaccctcaaggttattcagccggtagaggcaacggatcgtttttctccaacaactcctggcgtggcacttccggtgatggaggcgattgcgcgttctcgtgacccgcagaggcctggaatcggggagattatggaaacgttgggggcagacgtcgtcaagctttctggtgcgcgtgctggcgcattgagcccggctgtgcgcgttgccctggcgcgcatcgggggccggcccgtggtgctgattgggcaggatcgccgcttcacgcttgggccgcaggagctgcgttttgcgcgtcgtggcatttcgctggcgcgcgagctaaacctgccgatcgtgtccatcatcgacacctccggcgccgaattgtcgcaggcggctgaggagctcggcatcgcaagctcgattgcgcgcaccttgtccaagcttatcgacgctcccctccccaccgtttcggtcattattggtcagggcgttggcggtggcgcgctggccatgctgcccgccgatctggtctacgcggccgaaaacgcgtggctgtccgcattgccaccagagggcgcctcggccatcctcttccgcgacaccaaccacgccgcggaaatcatagagcgacaaggcgtgcaggcgcacgcacttttaagccaagggcttatcgacgggatcgtcgccgaaaccgagcactttgttgaagaaattctcggcacaatcagcaacgccctctccgaattggataacaatccggagagggcgggacgcgacagtcgcttcacacgatttgagcgtttagcgcagtaa

SEQ ID NO: 207 - protein, 484 aa, Corynebacterium glutamicum
MVWGMEHTSALTLIDSVLDPDSFISWNETPQYDNLNQGYAETLERARSKAKCDESVITGEGTVEGIPVAVILSDFSFLGGSLGTVASVRIMKAIHRATELKLPLLVSPASGGARMQEDNRAFVMMVSITAAVQRHREAHLPFLVYLRNPTMGGAMASWGSSGHLTFAEPGAQIGFLGPRVVELTTGHALPDGVQQAENLVKTGVIDGIVSPLQLRAAVAKTLKVIQPVEATDRFSPTTPGVALPVMEAIARSRDPQRPGIGEIMETLGADVVKLSGARAGALSPAVRVALARIGGRPVVLIGQDRRFTLGPQELRFARRGISLARELNLPIVSIIDTSGAELSQAAEELGIASSIARTLSKLIDAPLPTVSVIIGQGVGGGALAMLPADLVYAAENAWLSALPPEGASAILFRDTNHAAEIIERQGVQAHALLSQGLIDGIVAETEHFVEEILGTISNALSELDNNPERAGRDSRFTRFERLAQ

SEQ ID NO: 208 - DNA, 216 bp, Corynebacterium glutamicum
  source 1..216: mol_type = genomic DNA
ttagttctttgcctccaattcgaggagttcatcgccgccggtgacgtcggagaaatcatcaaaagaaaccttagctacgatgccaggtcccggtgccatgattggtgcctcgagtttgatggtttctaccgttcccagttgcatgccggtttccacgggatcgccttcatcgacaaaatagtggacgattccagcaaaaggtgcgtagatcttcat

SEQ ID NO: 209 - protein, 71 aa, Corynebacterium glutamicum
MKIYAPFAGIVHYFVDEGDPVETGMQLGTVETIKLEAPIMAPGPGIVAKVSFDDFSDVTGGDELLELEAKN

SEQ ID NO: 210 - DNA, 1464 bp, Corynebacterium glutamicum
  source 1..1464: mol_type = genomic DNA
atggccacggccgagaacacaacacaggagaatcggaaaatcctgttcaacgcatttgatatgaactgcgttgcgcatcagtccccaggactgtggacacacccgaaggataaggcgcgagactacaacactcttgattactgggtgcaccttgccaagactttggagaagggccttttcgacggccttttcatcgcagatgtgcttggaacttacgatgtttatggttctagtaatgaagcggcgttgagcagtggtgcgcaggtgcctgtcaatgatccgatccttcttgtttctgcgatggcctatgccacaaagaacctcgggtttggcattactgcaggtactgcctatgagcacccgtatccttttgcgcggcgtctggccacacttgatcacctgactaatgggcgtgtggggtggaatgtggttactggctatcttccctctgctgctcaaaacatgggtgacaccgatcagctgccacatgatgagcgctatgacaaagcagatgaatacctggaagtgatctacaagcttctcgagggctcctgggaagacgatgctgttcaaaacaatacggagacgagtgtctttacggactcctccaaagtgcacgccattaatcatcatggcaagtactttgatgtgccgggcattgccatcactgagccgagtgtgcagcgtacgccggtgatctaccaggcgggtgcatcgccgcgcggattgaaattcgctggtgagaatgcagaagcagtgtttatcaattccagcaccgtggaggcaatcaccaagactgtcgcaaaaattcgcgctgctgcggtcgctgcgggacgtgatccacatgcggtgaagatctttgcgatgcaaaccatcatcactggtgaaacagaagcagatgcgcaggcaaagctggaggaatacagtcgctatatcgatcctgtcggtggtctgaccttgatgtctggatggaccggcgcggatctgtcgcagtatgacctggatgaaccgatcaccaatattgagtcaaacgctattcagtccactgcagccaccattagcaacggcaccggtgaaggtgcgtggacggtacgcaaactgggtgaggcaaccggcatcggcggcttcggaccagtgcttgtgggatctggcgctaacgttgccgcggaacttgcacgcatccaggatctcagcgatgttgatggtttcaaccttgcttatgccatcaccccaggaacttttgaagatgtcgtggactttgtggtgcctgagctgcaaaaacttagccgctacaagacggaatacgcgccgggttccttgcgcaacaaattgctcggtaaaggtgatcgcctggacgatacccaccgcggcgcaagctaccgcctaggcgctcggaactccaccgccactattgatctcagttccatatccgcccaactagtttcccagggagcccactcatga

SEQ ID NO: 211 - protein, 487 aa, Corynebacterium glutamicum
MATAENTTQENRKILFNAFDMNCVAHQSPGLWTHPKDKARDYNTLDYWVHLAKTLEKGLFDGLFIADVLGTYDVYGSSNEAALSSGAQVPVNDPILLVSAMAYATKNLGFGITAGTAYEHPYPFARRLATLDHLTNGRVGWNVVTGYLPSAAQNMGDTDQLPHDERYDKADEYLEVIYKLLEGSWEDDAVQNNTETSVFTDSSKVHAINHHGKYFDVPGIAITEPSVQRTPVIYQAGASPRGLKFAGENAEAVFINSSTVEAITKTVAKIRAAAVAAGRDPHAVKIFAMQTIITGETEADAQAKLEEYSRYIDPVGGLTLMSGWTGADLSQYDLDEPITNIESNAIQSTAATISNGTGEGAWTVRKLGEATGIGGFGPVLVGSGANVAAELARIQDLSDVDGFNLAYAITPGTFEDVVDFVVPELQKLSRYKTEYAPGSLRNKLLGKGDRLDDTHRGASYRLGARNSTATIDLSSISAQLVSQGAHS

SEQ ID NO: 212 - DNA, 1083 bp, Corynebacterium glutamicum
  source 1..1083: mol_type = genomic DNA
atgatctcaccgcaaacaatcatcgacaatcttgctccagtcctcgcggagatcgcagcaacggctgcgcagcgcgaacaggatcgagaattcagccgtgatttggcaaagcagcttagtgccggtgggttcactaaactccgcattccagttgaatttggcggtttgggttttagccttccagaagcatttgaggtgctggtggcagcggccgctgcagattcaaatatcgcgcagggattgcggcctcacttcttggcagtggagagcttattgatcgctccttattcagagcaccgcaccaagtggctgcgaaaaatcgctgagaaaggcgtggtcattggcaatgcgttgactgaagtgggaaacaagccaggtgagctgaaaaccaagatccgaaaggaaggcgagtcttacgttctcagcgacaccaagttctattccacgggcagtctttacgccgactggattcaagtacatgcgaaagatgaggaagatcaagatgttttcgcctttgttgatcgcgacgcatcaggcgttgttttggtggattactgggacgaatttgggcaacaactatttgcctccggaaccagcttctttgaaaaagtcgtggtagatccactggacattgtcacccgtgattacaccgcacccagcgctttccaggcgctggcgcagtcacatcatttgtctacgttgactggtatttcgcaggcaatcacccgtgacattgttacctatgtacaaaaccgcacccgtattttcagccacggaagtggtgaccttccacgctttgatccgcaggtgcaacaggtggtgggtgaggtgaaagccaagtcgtatgcagtcgagaaaatctttcagggtttcgcacaagaactggatcttgtcgtcgataaggcaaaagccggcactgctacggaggtcgatctggccgctgtcgacctgagcgcctaccaagcccagttagcggtggcacctttggtgctgagccaggccacccaggcctttgaggttggcgcagatgccttaaacggcggccacgtggctgcccaatacacgatcggatccttgtcatga

SEQ ID NO: 213 - protein, 360 aa, Corynebacterium glutamicum
MISPQTIIDNLAPVLAEIAATAAQREQDREFSRDLAKQLSAGGFTKLRIPVEFGGLGFSLPEAFEVLVAAAAADSNIAQGLRPHFLAVESLLIAPYSEHRTKWLRKIAEKGVVIGNALTEVGNKPGELKTKIRKEGESYVLSDTKFYSTGSLYADWIQVHAKDEEDQDVFAFVDRDASGVVLVDYWDEFGQQLFASGTSFFEKVVVDPLDIVTRDYTAPSAFQALAQSHHLSTLTGISQAITRDIVTYVQNRTRIFSHGSGDLPRFDPQVQQVVGEVKAKSYAVEKIFQGFAQELDLVVDKAKAGTATEVDLAAVDLSAYQAQLAVAPLVLSQATQAFEVGADALNGGHVAAQYTIGSLS

SEQ ID NO: 214 - DNA, 1200 bp, Corynebacterium glutamicum
  source 1..1200: mol_type = genomic DNA
atgagccaagaaattttgagccattttgcacccgcattagagcgcattcgaagcggcgccgtcgagcgcgaacagcagcgcgccttgccagtggaagagattaaagagctggtagagctaggttttactgggcttcgagtgcccgaagaactaggcggtgcgggcgcttccctggaaagcgtagttgagttactgatcgagatcgcgggcgccgattccaatatcgcccaagccctgcgcggacattttgccttcgtggaactactcctggaagcgccggagagcgaattccgcacccattggctgcgcgaagtcgccaccggaagacttgtgggcaacgccgaaagtgagaaacgcggcgtttacggcgatccgcagaccttcatcgatgaggtggagactgaaaacggaccgattttcgtgctcaacggcactaagttttataccaccggcacctattttgcggactacacctggaccaccgcgctgctgcgcaacctcaacggccaagaaactttggtcagtttgccggtcgatctgcacgcgccgggcgtggatgttgttgatgattggagcgggtttgggcaaaagctcaccgcctctggaacgaccacgtttaaagacctggaggtggatccgcggtggatcattccacgcactgatgcgcccacgctggtgtggacgtatctgcagctgagcctgctgaccgtgctggttggcagtgccgcagcagctgtcgatgaggtggttgcccgcgcccaatcctccaccagaaatgcgtggaaccctggcgtcgagcgccgcagcgatccggccgcaaccatagcgatcggcgacgcacgcagccgagtcaccgttattcgtggagcgcttctcgacgccacccgccacgtttccaacgccgccacgatcgtaacccccgaagccttcaacgaggcggacgctattgttgcagcgctctggcccatcgtctccggacaagctttggtggtgacatccaacgttttcgatgcggtgggtgcatctgcagtgcttggtgagcattccattgatcgccactggcgcaatgtgcgtaccgtgtcctcaaacaacccggtgttcctggccaagaatgcagtgggggagtatgccctcaacggcactcctgtgggtaccaacattggtaaagcactgagccgtccggtgagcctaagcagctag

SEQ ID NO: 215 - protein, 399 aa, Corynebacterium glutamicum
MSQEILSHFAPALERIRSGAVEREQQRALPVEEIKELVELGFTGLRVPEELGGAGASLESVVELLIEIAGADSNIAQALRGHFAFVELLLEAPESEFRTHWLREVATGRLVGNAESEKRGVYGDPQTFIDEVETENGPIFVLNGTKFYTTGTYFADYTWTTALLRNLNGQETLVSLPVDLHAPGVDVVDDWSGFGQKLTASGTTTFKDLEVDPRWIIPRTDAPTLVWTYLQLSLLTVLVGSAAAAVDEVVARAQSSTRNAWNPGVERRSDPAATIAIGDARSRVTVIRGALLDATRHVSNAATIVTPEAFNEADAIVAALWPIVSGQALVVTSNVFDAVGASAVLGEHSIDRHWRNVRTVSSNNPVFLAKNAVGEYALNGTPVGTNIGKALSRPVSLSS

SEQ ID NO: 216 - DNA, 1296 bp, Corynebacterium glutamicum
  source 1..1296: mol_type = genomic DNA
gtgtctgagaaaagtccaggagtgatggctagcggagcggagctgattcgtgccgccgacattcaaacggcgcaggcacgaatttcctccgtcattgcaccaactccattgcagtattgccctcgtctttctgaggaaaccggagcggaaatctaccttaagcgtgaggatctgcaggatgttcgttcctacaagatccgcggtgcgctgaactctggagcgcagctcacccaagagcagcgcgatgcaggtatcgttgccgcatctgcaggtaaccatgcccagggcgtggcctatgtgtgcaagtccttgggcgttcagggacgcatctatgttcctgtgcagactccaaagcaaaagcgtgaccgcatcatggttcacggcggagagtttgtctccttggtggtcactggcaataacttcgacgaagcatcggctgcagcgcatgaagatgcagagcgcaccggcgcaacgctgatcgagcctttcgatgctcgcaacaccgtcatcggtcagggcaccgtggctgctgagatcttgtcgcagctgacttccatgggcaagagtgcagatcacgtgatggttccagtcggcggtggcggacttcttgcaggtgtggtcagctacatggctgatatggcacctcgcactgcgatcgttggtatcgaaccagcgggagcagcatccatgcaggctgcattgcacaatggtggaccaatcactttggagactgttgatccctttgtggacggcgcagcagtcaaacgtgtcggagatctcaactacaccatcgtggagaagaaccagggtcgcgtgcacatgatgagcgcgaccgagggcgctgtgtgtactgagatgctcgatctttaccaaaacgaaggcatcatcgcggagcctgctggcgcgctgtctatcgctgggttgaaggaaatgtcctttgcacctggttctgtcgtggtgtgcatcatctctggtggcaacaacgatgtgctgcgttatgcggaaatcgctgagcgctccttggtgcaccgcggtttgaagcactacttcttggtgaacttcccgcaaaagcctggtcagttgcgtcacttcctggaagatatcctgggaccggatgatgacatcacgctgtttgagtacctcaagcgcaacaaccgtgagaccggtactgcgttggtgggtattcacttgagtgaagcatcaggattggattctttgctggaacgtatggaggaatcggcaattgattcccgtcgcctcgagccgggcacccctgagtacgaatacttgacctaa

SEQ ID NO: 217 - protein, 431 aa, Corynebacterium glutamicum
MSEKSPGVMASGAELIRAADIQTAQARISSVIAPTPLQYCPRLSEETGAEIYLKREDLQDVRSYKIRGALNSGAQLTQEQRDAGIVAASAGNHAQGVAYVCKSLGVQGRIYVPVQTPKQKRDRIMVHGGEFVSLVVTGNNFDEASAAAHEDAERTGATLIEPFDARNTVIGQGTVAAEILSQLTSMGKSADHVMVPVGGGGLLAGVVSYMADMAPRTAIVGIEPAGAASMQAALHNGGPITLETVDPFVDGAAVKRVGDLNYTIVEKNQGRVHMMSATEGAVCTEMLDLYQNEGIIAEPAGALSIAGLKEMSFAPGSVVVCIISGGNNDVLRYAEIAERSLVHRGLKHYFLVNFPQKPGQLRHFLEDILGPDDDITLFEYLKRNNRETGTALVGIHLSEASGLDSLLERMEESAIDSRRLEPGTPEYEYLT

SEQ ID NO: 218 - DNA, 1536 bp, Escherichia coli
  source 1..1536: mol_type = genomic DNA
atggctacatcagtacagacaggtaaagctaagcagctcacattacttggattctttgccataacggcatcgatggtaatggctgtttatgaataccctaccttcgcaacatcgggcttttcattagtcttcttcctgctattaggcgggattttatggtttattcccgtgggactttgtgctgcggaaatggccaccgtcgacggctgggaagaaggtggtgtcttcgcctgggtatcaaatactctggggccgagatggggatttgcagcgatctcatttggctatctgcaaatcgccattggttttattccgatgctctatttcgtgttaggggcactctcctacatcctgaaatggccagcgctgaatgaagaccccattaccaaaactattgcagcactcatcattctttgggcgctggcattaacgcagtttggtggcacgaaatacacggcgcgaattgctaaagttggcttcttcgccggtatcctgttacctgcatttattttgatcgcattagcggctatttatctgcactccggtgcccccgttgctatcgaaatggattcgaagaccttcttccctgacttctctaaagtgggcaccctggtagtatttgttgccttcattttgagttatatgggcgtagaagcatccgcaacccacgtcaatgaaatgagcaacccagggcgcgactatccgttggctatgttactgctgatggtggcggcaatctgcttaagctctgttggtggtttgtctattgcgatggtcattccgggtaatgaaatcaacctctccgcaggggtaatgcaaacctttaccgttctgatgtcccatgtggcaccagaaattgagtggacggttcgcgtgatctccgcactgctgttgctgggtgttctggcggaaatcgcctcctggattgttggtccttctcgcgggatgtatgtaacagcgcagaaaaacctgctgccagcggcattcgctaaaatgaacaaaaatggcgtaccggtaacgctggtcatttcgcagctggtgattacgtctatcgcgttgatcatcctcaccaataccggtggcggtaacaacatgtccttcctgatcgcactggcgctgacggtggtgatttatctgtgtgcttatttcatgctgtttattggctacattgtgttggttcttaaacatcctgacttaaaacgcacatttaatatccctggtggtaaaggggtgaaactggtcgtggcaattgtcggtctgctgacttcaattatggcgtttattgtttccttcctgccgccggataacatccagggtgattctaccgatatgtatgttgaattactggttgttagtttcctggtggtacttgccctgccctttattctctatgctgttcatgatcgtaaaggcaaagcaaataccggcgtcactctggagccaatcaacagtcagaacgcaccaaaaggtcacttcttcctgcacccgcgtgcacgttcaccacactatattgtgatgaatgacaagaaacactaa

SEQ ID NO: 219 - protein, 511 aa, Escherichia coli
MATSVQTGKAKQLTLLGFFAITASMVMAVYEYPTFATSGFSLVFFLLLGGILWFIPVGLCAAEMATVDGWEEGGVFAWVSNTLGPRWGFAAISFGYLQIAIGFIPMLYFVLGALSYILKWPALNEDPITKTIAALIILWALALTQFGGTKYTARIAKVGFFAGILLPAFILIALAAIYLHSGAPVAIEMDSKTFFPDFSKVGTLVVFVAFILSYMGVEASATHVNEMSNPGRDYPLAMLLLMVAAICLSSVGGLSIAMVIPGNEINLSAGVMQTFTVLMSHVAPEIEWTVRVISALLLLGVLAEIASWIVGPSRGMYVTAQKNLLPAAFAKMNKNGVPVTLVISQLVITSIALIILTNTGGGNNMSFLIALALTVVIYLCAYFMLFIGYIVLVLKHPDLKRTFNIPGGKGVKLVVAIVGLLTSIMAFIVSFLPPDNIQGDSTDMYVELLVVSFLVVLALPFILYAVHDRKGKANTGVTLEPINSQNAPKGHFFLHPRARSPHYIVMNDKKH

SEQ ID NO: 220 - DNA, 2847 bp, Corynebacterium glutamicum
  source 1..2847: mol_type = genomic DNA
ttattgtgccatgttgttttgctccaagatgcttaagaccacctctgcaaggtgcgttgcttgttttccggtgccttgttcgatctgggtgcggcaggagaaaccgtcagcaatcacatgtccttctgcttttctgaccttggggaacagctctcgttcaccaagtgcgaaggacatttcagcgtggtctttttcaaagccccagtttccggcaagcccgcaacaaccagtggcaatttgttcatcttttacacccaaagcttcaagaacgagtgccgattgttgtgggtcgcctagcgaacgctcgtggcagtgcacctgggtaagcgcagttgattctgttagctggaggcttccagactcgactagctcggtgatctttggtgcgatgacctcagcgaatggtttggtcagtgctgcaaggcgtgccagatcagggttatcggagagttctgttgcctcatcttgaagcatgacggtgcacgaaggttccaaaccaacgactgttagaccttggtccaggtagggtttcatcactttcgccgtttgttctaggactttctttgtcatgctcaattggccggtggaatgccaggtgagtccacagcagacgaagccatctgggatgaccacgttgtaaccgagggcttcaagagttttgatcgctgcgtgagctggtccggtgtcgaggttggtgttgaaggaatcgggccacagcaccaccgtttcacctgagtttttcttcggcttgtacttgcgcagcgagcggtgggcgaaggaaatcaacgggcgatcagcgagcccgccgaccttgcgcaccactggggcggtgagtgctgactgcatggtggcgtcgataagcgtaggaagaagcggtattttatgggcaacgtgtcccagaagcggcagccagcccatgacgtaatgggccatggggcgaagtcggccggcgtagtgtttgtccaggaattcggctttgtaggtggacatgtcgacgttgactggacattccgatgcgcatgctttgcaggaaaggcacaggtcaagggcttcattgacttcttcgctgcggtagccgtcggcgatggattcaccgcggaacatctcagagagcaagcgggcgcggcctctggtggaatgtacttcgtcgccggtgatttggaaggacgggcacatcgcgtcggattctgagcggcatgcggatacacccacgcagcgattcaccgcgttgatcatggaacctttgtctttagagaatttgtgtacgggcgtgatgtcgagggcgcgctggcccgggtccatgcgaagtccttgcatgacaggatctgcccagaccaacactcccggattgaagatgcgctggggatcgaaaatcagcttgaattcttcgaagagtgcacgcatttctgctgaatacatgcggtcaaggaaggatgagcgggcgcgaccgtctccatgctcgcccgagaggctgccaccataagacgccaccaaggtggaggcttcattcatgaacgcctcgaatttcttcaggccttccttggtagagaaatcaaaactgatgcgaacgtggacgcagccttctccaaagtgtccaaatggaataccctggtaatcgaacttatccatcagcgcataaagatcgcggagataatcagctaaattctctggaggcaccgccgagtcttcccaattcggccacgcttccccaccatcagctaagcgcgtgacaatgcccgccgaggattcacggatgcgccacaattcccgcatttcagaaggctcagacacgaccacgtaatcaatggtgtcaacggcggttgcgacttcctcggcggcttgtaccgcggcctgcagggtgtcacttcctgtctcgcagtacaaccatccgccggcttcaatgccgatgcggtttcctggaagattctgcccagcttctgattgtccctgtttactgcgcagcgcagcgaggagatctccgcccatgccttcaatggttgctaccccaggcagtcgcaatttggcggctgctcgggcggcgtcaaaaaccgtgtcgaaagccaggacagcaagcgctttcactttgggtgttggaaccaacttcaccgtcaaccgagtaatgattccaatggttccctcggtgcccgccattgctttggccatgtcgtgggcaagataatgcaaaccgtagcccgacacttggcgagggaaacgacccagttctttactaataaggtcctgattcttggacgctaagtcggtgagcttctgattgatctcagcatcatcgcagccatcttttgtcacagtgacttctcggccatcgctgagcatgagcgtgacatccacgagattttccgcagctgtaccgaatgcaaccgagtgtgaaccacacgcattgttggcaaccatgccaccgatcgtgcaccgggaatgcgtggaaggatccgggccgtaagttaatccgaattctgcggctgcatcgcgcaaggcatcacacaccacaccgggttccacaactgcagtttgtgcaactggatcaatatctaaaatgcggttgaaatagcgtgacgtatcgatgatcaaaccttcaccgatcgcatttccagcaaccgagcttcctccaccgcgcccaacaacagaccaccctcgtgccaccgcaacagcaatggcatcacgaatttgttccacattttctggttcagctacagctgcagggacgcgtctaaaaattcctgcatcagaagaatatgctgcacgggttgtcatatccgtgcgcagcttcgcgcgagaatgctggctaaatacttctgggtcgagtctgttgaatttgatggtgtgagtcat

SEQ ID NO: 221 - protein, 948 aa, Corynebacterium glutamicum
MTHTIKFNRLDPEVFSQHSRAKLRTDMTTRAAYSSDAGIFRRVPAAVAEPENVEQIRDAIAVAVARGWSVVGRGGGSSVAGNAIGEGLIIDTSRYFNRILDIDPVAQTAVVEPGVVCDALRDAAAEFGLTYGPDPSTHSRCTIGGMVANNACGSHSVAFGTAAENLVDVTLMLSDGREVTVTKDGCDDAEINQKLTDLASKNQDLISKELGRFPRQVSGYGLHYLAHDMAKAMAGTEGTIGIITRLTVKLVPTPKVKALAVLAFDTVFDAARAAAKLRLPGVATIEGMGGDLLAALRSKQGQSEAGQNLPGNRIGIEAGGWLYCETGSDTLQAAVQAAEEVATAVDTIDYVVVSEPSEMRELWRIRESSAGIVTRLADGGEAWPNWEDSAVPPENLADYLRDLYALMDKFDYQGIPFGHFGEGCVHVRISFDFSTKEGLKKFEAFMNEASTLVASYGGSLSGEHGDGRARSSFLDRMYSAEMRALFEEFKLIFDPQRIFNPGVLVWADPVMQGLRMDPGQRALDITPVHKFSKDKGSMINAVNRCVGVSACRSESDAMCPSFQITGDEVHSTRGRARLLSEMFRGESIADGYRSEEVNEALDLCLSCKACASECPVNVDMSTYKAEFLDKHYAGRLRPMAHYVMGWLPLLGHVAHKIPLLPTLIDATMQSALTAPVVRKVGGLADRPLISFAHRSLRKYKPKKNSGETVVLWPDSFNTNLDTGPAHAAIKTLEALGYNVVIPDGFVCCGLTWHSTGQLSMTKKVLEQTAKVMKPYLDQGLTVVGLEPSCTVMLQDEATELSDNPDLARLAALTKPFAEVIAPKITELVESGSLQLTESTALTQVHCHERSLGDPQQSALVLEALGVKDEQIATGCCGLAGNWGFEKDHAEMSFALGERELFPKVRKAEGHVIADGFSCRTQIEQGTGKQATHLAEVVLSILEQNNMAQ

SEQ ID NO: 222 - DNA, 1881 bp, Corynebacterium glutamicum
  source 1..1881: mol_type = genomic DNA
gtgaatgtggcagcttctcaacagcccactcccgccacggttgcaagccgtggtcgatccgccgcccctgagcggatgacaggtgcaaaggcaattgttcgatcgctcgaggagcttaacgccgacatcgtgttcggtattcctggtggtgcggtgctaccggtgtatgacccgctctattcctccacaaaggtgcgccacgtcttggtgcgccacgagcagggcgcaggccacgcagcaaccggctacgcgcaggttactggacgcgttggcgtctgcattgcaacctctggcccaggagcaaccaacttggttaccccaatcgctgatgcaaacttggactccgttcccatggttgccatcaccggccaggtcggaagtggcctgctgggtaccgacgctttccaggaagccgatatccgcggcatcaccatgccagtgaccaagcacaacttcatggtcaccaaccctaacgacattccacaggcattggctgaggcattccacctcgcgattactggtcgccctggccctgttctggtggatattcctaaggatgtccagaacgctgaattggatttcgtctggccaccaaagatcgacctgccaggctaccgcccagtttcaacaccacatgctcgccagatcgagcaggcagtcaagctgatcggtgaggccaagaagcccgtcctttacgttggtggtggcgtaatcaaggctgacgcacacgaagagcttcgtgcgttcgctgagtacaccggcatcccagttgtcaccaccttgatggctttgggtactttcccagagtctcacgagctgcacatgggtatgccaggcatgcatggcactgtgtccgctgttggtgcactgcagcgcagcgacctgctgattgctatcggctcccgctttgatgaccgcgtcaccggtgacgttgacaccttcgcgcctgacgccaagatcattcacgccgacattgatcctgccgaaatcggcaagatcaagcaggttgaggttccaatcgtgggcgatgcccgcgaagttcttgctcgtctgctggaaaccaccaaggcaagcaaggcagagaccgaggacatctccgagtgggttgactacctcaagggcctcaaggcacgtttcccgcgtggctacgacgagcagccaggcgatctgctggcaccacagtttgtcattgaaaccctgtccaaggaagttggccccgacgcaatttactgcgccggcgttggccagcaccaaatgtgggcagctcagttcgttgactttgaaaagccacgcacctggctcaactccggtggactgggcaccatgggctacgcagttcctgcggcccttggagcaaaggctggcgcacctgacaaggaagtctgggctatcgacggcgacggctgtttccagatgaccaaccaggaactcaccaccgccgcagttgaaggtttccccattaagatcgcactaatcaacaacggaaacctgggcatggttcgccaatggcagaccctattctatgaaggacggtactcaaatactaaacttcgtaaccagggcgagtacatgcccgactttgttaccctttctgagggacttggctgtgttgccatccgcgtcaccaaagcggaggaagtactgccagccatccaaaaggctcgagagatcaacgaccgcccagtagtcatcgacttcatcgtcggtgaagacgcacaggtatggccaatggtgtctgctggatcatccaactccgatatccagtacgcactcggattgcgcccattctttgatggtgatgaatctgcagcagaagatcctgccgacattcacgaagccgtcagcgacattgatgccgccgttgaatcgaccgaggcataa

SEQ ID NO: 223 - protein, 626 aa, Corynebacterium glutamicum
MNVAASQQPTPATVASRGRSAAPERMTGAKAIVRSLEELNADIVFGIPGGAVLPVYDPLYSSTKVRHVLVRHEQGAGHAATGYAQVTGRVGVCIATSGPGATNLVTPIADANLDSVPMVAITGQVGSGLLGTDAFQEADIRGITMPVTKHNFMVTNPNDIPQALAEAFHLAITGRPGPVLVDIPKDVQNAELDFVWPPKIDLPGYRPVSTPHARQIEQAVKLIGEAKKPVLYVGGGVIKADAHEELRAFAEYTGIPVVTTLMALGTFPESHELHMGMPGMHGTVSAVGALQRSDLLIAIGSRFDDRVTGDVDTFAPDAKIIHADIDPAEIGKIKQVEVPIVGDAREVLARLLETTKASKAETEDISEWVDYLKGLKARFPRGYDEQPGDLLAPQFVIETLSKEVGPDAIYCAGVGQHQMWAAQFVDFEKPRTWLNSGGLGTMGYAVPAALGAKAGAPDKEVWAIDGDGCFQMTNQELTTAAVEGFPIKIALINNGNLGMVRQWQTLFYEGRYSNTKLRNQGEYMPDFVTLSEGLGCVAIRVTKAEEVLPAIQKAREINDRPVVIDFIVGEDAQVWPMVSAGSSNSDIQYALGLRPFFDGDESAAEDPADIHEAVSDIDAAVESTEA

SEQ ID NO: 224 - DNA, 519 bp, Corynebacterium glutamicum
  source 1..519: mol_type = genomic DNA
atggctaattctgacgtcacccgccacatcctgtccgtactcgttcaggacgtagacggaatcatttcccgcgtatcaggtatgttcacccgacgcgcattcaacctcgtgtccctcgtgtctgcaaagaccgaaacacacggcatcaaccgcatcacggttgttgtcgacgccgacgagctcaacattgagcagatcaccaagcagctcaacaagctgatccccgtgctcaaagtcgtgcgacttgatgaagagaccactatcgcccgcgcaatcatgctggttaaggtctctgcggacagcaccaaccgtccgcagatcgtcgacgccgcgaacatcttccgcgcccgagtcgtcgacgtggctccagactctgtggttattgaatccacaggcaccccaggcaagctccgcgcactgcttgacgtgatggaaccattcggaatccgcgaactgatccaatccggacagattgcactcaaccgcggtccgaagaccatggctccggccaagatctaa

SEQ ID NO: 225 - protein, 172 aa, Corynebacterium glutamicum
MANSDVTRHILSVLVQDVDGIISRVSGMFTRRAFNLVSLVSAKTETHGINRITVVVDADELNIEQITKQLNKLIPVLKVVRLDEETTIARAIMLVKVSADSTNRPQIVDAANIFRARVVDVAPDSVVIESTGTPGKLRALLDVMEPFGIRELIQSGQIALNRGPKTMAPAKI

SEQ ID NO: 226 - DNA, 1017 bp, Corynebacterium glutamicum
  source 1..1017: mol_type = genomic DNA
atggctattgaactgctttatgatgctgacgctgacctctccttgatccagggccgtaaggttgccatcgttggctacggctcccagggccacgcacactcccagaacctccgcgattctggcgttgaggttgtcattggtctgcgcgagggctccaagtccgcagagaaggcaaaggaagcaggcttcgaggtcaagaccaccgctgaggctgcagcttgggctgacgtcatcatgctcctggctccagacacctcccaggcagaaatcttcaccaacgacatcgagccaaacctgaacgcaggcgacgcactgctgttcggccacggcctgaacattcacttcgacctgatcaagccagctgacgacatcatcgttggcatggttgcgccaaagggcccaggccacttggttcgccgtcagttcgttgatggcaagggtgttccttgcctcatcgcagtcgaccaggacccaaccggaaccgcacaggctctgaccctgtcctacgcagcagcaatcggtggcgcacgcgcaggcgttatcccaaccaccttcgaagctgagaccgtcaccgacctcttcggcgagcaggctgttctctgcggtggcaccgaggaactggtcaaggttggcttcgaggttctcaccgaagctggctacgagccagagatggcatacttcgaggttcttcacgagctcaagctcatcgttgacctcatgttcgaaggtggcatcagcaacatgaactactctgtttctgacaccgctgagttcggtggctacctctccggcccacgcgtcatcgatgcagacaccaagtcccgcatgaaggacatcctgaccgatatccaggacggcaccttcaccaagcgcctcatcgcaaacgttgagaacggcaacaccgagcttgagggccttcgtgcttcctacaacaaccacccaatcgaggagaccggcgctaagctccgcgacctcatgagctgggtcaaggttgacgctcgcgcagaaaccgcttaa

SEQ ID NO: 227 - protein, 338 aa, Corynebacterium glutamicum
MAIELLYDADADLSLIQGRKVAIVGYGSQGHAHSQNLRDSGVEVVIGLREGSKSAEKAKEAGFEVKTTAEAAAWADVIMLLAPDTSQAEIFTNDIEPNLNAGDALLFGHGLNIHFDLIKPADDIIVGMVAPKGPGHLVRRQFVDGKGVPCLIAVDQDPTGTAQALTLSYAAAIGGARAGVIPTTFEAETVTDLFGEQAVLCGGTEELVKVGFEVLTEAGYEPEMAYFEVLHELKLIVDLMFEGGISNMNYSVSDTAEFGGYLSGPRVIDADTKSRMKDILTDIQDGTFTKRLIANVENGNTELEGLRASYNNHPIEETGAKLRDLMSWVKVDARAETA

SEQ ID NO: 228 - DNA, 1842 bp, Corynebacterium glutamicum
  source 1..1842: mol_type = genomic DNA
ttagtcgacctgacggactgcacccttatcagcggaggtagccatctttgcgtatgcgcgcagtgccttggtgacaacgcggttacggttgactggctgccatggcttctcggaggcgttcatagcgtcgcggcggcgctggagttcctcgtcggagacctgaacttcgagcttgcggttgtgaacgtcgatggagacgatgtcgccgttttcgatcagaccaatgactccgccgtgtgctgcttctggggagacgtggccgatggacagtcctgaggaacctccggagaaacggccgtcggtgatcagtgcacacttcttgcccaggccggatcccttgaggaatgcggttgggtgaagcatttcctgcatgcctggtccacctgatgggccttcgtagcggacgaccagaacttcgccagcttggatggtcttggtcaggatgacagagactgcctcttcctggctttcgacaactcgtgctggtccggtgaagttccacagctcttcttcgatacctgcggacttgatcactgcgccgtcaggggagatgttgccgcgaagaacaaccaggccgccgtcggcggtgtaggcgtgttcaacgtcgcggatgcagcccttggcagcgtcggtgtcgagttcgtcccagcggttctcggtggagaatgcttcggtggtgcggatgccacctggggctgcgtggaagagttcggttgctacttcggtggtcttgccagagcggatatcccagtcatccaaccaaccttcaaggtcgttggagtgaacggagtggacgtccttgttcagcaggccaccgcggttgagctcgccgagcagtgctggaatgccaccggcgcggtggacgtcttccatgtggtagtcggagtttggtgcaaccttggacaggcaggggacgtttttggacagttcgtcgatgtctgcgaggtcgaagtcaacttcgccttcctgggcagctgcgaggatgtggaggatggtgttggtggatccacccatggccatatccagtgccattgcgttttcgaatgccttcttggtggcaatgccacgtggcagaacggattcgtcttcttcaccgtagtagcggcggcacagttcaacgacggtttcgccggccttttcaaacagtgcgcgacgtgctgcgtgggttgccagagtggagccgttgcccgggagagaaagtcccagagcttcggtgaggcagttcatggagttcgcggtgaacataccggagcaggagccacaggttgggcatgcggatgcttcaacggctgcaaggcctgcgtcgtcgactgcatcgcttgcggatgcggagatcgcggtgatgaggtcggttggtgcgtgtgcaacgccgtcaacgacgacagccttgccagcttccattgggccaccggaaacgaagaccactgggatgttcaggcgcattgctgcgttgagcatgcctggggtgatcttgtcacagttggagatacacaccatggcgtcggcggtgtgtgcgttgaccatgtattcgacggagtcggcgatgatttcacgggatggcagggagtacagcatgccgccgtgtcccatggcgatgccgtcatcgacggcgatggtgttgaattcctttggaacgccaccggctttgcgcactgcatctgccacaatatcgccgacgttcttaaggtgaacgtgtccgggcacgaactgggtgtaggagtttacgatggcaacaattggcttgccgaactcattttccttggtgccggtggcacgccaaagggcgcgagcgccagctgcattgcgaccgacggtggtgacttttgaacgaagtgggatcat

SEQ ID NO: 229 - protein, 613 aa, Corynebacterium glutamicum
MIPLRSKVTTVGRNAAGARALWRATGTKENEFGKPIVAIVNSYTQFVPGHVHLKNVGDIVADAVRKAGGVPKEFNTIAVDDGIAMGHGGMLYSLPSREIIADSVEYMVNAHTADAMVCISNCDKITPGMLNAAMRLNIPVVFVSGGPMEAGKAVVVDGVAHAPTDLITAISASASDAVDDAGLAAVEASACPTCGSCSGMFTANSMNCLTEALGLSLPGNGSTLATHAARRALFEKAGETVVELCRRYYGEEDESVLPRGIATKKAFENAMALDMAMGGSTNTILHILAAAQEGEVDFDLADIDELSKNVPCLSKVAPNSDYHMEDVHRAGGIPALLGELNRGGLLNKDVHSVHSNDLEGWLDDWDIRSGKTTEVATELFHAAPGGIRTTEAFSTENRWDELDTDAAKGCIRDVEHAYTADGGLVVLRGNISPDGAVIKSAGIEEELWNFTGPARVVESQEEAVSVILTKTIQAGEVLVVRYEGPSGGPGMQEMLHPTAFLKGSGLGKKCALITDGRFSGGSSGLSIGHVSPEAAHGGVIGLIENGDIVSIDVHNRKLEVQVSDEELQRRRDAMNASEKPWQPVNRNRVVTKALRAYAKMATSADKGAVRQVD

SEQ ID NO: 230 - DNA, 3474 bp, Corynebacterium glutamicum
  source 1..3474: mol_type = genomic DNA
gtgactgctatcacccttggcggtctcttgttgaaaggaataattactctagtgtcgactcacacatcttcaacgcttccagcattcaaaaagatcttggtagcaaaccgcggcgaaatcgcggtccgtgctttccgtgcagcactcgaaaccggtgcagccacggtagctatttacccccgtgaagatcggggatcattccaccgctcttttgcttctgaagctgtccgcattggtaccgaaggctcaccagtcaaggcgtacctggacatcgatgaaattatcggtgcagctaaaaaagttaaagcagatgccatttacccgggatacggcttcctgtctgaaaatgcccagcttgcccgcgagtgtgcggaaaacggcattacttttattggcccaaccccagaggttcttgatctcaccggtgataagtctcgcgcggtaaccgccgcgaagaaggctggtctgccagttttggcggaatccaccccgagcaaaaacatcgatgagatcgttaaaagcgctgaaggccagacttaccccatctttgtgaaggcagttgccggtggtggcggacgcggtatgcgttttgttgcttcacctgatgagcttcgcaaattagcaacagaagcatctcgtgaagctgaagcggctttcggcgatggcgcggtatatgtcgaacgtgctgtgattaaccctcagcatattgaagtgcagatccttggcgatcacactggagaagttgtacacctttatgaacgtgactgctcactgcagcgtcgtcaccaaaaagttgtcgaaattgcgccagcacagcatttggatccagaactgcgtgatcgcatttgtgcggatgcagtaaagttctgccgctccattggttaccagggcgcgggaaccgtggaattcttggtcgatgaaaagggcaaccacgtcttcatcgaaatgaacccacgtatccaggttgagcacaccgtgactgaagaagtcaccgaggtggacctggtgaaggcgcagatgcgcttggctgctggtgcaaccttgaaggaattgggtctgacccaagataagatcaagacccacggtgcagcactgcagtgccgcatcaccacggaagatccaaacaacggcttccgcccagataccggaactatcaccgcgtaccgctcaccaggcggagctggcgttcgtcttgacggtgcagctcagctcggtggcgaaatcaccgcacactttgactccatgctggtgaaaatgacctgccgtggttccgactttgaaactgctgttgctcgtgcacagcgcgcgttggctgagttcaccgtgtctggtgttgcaaccaacattggtttcttgcgtgcgttgctgcgggaagaggacttcacttccaagcgcatcgccaccggattcattgccgatcacccgcacctccttcaggctccacctgctgatgatgagcagggacgcatcctggattacttggcagatgtcaccgtgaacaagcctcatggtgtgcgtccaaaggatgttgcagctcctatcgataagctgcctaacatcaaggatctgccactgccacgcggttcccgtgaccgcctgaagcagcttggcccagccgcgtttgctcgtgatctccgtgagcaggacgcactggcagttactgataccaccttccgcgatgcacaccagtctttgcttgcgacccgagtccgctcattcgcactgaagcctgcggcagaggccgtcgcaaagctgactcctgagcttttgtccgtggaggcctggggcggcgcgacctacgatgtggcgatgcgtttcctctttgaggatccgtgggacaggctcgacgagctgcgcgaggcgatgccgaatgtaaacattcagatgctgcttcgcggccgcaacaccgtgggatacaccccgtacccagactccgtctgccgcgcgtttgttaaggaagctgccagctccggcgtggacatcttccgcatcttcgacgcgcttaacgacgtctcccagatgcgtccagcaatcgacgcagtcctggagaccaacaccgcggtagccgaggtggctatggcttattctggtgatctctctgatccaaatgaaaagctctacaccctggattactacctaaagatggcagaggagatcgtcaagtctggcgctcacatcttggccattaaggatatggctggtctgcttcgcccagctgcggtaaccaagctggtcaccgcactgcgccgtgaattcgatctgccagtgcacgtgcacacccacgacactgcgggtggccagctggcaacctactttgctgcagctcaagctggtgcagatgctgttgacggtgcttccgcaccactgtctggcaccacctcccagccatccctgtctgccattgttgctgcattcgcgcacacccgtcgcgataccggtttgagcctcgaggctgtttctgacctcgagccgtactgggaagcagtgcgcggactgtacctgccatttgagtctggaaccccaggcccaaccggtcgcgtctaccgccacgaaatcccaggcggacagttgtccaacctgcgtgcacaggccaccgcactgggccttgcggatcgtttcgaactcatcgaagacaactacgcagccgttaatgagatgctgggacgcccaaccaaggtcaccccatcctccaaggttgttggcgacctcgcactccacctcgttggtgcgggtgtggatccagcagactttgctgccgatccacaaaagtacgacatcccagactctgtcatcgcgttcctgcgcggcgagcttggtaaccctccaggtggctggccagagccactgcgcacccgcgcactggaaggccgctccgaaggcaaggcacctctgacggaagttcctgaggaagagcaggcgcacctcgacgctgatgattccaaggaacgtcgcaatagcctcaaccgcctgctgttcccgaagccaaccgaagagttcctcgagcaccgtcgccgcttcggcaacacctctgcgctggatgatcgtgaattcttctacggcctggtcgaaggccgcgagactttgatccgcctgccagatgtgcgcaccccactgcttgttcgcctggatgcgatctctgagccagacgataagggtatgcgcaatgttgtggccaacgtcaacggccagatccgcccaatgcgtgtgcgtgaccgctccgttgagtctgtcaccgcaaccgcagaaaaggcagattcctccaacaagggccatgttgctgcaccattcgctggtgttgtcaccgtgactgttgctgaaggtgatgaggtcaaggctggagatgcagtcgcaatcatcgaggctatgaagatggaagcaacaatcactgcttctgttgacggcaaaatcgatcgcgttgtggttcctgctgcaacgaaggtggaaggtggcgacttgatcgtcgtcgtttcctaa

SEQ ID NO: 231 - protein, 1157 aa, Corynebacterium glutamicum
MTAITLGGLLLKGIITLVSTHTSSTLPAFKKILVANRGEIAVRAFRAALETGAATVAIYPREDRGSFHRSFASEAVRIGTEGSPVKAYLDIDEIIGAAKKVKADAIYPGYGFLSENAQLARECAENGITFIGPTPEVLDLTGDKSRAVTAAKKAGLPVLAESTPSKNIDEIVKSAEGQTYPIFVKAVAGGGGRGMRFVASPDELRKLATEASREAEAAFGDGAVYVERAVINPQHIEVQILGDHTGEVVHLYERDCSLQRRHQKVVEIAPAQHLDPELRDRICADAVKFCRSIGYQGAGTVEFLVDEKGNHVFIEMNPRIQVEHTVTEEVTEVDLVKAQMRLAAGATLKELGLTQDKIKTHGAALQCRITTEDPNNGFRPDTGTITAYRSPGGAGVRLDGAAQLGGEITAHFDSMLVKMTCRGSDFETAVARAQRALAEFTVSGVATNIGFLRALLREEDFTSKRIATGFIADHPHLLQAPPADDEQGRILDYLADVTVNKPHGVRPKDVAAPIDKLPNIKDLPLPRGSRDRLKQLGPAAFARDLREQDALAVTDTTFRDAHQSLLATRVRSFALKPAAEAVAKLTPELLSVEAWGGATYDVAMRFLFEDPWDRLDELREAMPNVNIQMLLRGRNTVGYTPYPDSVCRAFVKEAASSGVDIFRIFDALNDVSQMRPAIDAVLETNTAVAEVAMAYSGDLSDPNEKLYTLDYYLKMAEEIVKSGAHILAIKDMAGLLRPAAVTKLVTALRREFDLPVHVHTHDTAGGQLATYFAAAQAGADAVDGASAPLSGTTSQPSLSAIVAAFAHTRRDTGLSLEAVSDLEPYWEAVRGLYLPFESGTPGPTGRVYRHEIPGGQLSNLRAQATALGLADRFELIEDNYAAVNEMLGRPTKVTPSSKVVGDLALHLVGAGVDPADFAADPQKYDIPDSVIAFLRGELGNPPGGWPEPLRTRALEGRSEGKAPLTEVPEEEQAHLDADDSKERRNSLNRLLFPKPTEEFLEHRRRFGNTSALDDREFFYGLVEGRETLIRLPDVRTPLLVRLDAISEPDDKGMRNVVANVNGQIRPMRVRDRSVESVTATAEKADSSNKGHVAAPFAGVVTVTVAEGDEVKAGDAVAIIEAMKMEATITASVDGKIDRVVVPAATKVEGGDLIVVVS

SEQ ID NO: 232 - DNA, 1233 bp, Corynebacterium glutamicum
  source 1..1233: mol_type = genomic DNA
ttaacgcagcggatcgaacggcttgatgatcgggttggtgttgcccgtagccatcagctccgccaaatacttaccggtggcagggcccagcaccacaccccacatgccgtgaccaccggcaacgtaaatgttctccgccttggtctgcccaatcaacggacgcccatccggagtgaccgggcgggaacccacccactcatcctgacgatcgttgaaatcaatacctctcataaccttcttcgcctgcgaaacaatcgcatccacccggccctgctggaacggctcatccggaccgcggaactccatggtgcctgcaatgcggaaacggccctcatacggcgtgcaggccatgcggtggtggggaaggtacacagaatgcttggcaggaatatccgttgccacagagaaggaatagccacgaccagcctgaacaagagttttcacaccgtattcacgcgttagacccggcagccaggcacccgttgccacaaccaccttgtccgcttcttcacggctaccatccgccaaaatgaccgcgggacgatcacccttcgccacatgcacaacttctgccccggcgcggatcacgccaccacgcttcaccacagcatccgccaatgactgcacgtatggacccggctcgatgaaacgctggccttccaaacggtaagccacctgaatttgctcattcagcatcggcgccaactcttgtggattctccagtcgagacatctccactttctggccgtgcctgctcacgccatcaatttccttacggaaacccgccgattggcgctcttcctcaaaaccaataacaaatggaccttcatgggtgaggccttccacgccaccgatcgacagttcatcaaaagcttcgagcgcgaccttatcgatctccgtgaggtccgccatcgtggaatcccacttgcgttgaaaagcctgcgccataaattgcgccaagaaaagccacagtttgggatccacacgaagtggcatatgcatcggcgacaccggattgaacagctctttcggaccatacgtccacagccccggctccgacaacggaatagttttcgccggcgctaaccaaccagcattaccccacgaagaacctgcagcgacaccatcccgatcaaggacgctcacctcgaacccgtgctcctgtaaatgccaggcggtggcaagacccactatgccggctccaacaacaatgacttttccagacgaactcat

SEQ ID NO: 233 - protein, 410 aa, Corynebacterium glutamicum
MSSSGKVIVVGAGIVGLATAWHLQEHGFEVSVLDRDGVAAGSSWGNAGWLAPAKTIPLSEPGLWTYGPKELFNPVSPMHMPLRVDPKLWLFLAQFMAQAFQRKWDSTMADLTEIDKVALEAFDELSIGGVEGLTHEGPFVIGFEEERQSAGFRKEIDGVSRHGQKVEMSRLENPQELAPMLNEQIQVAYRLEGQRFIEPGPYVQSLADAVVKRGGVIRAGAEVVHVAKGDRPAVILADGSREEADKVVVATGAWLPGLTREYGVKTLVQAGRGYSFSVATDIPAKHSVYLPHHRMACTPYEGRFRIAGTMEFRGPDEPFQQGRVDAIVSQAKKVMRGIDFNDRQDEWVGSRPVTPDGRPLIGQTKAENIYVAGGHGMWGVVLGPATGKYLAELMATGNTNPIIKPFDPLR

SEQ ID NO: 234 - DNA, 972 bp, Corynebacterium glutamicum
  source 1..972: mol_type = genomic DNA
ctagatcttgcgcatcctcacccgttccaccaagtgatcgctaccttttttcagtaccaacgacgcgcgaactcgggtgggaagaatattctccaccaagttgggcaggttgatcgattgccacagttctcgagcgacggcgatggactctggatcagccatgtcggcgtaatgggagaagtgggcaccggggcgacggaatgcagtgtcgcggagtttgaggaagcggtcgatgtaccatttttcgatatcttcggtgcgggcatctacgtagacgctgaagtcgaaaaggtcactgaccatcaatgttgggccagtttggaggacgtttaagccttcgacgatcaaaatgtcgggttggcggactgtggtgaattcgcctggaactcggtcgtacgcggtgtgggagtagacaggtgcgttgacttcgagttttccggattttacgtcggtgacaaagcggaggagtgcacgttggtcgtagctttcggggaatccttttcgggacattaatccgcggcggattagttccgcgccgggatagaggaatccgtcggtggtgacgaggtccacgcgggggtgggaattccagcgctgaagcagaacttggaggagtcgggcggtggttgatttaccgacggcgacggatcccgcgacaccaatgacaaacggcacagagatagagggggaagttccgaggaaggtttcggtggctgcagtaagttgctgtcgggccgctacctggaggtgaatcagacgggacagcggaaggtagacttctgccacttcagcgaggtcaatgttttctccgatgcctcgaagttcaatgacttctttttgggtcagcacctgaggcattgagtttctcagctcgcgccattgtgcgcggtcgaaatcaaggtaggggctgaaatctggtgtgcgtggggaaggtttcacaccagttgtgcttgcagcgttttgctctgccat

SEQ ID NO: 235 - protein, 323 aa, Corynebacterium glutamicum
MAEQNAASTTGVKPSPRTPDFSPYLDFDRAQWRELRNSMPQVLTQKEVIELRGIGENIDLAEVAEVYLPLSRLIHLQVAARQQLTAATETFLGTSPSISVPFVIGVAGSVAVGKSTTARLLQVLLQRWNSHPRVDLVTTDGFLYPGAELIRRGLMSRKGFPESYDQRALLRFVTDVKSGKLEVNAPVYSHTAYDRVPGEFTTVRQPDILIVEGLNVLQTGPTLMVSDLFDFSVYVDARTEDIEKWYIDRFLKLRDTAFRRPGAHFSHYADMADPESIAVARELWQSINLPNLVENILPTRVRASLVLKKGSDHLVERVRMRKI

SEQ ID NO: 236 - DNA, 1263 bp, Corynebacterium glutamicum
  source 1..1263: mol_type = genomic DNA
ctagacttcgcgatacgccagtgccgcgtcccaaatttgcgcagcaacctcgattttgctgccgtgctccacatcgactaccccaccgtgagcatccaaaatccagccctcattgtgcttttgcccaaacactttgcccatgcccacctcattacacatgaggaggtcgcagcccttcttctgcagtttcttgcgcgcatactccaaggcggtggtgtgctcgtctccagtttccgcagcaaaacccacgatgacaggattactgtccagctctcctgcttcacgacgcttcaccgtggtagccaaaatatccgggttttccaccaacgagatggtgctcaatgcgtcttcatcagaatctgagcccttcttcaacttcgatgtcgcctgcgatgcgggcgtgaaatcagctaccgctgccgccatgacgatgaaatcagattggccagctcgttcctggactgcatcaaacatgtcttgtgtggacaccaccggcacgatctctgcgcctgccggagtgggcagctccgcagcatttcccgccacgatgctgacatgagcacctcgctgtgctgcgatttcacccaacgcaaaaccttgacggcccgaggaactattgccaataaagcgcacaggatcaatatgctcatgcgtgccaccagcagtgatcagcactttcttgcccgccaaatcctgaggcaacctcgccccggcgtgcaccgcattggctaaatcaacaatctgctctggatccggcagccggccagggcctgtatctttaccggtgagtcgaccatgcgcaggctcaatcacggtaatcccccgctgcctcagcgttgccacattggctacggtagccggattaaaccacatctccgtatgcatggccggcgcaataaccacggggcacgttgccaccagcagcgtcgccgccaacagatcgtcaccgcgacctgccaccacacgcgccatcaaatcggctgtcgccggcgcaatcacgatcaaatcagcctcctggccaactttcacatgctgcaccgaatccaccgcatcaaacaccgttgtagacaccggattgccagacaacgcttcaaacgtcgccttccccacaaacttcaacgcggattccgtaggaaccacccgcacattatcgcccgcttctttaaacgcgcgcacgatgtgacacgccttgtacgctgcgattccgccggcaacaccaacaaccacgttgcggggtgcggaggggcttgaaacatgagtcat

SEQ ID NO: 237 - protein, 420 aa, Corynebacterium glutamicum
MTHVSSPSAPRNVVVGVAGGIAAYKACHIVRAFKEAGDNVRVVPTESALKFVGKATFEALSGNPVSTTVFDAVDSVQHVKVGQEADLIVIAPATADLMARVVAGRGDDLLAATLLVATCPVVIAPAMHTEMWFNPATVANVATLRQRGITVIEPAHGRLTGKDTGPGRLPDPEQIVDLANAVHAGARLPQDLAGKKVLITAGGTHEHIDPVRFIGNSSSGRQGFALGEIAAQRGAHVSIVAGNAAELPTPAGAEIVPVVSTQDMFDAVQERAGQSDFIVMAAAVADFTPASQATSKLKKGSDSDEDALSTISLVENPDILATTVKRREAGELDSNPVIVGFAAETGDEHTTALEYARKKLQKKGCDLLMCNEVGMGKVFGQKHNEGWILDAHGGVVDVEHGSKIEVAAQIWDAALAYREV

SEQ ID NO: 238 - DNA, 483 bp, Corynebacterium glutamicum
  source 1..483: mol_type = genomic DNA
atgaaagctgtttgcccaggatcattcgaccccatcaccctcggccacctcgacatcgtgacccgcgccgccgcgcaatttagcgaagtcaccatcttggtcaccgccaaccccaacaaaaactcaggactgttcaccgtcgcagaacgcatggatctcatccgcgaatccaccgcacacctcgacaacgtaaaagtagacacctgggcctcactgctcgtggattacaccaccgaacatggcatcggcgccctagtcaaaggcctgcgaagctccctcgactacgaatacgagctccccatggcacagatgaacaggcgactcacgggcgtagacaccttcttcctcctcaccgacgagaaatacggctacgtcagctccacactgtgcaaagaagttgcccgcttcggtggcgatgtctccggcctcctacctgaggtagtggcaaaagccgtgacagaaaaatacagcaaccagcactag

SEQ ID NO: 239 - protein, 160 aa, Corynebacterium glutamicum
MKAVCPGSFDPITLGHLDIVTRAAAQFSEVTILVTANPNKNSGLFTVAERMDLIRESTAHLDNVKVDTWASLLVDYTTEHGIGALVKGLRSSLDYEYELPMAQMNRRLTGVDTFFLLTDEKYGYVSSTLCKEVARFGGDVSGLLPEVVAKAVTEKYSNQH

SEQ ID NO: 240 - DNA, 603 bp, Corynebacterium glutamicum
  source 1..603: mol_type = genomic DNA
atgttgcgcattggactaacaggagggatcggcagcggtaaatctaccgttgccgatcttttgtcatctgaaggatttctcatcgtcgacgcggaccaagttgcccgcgatatcgtcgaacccggacaaccggcattagcagagctagctgaagcttttggccaagacatcttaaaacccgacggcactctagaccgcgcgggattagcagccaaagcatttgtcagcgaagaacaaacagcgctgctcaatgccattacccaccctcgtatcgccgaagagtcagctcgtcgattcaacgaagccgaagatcaaggcgccaaagttgcggtttatgacatgcctttgcttgtagaaaaaggccttgaccgcaagatggaccttgtcgtcgtagttgatgttgacgtagaggaacgcgtccgcagacttgtggaaaaacgtggcctcacagaggacgacgtgcggcgtcgaatcgcttctcaagtgcccgacgacgtcagacttaaagccgctgacatcgttgtggacaataacggcacgctagaggaccttcatgctgaagcaagcaagctgattgctgagattcttagtcgcgtgaattag

SEQ ID NO: 241 - protein, 200 aa, Corynebacterium glutamicum
MLRIGLTGGIGSGKSTVADLLSSEGFLIVDADQVARDIVEPGQPALAELAEAFGQDILKPDGTLDRAGLAAKAFVSEEQTALLNAITHPRIAEESARRFNEAEDQGAKVAVYDMPLLVEKGLDRKMDLVVVVDVDVEERVRRLVEKRGLTEDDVRRRIASQVPDDVRLKAADIVVDNNGTLEDLHAEASKLIAEILSRVN

SEQ ID NO: 242 - DNA, 1054 bp, Escherichia coli
  source 1..1054: mol_type = genomic DNA
agtgaacatatatatcgggtggcttttcaaattaatccctttgattatgggcttaatttgtatcgcgttaggtggctttgtgctggaaagttcagggcaaagcgagtatttcgtcgcgggtcatgtgctgatttctctggcggccatatgcctggcattattcactaccgcatttattatcatttcgcagctcacgcgcggcgttaatacgttttacaatacattgttccccattattggctatgcggggtcaattatcaccatgatatggggttgggcactgttagcaggcaatgatgtgatggcagacgagtttgtcgccggccatgttattttcggcgttggtatgattgccgcctgtgtatcgacggtggcagcgtcatccggtcactttctgctcattcccaaaaatgcagcggggagcaagagcgacggaacaccggtacaggcttattcttcattaatcggtaactgcctcattgccgttcccgttttactcaccctgctcggtttcatttggtctattacgctgttacgtagtgctgacataactccgcattatgtcgcgggtcacgtattgcttgggttaaccgcaatctgtgcctgtctaattggccttgttgccacaattgtccatcaaacacgtaatacgttttcaactaaagaacactggctgtggtgttattgggttatttttctcggctcaatcacggtactgcaggggatatacgtcttagtcagttccgatgcaagcgcccgactggctcccggcattattcttatttgcctcggaatgatctgttacagcatattctcaaaagtctggctactggcactggtatggagacgtacctgttcgttagccaacagaataccgatgattcccgtcttcacctgcctgttttgccttttcctggcatcgtttcttgcggaaatggcgcagaccgacatgggatattttattccttcgcgagttctggtcggtttgggagcggtatgctttacgttgttctcaatcgtttcaatattagaagcgggttctgctaaaaaataa

SEQ ID NO: 243 - protein, 350 aa, Escherichia coli
MNIYIGWLFKLIPLIMGLICIALGGFVLESSGQSEYFVAGHVLISLAAICLALFTTAFIIISQLTRGVNTFYNTLFPIIGYAGSIITMIWGWALLAGNDVMADEFVAGHVIFGVGMIAACVSTVAASSGHFLLIPKNAAGSKSDGTPVQAYSSLIGNCLIAVPVLLTLLGFIWSITLLRSADITPHYVAGHVLLGLTAICACLIGLVATIVHQTRNTFSTKEHWLWCYWVIFLGSITVLQGIYVLVSSDASARLAPGIILICLGMICYSIFSKVWLLALVWRRTCSLANRIPMIPVFTCLFCLFLASFLAEMAQTDMGYFIPSRVLVGLGAVCFTLFSIVSILEAGSAKK

SEQ ID NO: 244 - DNA, 815 bp, Corynebacterium glutamicum
  source 1..815: mol_type = genomic DNA
ttaaaaggactccgcttcgcctgggaaggtacccgcgtggatatcggcgatgtaggcctgcgcggcgtcgtgcaaggaatcgcccaaggtggcgtactcgcggacgaagcgtggcttcttgccgcggttgaggccgaaggcatcctgccacaccaaaacctgcccatctgtgccattgccggcaccgattccgatagtggtgatggaaagatcctcggtaacctcgcgcgctgcctctgctggaaccatctccaacacaaccgcaaacgcacccgcctgctccaacgcgcgggcatcggcgatgagctttccagaactcgcgccacgaccctgaaccacgtggccgcccaaggaatgctcggactgcggggtgtacccgatgtggccgacaaccggaattccagcatcaacaatgcgtcgaatcgtctgcgcgatctccacgccaccctcgatcttcaccgcagccgcacccgtttcacgcatgacccggatcgcggactccaccgcctgatttgggccacctcataggtaccaaacggcagatcaaccaccacaagcgcacgcttcgtagcgatcgtcaccgccttggccagcacaatcatctcatccaaggtgatcgacaaggtggtatcgcgacccagcacaacgttggcagcggaatcaccaacaaggagcatatcgacgccagcctcatcaaaaatgcgcgccgaaagcgcatcatagctggtgagaaccgaaactttctggccgtttactttagcttcgcggaaatgacgggtgcggattttctttgcatcaatgcctgacatgggcat

SEQ ID NO: 245 - protein, 271 aa, Corynebacterium glutamicum
MPMSGIDAKKIRTRHFREAKVNGQKVSVLTSYDALSARIFDEAGVDMLLVGDSAANVVLGRDTTLSITLDEMIVLAKAVTIATKRALVVVDLPFGTYEVSPNQAVESAIRVMRETGAAAVKIEGGVEIAQTIRRIVDAGIPVVGHIGYTPQSEHSLGGHVVQGRGASSGKLIADARALEQAGAFAVVLEMVPAEAAREVTEDLSITTIGIGAGNGTDGQVLVWQDAFGLNRGKKPRFVREYATLGDSLHDAAQAYIADIHAGTFPGEAESF

SEQ ID NO: 246 - DNA, 840 bp, Corynebacterium glutamicum
  source 1..840: mol_type = genomic DNA
ctagagctcgatattgtcgatcaaccgcaccggccccacgaaaatcgcgccgaccaccaacgctggttgggtgagcaggccgtcgatttctaatggttcgagggtggctggatcgacaatttccaggtgatccaagcgcacgccgtcggcgctggccaaggtgtcgcgcgcaccttggatatctagcgcttcaccagctgcttttcgacgctgcaacccactcaacacctgcggcagcaccagagcttgcgctcgctgatccgcagaaagacgttgattgcggctggattcggctaagccatcggcgccacgaataatcggaacgggacgaatctccacgggaatgtctagatcggcaaccaatcgccgaatcaccgcaacctgctgagcatctttttgtccaaaatatgcacgatcagggcgcaccaaattgaacagcttcgccaccacggtagccacaccatcgaaatggccaggcctgctggcaccctccaattttgttccgatggaaccggtgcgcgcccacactagtggcaagccaccggggtacatttcctccacatcgggtgcgaacacaatatccacacctgcctcttcaagcagtgctaaatcggcgtcgagttggcggggatagttgcggtaatcatcgcaatcaccgagtgcttcaaactgcaggggattgacaaaaatactggctacaacagtgtcgttttcagcgcgtgctgctttaaccaacgaggcgtgtccgctgtgtagcgcacccatggtggggacgagcccgacggatttgtggtggaggagggcgtcgataagcgcctgctttgtggttgctacctgcat

SEQ ID NO: 247 - protein, 279 aa, Corynebacterium glutamicum
MQVATTKQALIDALLHHKSVGLVPTMGALHSGHASLVKAARAENDTVVASIFVNPLQFEALGDCDDYRNYPRQLDADLALLEEAGVDIVFAPDVEEMYPGGLPLVWARTGSIGTKLEGASRPGHFDGVATVVAKLFNLVRPDRAYFGQKDAQQVAVIRRLVADLDIPVEIRPVPIIRGADGLAESSRNQRLSADQRAQALVLPQVLSGLQRRKAAGEALDIQGARDTLASADGVRLDHLEIVDPATLEPLEIDGLLTQPALVVGAIFVGPVRLIDNIEL

SEQ ID NO: 248 - DNA, 411 bp, Corynebacterium glutamicum
  source 1..411: mol_type = genomic DNA
ctaaatgcttctcgacgtcaaaagcccggatccaggtagtgcttccgcaagatcgttgccgagcgcaacgatgcggttgtcggcgtccacgtgcacaatctttggctcatacgccttggcttccgcatcagttgcctgaaggtagctcatgatgatcacaagatcgccaggattaataaggtgtgcagcggcaccattgatgcaaatattgcccgttccggcgtcgcccacaatgacataagtttccagacgagcgccgttggtgatgtctacgatggcaactttttcgccttcgatcaatccggcggcgtgaaccaggtcggcgtcgatggttacagagccaacataatctagatcagcttgagtgacagtggctcggtgaatcttacttccgaggatggtgcgcagcat

SEQ ID NO: 249 - protein, 136 aa, Corynebacterium glutamicum
MLRTILGSKIHRATVTQADLDYVGSVTIDADLVHAAGLIEGEKVAIVDITNGARLETYVIVGDAGTGNICINGAAAHLINPGDLVIIMSYLQATDAEAKAYEPKIVHVDADNRIVALGNDLAEALPGSGLLTSRSI

SEQ ID NO: 250 - DNA, 903 bp, Corynebacterium glutamicum
  source 1..903: mol_type = genomic DNA
atgaaaattgcgatcgttggcgctggtgcagttggtggatatttcggagcgttgttacaagaatctggtgcagatatcacgatggttgcacgtggacgaacattagaagccttgaagtctaaaggactccacatcaacgatgcaagaggcgaacgctacgtaccaattcctgcagttgcgagcgtgcaagaactaaaagatgcagatgtagtgatgattgctactaaagcattatcgcggtctttagatctcgctgaacttttgggtgggatacctgcgaattcggtggtcgcgattactcagaattcgattgaatctgctgatctagcagcgaagagtatcggtgctgatcgtgtgtggcctggtgtggttcgtgggttctttgttcatgaggggccagcctcagtgtcatacaagggaggcccactgtcctacacgtttggtgattctggtgaactttctaggcaattcgcaagcactcttgaacaggccggtattgacggagttctgcatcccgatattttggtggatgtgtgggagaaagccatgttcgtagaggttttcggcgggttgggggctttcgtcgaaaagcaattaggtaccttgcgtacgcattttagggcttccctggaagccttgatggaagaggtggctgaggtggctcgcgcggcaggtgttgcgttgccgagcgatgcggtggagcgcaccatgaattttgcggatcggatgcctgagaattcgacgagttcgatgcagcgtgatttggccgcgggagtggctagtgagcttgaggctcagacaggtgcaattgtgcgggcagcgcacaaagtgggtgtgaaaactccgcttcatgaccttatttatgctggtcttaagctgaaagaagaggaaaattcactttag

SEQ ID NO: 251 - protein, 300 aa, Corynebacterium glutamicum
MKIAIVGAGAVGGYFGALLQESGADITMVARGRTLEALKSKGLHINDARGERYVPIPAVASVQELKDADVVMIATKALSRSLDLAELLGGIPANSVVAITQNSIESADLAAKSIGADRVWPGVVRGFFVHEGPASVSYKGGPLSYTFGDSGELSRQFASTLEQAGIDGVLHPDILVDVWEKAMFVEVFGGLGAFVEKQLGTLRTHFRASLEALMEEVAEVARAAGVALPSDAVERTMNFADRMPENSTSSMQRDLAAGVASELEAQTGAIVRAAHKVGVKTPLHDLIYAGLKLKEEENSL

SEQ ID NO: 252 - DNA, 1290 bp, Corynebacterium glutamicum
  source 1..1290: mol_type = genomic DNA
atggaaacgactcaaaccagcacgattgcgtcgaaagactctcgtagtgcctggcgcaagacagacaccatgtggatgctgggcctttacggcacggcaatcggcgcgggcgtgctgttcctgccaatcaatgccggtgttggcggtatgatcccgctgatcatcatggctatccttgcgttcccgatgacattttttgctcaccgcggcctgactcgcttcgtactgtctggtaaaaacccaggcgaagacatcaccgaggttgtagaagaacactttggtattggcgcaggtaaactgattaccctgctctacttcttcgctatctacccgatcctgctggtttatagcgtggcaatcaccaatacagttgaaagcttcatgtctcaccagctgggtatgacaccaccgccgcgtgcgattctgtcgctgatcctgatcgtgggtatgatgaccatcgttcgcttcggtgagcagatgatcgttaaagcgatgagtattctggtattcccgtttgttggcgtactgatgctgctggctctgtacctgatcccgcagtggaacggcgcagcactggaaacgctgtctctggacactgcatcggcaaccggaaacggtctgtggatgaccctgtggctggcaattccggtaatggtgttctcgttcaaccactctccgatcatctcttctttcgccgttgcgaagcgtgaagagtacggcgatatggcagaacagaaatgctcgaagatccttgcattcgcacacatcatgatggtgctgaccgtaatgttcttcgtcttcagctgcgtactgagcctgactccggcagacctggctgcggctaaagagcagaacatctcgattctgtcttacctggctaaccactttaacgcaccggttatcgcgtggatggcaccgattatcgcgattatcgctatcaccaaatccttcctcggccactacctgggcgcacgtgaaggcttcaacggtatggtggttaaatctctgcgtggtaaaggtaagtctatcgaaatcaacaagctgaaccgtatcactgcgctgttcatgctggtaacgacctggattgttgccaccctgaacccgagcatcctgggtatgattgaaaccctgggcggcccaatcatcgcgatgatcctgttcctgatgccgatgtacgcaattcagaaagtaccggcaatgcgtaagtacagcggtcacatcagcaacgtattcgttgtcgtgatgggtctgattgcaatctccgcaatcttctactctctgttcagctaa

SEQ ID NO: 253 - protein, 429 aa, Corynebacterium glutamicum
METTQTSTIASKDSRSAWRKTDTMWMLGLYGTAIGAGVLFLPINAGVGGMIPLIIMAILAFPMTFFAHRGLTRFVLSGKNPGEDITEVVEEHFGIGAGKLITLLYFFAIYPILLVYSVAITNTVESFMSHQLGMTPPPRAILSLILIVGMMTIVRFGEQMIVKAMSILVFPFVGVLMLLALYLIPQWNGAALETLSLDTASATGNGLWMTLWLAIPVMVFSFNHSPIISSFAVAKREEYGDMAEQKCSKILAFAHIMMVLTVMFFVFSCVLSLTPADLAAAKEQNISILSYLANHFNAPVIAWMAPIIAIIAITKSFLGHYLGAREGFNGMVIKSLRGKGKSIEINKLNRITALFMLVTTWIVATLNPSILGMIETLGGPIIAMILFLMPMYAIQKVPAMRKYSGHISNVFVVVMGLIAISAIFYSLFS

SEQ ID NO: 254 - DNA, 1281 bp, Corynebacterium glutamicum
  source 1..1281: mol_type = genomic DNA
atgagttcagtttcgctgcaggattttgatgcagagcgaattggtttgttccacgaggacattaagcgcaagtttgatgagctcaagtcaaaaaatctgaagctggatcttactcgcggtaagccttcgtcggagcagttggatttcgctgatgagttgttggcgttgcctggtaagggtgatttcaaggctgcggatggtactgatgtccgtaactatggcgggctggatggcatcgttgatattcgccagatttgggcggatttgctgggtgttcctgtggagcaggtcttggcgggggatgcttcgagcttgaacatcatgtttgatgtgatcagctggtcgtacattttcggtaacaatgattcggttcagccttggtcgaaggaagaaaccgttaagtggatttgccctgttccgggctatgatcgccatttctccatcacggagcgtttcggctttgagatgatttctgtgccaatgaatgaagacggccctgatatggatgctgttgaggaattggtgaagaatccgcaggttaagggcatgtgggttgttccggtgttttctaacccgactggtttcacggtgacagaagacgtcgcaaagcgtctaagcgcaatggaaaccgcagctccggacttccgcgttgtgtgggataatgcctacgccgttcatacgctgaccgatgaattccctgaggttatcgatatcgtcgggcttggtgaggccgctggcaacccgaaccgtttctgggcgttcacttctacttcgaagatcactctcgcgggtgcgggcgtgtcgttcttcctcacctctgcggagaaccgcaagtggtacaccggccatgcgggtatccgtggcattggccctaacaaggtcaatcagttggctcatgcgcgttactttggcgatgctgagggagtgcgcgcggtgatgcgtaagcatgctgcgtcgttggctccgaagttcaacaaggttctggagattctggattctcgccttgctgagtacggtgtcgcgcagtggactgtccctgcgggcggttacttcatttcccttgatgtggttcctggtacggcgtctcgcgtggctgagttggctaaggaagccggcatcgcgttgacgggtgcgggttcttcttacccgctgcgtcaggatccggagaacaaaaatctccgtttggcaccgtcgctgcctccagttgaggaacttgaggttgccatggatggcgtggctacctgtgtgctgttggcagcagcggagcattacgctaactaa

SEQ ID NO: 255 - protein, 429 aa, Corynebacterium glutamicum; identical to SEQ ID NO: 253

SEQ ID NO: 256 - DNA, 1503 bp, Corynebacterium glutamicum
  source 1..1503: mol_type = genomic DNA
ttaggcttcctcaagcttcagggtcttctgggtgcgtgcccactgctgctcaaacagtgctggctcggaagcaagcttcttgccgtaggaagggatcatgtccttcagcttgtcgccccactcgatcatgcggtcaccgaagcaacgctcaagcagctcgatcattgcggaaggtgcgatggatgctccaggggaagcaccgagcaatccggcgatggtgccttcggagttgttgatcaaggtggtgccgaattccagggaaccgaacttagggaatcctgcaggcttaataacctgaacacgctgtccggcaacgatggtctcccaatcgccgttttgtgcctctggcatgtactcgcgaagagcatccatacgcttgtcctggtccttgagaacttcagtgacaaggtacttggtcagatcaaattcctgagcagcaacgccaaggtaggaaggaatgttgtctgggcggatggacttgaacaggtccaggtaggagccttccttcaagaacttaggggtccagccaccgtaaggtccaaagagcagacccttttcaccctcgataacgcgggtgtcaaggtgaggaacagacattggaggagcgccaacagatgccttgccatataccttggctgcgtgctgctcgatcagttcctcgttggtgcaacgaagccacaggccggatactgggaatccagcgaagcccttgacctgtgggatgcctgcgctgcgaagcagatccagtgcgtatccgcctgcgccgacgaacacgaagtttgccttgatggtcttggtgtcgccagtgtgtacgttcttgacggtcacgatccactttgcgccatcagccttgatgctcttgacttcgtggccatagcggatttcagtgccttcaacttcagctgcatccaggtactgcttggtctgagcaccgtagttgatgtcggtgccttcatcgatccaagagattgctactggatcagagaagtcacggccctttgccatcaaaggcagcttctcggtgaaggtagcttcatcgtcagcgtaggtcatgccctggaagagtgggtgatccttcaaagcttcgtagcgagccttgatgtatgcaacctgatctgcgccctggccgaaagatacgtgaggaacagggttgatgaattccttaggatcagacagcactccctcttcaacgaggtgagaccagaactgacgggaaacctggaacttctcgttgattcctacagccttggcaatttcaaccttgcccttaacctctggggtgtagttcagctcgcatagagcagagtggccggttcctgcattgttccacggggaggacgactcttgtgccggtccatccaaacgctcgaagacgatctgagtccagcttggctccagctgacgcagcattgcacccagcgtggagctcatgataccggcaccaatgagaactacatctgcctcatcggtaatcctcggtgcgttcttcggggaatctgacat

SEQ ID NO: 257 - protein, 500 aa, Corynebacterium glutamicum
MSDSPKNAPRITDEADVVLIGAGIMSSTLGAMLRQLEPSWTQIVFERLDGPAQESSSPWNNAGTGHSALCELNYTPEVKGKVEIAKAVGINEKFQVSRQFWSHLVEEGVLSDPKEFINPVPHVSFGQGADQVAYIKARYEALKDHPLFQGMTYADDEATFTEKLPLMAKGRDFSDPVAISWIDEGTDINYGAQTKQYLDAAEVEGTEIRYGHEVKSIKADGAKWIVTVKNVHTGDTKTIKANFVFVGAGGYALDLLRSAGIPQVKGFAGFPVSGLWLRCTNEELIEQHAAKVYGKASVGAPPMSVPHLDTRVIEGEKGLLFGPYGGWTPKFLKEGSYLDLFKSIRPDNIPSYLGVAAQEFDLTKYLVTEVLKDQDKRMDALREYMPEAQNGDWETIVAGQRVQVIKPAGFPKFGSLEFGTTLINNSEGTIAGLLGASPGASIAPSAMIELLERCFGDRMIEWGDKLKDMIPSYGKKLASEPALFEQQWARTQKTLKLEEA

SEQ ID NO: 258 - DNA, 1302 bp, Corynebacterium glutamicum
  source 1..1302: mol_type = genomic DNA
atggggtccggcaagacgatctccgttgcggggccatcagtaattttggtgtacgccattattggtttcatgcttttcttcgtcatgcgtgccatgggagagctgctgctcgccaatttgaattacaaatctttgcgcgatgcggtctctgatattttgggtcctggcgcaggttttgtcaccggctggacatattggttctgctggattgccacaggcatggcggacatcgtggcgatcactggatacacccaatactggtggcctgagatcccattgtggcttccaggtgtgctcaccattgcgttgctgtttgccctgaacttggctgcggtacgactgttcggtgagatggagttttggttcgccatcatcaaaatcgtggctatcgtgtccttgatcgtcgtgggacttttcatggtggtcacagcctttgaatcacctaatggcaccaccgcgcagttcaacaacctcattgagcatggcggatttttccccaacggcatcaccggtttcttggctggtttccagatcgctatctttgcgttcgtcgggattgaacttgccggcactgcagctgcagagactgagaatcccaccaagacgcttcctcgggcaatcaactccattcccatccgcatcgtggtgttctatgttttggcgttggctgtcatcatgatggtcaccccatgggatcaggtccgtgctgacaacagcccattcgtgcagatgttcgcgctggcaggaatcccagcggcggcaggcatcattaactttgtggtcatcacttctgcagcgtcgtctgccaacagtggtattttctccacctcccgcatgttgtatggattgtctttggaaggcgcagctccgaaacggtggagccggttgtccaagaacttggtgccagccaggggattgactttttctgtgatttgcctcattccagcggtgggtttgctgtacgctggcggcactgtcatcgaggcattcacactgatcaccacggtttcttcggtgttgttcatggtggtgtggtcctacattttggtggcttatatcgtctaccgccgcaacagcccggaattacacaaaaagtcgattttcaaaatgcctggcggcgtggtcatggcagttgtggtgttggtgttcttcgcagcgatgttggtggtgctgtccctggagccggatacccgtgcagcgctcatcgcgacgccagtgtggttcatcattttgggtatcggttggttgtccatcggtggagctaagggcgctaagcatcgcagccaaataacctcccactaa

SEQ ID NO: 259 - protein, 433 aa, Corynebacterium glutamicum
MGSGKTISVAGPSVILVYAIIGFMLFFVMRAMGELLLANLNYKSLRDAVSDILGPGAGFVTGWTYWFCWIATGMADIVAITGYTQYWWPEIPLWLPGVLTIALLFALNLAAVRLFGEMEFWFAIIKIVAIVSLIVVGLFMVVTAFESPNGTTAQFNNLIEHGGFFPNGITGFLAGFQIAIFAFVGIELAGTAAAETENPTKTLPRAINSIPIRIVVFYVLALAVIMMVTPWDQVRADNSPFVQMFALAGIPAAAGIINFVVITSAASSANSGIFSTSRMLYGLSLEGAAPKRWSRLSKNLVPARGLTFSVICLIPAVGLLYAGGTVIEAFTLITTVSSVLFMVVWSYILVAYIVYRRNSPELHKKSIFKMPGGVVMAVVVLVFFAAMLVVLSLEPDTRAALIATPVWFIILGIGWLSIGGAKGAKHRSQITSH

SEQ ID NO: 260 - DNA, 987 bp, Corynebacterium glutamicum
  source 1..987: mol_type = genomic DNA
ttagagcaagtcgcgcactgcctcgcgctcggcctgcaattcctgagcattcgcgtcgatgcgggcgcgctggaaatcggaaatctccaggccttcaacgatttcccactcaccgttgcgggatacggttggcagaccgacaaaaatgccctcaggaatgccgtatgcaccggtggaaggaattgccgcagaggaccacgcctcggtgccctgtacccaatcgcgcatgtgatcaatcgcagaggatgctgcagaagctgcagaagactttccacggacctcaatgatttcagcgccacggttagccacgcgaggaatgaactcctccacataccaatcgtgatcaaccaggtcagtgaccttttctccaccaacggttgcgtaggtgatgtctgggaactgggttgcggagtgatttccccagaccacaatgttgttaaattccgcagatccacggccaagcttggtggccagctgggagatcgcacggttgtgatcaaggcgcatcattgcgttgaagcgggatgctggaacatctggggccgcagctgaagcaatcaacgcgttggtgttcgctgggtttccaacaactaggacacgaatgtcatctgcggcgttgtcattgatagctttaccttgaggtccgaaaatcttgccgttgttagccagcaaatctgcgcgctcttcgccttttccgcgaggcttcgcaccgaccaaaaacgccgcattagcgccgtcgaatgcctcattggcatccgcggtgatggtgatgtttcgcaggagggggaaggcagaatccagaagttccatagccacaccctctgccccgccaagagcctgagggatctccagaagtttcagttctacaggggtgtcggtgccgaatacttcaccgttggcgatgcgccacaacagtgaataagagatttgaccagctgcgccggtgacggtgaccttcttggtggagacgttctgcggggaattcat

SEQ ID NO: 261 - protein, 328 aa, Corynebacterium glutamicum
MNSPQNVSTKKVTVTGAAGQISYSLLWRIANGEVFGTDTPVELKLLEIPQALGGAEGVAMELLDSAFPLLRNITITADANEAFDGANAAFLVGAKPRGKGEERADLLANNGKIFGPQGKAINDNAADDIRVLVVGNPANTNALIASAAAPDVPASRFNAMMRLDHNRAISQLATKLGRGSAEFNNIVVWGNHSATQFPDITYATVGGEKVTDLVDHDWYVEEFIPRVANRGAEIIEVRGKSSAASAASSAIDHMRDWVQGTEAWSSAAIPSTGAYGIPEGIFVGLPTVSRNGEWEIVEGLEISDFQRARIDANAQELQAEREAVRDLL

SEQ ID NO: 262 - DNA, 3898 bp, Choroflexus aurantiacus
  source 1..3898: mol_type = genomic DNA
aacatcatggtatactatacctatcgataattcttcaactaattgcataacagaacagcgatggcgacgggggagtccatgagcggaacaggacgactggcaggaaagattgcgttaattaccggtggcgccggcaatatcggcagtgaattgacacgtcgctttctcgcagagggagcgacggtcattattagtggacggaatcgggcgaagttgaccgcactggccgaacggatgcaggcagaggcaggagtgccggcaaagcgcatcgatctcgaagtcatggatgggagtgatccggtcgcggtacgtgccggtatcgaagcgattgtggcccgtcacggccagatcgacattctggtcaacaatgcaggaagtgccggtgcccagcgtcgtctggccgagattccactcactgaagctgaattaggccctggcgccgaagagacgcttcatgccagcatcgccaatttacttggtatgggatggcatctgatgcgtattgcggcacctcatatgccggtaggaagtgcggtcatcaatgtctcgaccatcttttcacgggctgagtactacgggcggattccgtatgtcacccctaaagctgctcttaatgctctatctcaacttgctgcgcgtgagttaggtgcacgtggcatccgcgttaatacgatctttcccggcccgattgaaagtgatcgcatccgtacagtgttccagcgtatggatcagctcaaggggcggcccgaaggcgacacagcgcaccattttttgaacaccatgcgattgtgtcgtgccaacgaccagggcgcgcttgaacgtcggttcccctccgtcggtgatgtggcagacgccgctgtctttctggccagtgccgaatccgccgctctctccggtgagacgattgaggttacgcacggaatggagttgccggcctgcagtgagaccagcctgctggcccgtactgatctgcgcacgattgatgccagtggccgcacgacgctcatctgcgccggcgaccagattgaagaggtgatggcgctcaccggtatgttgcgtacctgtgggagtgaagtgatcatcggcttccgttcggctgcggcgctggcccagttcgagcaggcagtcaatgagagtcggcggctggccggcgcagactttacgcctcccattgccttgccactcgatccacgcgatccggcaacaattgacgctgtcttcgattggggggccggcgagaataccggcgggattcatgcagcggtgattctgcctgctaccagtcacgaaccggcaccgtgcgtgattgaggttgatgatgagcgggtgctgaattttctggccgatgaaatcaccgggacaattgtgattgccagtcgcctggcccgttactggcagtcgcaacggcttacccccggcgcacgtgcgcgtgggccgcgtgtcatttttctctcgaacggtgccgatcaaaatgggaatgtttacggacgcattcaaagtgccgctatcggtcagctcattcgtgtgtggcgtcacgaggctgaacttgactatcagcgtgccagcgccgccggtgatcatgtgctgccgccggtatgggccaatcagattgtgcgcttcgctaaccgcagccttgaagggttagaatttgcctgtgcctggacagctcaattgctccatagtcaacgccatatcaatgagattaccctcaacatccctgccaacattagcgccaccaccggcgcacgcagtgcatcggtcggatgggcggaaagcctgatcgggttgcatttggggaaagttgccttgattaccggtggcagcgccggtattggtgggcagatcgggcgcctcctggctttgagtggcgcgcgcgtgatgctggcagcccgtgatcggcataagctcgaacagatgcaggcgatgatccaatctgagctggctgaggtggggtataccgatgtcgaagatcgcgtccacattgcaccgggctgcgatgtgagtagcgaagcgcagcttgcggatcttgttgaacgtaccctgtcagcttttggcaccgtcgattatctgatcaacaacgccgggatcgccggtgtcgaagagatggttatcgatatgccagttgagggatggcgccataccctcttcgccaatctgatcagcaactactcgttgatgcgcaaactggcgccgttgatgaaaaaacagggtagcggttacatccttaacgtctcatcatactttggcggtgaaaaagatgcggccattccctaccccaaccgtgccgattacgccgtctcgaaggctggtcagcgggcaatggccgaagtctttgcgcgcttccttggcccggagatacagatcaatgccattgcgccgggtccggtcgaaggtgatcgcttgcgcggtaccggtgaacgtcccggcctctttgcccgtcgggcgcggctgattttggagaacaagcggctgaatgagcttcacgctgctcttatcgcggctgcgcgcaccgatgagcgatctatgcacgaactggttgaactgctcttacccaatgatgtggccgcactagagcagaatcccgcagcacctaccgcgttgcgtgaactggcacgacgttttcgcagcgaaggcgatccggcggcatcatcaagcagtgcgctgctgaaccgttcaattgccgctaaattgctggctcgtttgcataatggtggctatgtgttgcctgccgacatctttgcaaacctgccaaacccgcccgatcccttcttcacccgagcccagattgatcgcgaggctcgcaaggttcgtgacggcatcatggggatgctctacctgcaacggatgccgactgagtttgatgtcgcaatggccaccgtctattaccttgccgaccgcaatgtcagtggtgagacattccacccatcaggtggtttgcgttacgaacgcacccctaccggtggcgaactcttcggcttgccctcaccggaacggctggcggagctggtcggaagcacggtctatctgataggtgaacatctgactgaacaccttaacctgcttgcccgtgcgtacctcgaacgttacggggcacgtcaggtagtgatgattgttgagacagaaaccggggcagagacaatgcgtcgcttgctccacgatcacgtcgaggctggtcggctgatgactattgtggccggtgatcagatcgaagccgctatcgaccaggctatcactcgctacggtcgcccagggccggtcgtctgtacccccttccggccactgccgacggtaccactggtcgggcgtaaagacagtgactggagcacagtgttgagtgaggctgaatttgccgagttgtgcgaacaccagctcacccaccatttccgggtagcgcgcaagattgccctgagtgatggtgccagtctcgcgctggtcactcccgaaactacggctacctcaactaccgagcaatttgctctggctaacttcatcaaaacgacccttcacgcttttacggctacgattggtgtcgagagcgaaagaactgctcagcgcattctgatcaatcaagtcgatctgacccggcgtgcgcgtgccgaagagccgcgtgatccgcacgagcgtcaacaagaactggaacgttttatcgaggcagtcttgctggtcactgcaccactcccgcctgaagccgatacccgttacgccgggcggattcatcgcggacgggcgattaccgtgtaaattctacgccacaggaaccactaccaaaccagcatagtaagagaacgatagagacgttgcaatgcgacgtctctatcatatttccggccccccctagacaaacccccacgtcttcgtgtagactagaaacaggaggctgtatgcacgtccaacaaga

SEQ ID NO: 263 - protein, 1220 aa, Choroflexus aurantiacus
MSGTGRLAGKIALITGGAGNIGSELTRRFLAEGATVIISGRNRAKLTALAERMQAEAGVPAKRIDLEVMDGSDPVAVRAGIEAIVARHGQIDILVNNAGSAGAQRRLAEIPLTEAELGPGAEETLHASIANLLGMGWHLMRIAAPHMPVGSAVINVSTIFSRAEYYGRIPYVTPKAALNALSQLAARELGARGIRVNTIFPGPIESDRIRTVFQRMDQLKGRPEGDTAHHFLNTMRLCRANDQGALERRFPSVGDVADAAVFLASAESAALSGETIEVTHGMELPACSETSLLARTDLRTIDASGRTTLICAGDQIEEVMALTGMLRTCGSEVIIGFRSAAALAQFEQAVNESRRLAGADFTPPIALPLDPRDPATIDAVFDWGAGENTGGIHAAVILPATSHEPAPCVIEVDDERVLNFLADEITGTIVIASRLARYWQSQRLTPGARARGPRVIFLSNGADQNGNVYGRIQSAAIGQLIRVWRHEAELDYQRASAAGDHVLPPVWANQIVRFANRSLEGLEFACAWTAQLLHSQRHINEITLNIPANISATTGARSASVGWAESLIGLHLGKVALITGGSAGIGGQIGRLLALSGARVMLAARDRHKLEQMQAMIQSELAEVGYTDVEDRVHIAPGCDVSSEAQLADLVERTLSAFGTVDYLINNAGIAGVEEMVIDMPVEGWRHTLFANLISNYSLMRKLAPLMKKQGSGYILNVSSYFGGEKDAAIPYPNRADYAVSKAGQRAMAEVFARFLGPEIQINAIAPGPVEGDRLRGTGERPGLFARRARLILENKRLNELHAALIAAARTDERSMHELVELLLPNDVAALEQNPAAPTALRELARRFRSEGDPAASSSSALLNRSIAAKLLARLHNGGYVLPADIFANLPNPPDPFFTRAQIDREARKVRDGIMGMLYLQRMPTEFDVAMATVYYLADRNVSGETFHPSGGLRYERTPTGGELFGLPSPERLAELVGSTVYLIGEHLTEHLNLLARAYLERYGARQVVMIVETETGAETMRRLLHDHVEAGRLMTIVAGDQIEAAIDQAITRYGRPGPVVCTPFRPLPTVPLVGRKDSDWSTVLSEAEFAELCEHQLTHHFRVARKIALSDGASLALVTPETTATSTTEQFALANFIKTTLHAFTATIGVESERTAQRILINQVDLTRRARAEEPRDPHERQQELERFIEAVLLVTAPLPPEADTRYAGRIHRGRAITV

SEQ ID NO: 264 - DNA, 1266 bp, Escherichia coli
  source 1..1266: mol_type = genomic DNA
atgagcaacaatgaattccatcagcgtcgtctttctgccactccgcgcggggttggcgtgatgtgtaacttcttcgcccagtcggctgaaaacgccacgctgaaggatgttgagggcaacgagtacatcgatttcgccgcaggcattgcggtgctgaataccggacatcgccaccctgatctggtcgcggcggtggagcagcaactgcaacagtttacccacaccgcgtatcagattgtgccgtatgaaagctacgtcaccctggcggagaaaatcaacgcccttgccccggtgagcgggcaggccaaaaccgcgttcttcaccaccggtgcggaagcggtggaaaacgcggtgaaaattgctcgcgcccataccggacgccctggcgtgattgcgtttagcggcggctttcacggtcgtacgtatatgaccatggcgctgaccggaaaagttgcgccgtacaaaatcggcttcggcccgttccctggttcggtgtatcacgtaccttatccgtcagatttacacggcatttcaacacaggactccctcgacgccatcgaacgcttgtttaaatcagacatcgaagcgaagcaggtggcggcgattattttcgaaccggtgcagggcgagggcggtttcaacgttgcgccaaaagagctggttgccgctattcgccgcctgtgcgacgagcacggtattgtgatgattgctgatgaagtgcaaagcggctttgcgcgtaccggtaagctgtttgccatggatcattacgccgataagccggatttaatgacgatggcgaaaagcctcgcgggcgggatgccgctttcgggcgtggtcggtaacgcgaatattatggacgcacccgcgccgggcgggcttggcggcacctacgccggtaacccgctggcggtggctgccgcgcacgcggtgctcaacattatcgacaaagaatcactctgcgaacgcgcgaatcaactgggccagcgtctcaaaaacacgttgattgatgccaaagaaagcgttccggccattgctgcggtacgcggcctggggtcgatgattgcggtagagtttaacgatccgcaaacgggcgagccgtcagcggcgattgcacagaaaatccagcaacgcgcgctggcgcaggggctgctcctgctgacctgtggcgcatacggcaacgtgattcgcttcctgtatccgctgaccatcccggatgcgcaattcgatgcggcaatgaaaattttgcaggatgcgctgagcgattaa

SEQ ID NO: 265 - protein, 421 aa, Escherichia coli
MSNNEFHQRRLSATPRGVGVMCNFFAQSAENATLKDVEGNEYIDFAAGIAVLNTGHRHPDLVAAVEQQLQQFTHTAYQIVPYESYVTLAEKINALAPVSGQAKTAFFTTGAEAVENAVKIARAHTGRPGVIAFSGGFHGRTYMTMALTGKVAPYKIGFGPFPGSVYHVPYPSDLHGISTQDSLDAIERLFKSDIEAKQVAAIIFEPVQGEGGFNVAPKELVAAIRRLCDEHGIVMIADEVQSGFARTGKLFAMDHYADKPDLMTMAKSLAGGMPLSGVVGNANIMDAPAPGGLGGTYAGNPLAVAAAHAVLNIIDKESLCERANQLGQRLKNTLIDAKESVPAIAAVRGLGSMIAVEFNDPQTGEPSAAIAQKIQQRALAQGLLLLTCGAYGNVIRFLYPLTIPDAQFDAAMKILQDALSD

SEQ ID NO: 266 - DNA, 1503 bp, Rattus norvegicus
  source 1..1503: mol_type = genomic DNA
atggccttcttgttgactacccgacggctggtctgcagttcccagaaaaacctccacctcttcacacctggatccagatacatcagccaagctgctgccaaagttgactttgagtttgattatgatggaccactcatgaagacagaagtcccgggccctagatctcaggagctaatgaaacagctgaacacaatccagaatgcagaggccgtgcactttttctgcaactacgaagagagccgaggcaactacctcgtggacgtggatggcaaccgcatgttggacctgtattctcagatctcctctgtacccatcggttacaaccatccggctctggcgaaactcgttcaacagcctcaaaacgcgagcactttcatcaacagacctgccctgggcatcctgcctccagagaactttgtggacaagctccgggagtccttgatgtcggtggcgcccaaaggcatgtgtcagctcatcacgatggcctgcgggtcctgctccaatgagaatgcattcaagaccatcttcatgtggtaccggagtaaagaacgaggtcagagaggtttctccaaagaggagctggagacttgcatggttaaccagagtcctggatgcccagactacagcatcctctccttcatgggtgctttccacgggaggaccatgggttgcttagcgaccacacactccaaagcaattcacaagattgacatcccttcctttgactggcccattgctccattcccacggctgaaatatcccctggaggagtttgtgacggacaatcagcaagaggaggcccgctgtctagaagaggtggaggatctaattgtgaaatatcggaaaaagaagagaacagtggctgggatcatcgtggagcccatccagtccgaaggtggagacaaccacgcatcagatgacttcttccggaagctgagagacatagccaggaagcatggctgtgccttcttggtggacgaggttcagactggaggaggctgtacaggcaagttctgggcccatgaacactggggcttggatgacccagccgacgtgatgtcgttcagcaagaagatgatgactgggggcttcttccacaaggaggagtttcgaccaagtgctccttaccggatcttcaacacctggctgggggacccatccaagaacttgctgctggctgaggtcatcaacatcatcaagcgggaagacctgctcaacaacgtggcccatgccgggaagaccctactgaccgggctgctggacctccaggcccagtacccccagttcgtcagccgggtgaggggacgaggcaccttctgttccttcgacactcccgacaaagccatacggaataaactcatcctaattgccaggaacaaaggtgtggtactggggggctgcggtgacaaatccatacgtttccgtcccacgctggtcttcagggatcaccatgcccacttgttcctcaacattttcagtggtatcttagcagacttcaagtaa

SEQ ID NO: 267 - protein, 500 aa, Rattus norvegicus
MAFLLTTRRLVCSSQKNLHLFTPGSRYISQAAAKVDFEFDYDGPLMKTEVPGPRSQELMKQLNTIQNAEAVHFFCNYEESRGNYLVDVDGNRMLDLYSQISSVPIGYNHPALAKLVQQPQNASTFINRPALGILPPENFVDKLRESLMSVAPKGMCQLITMACGSCSNENAFKTIFMWYRSKERGQRGFSKEELETCMVNQSPGCPDYSILSFMGAFHGRTMGCLATTHSKAIHKIDIPSFDWPIAPFPRLKYPLEEFVTDNQQEEARCLEEVEDLIVKYRKKKRTVAGIIVEPIQSEGGDNHASDDFFRKLRDIARKHGCAFLVDEVQTGGGCTGKFWAHEHWGLDDPADVMSFSKKMMTGGFFHKEEFRPSAPYRIFNTWLGDPSKNLLLAEVINIIKREDLLNNVAHAGKTLLTGLLDLQAQYPQFVSRVRGRGTFCSFDTPDKAIRNKLILIARNKGVVLGGCGDKSIRFRPTLVFRDHHAHLFLNIFSGILADFK

SEQ ID NO: 268 - DNA, 1368 bp, Bacillus smithii
  source 1..1368: mol_type = genomic DNA
atgaaaacgatggatgtgattcaaacaaaaaaagaagatcaacagtatgtcttgcattcatggtccaaacaaaagaatattcatccgaaagtcatcacgaaagctgaaggcatttttttctgggatgatgaaaaccgaaaatactatgatatgtgttcacaactcgtttacttgaatgttggacatcagcatcctaaattgttggaagaatttaagcatatggatgaaattcctatcgctgcaccttcatttgcgacaccatcaaaatcagaactcgctcgcaaaattattgaagttgcgccggacaacatggcgaaagtattttttgcaaacggtggagcagattcaaatgatcatgctattaaaatcgcaaggatggtcacaggacgttataaaattttttcaaggtatcgttcgtaccacggtgccacattcggcgccggcaatttaaccggtgaaaatcgtcgatttgctgttgaacctgggatgccgggttttattaaatttgatacaccttatttgtaccgggagtctattaattttgaaaatgaagaagcggcagcaaagttttaccttgatcggttgcacaatcaaatgctatatgaaggaccggaatccattgctgctgtatttcttgaacccattccgggcagcaatggtgttctcattccgccaaaaggatatcttgaaggggtgcgtgaattatgtgaccgctacggaattcttatggtttgtgatgaagtgatgtcgggatttggaagaacaggaaaaatgttcgcggtcgatcattttaacttccaaccagatatgatgacgtttgctaaaggggtaacaagtggttatagcccgcttggcggtgtgattgtcagccgaaaagtggcggagtttttcgatgaccatgtattaatgacaggtttaacatacagcggacataccgtttcaacccaaatcgggtgtgcaacgcttgatatttataaagaagaaggtctcattgaaaatgcttctgaagtaggtgaagtgctaggagcaagattggaaaaactaagagcgtttaactcagttggtgatgttcgtcatatcggtttattcgcagccgttgaacttgttagagacaaaaagacaaaggaaccgattatatcgtatgggatggattatggaaaagatgaagcgggattaatgaaaacattcattcatttattgaatgaaaagggattttatacgtactcgcatgaatccagtgtgatcattgcacctccattaatcattacagaacaacagataaatgaggcgatggatcttttcgaagaagcattgaccaagtttgaaaaggagcatttattctctgctggaacattggtaactaaataa

SEQ ID NO: 269 - protein, 455 aa, Bacillus smithii
MKTMDVIQTKKEDQQYVLHSWSKQKNIHPKVITKAEGIFFWDDENRKYYDMCSQLVYLNVGHQHPKLLEEFKHMDEIPIAAPSFATPSKSELARKIIEVAPDNMAKVFFANGGADSNDHAIKIARMVTGRYKIFSRYRSYHGATFGAGNLTGENRRFAVEPGMPGFIKFDTPYLYRESINFENEEAAAKFYLDRLHNQMLYEGPESIAAVFLEPIPGSNGVLIPPKGYLEGVRELCDRYGILMVCDEVMSGFGRTGKMFAVDHFNFQPDMMTFAKGVTSGYSPLGGVIVSRKVAEFFDDHVLMTGLTYSGHTVSTQIGCATLDIYKEEGLIENASEVGEVLGARLEKLRAFNSVGDVRHIGLFAAVELVRDKKTKEPIISYGMDYGKDEAGLMKTFIHLLNEKGFYTYSHESSVIIAPPLIITEQQINEAMDLFEEALTKFEKEHLFSAGTLVTK

(19) United States
(12) Patent Application Publication
(10) Pub. No.: US 2026/0015622 A1
(43) Pub. Date: Jan. 15, 2026

(54) TAURINE BIOSYNTHESIS USING GENETICALLY MODIFIED BACTERIA

(71) Applicant: Chemical Evolution Ltd., Calgary (CA)

(72) Inventors: Alejandra ENRIQUEZ, Calgary (CA); Trevor RANDALL, Calgary (CA); Dustin LILLICO, Calgary (CA); Markus WEISSENBERGER, Calgary (CA)

(21) Appl. No.: 19/020,873

(22) Filed: Jan. 14, 2025

(30) Foreign Application Priority Data
Jul. 12, 2024 (CA) 3248307

(51) Int. Cl. C12N 15/77; C12N 1/20; C12N 9/02; C12N 9/80; C12P 13/04; C12R 1/15

(52) U.S. Cl. CPC C12N 15/77; C12N 1/20; C12N 9/0069; C12N 9/0071; C12N 9/80; C12P 13/04; C12R 2001/15; C12Y 113/11019; C12Y 114/13148; C12Y 305/01092

(57) ABSTRACT

A genetically modified prokaryotic cell comprising:
at least one of the following:
   i. an addition, deletion and/or alteration of at least one gene to promote to taurine production; and
   ii. an addition, deletion and/or alteration of at least one gene related to taurine cellular transportation; and
at least one of the following polynucleotide sequences:
   i. a vanin (vnn) polynucleotide sequence selected from the group consisting of:
      vanin-1 (vnn1), wherein said vnn1 polynucleotide sequence has at least 70% sequence coverage to SEQ 3 or SEQ 98, and at least 70% sequence identity to SEQ 3 or SEQ 98;
      vanin-2 (vnn2), wherein said vnn2 polynucleotide sequence has at least 70% sequence coverage to SEQ 100, and at least 70% sequence identity to SEQ 100; and
      vanin-3 (vnn3), wherein said vnn3 polynucleotide sequence has at least 70% sequence coverage to SEQ 141, and at least 70% sequence identity to SEQ 141;
   ii. a cysteamine dioxygenase (ado) polynucleotide sequence which has at least 70% sequence coverage to SEQ 1, and at least 70% sequence identity to SEQ 1; and
   iii. a flavin-containing monooxygenase 1 (fmo1) polynucleotide sequence which has at least 70% sequence coverage to SEQ 5 or SEQ 99, and at least 70% of sequence identity to SEQ 5 or SEQ 99.

### REFERENCE TO AN ELECTRONIC SEQUENCE LISTING

The contents of the electronic sequence listing (2025-08-04_SequenceListing_ST26_23156-202US1.xml; Size: 446,422 bytes; and Date of Creation: Aug. 3, 2025) is herein incorporated by reference in its entirety.

### FIELD OF THE INVENTION

The present invention relates to a method to promote the production of taurine through genetically modified bacteria using naturally present or added metabolic pathways.

### BACKGROUND OF THE INVENTION:

Taurine is an amino acid that has been shown to be beneficial to human and animal health and development, and thus, it is commonly found supplemented into animal feed for livestock, as a stimulant in energy drinks, sold directly as a supplement, and in baby formula. Currently, most of the taurine on the market is either extracted directly from protein hydrolysates or chemically synthesized from either ethylene oxide or monoethanolamine (MEA). Manufacturing taurine using ethylene oxide, although the most common, comes with several drawbacks including its toxicity, volatility, and explosive potential, in addition to the severe conditions of temperature and pressure under which the reaction takes place. On the other hand, the industrial production of taurine from MEA is a two-step batch process in which the first step is the reaction of MEA with sulfuric acid to produce the ester 2-aminoethyl hydrogen sulfate (AES) and then, a second step involving a subsequent reaction of AES with a sulfite reagent. This general manufacturing process has limitations which includes the yield of the intermediate esters involved within the above-mentioned reactions and the need for high temperatures and corrosive acids to achieve production.

To overcome these limitations, the proposed invention is intended to utilize a prokaryotic biological system for the production of taurine through specific genetic modifications that incorporate pathways exclusively associated with eukaryotes. Many prior art patents and patent applications have delved into the modification of organisms to generate taurine, however, the pathways employed are different than the one being proposed herein.

U.S. patent application No. 2019/0153463 A1 describes an approach to produce or increase taurine and/or hypotaurine production in prokaryotes or eukaryotes. It describes a genetic transformation of organisms with algal, microalgal or fungal genes that encode proteins that catalyze the conversion of sulfur-containing compounds such as sulfate or cysteine to taurine. The application describes methods for the use of polynucleotides for cysteine dioxygenase-like (CDOL), sulfinoalanine decarboxylase-like (SADL), cysteine sulfate/decarboxylase or a portion of the cysteine synthetase/PLP decarboxylase (partCS/PLP-DC) polypeptide in bacteria, alga, yeast, or plants to produce or enhance taurine and/or hypotaurine formation. The preferred embodiment of the invention is said to be in plants, but other organisms may be used.

U.S. Pat. No. 10,874,625 B2 describes an approach to increase taurine or hypotaurine production in prokaryotes. More particularly, the invention relates to genetic transformation of organisms with genes that encode proteins that catalyze the conversion of cysteine to taurine, methionine to taurine, cysteamine to taurine, or alanine to taurine. The patent describes methods for the use of polynucleotides that encode cysteine dioxygenase (CDO) and sulfinoalanine decarboxylase (SAD) polypeptides in prokaryotes to increase taurine, hypotaurine or taurine precursor production. The preferred embodiment of the invention is in plants, but other organisms may be used. Increased taurine production in prokaryotes could be used as nutraceutical, pharmaceutical, or therapeutic compounds or as a supplement in animal feed.

U.S. Pat. No. 11,220,691 B2 describes an approach to produce or increase hypotaurine or taurine production in unicellular organisms. More particularly, the invention relates to genetic modification of unicellular organisms that include bacteria, algal, microalgal, diatoms, yeast, or fungi. The patent discloses methods to increase taurine levels in the cells by binding taurine or decreasing taurine degradation. The invention can be used in organisms that contain native or heterologous (transgenic) taurine biosynthetic pathways or cells that have taurine by enrichment. The invention also relates to methods to increase taurine levels in the cells and to use the said cells or extracts or purifications from the cells that contain the invention to produce plant growth enhancers, food, animal feed, aquafeed, food or drink supplements, animal-feed supplements, dietary supplements, health supplements or taurine.

In J. Agric. Food Chem. 2018. 66, 51, 13454-13463, Joo et al. reported, for the first time in bacteria, the production of taurine in metabolically engineered Corynebacterium glutamicum. The taurine-producing strain was developed by introducing CS. CDO1, and CSAD genes. Interestingly, while the control strain could not produce taurine, the engineered strains successfully produced taurine via the newly introduced metabolic pathway. Furthermore, the effect of a deletion strain of the transcriptional repressor McbR gene on taurine production was also studied which identified that sulfur accumulation and L-cysteine biosynthesis were reinforced causing a 2.3-fold increase in taurine production. As a result, sulfur accumulation and L-cysteine biosynthesis were reinforced by the MebR deletion strain, which further increased the taurine production by 2.3-fold. Taurine production of the final engineered strain Tau11 was higher than in other previously reported strains.

U.S. Pat. No. 11,326,171 B2 discloses non-naturally occurring microorganisms that produce taurine and/or taurine precursors, e.g., hypotaurine, sulfoacetaldehyde, or cysteate, utilizing exogenously added enzyme activities. The invention disclosed therein is said to relate to methods of producing taurine and/or taurine precursors in microbial cultures, and feed and nutritional supplement compositions that include taurine and/or taurine precursors produced in the microbial cultures, such as taurine- and/or taurine precursor-containing biomass, are also provided.

International patent application No. WO2023/146544 describes methods for the fermentative production of taurine-containing products from unicellular organisms. More particularly, the invention is said to relate to genetic modifications of the taurine and/or substrate biosynthetic pathways in unicellular organisms that include bacteria, algae, microalgae, diatoms, yeast, or fungi. The invention is also said to relate to fermentation and processing methods for the production of various taurine-containing products. The invention also relates to the use of the cells, fermentation broth or extracts that contain taurine to produce products for use in food, feed, beverages, dietary and health supplements, cosmetics, personal care, pharmaceuticals, or agricultural production.

Korean patent KR 1020100130564 discloses a recombinant microorganism with taurine producing ability and a method for preparing taurine using the same are provided to industrially use as a microorganism for taurine. A recombinant microorganism for producing taurine has a gene encoding enzyme relating to taurine biosynthesis. The microorganism also has attenuated or deleted gene encoding acetate kinase, taurine dioxygenase, gamma-glutamyltranspeptidase, lactate dehydrogenase or taurine transporter. A method for preparing taurine comprises: a step of culturing recombinant E. coli WADTLG3110/pTrcTpaXscPta to produce taurine; and a step for collecting taurine.

As illustrated in FIG. 1. Taurine production by eukaryotes is facilitated by very distinct sets of metabolic pathways which include the conversion of methionine and cysteine via cysteine sulfinic acid decarboxylase (CSAD) coupled with the oxidation of hypotaurine to generate taurine as the final step in the pathway. In contrast to the plethora of research conducted surrounding said process utilizing cysteine dioxygenase (CDO) and sulfinoalanine/cysteine sulfinic acid decarboxylase (SAD/CSAD), there are no commercial embodiments which employ the pathway converting coenzyme-A to cysteamine using vanin (VNN), cysteamine dioxygenase (ADO) which converts cysteamine to hypotaurine (ADO), followed by an oxidation reaction to generate taurine using flavin-containing monooxygenase 1 (FMO1). Therefore, the utilization of this pathway and, more importantly, its optimization to promote taurine production is necessary as there still exists a need to develop an improved optimized large scale, and environmentally conscious process for the production of taurine that is a cost-effective alternative to current chemical and biological processes utilized within this field.

### SUMMARY OF THE INVENTION

According to a preferred embodiment of the present invention, there is provided a genetically modified prokaryotic cell comprising:
at least one of the following:
   i. an addition, deletion and/or alteration of at least one gene to promote to taurine production; and
   ii. an addition, deletion and/or alteration of at least one gene related to taurine cellular transportation; and
at least one of the following polynucleotide sequences:
   i. a vanin (vnn) polynucleotide sequence selected from the group consisting of:
      vanin-1 (vnn1), wherein said vnn1 polynucleotide sequence has at least 70% sequence coverage to SEQ 3 or SEQ 98, and at least 70% sequence identity to SEQ 3 or SEQ 98;
      vanin-2 (vnn2), wherein said vnn2 polynucleotide sequence has at least 70% sequence coverage to SEQ 100, and at least 70% sequence identity to SEQ 100; and
      vanin-3 (vnn3), wherein said vnn3 polynucleotide sequence has at least 70% sequence coverage to SEQ 141, and at least 70% sequence identity to SEQ 141;
   ii. a cysteamine dioxygenase (ado) polynucleotide sequence which has at least 70% sequence coverage to SEQ 1, and at least 70% sequence identity to SEQ 1; and
   iii. a flavin-containing monooxygenase 1 (fmo1) polynucleotide sequence which has at least 70% sequence coverage to SEQ 5 or SEQ 99, and at least 70% of sequence identity to SEQ 5 or SEQ 99.

According to a second embodiment of the present invention, there is provided a prokaryotic cell comprising:
at least one of the following:
   i. an addition, deletion and/or alteration of at least one gene to promote taurine production; and
   ii. an addition, deletion and/or alteration of at least one gene related to taurine cellular transportation; and
at least one of the following polynucleotide sequences:
   i. a vanin (vnn) polynucleotide sequence selected from the group consisting of:
      a vnn1 polynucleotide sequence, wherein said vnn1 polynucleotide sequence is selected from the group consisting of: SEQ 3; SEQ 45, SEQ 46, SEQ, 47, SEQ 48, SEQ 49, SEQ 50, SEQ 51, SEQ 52, SEQ 53, SEQ 54, and SEQ 98;
      a vnn2 polynucleotide sequence, wherein said vnn2 polynucleotide sequence is selected from the group consisting of: SEQ 100; SEQ 101; SEQ 102; SEQ 103; SEQ 104; SEQ 105; SEQ 106; SEQ 107; SEQ 108; SEQ 109; SEQ 110; SEQ 111; SEQ 112; and SEQ 113; and
      a vnn3 polynucleotide sequence, wherein said vnn3 polynucleotide sequence is selected from the group consisting of: SEQ 141; SEQ 142; SEQ 143; SEQ 144; SEQ 145; SEQ 146; SEQ 147; SEQ 148; SEQ 149; SEQ 150; SEQ 151; SEQ 152; SEQ 153; SEQ 154; SEQ 155; SEQ 156; and SEQ 157;
   ii. a cysteamine dioxygenase (ado) polynucleotide sequence selected from the group consisting of: SEQ 1; SEQ 24; SEQ 25; SEQ 26; SEQ 27; SEQ 28; and SEQ 29; and
   iii. a flavin-containing monooxygenase 1 (fmo1) polynucleotide sequence selected from the group consisting of: SEQ 5; SEQ 71; SEQ 72; SEQ 73; SEQ 74; SEQ 75; SEQ 76; SEQ 77; SEQ 78; SEQ 79; SEQ 80; SEQ 81; and SEQ 99.

According to another embodiment of the present invention, there is provided a prokaryotic cell wherein, upon transcription and translation under the control of a native or synthetic promoter and Ribosomal binding site (RBS):
said vanin (vnn) polynucleotide sequence provides a vanin (VNN) polypeptide sequence selected from the group consisting of:
   i. a vanin-1 (VNN1) polypeptide sequence, wherein said VNN1 polypeptide sequence has at least 70% sequence coverage to SEQ 4, and at least 25% sequence identity to SEQ 4;
   ii. a vanin-2 (VNN2) polypeptide sequence, wherein said VNN2 polypeptide sequence has at least 70% sequence coverage to SEQ 114, and at least 25% sequence identity to SEQ 114; and
   iii. a vanin-3 (VNN3) polypeptide sequence, wherein said VNN3 polypeptide sequence has at least 70% sequence coverage to SEQ 158, and at least 25% sequence identity to SEQ 158; and
said cysteamine dioxygenase (ado) polynucleotide sequences provides a cysteamine dioxygenase (ADO) polypeptide sequence which has at least 70% sequence coverage to SEQ 2, and at least 25% sequence identity to SEQ 2; and
said flavin-containing monooxygenase 1 (fmo1) polynucleotide sequence provides a flavin-containing monooxygenase 1 (FMO1) polypeptide sequence which has at least 70% sequence coverage to SEQ 6, and at least 50% of sequence identity to SEQ 6.

According to another embodiment of the present invention, there is provided a prokaryotic cell wherein:
said vanin-1 (VNN1) polypeptide sequence is selected from the group consisting of: SEQ 4; SEQ 55, SEQ 56, SEQ, 57, SEQ 58, SEQ 59, SEQ 60, SEQ 61, SEQ 62, SEQ 63, SEQ 64, SEQ 65, SEQ, 66, SEQ 67, SEQ 68, SEQ 69, and SEQ 70;
said vanin-2 (VNN2) polypeptide sequence is selected from the group consisting of: SEQ 114; SEQ 115; SEQ 116; SEQ 117; SEQ 118; SEQ 119; SEQ 120; SEQ 121; SEQ 122; SEQ 123; SEQ 124; SEQ 125; SEQ 126; SEQ 127; SEQ 128; SEQ 129; SEQ 130; SEQ 131; SEQ 132; SEQ 133; SEQ 134; SEQ 135; SEQ 136; SEQ 137; SEQ 138; SEQ 139; and SEQ 140;
said vanin-3 (VNN3) polypeptide sequence is selected from the group consisting of SEQ 158; SEQ 159; SEQ 160; SEQ 161; SEQ 162; SEQ 163; SEQ 164; SEQ 165; SEQ 166; SEQ 167; SEQ 168; SEQ 169; SEQ 170; SEQ 171; SEQ 172; SEQ 173; SEQ 174; SEQ 175; SEQ 176; SEQ 177; SEQ 178; SEQ 179: SEQ 180; SEQ 181; SEQ 182; and SEQ 183;
said cysteamine dioxygenase (ADO) polypeptide sequence is selected from the group consisting of: SEQ 2; SEQ 30; SEQ 31; SEQ 32; SEQ 33; SEQ 34; SEQ 35; SEQ 36; SEQ 37; SEQ 38; SEQ 39; SEQ 40; SEQ 41; SEQ 42; SEQ 43; and SEQ 44; and
said flavin-containing monooxygenase 1 (FMO1) polypeptide sequence is selected from the group consisting of SEQ 6; SEQ 82; SEQ 83; SEQ 84; SEQ 85; SEQ 86; SEQ 87; SEQ 88; SEQ 89; SEQ 90; SEQ 91; SEQ 92; SEQ 93; SEQ 94; SEQ 95; SEQ 96; and SEQ 97.

According to a preferred embodiment of the present invention, there is provided a prokaryotic cell wherein said at least one gene to promote taurine production is selected from group consisting of: mcbR (SEQ 184), amtr (SEQ 186), xsc-like (SEQ 188), ssuI (SEQ 190), ssuD1 (SEQ 192), ssuD2 (SEQ 194), ssuR (SEQ 196), accA (SEQ 206), accB (SEQ 208), seuA (SEQ 210), seuB (SEQ 212), seuC (SEQ 214), ilvA (SEQ 216), gldc (SEQ 220), ilvB (SEQ 222), ilvN (SEQ 224), ilvC (SEQ 226), ilvD (SEQ 228), pyc (SEQ 230), dadA-like (SEQ 232), coaA (SEQ 234), coaBC (SEQ 236), coaD (SEQ 238), coaE (SEQ 240), panB (SEQ 244), panC (SEQ 246), panD (SEQ 248), panE (SEQ 250), aspB (SEQ 254), mqo (SEQ 256), and mdh (SEQ 260), mer (SEQ 262), puuE (SEQ 264), abat (SEQ 266), and pydD (SEQ 268).

According to a preferred embodiment of the present invention, there is provided a prokaryotic cell wherein upon transcription and translation under the control of a native or synthetic promoter and Ribosomal binding site (RBS), said at least one gene to promote taurine production generate a polypeptide sequence selected from the group consisting of: SEQ 185, SEQ 187, SEQ 189, SEQ 191, SEQ 193, SEQ 195, SEQ 197, SEQ 207, SEQ 209, SEQ 211, SEQ 213, SEQ 215, SEQ 217, SEQ 221, SEQ 223, SEQ 225, SEQ 227, SEQ 229, SEQ 231, SEQ 233, SEQ 235, SEQ 237, SEQ 239, SEQ 241, SEQ 245, SEQ 247, SEQ 249, SEQ 251, SEQ 255, SEQ 257, SEQ 261, SEQ 263, SEQ 265, SEQ 267, and SEQ 269.

According to another embodiment of the present invention, there is provided a prokaryotic cell wherein said at least one gene related to taurine cellular transportation is selected from the group consisting of: ssuA (SEQ 198), ssuB (SEQ 200), ssuC (SEQ 202), tauE (SEQ 204), gadC (SEQ 218), yhiM (SEQ 242), sdaC (SEQ 252), and cycA (SEQ 258).

According to a preferred embodiment of the present invention, there is provided a prokaryotic cell wherein upon transcription and translation under the control of a native or synthetic promoter and Ribosomal binding site (RBS), said at least one gene involved in the transport of taurine generate a polypeptide sequence selected from the group consisting of: SEQ 199, SEQ 201, SEQ 203, SEQ 205, SEQ 219, SEQ 243, SEQ, SEQ 253, and SEQ 259.

According to a preferred embodiment of the present invention, the prokaryotic cell further comprises a promoter and Ribosomal Binding Site (RBS) sequence which drives gene expression, wherein the genetic sequence for the promoter/RBS comprises a polypeptide sequence selected from the group consisting of: SEQ 7; SEQ 8; SEQ 9: SEQ 10; SEQ 11; SEQ 12; SEQ 13; SEQ 14; SEQ 15; SEQ 16; SEQ 17; SEQ 18; SEQ 19; SEQ 20; SEQ 21; SEQ 22; and SEQ 23.

According to a preferred embodiment of this invention, the prokaryotic cell is a bacterial cell. Preferably, the prokaryotic cell is selected from the group consisting of the genera: Brevibacterium, Bacillus, Corynebacterium, Escherichia, Lactococcus, Pseudomonas, Rhodococcus, and Serratia. More preferably, the prokaryotic belongs to the genus Corynebacterium.

### BRIEF DESCRIPTION OF THE ACCOMPANYING FIGURES

The invention may be more completely understood in consideration of the following description of various embodiments of the invention in connection with the accompanying figures, in which:

FIG. 1 is a schematic representation of various known metabolic pathways associated with the production of taurine.

FIG. 2 is a schematic representation of various genes that can be inserted, altered, and/or deleted to promote taurine production according to a preferred embodiment of the present invention, where (+) represents added and/or altered gene, (−) represents removed gene and A represents biological process being modified.

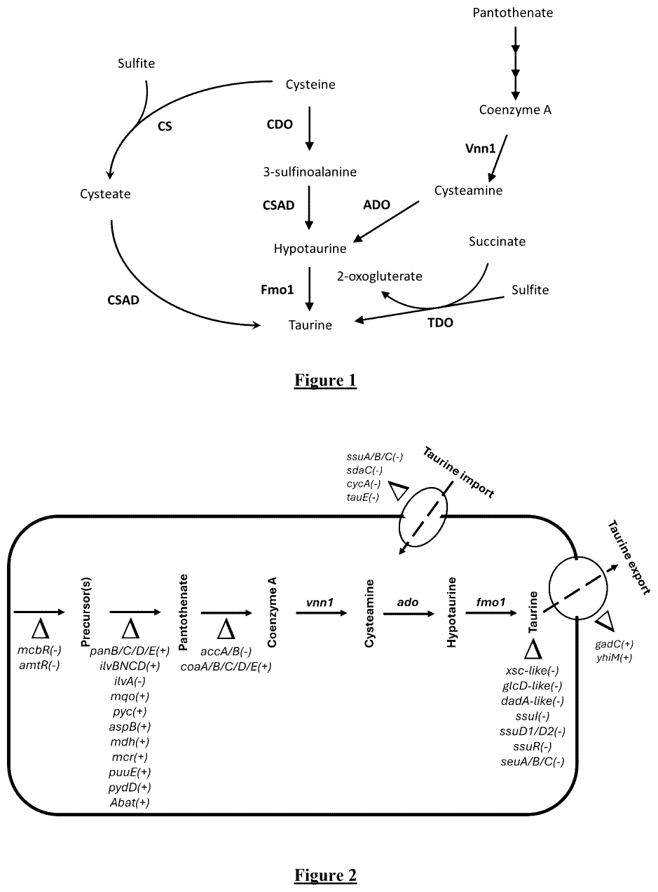

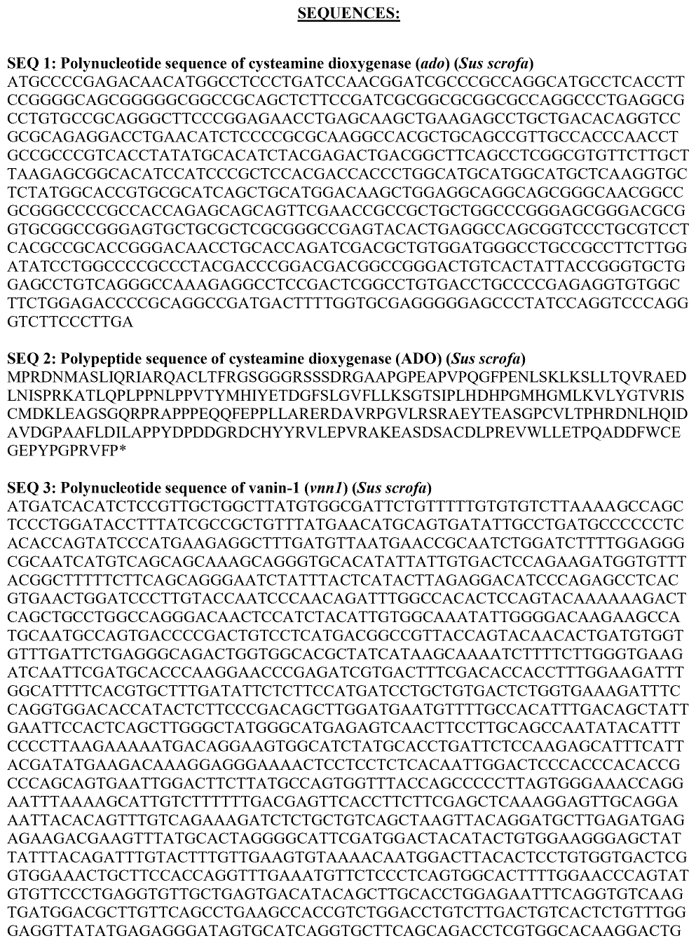

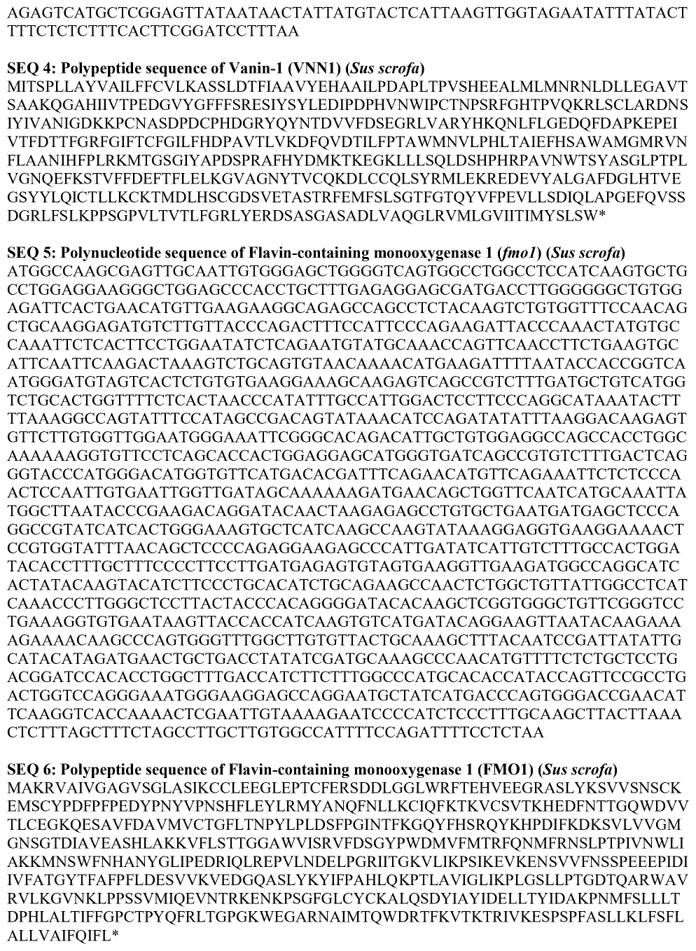

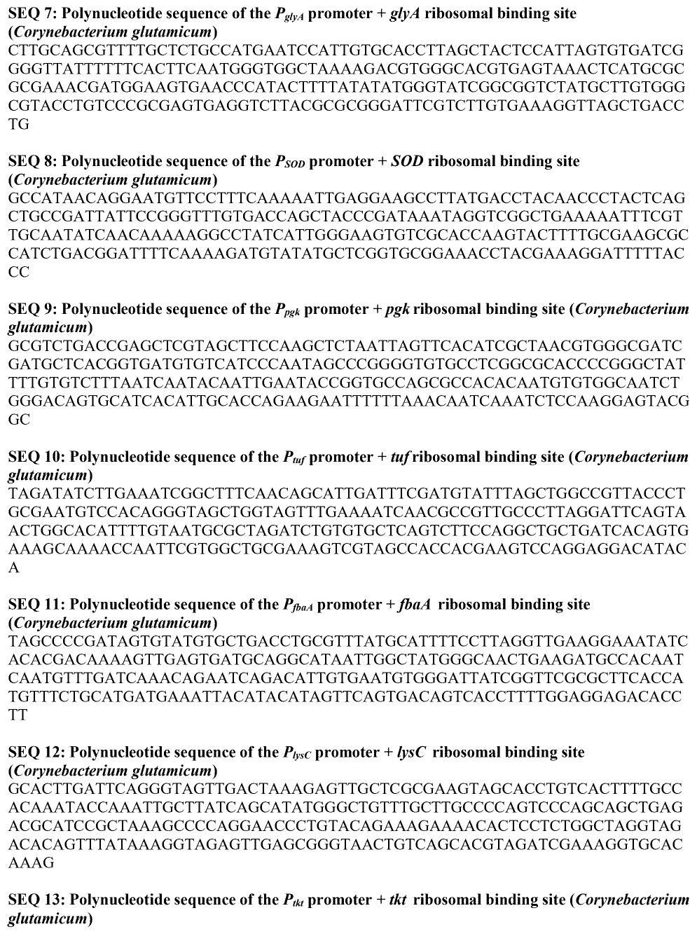

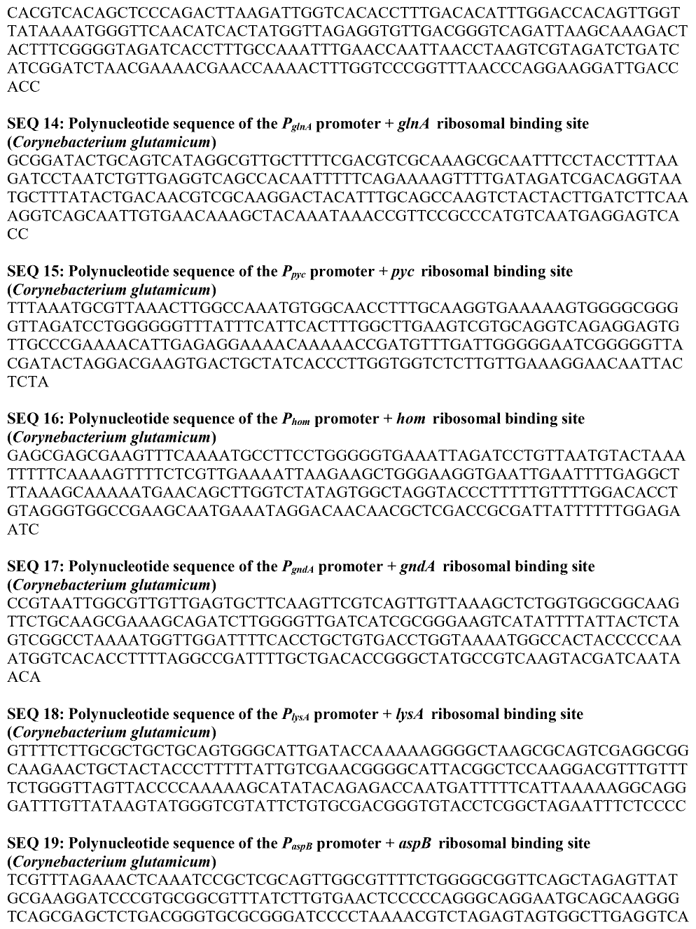

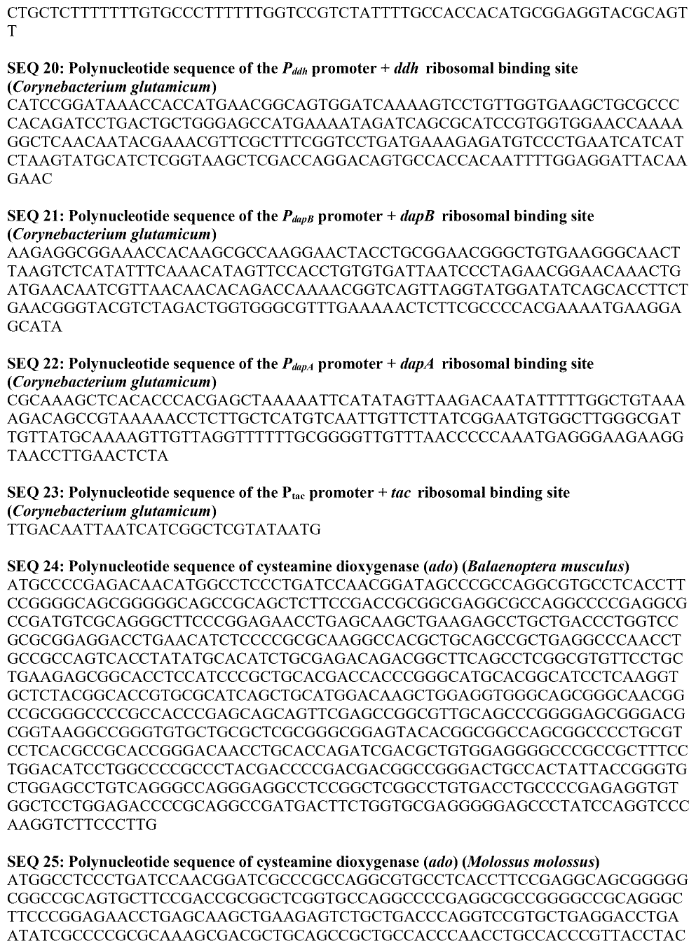

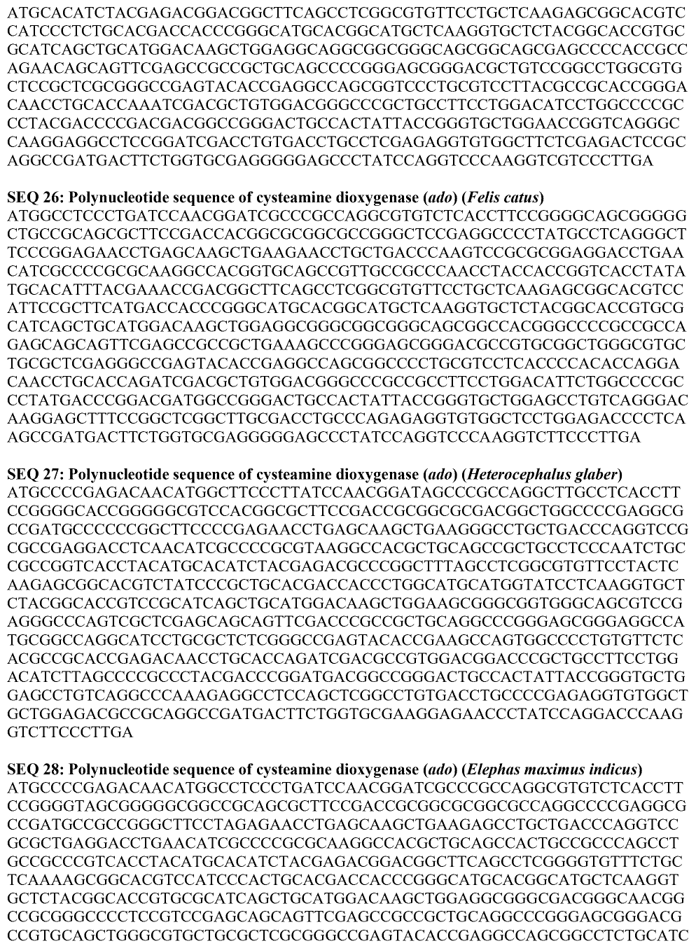

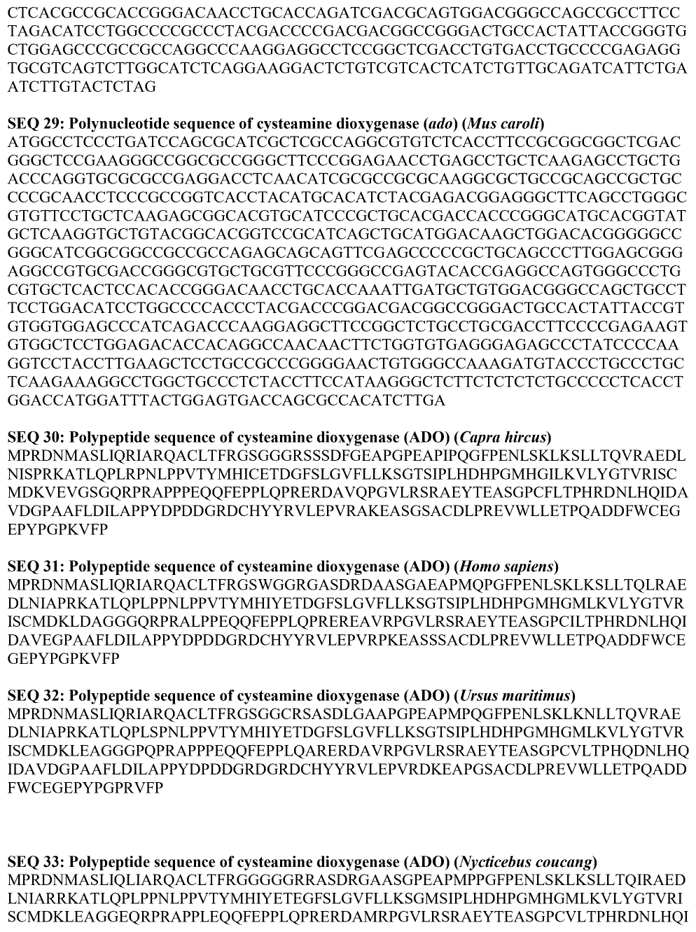

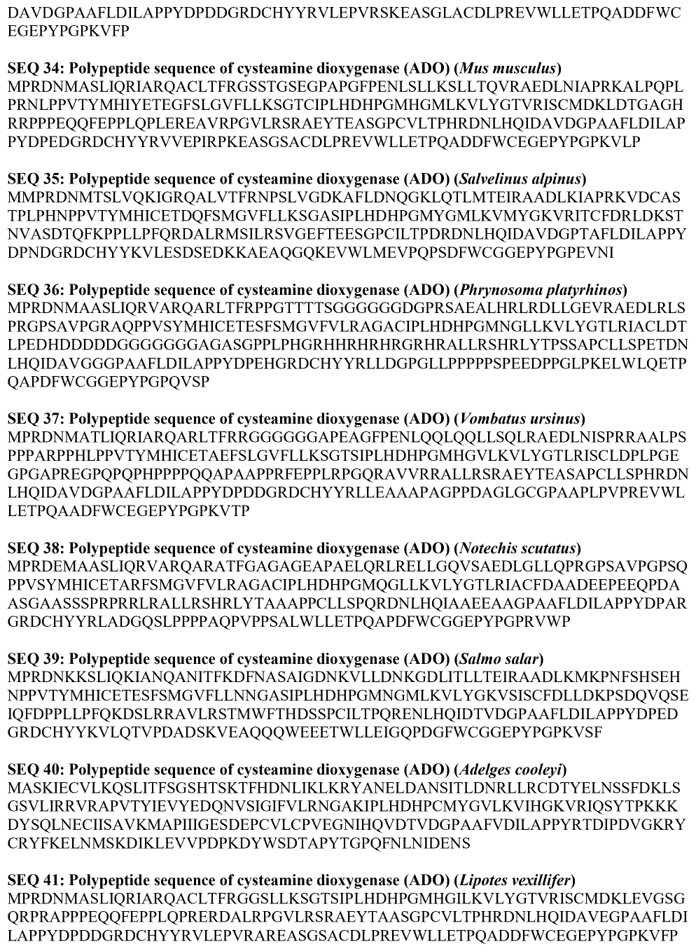

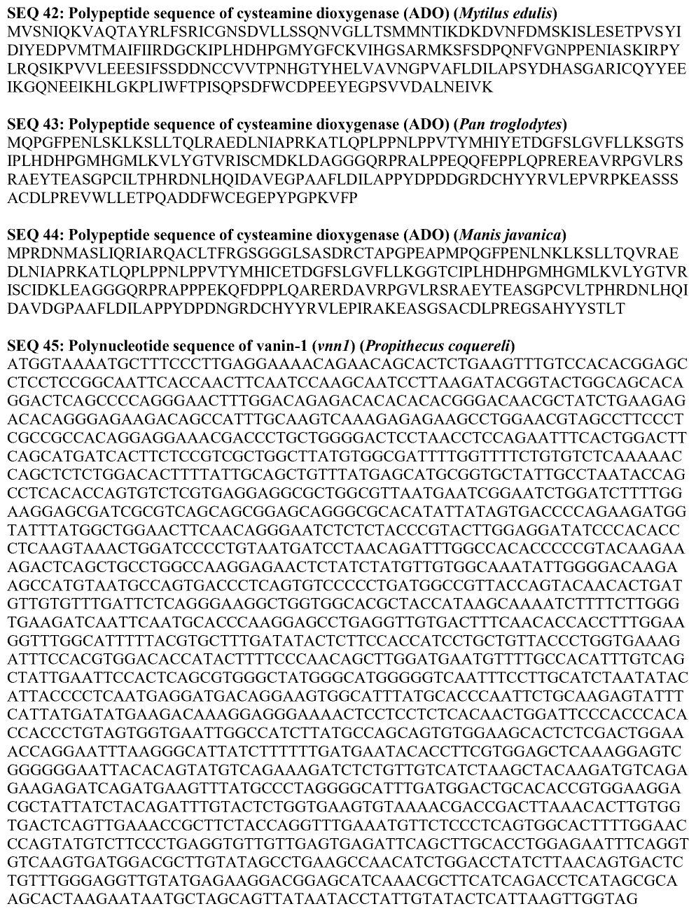

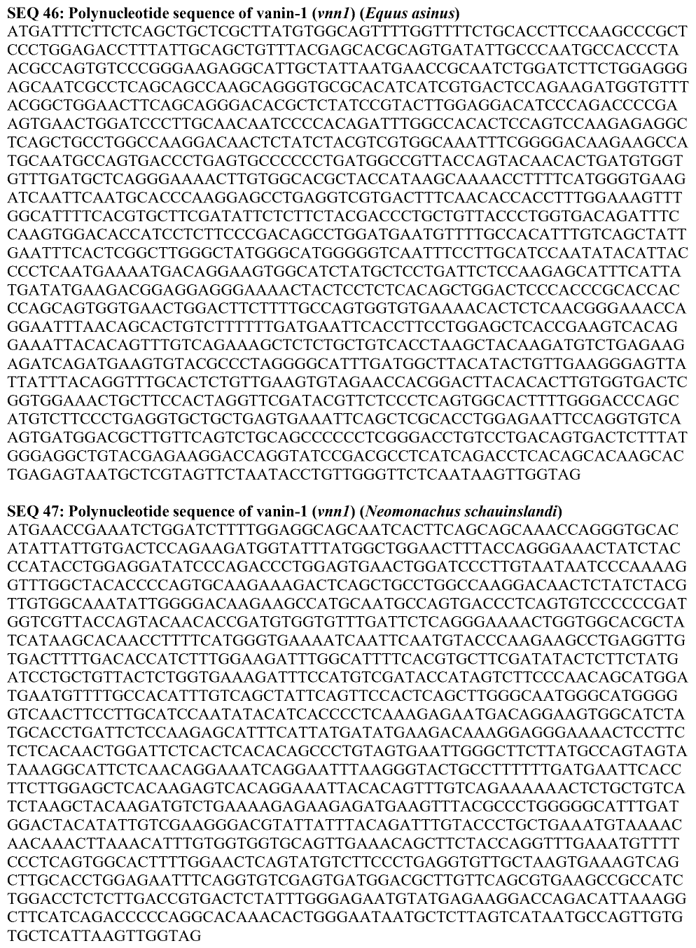

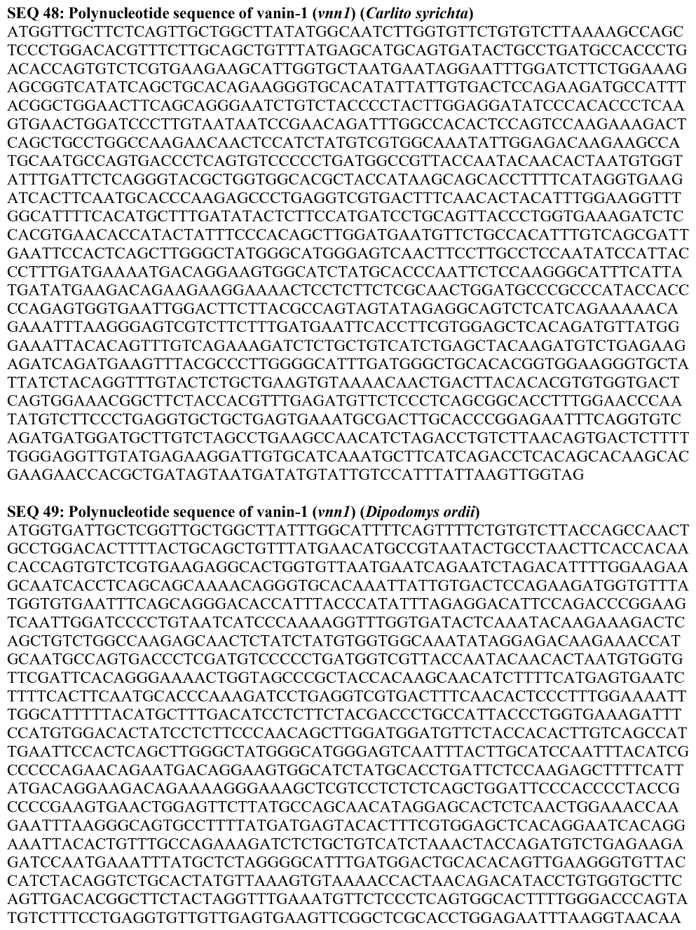

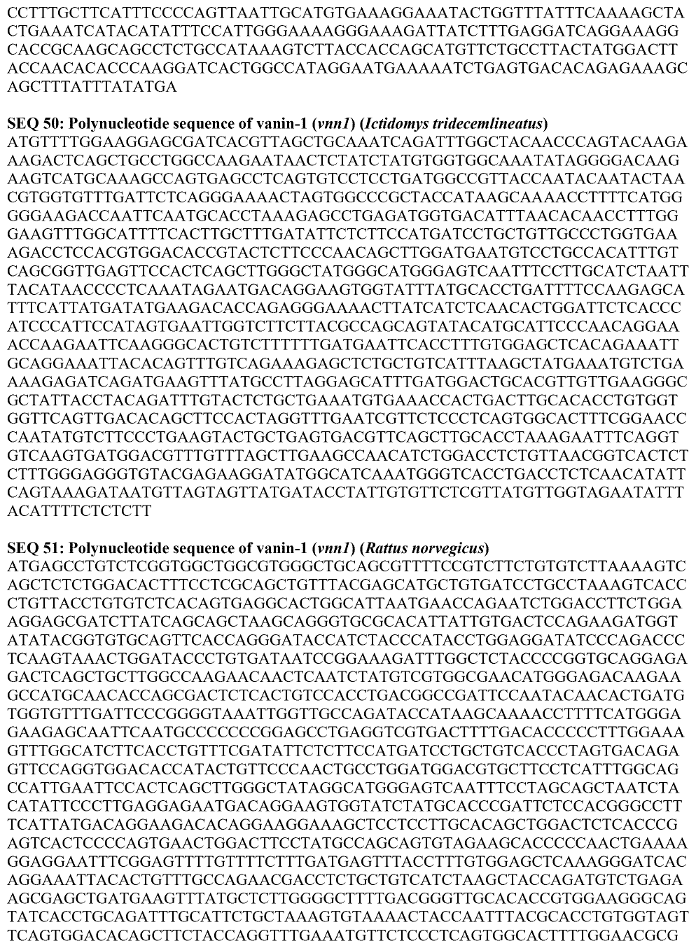

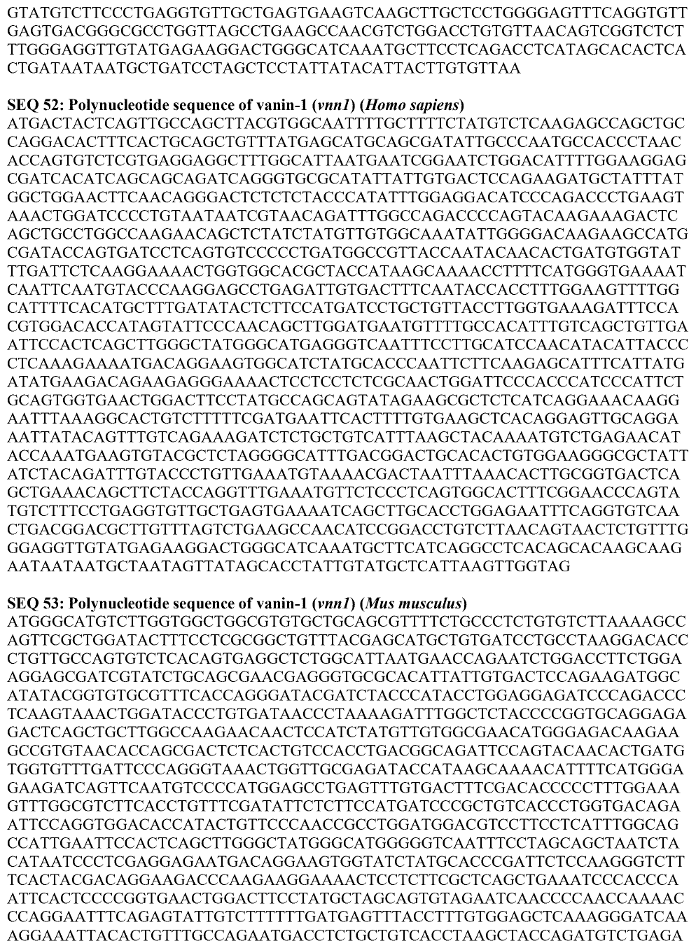

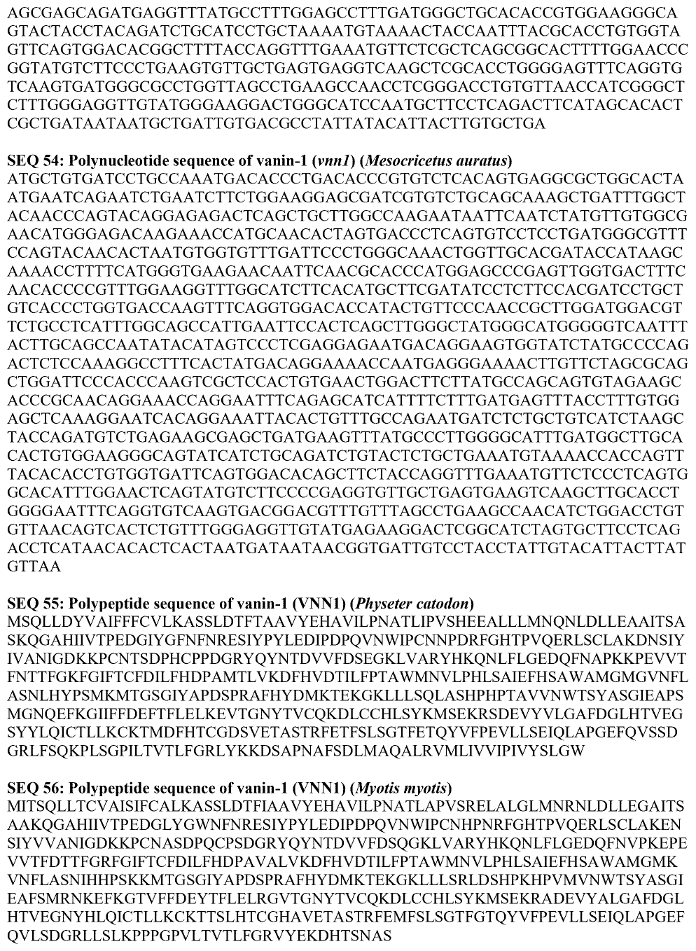

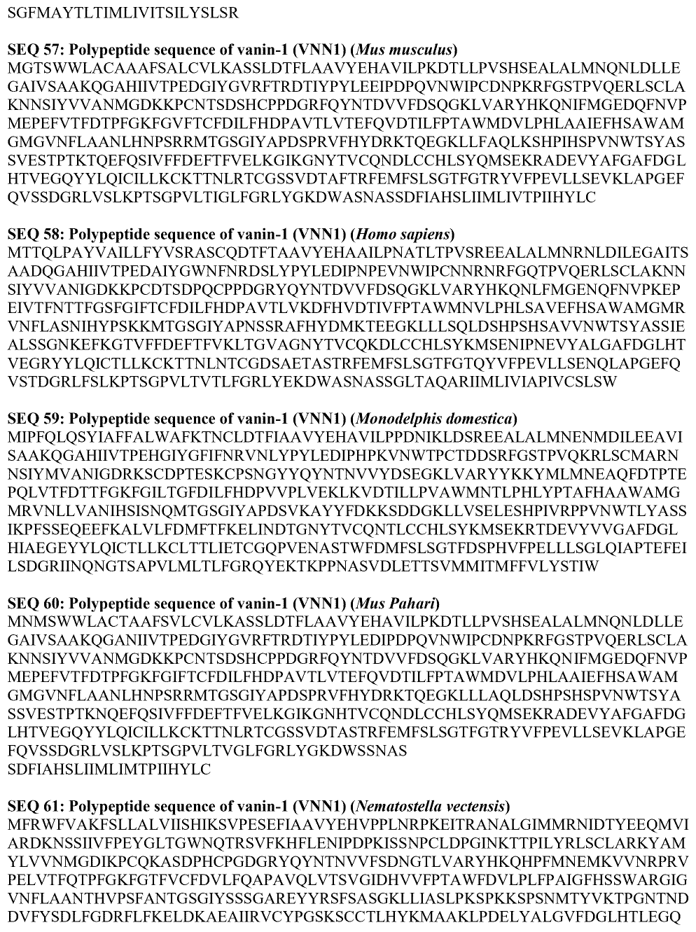

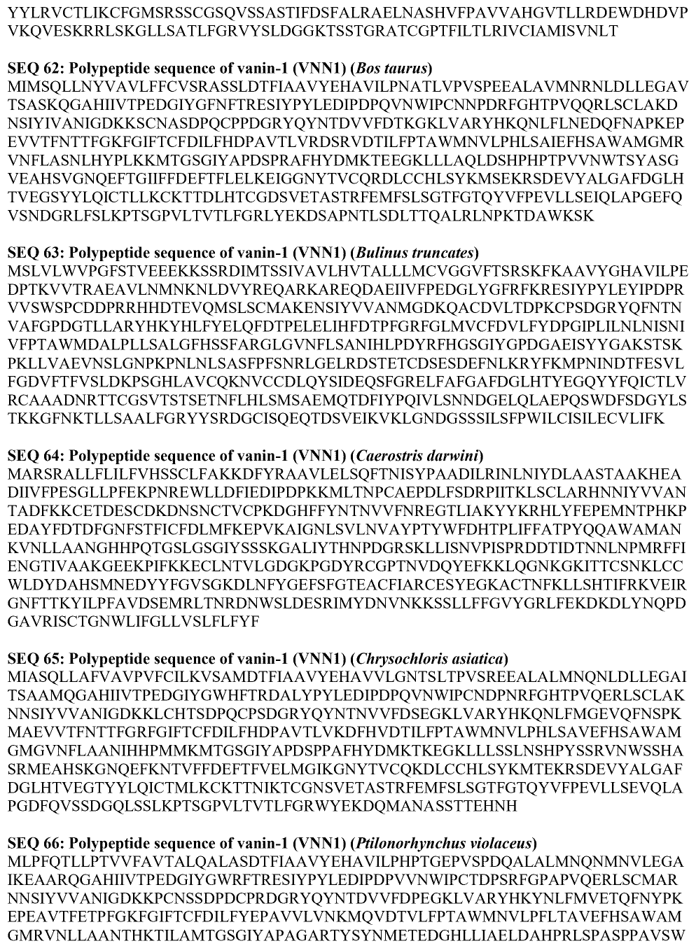

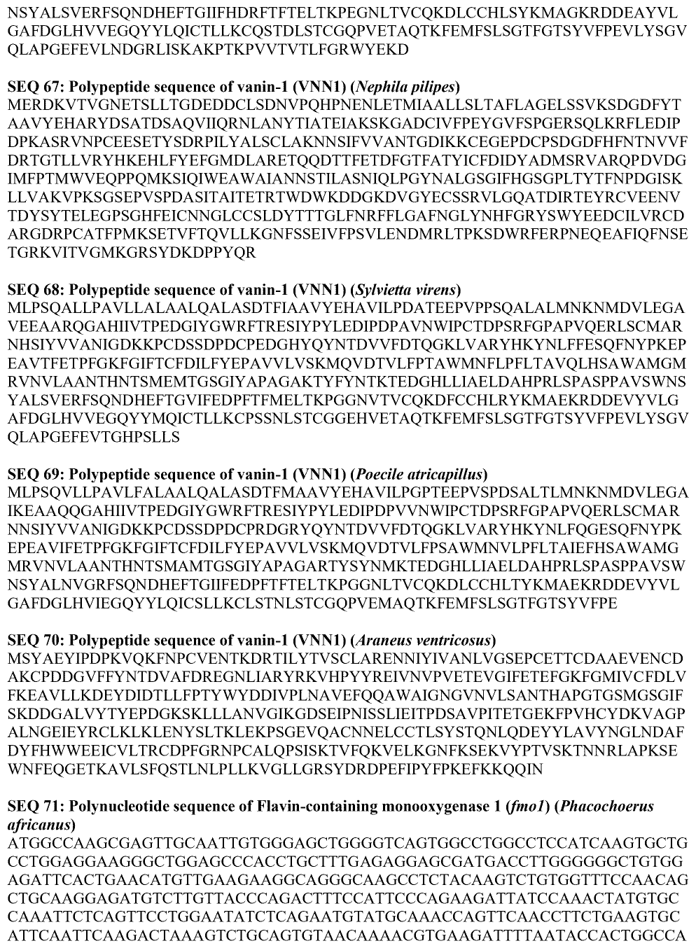

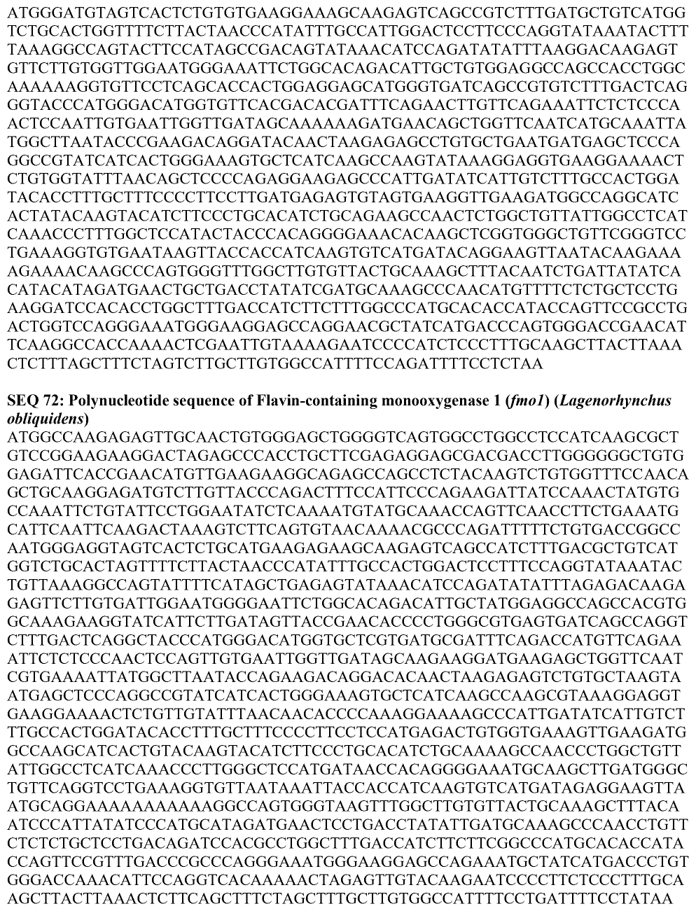

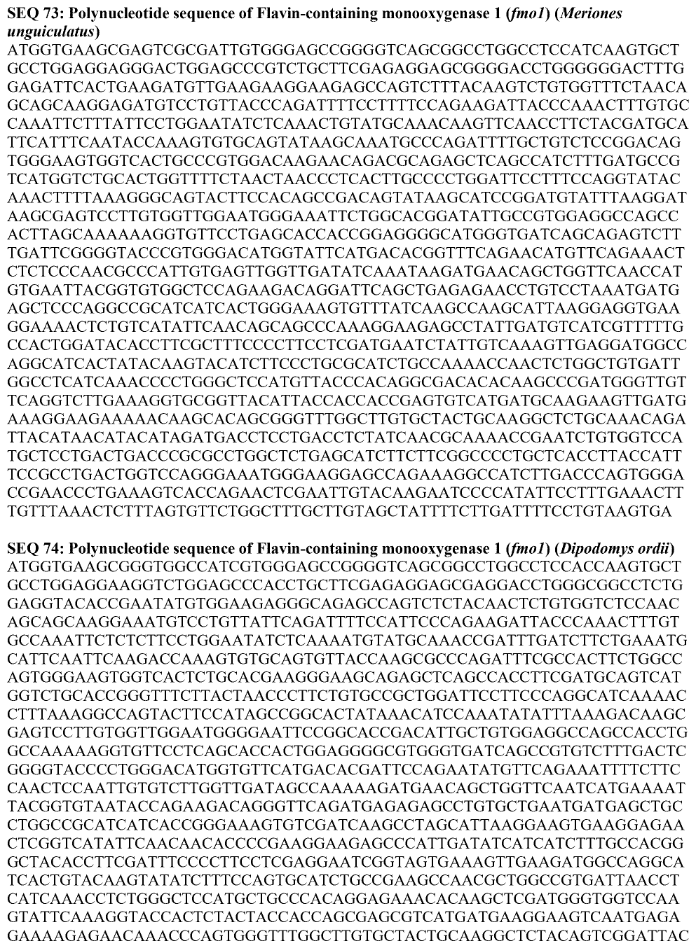

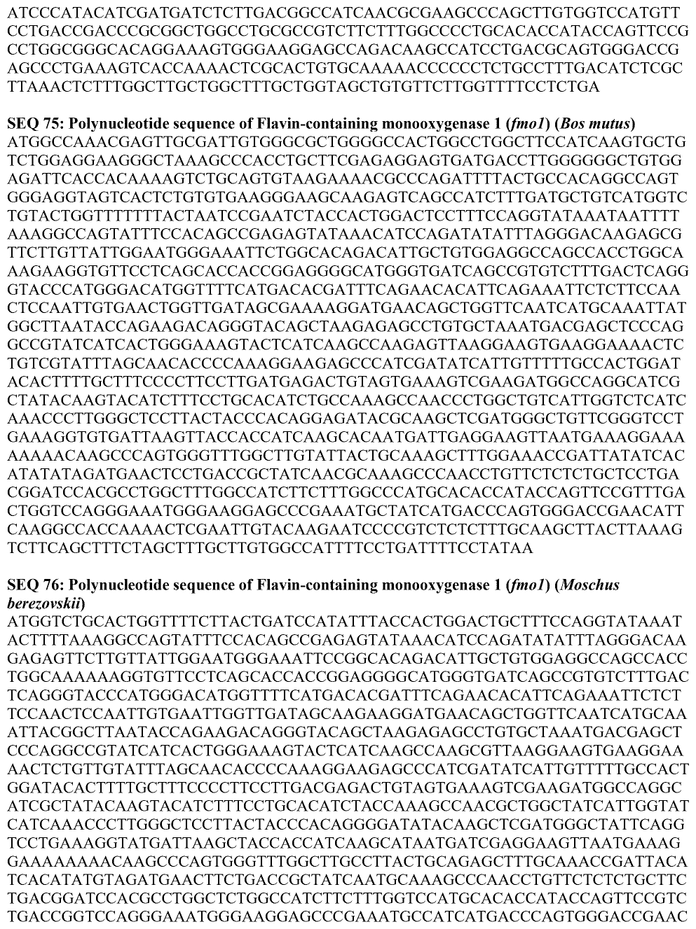

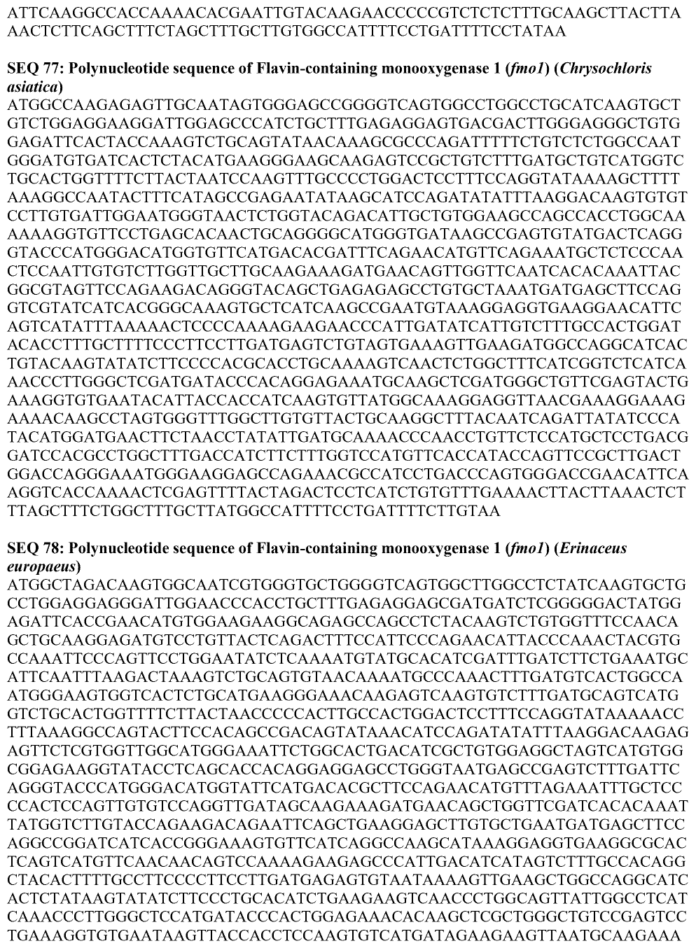

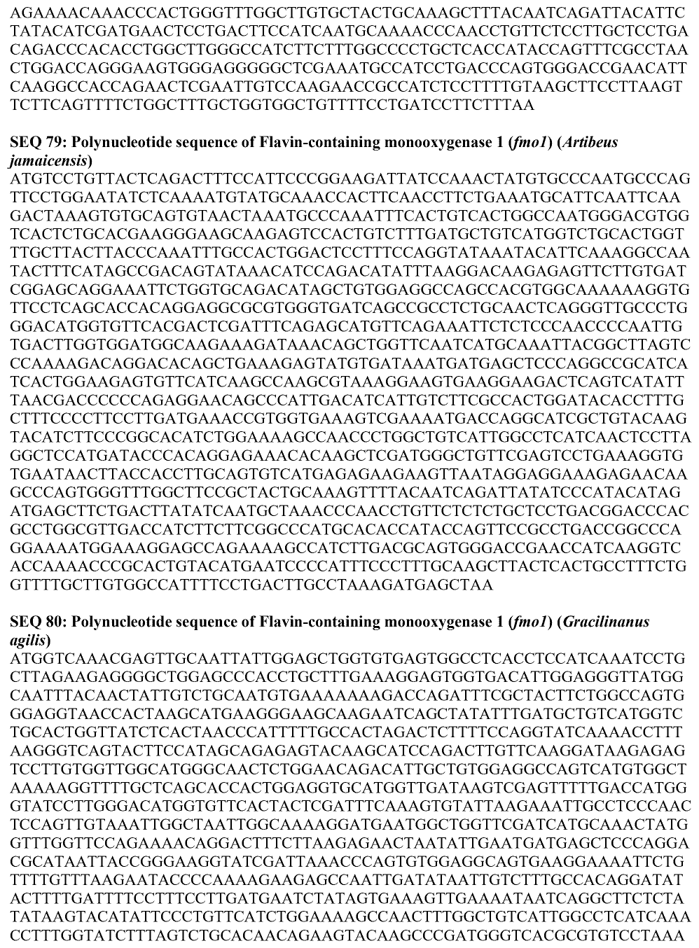

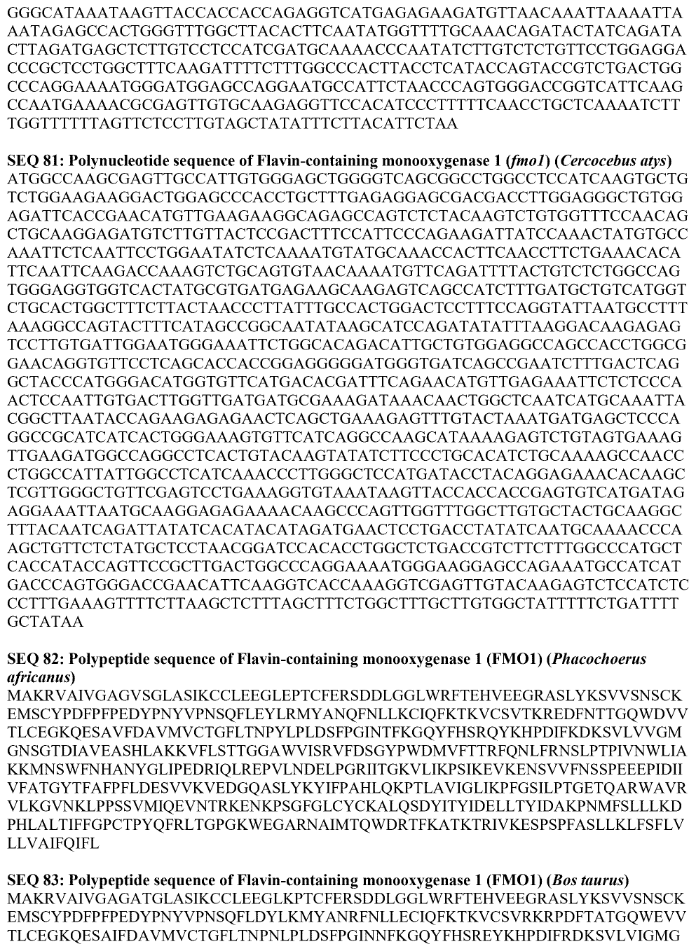

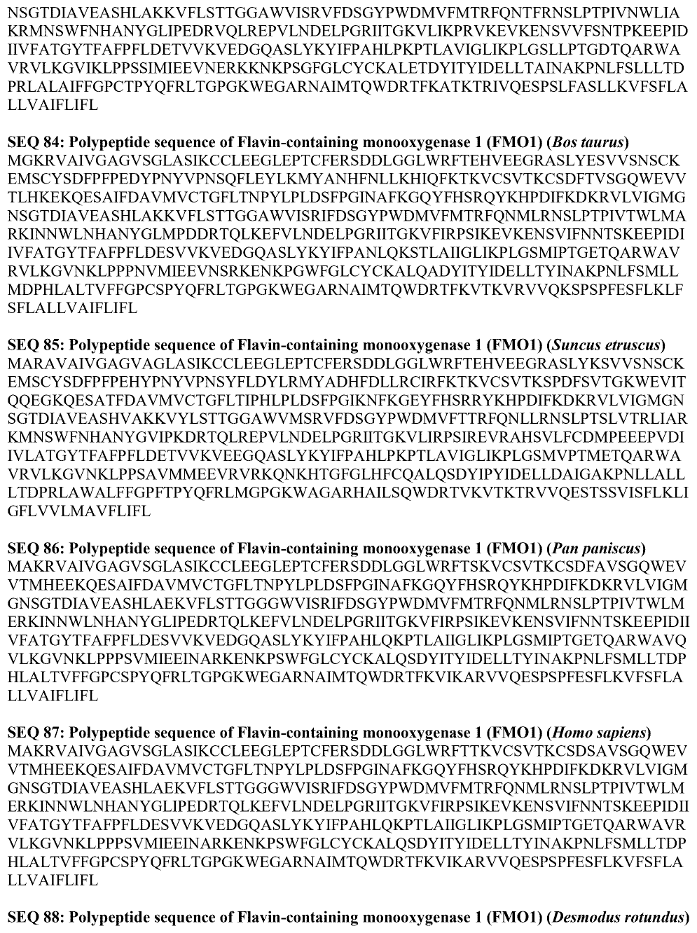

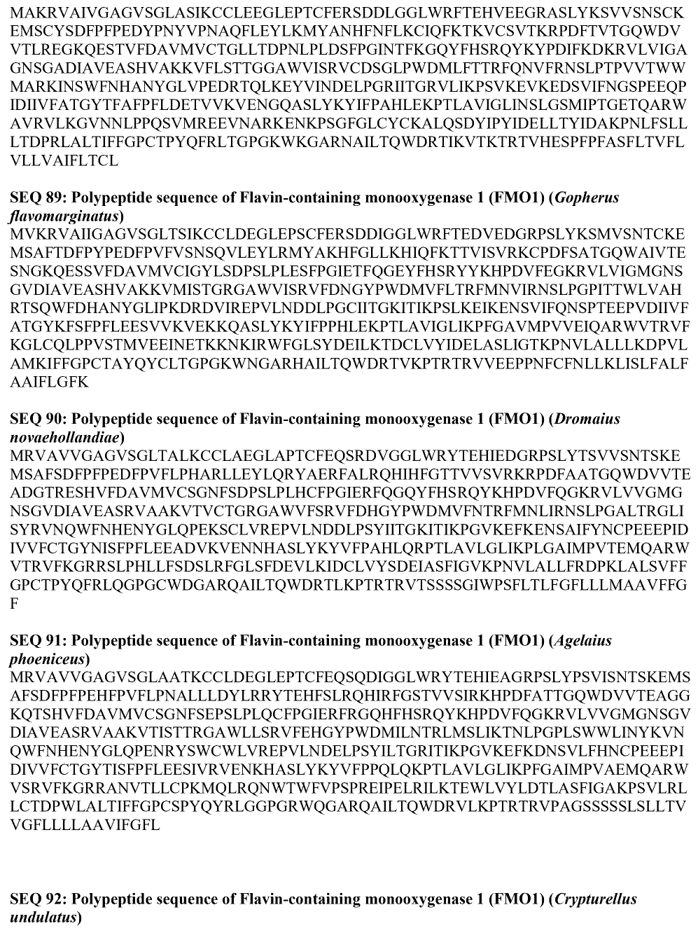

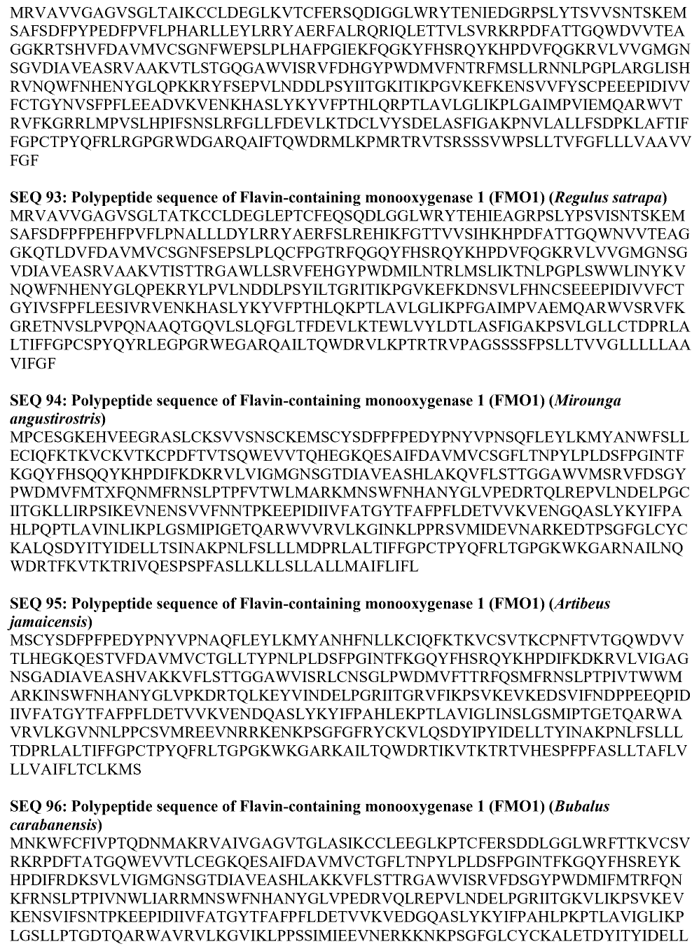

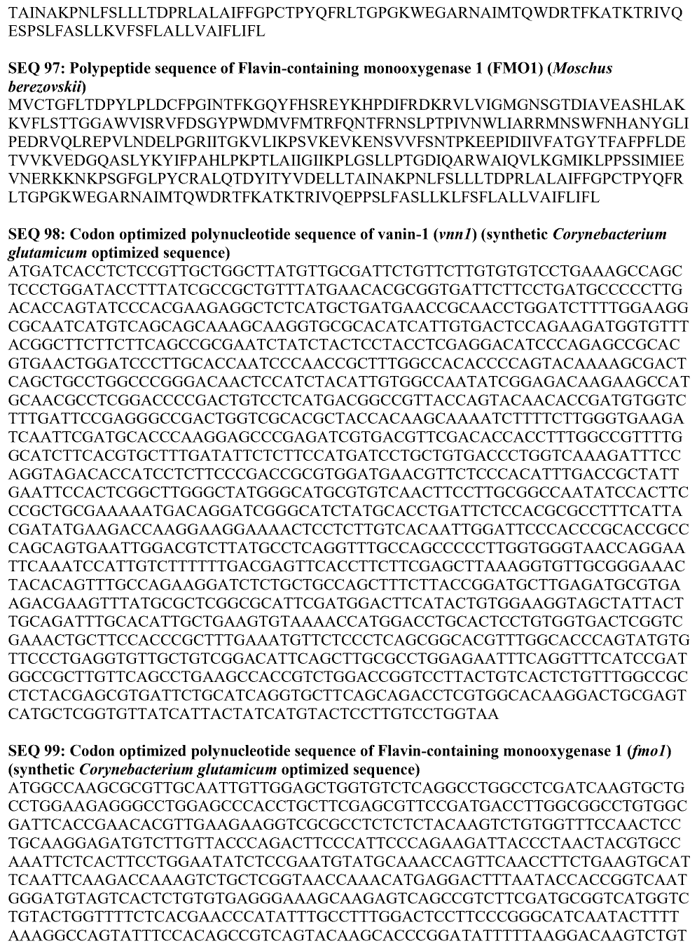

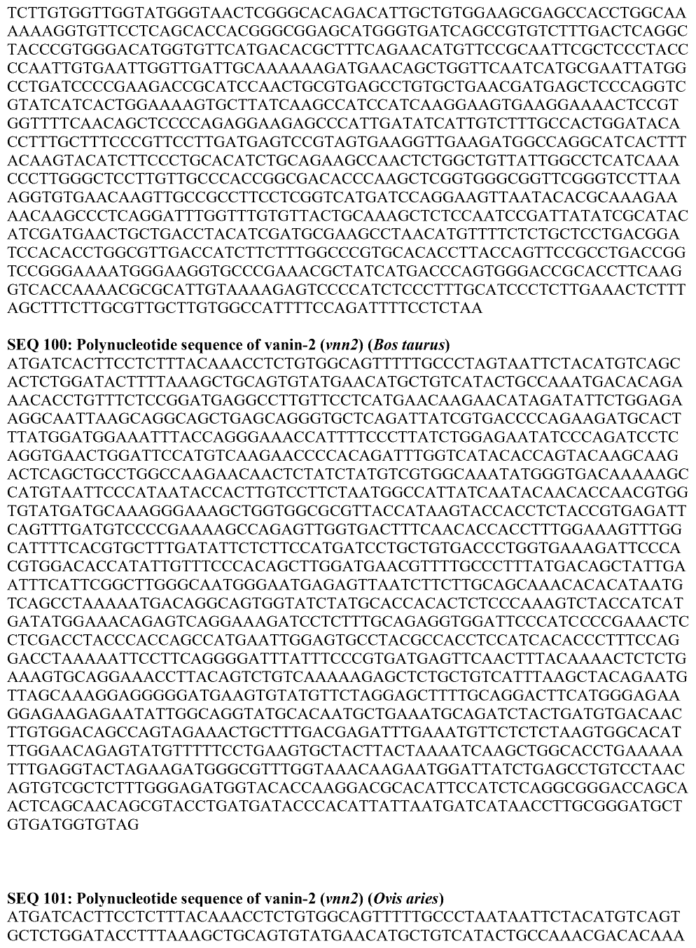

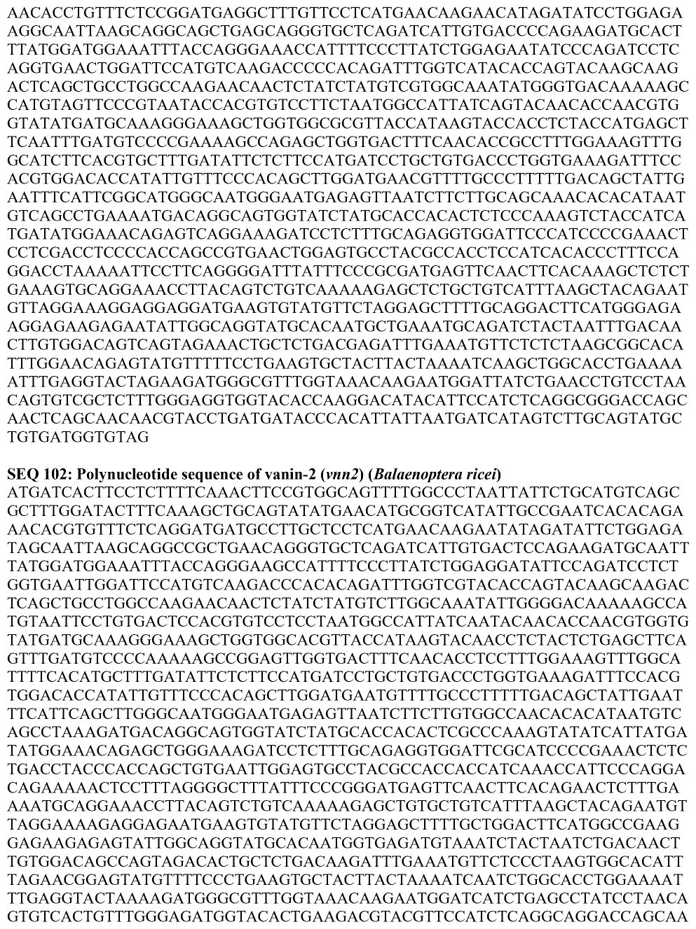

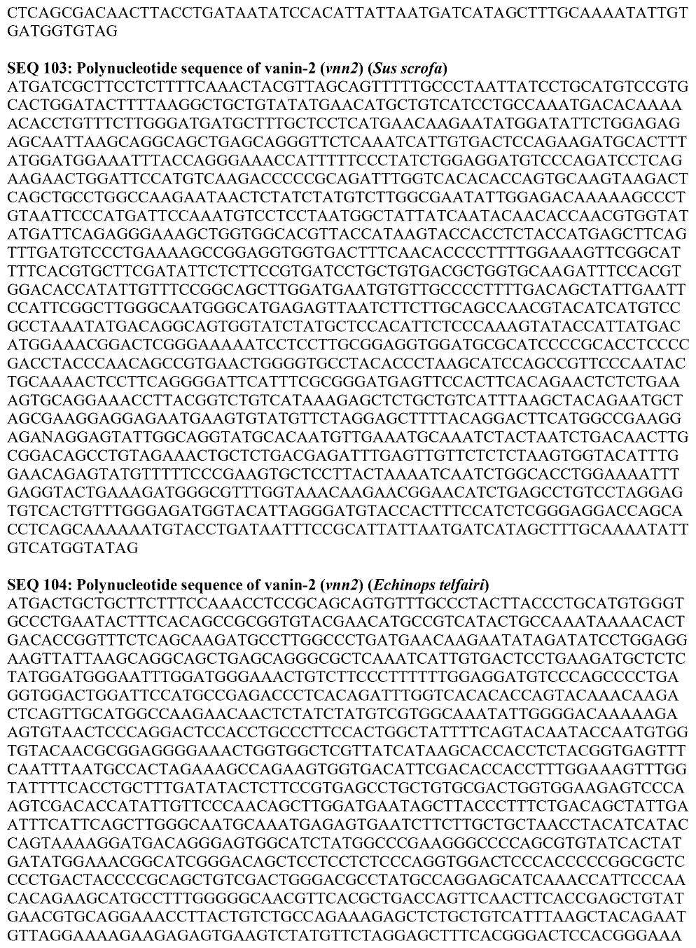

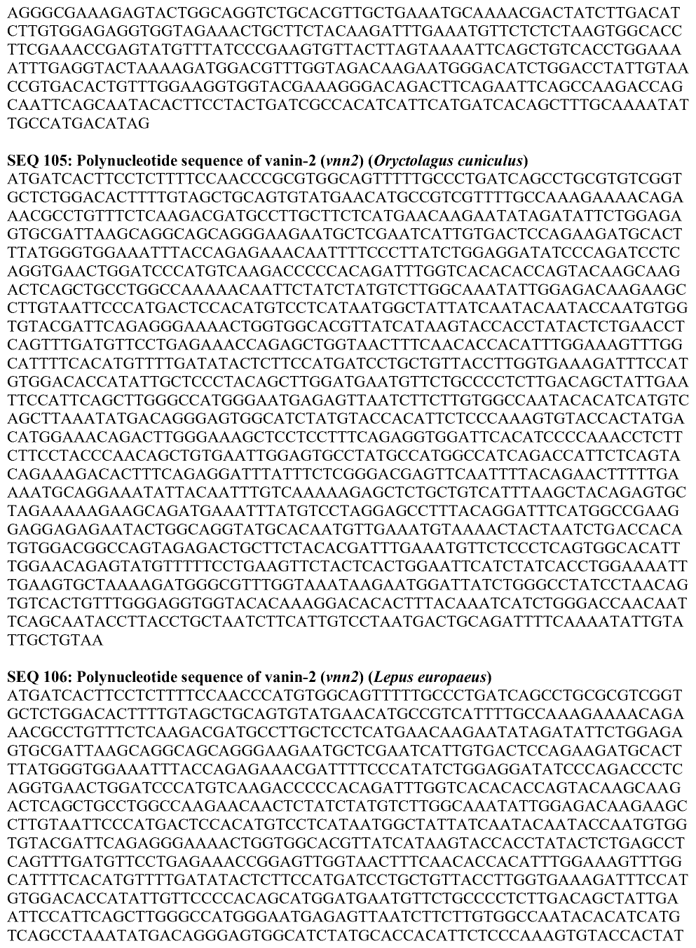

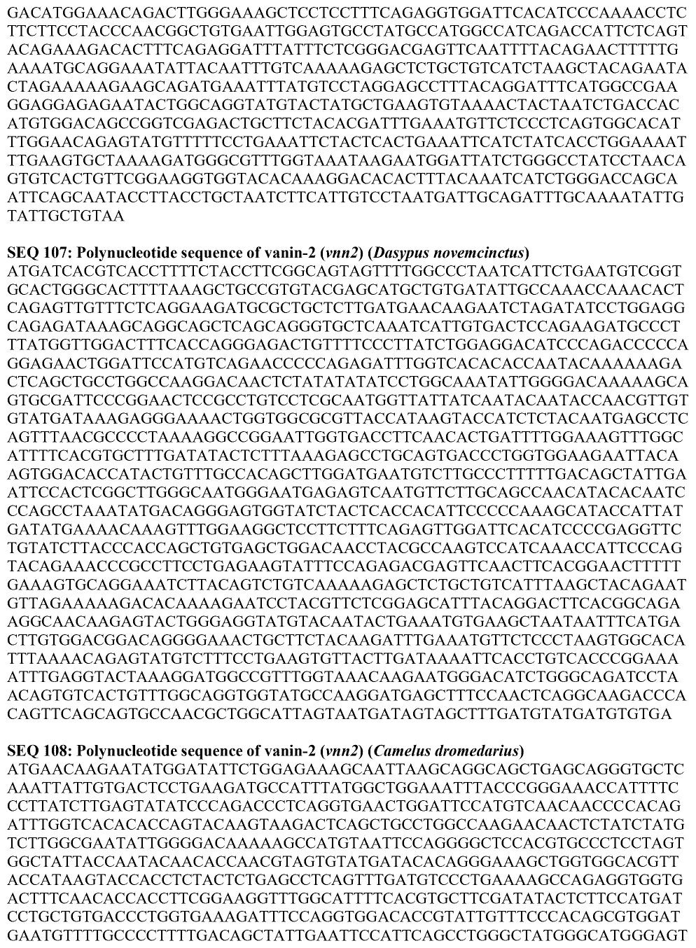

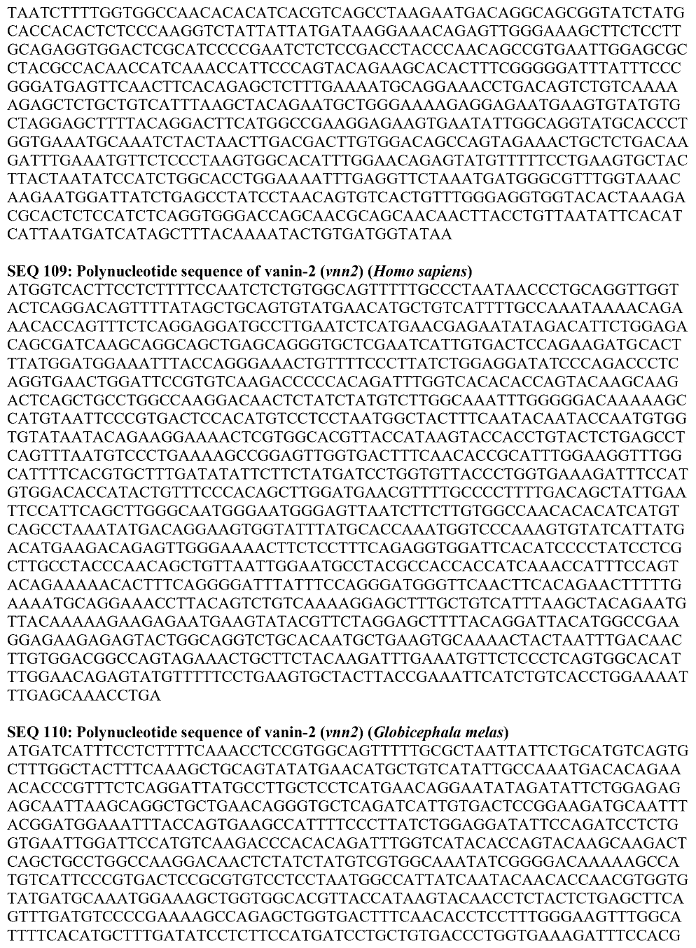

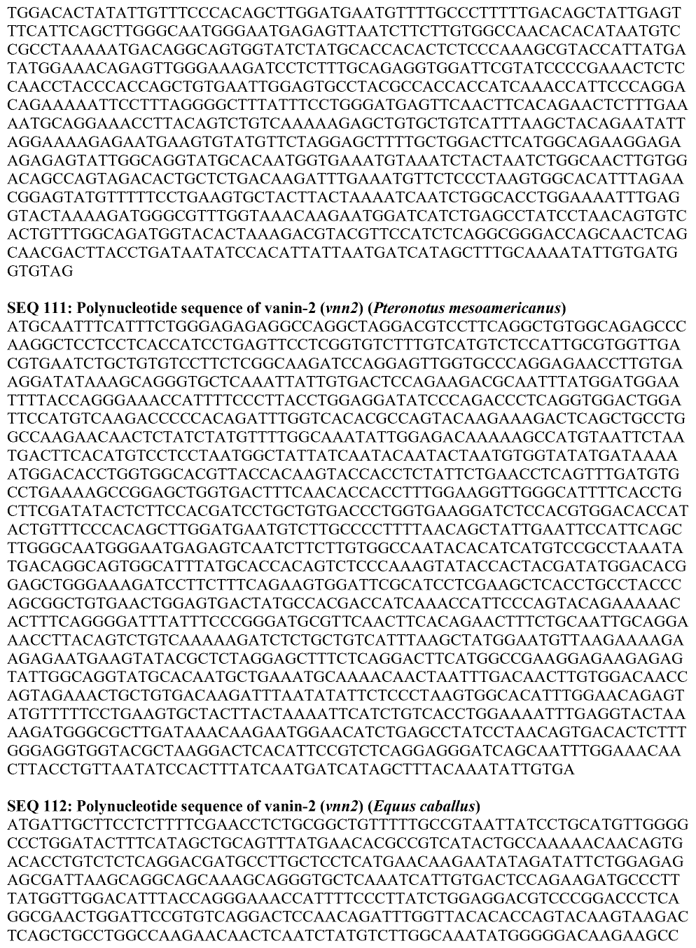

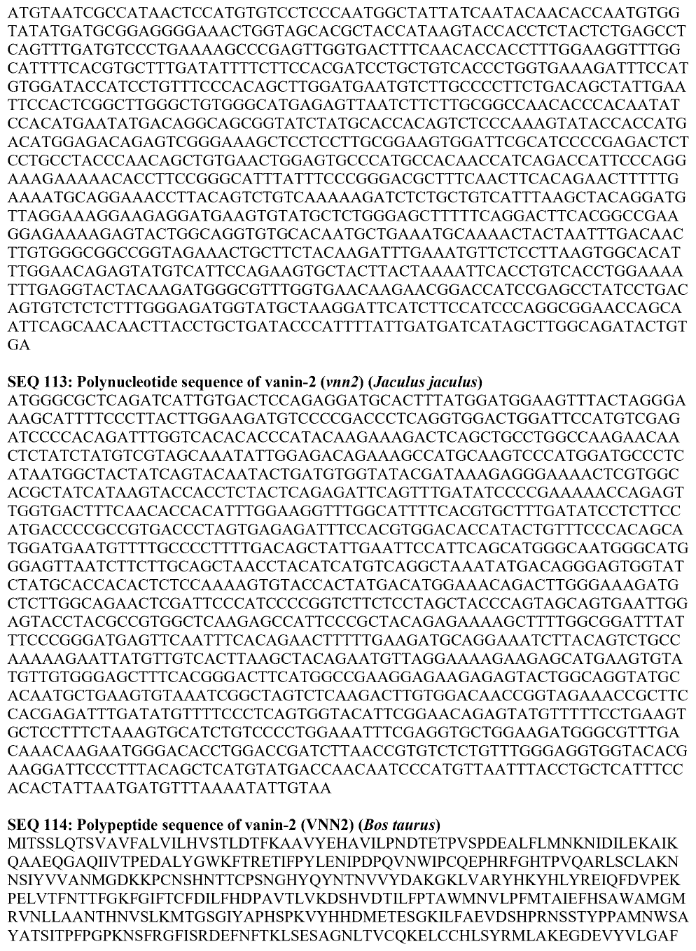

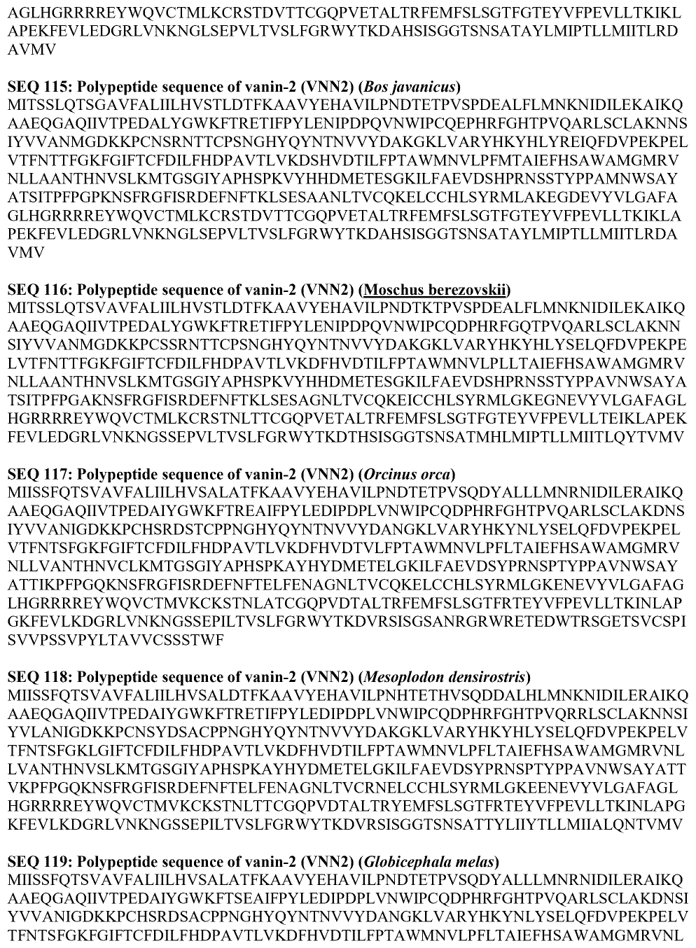

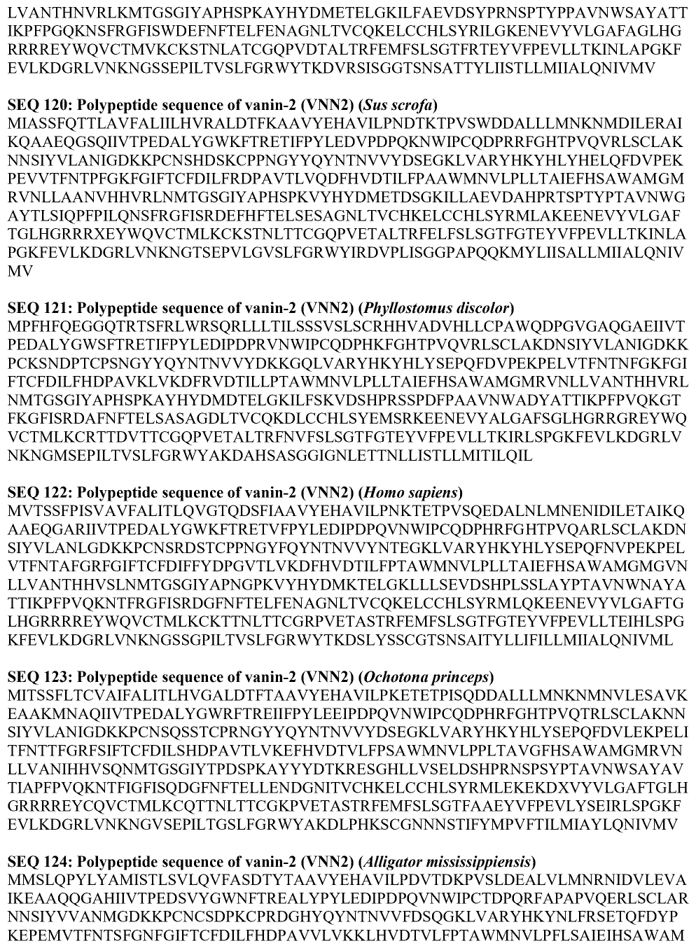

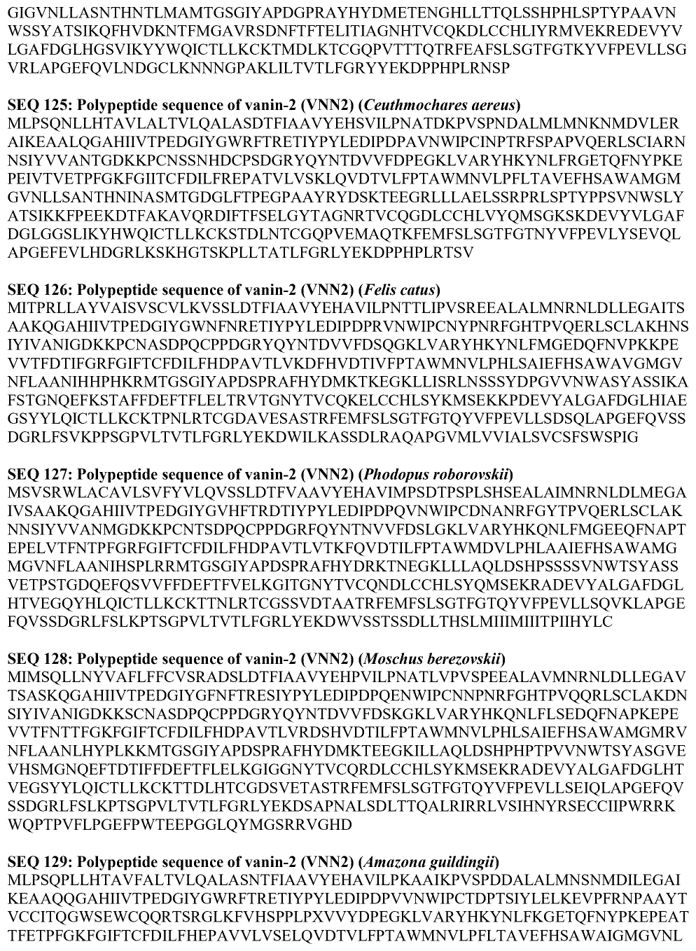

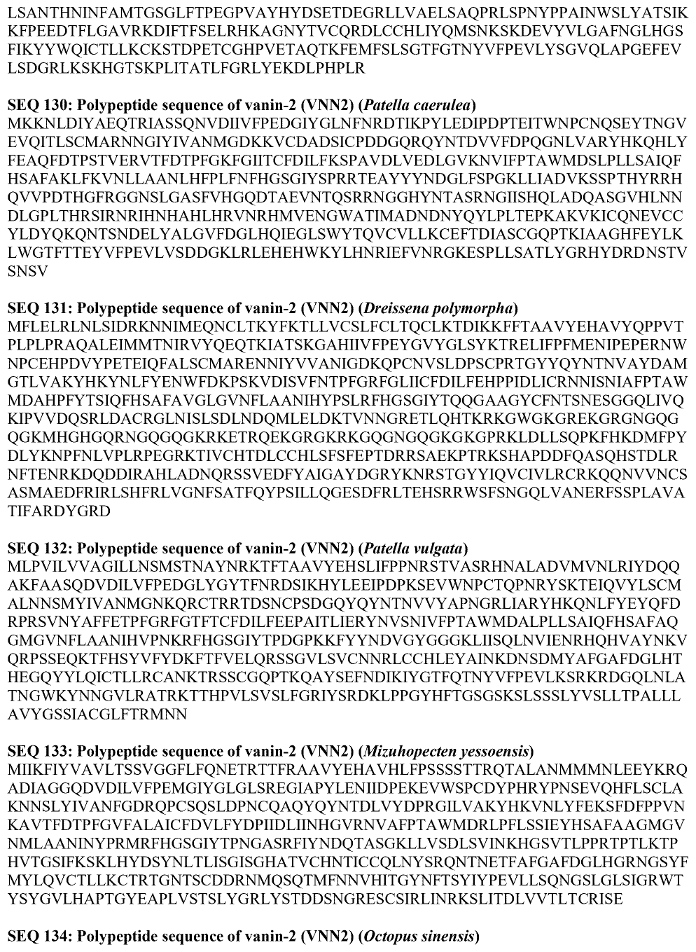

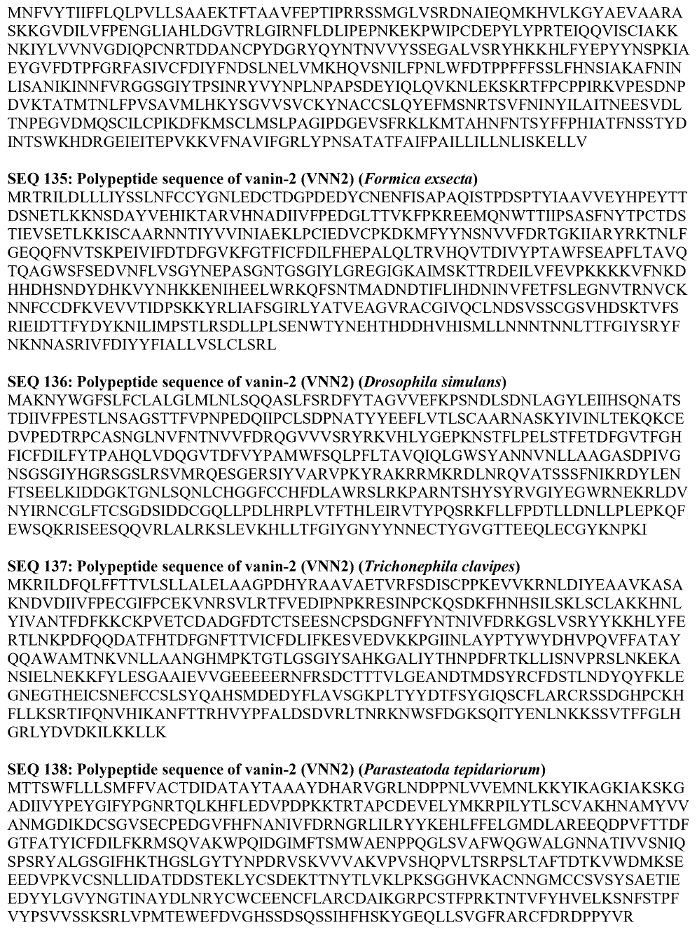

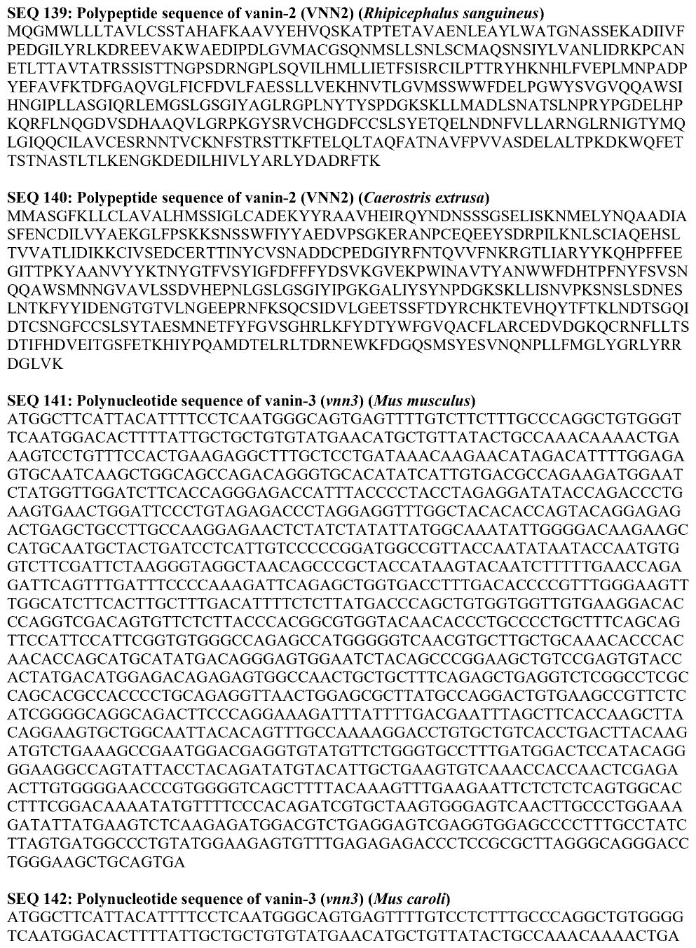

### DETAILED DESCRIPTION OF THE INVENTION

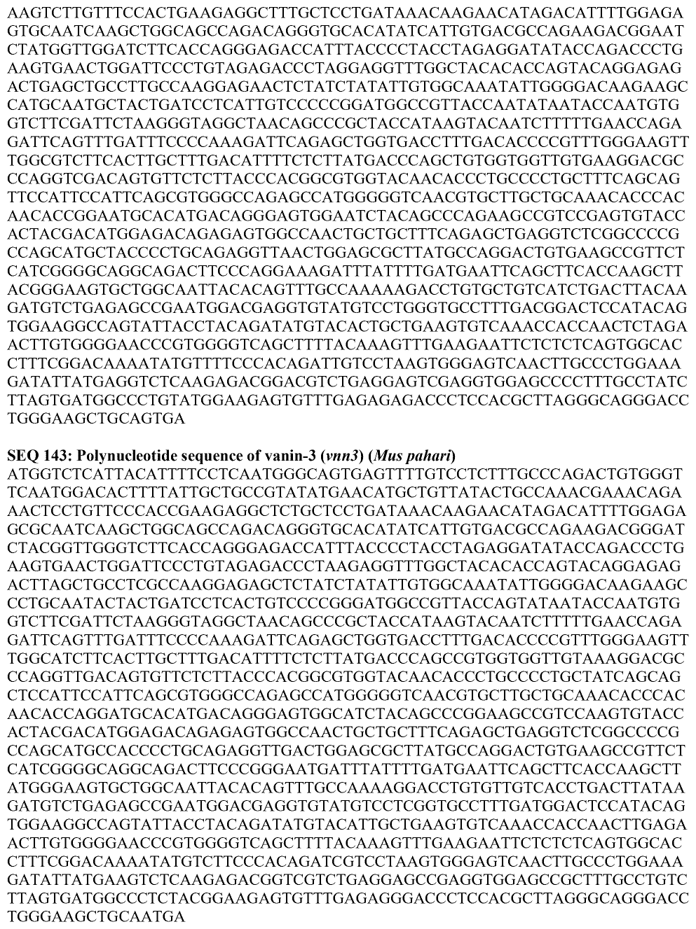

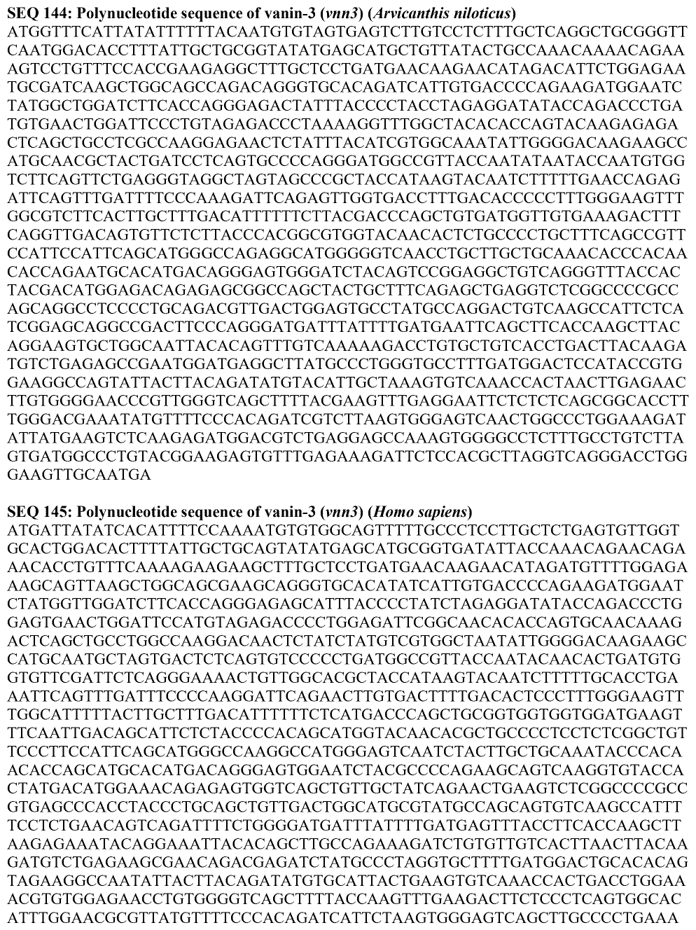

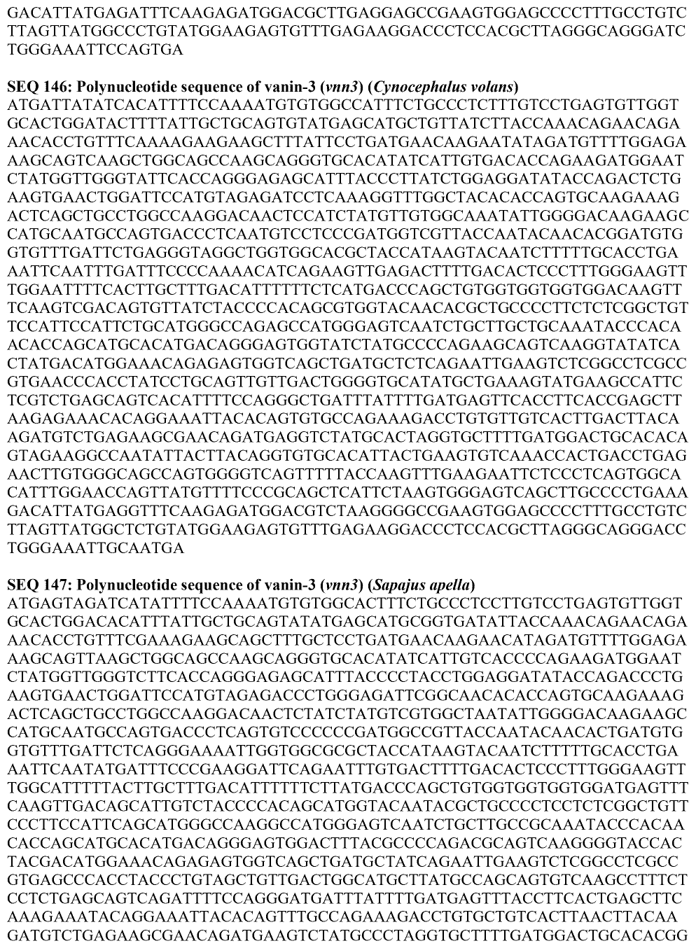

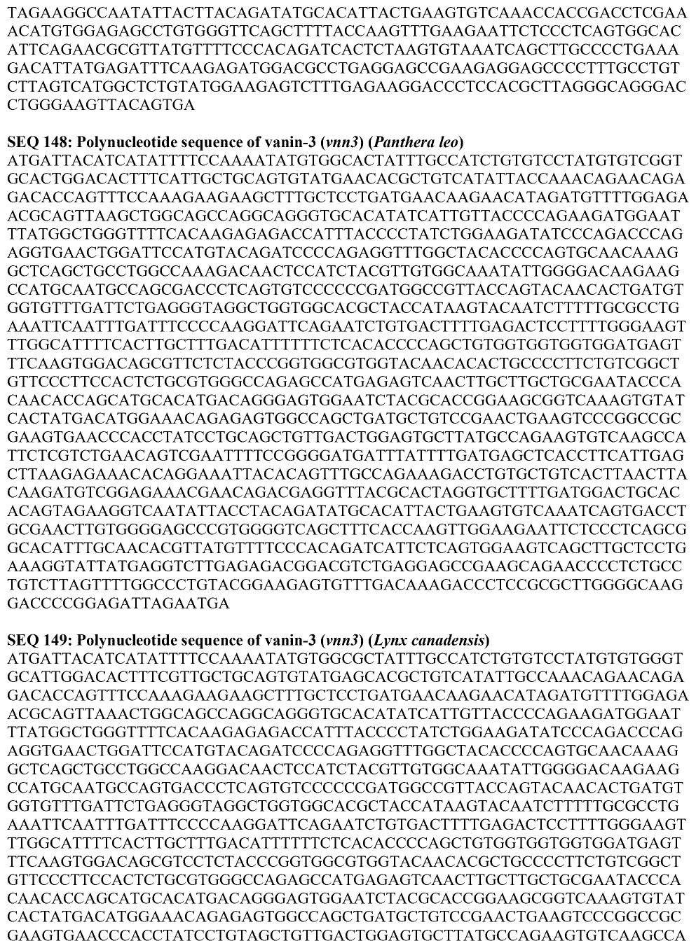

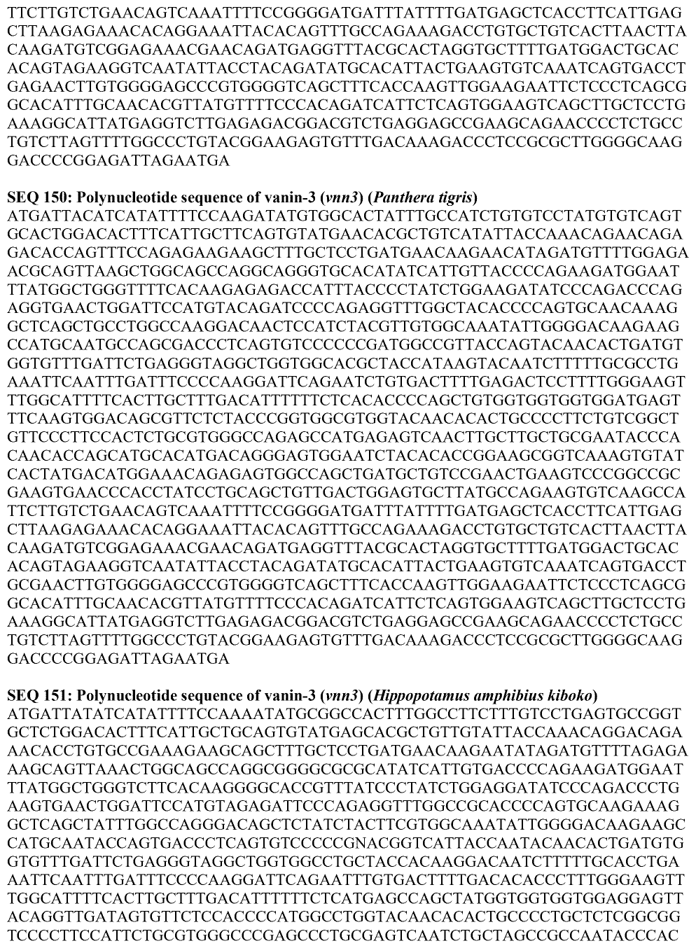

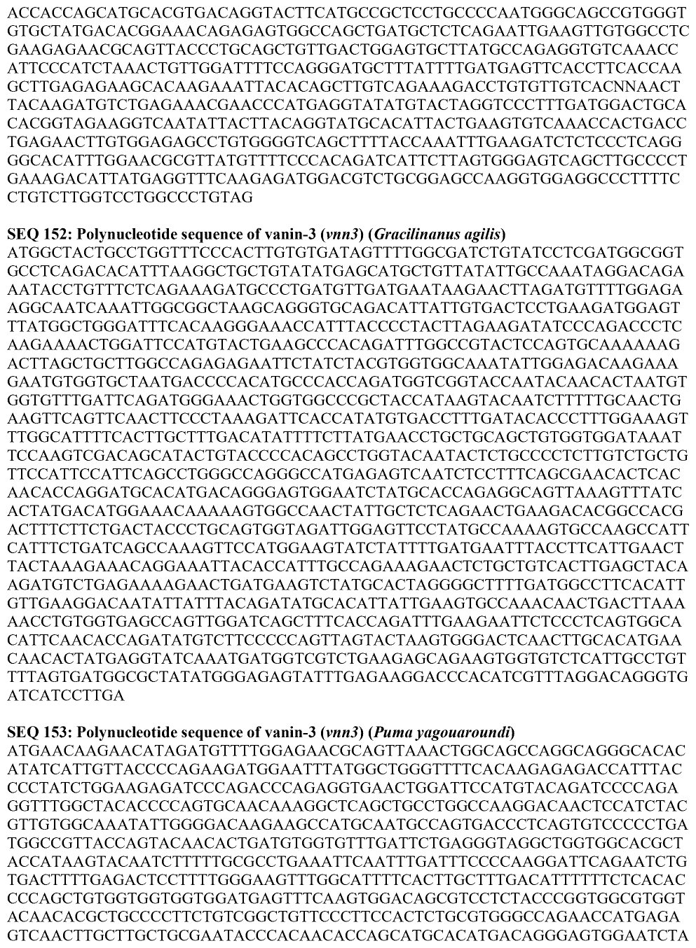

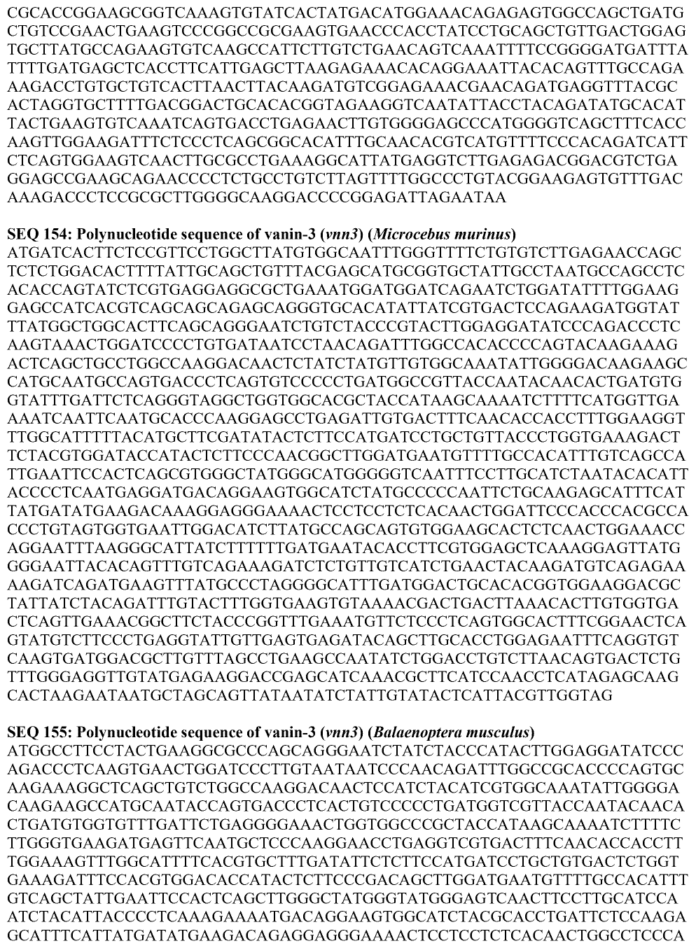

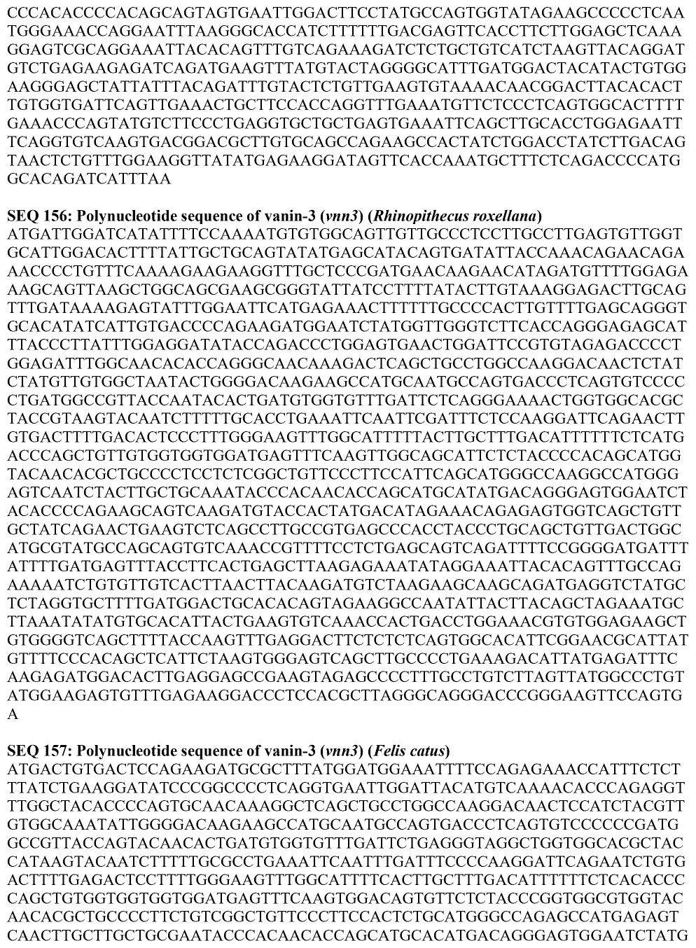

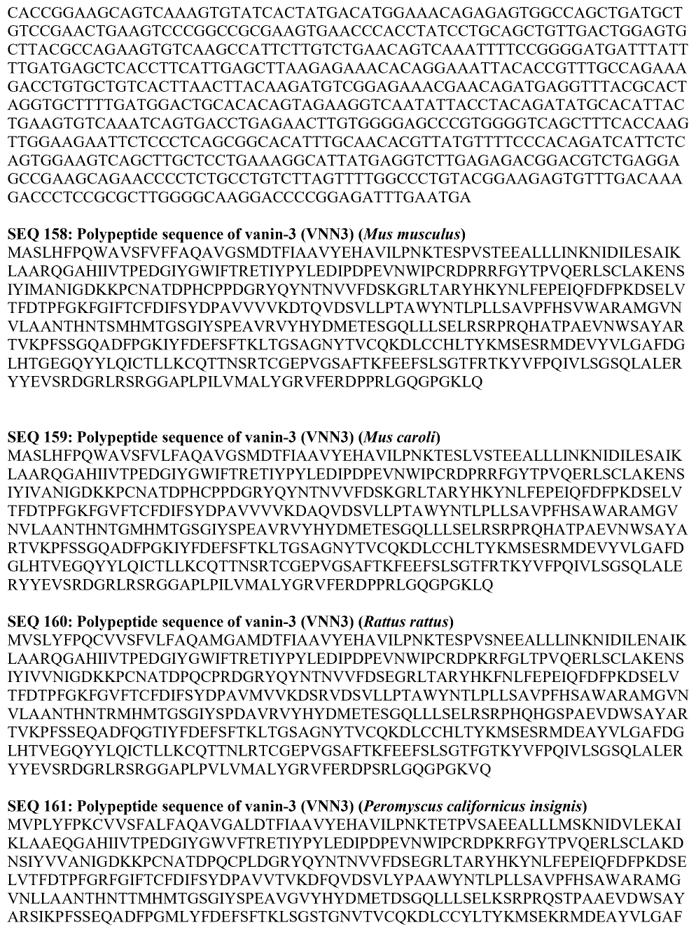

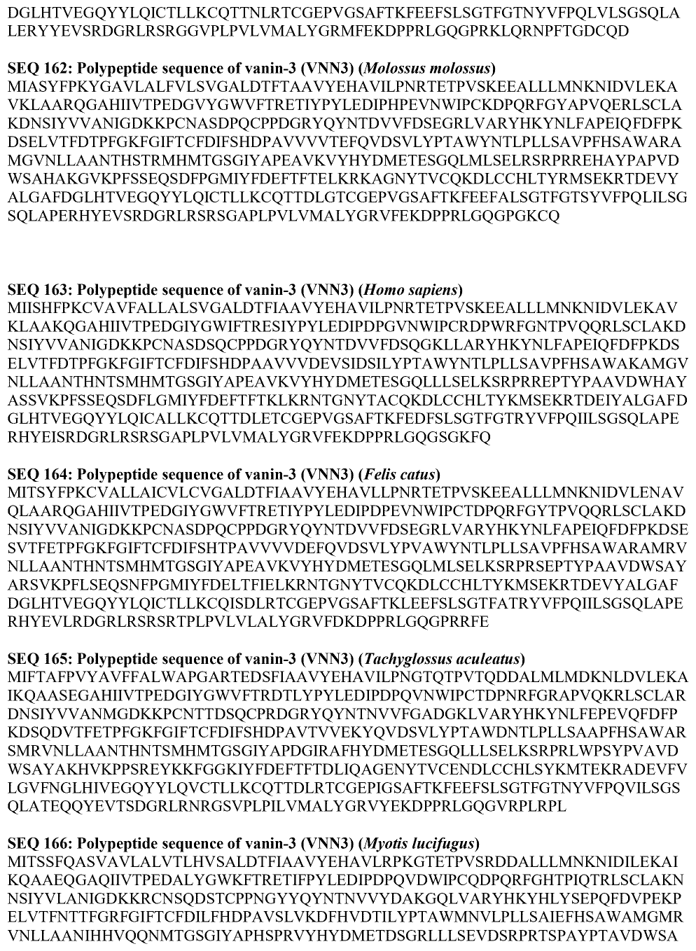

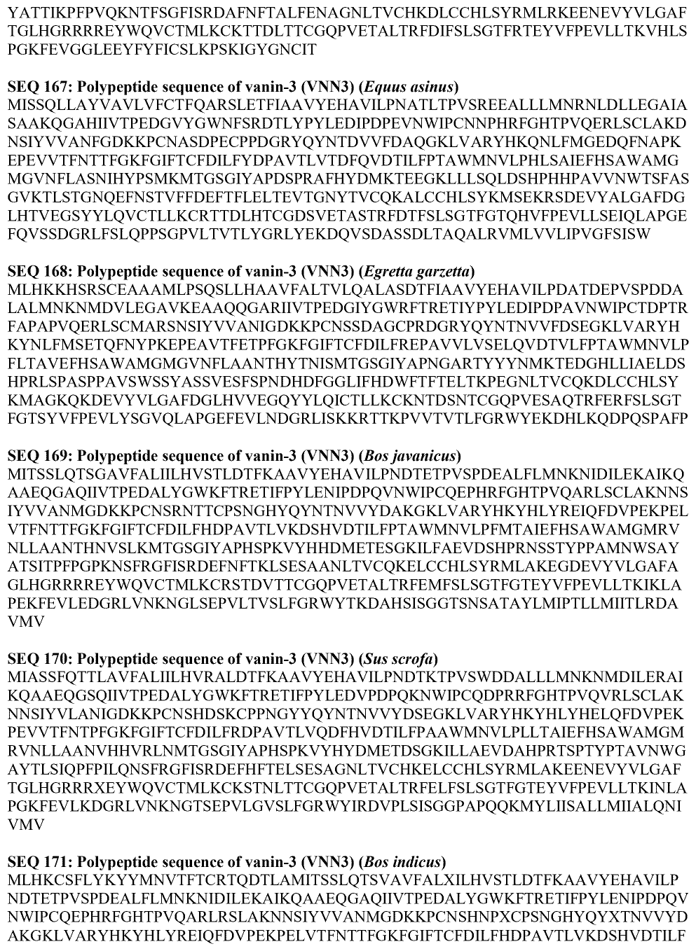

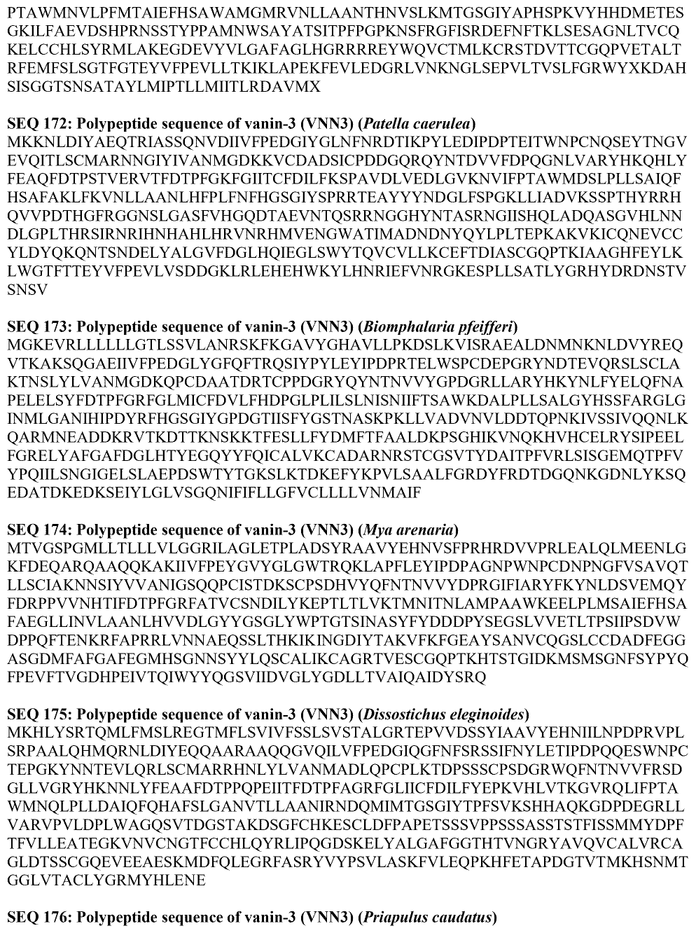

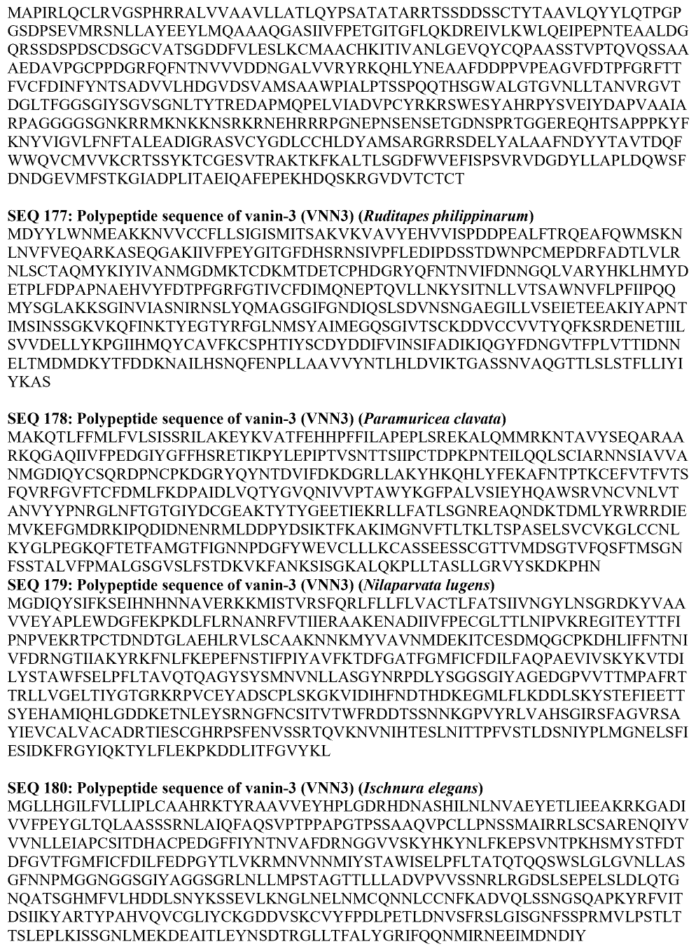

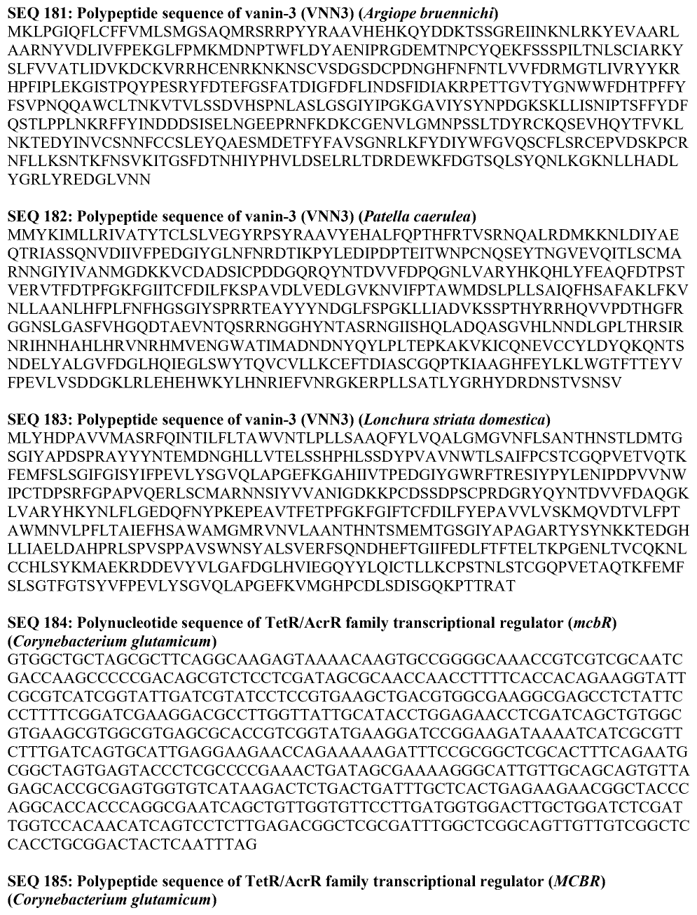

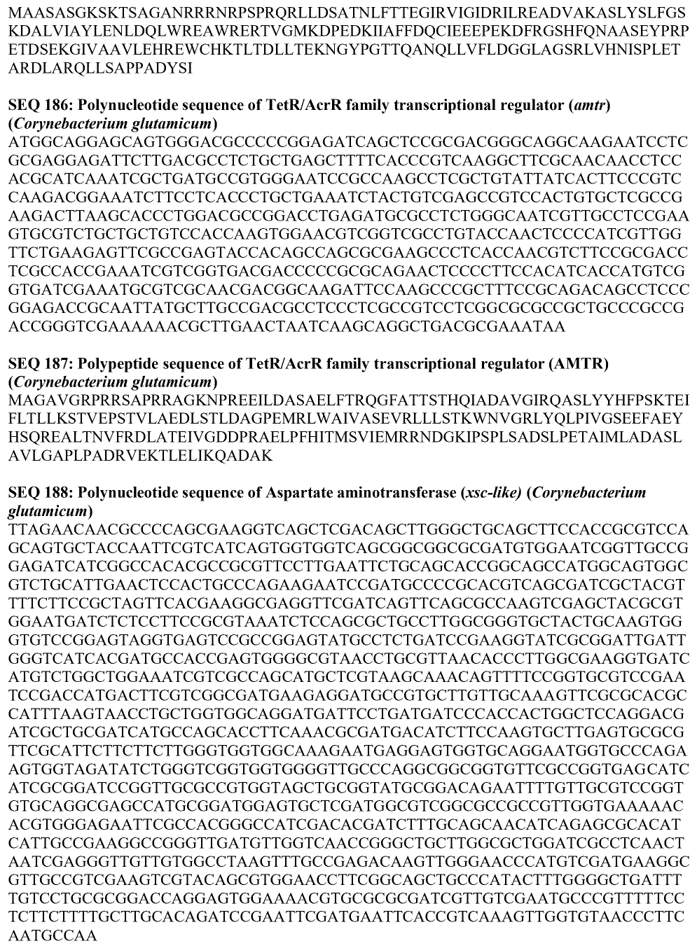

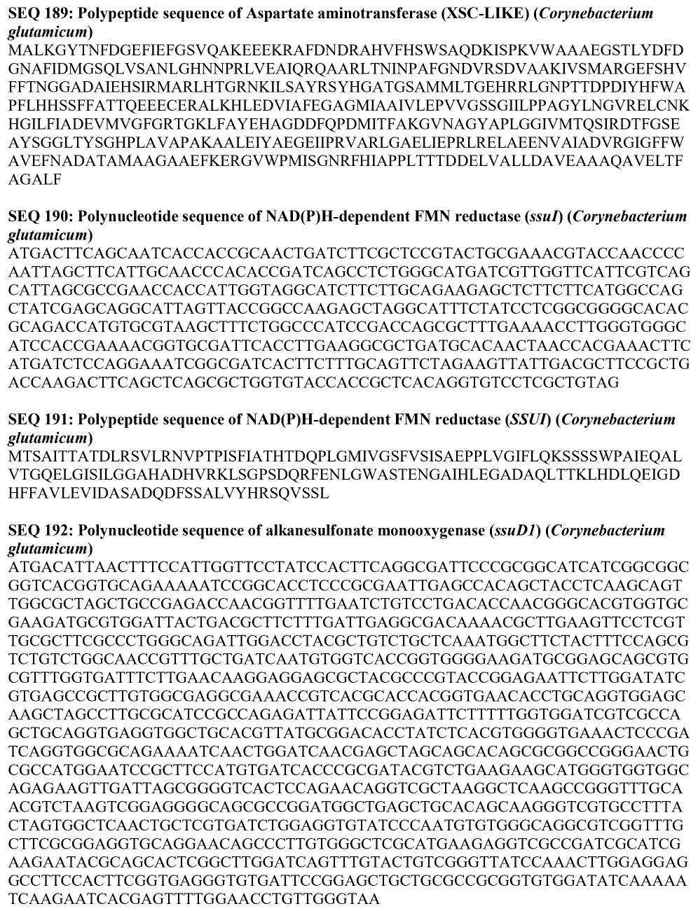

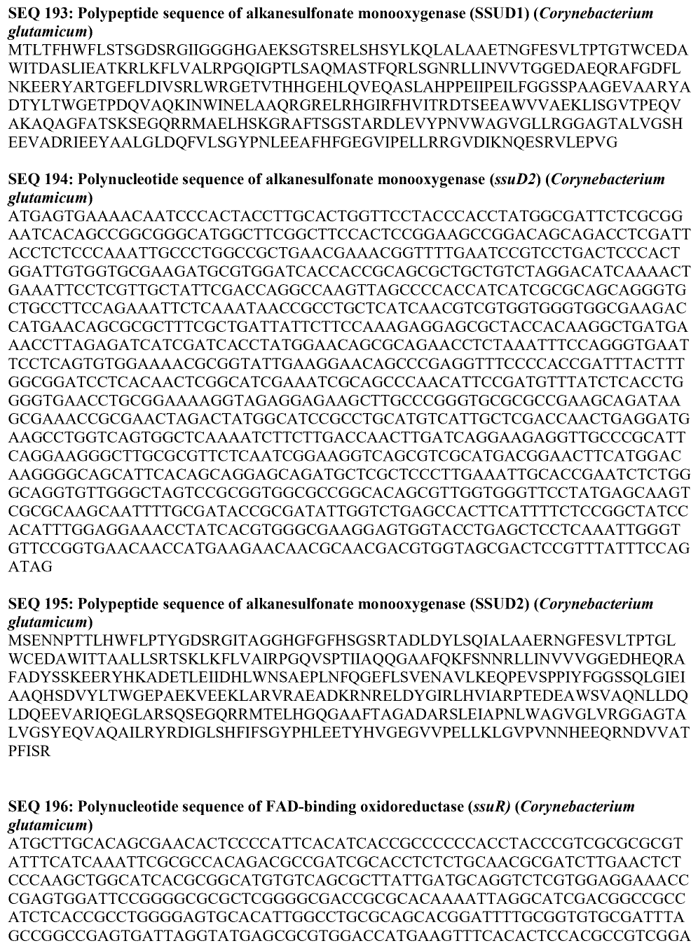

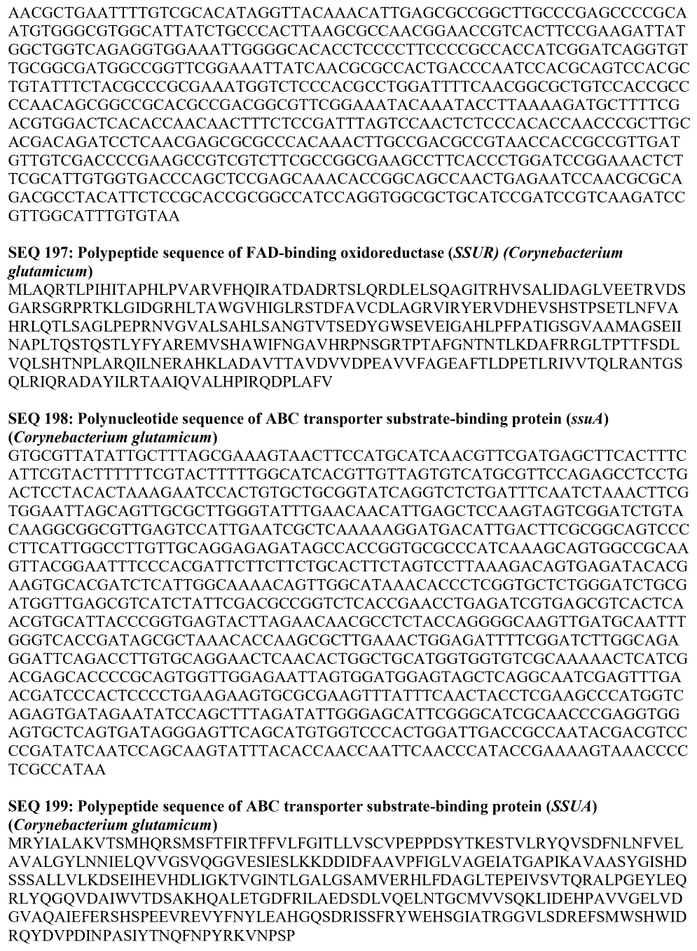

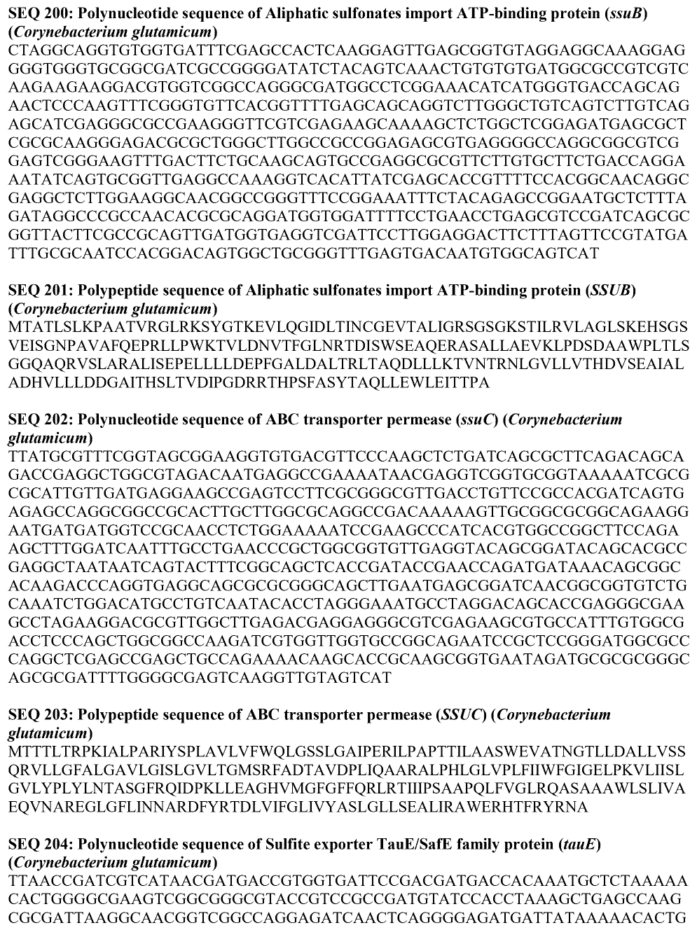

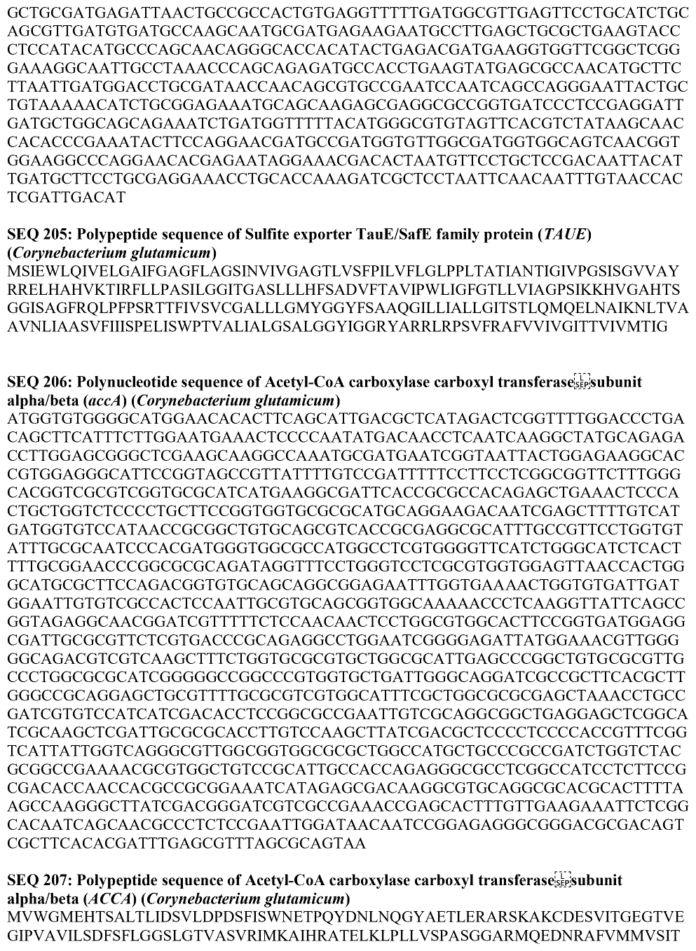

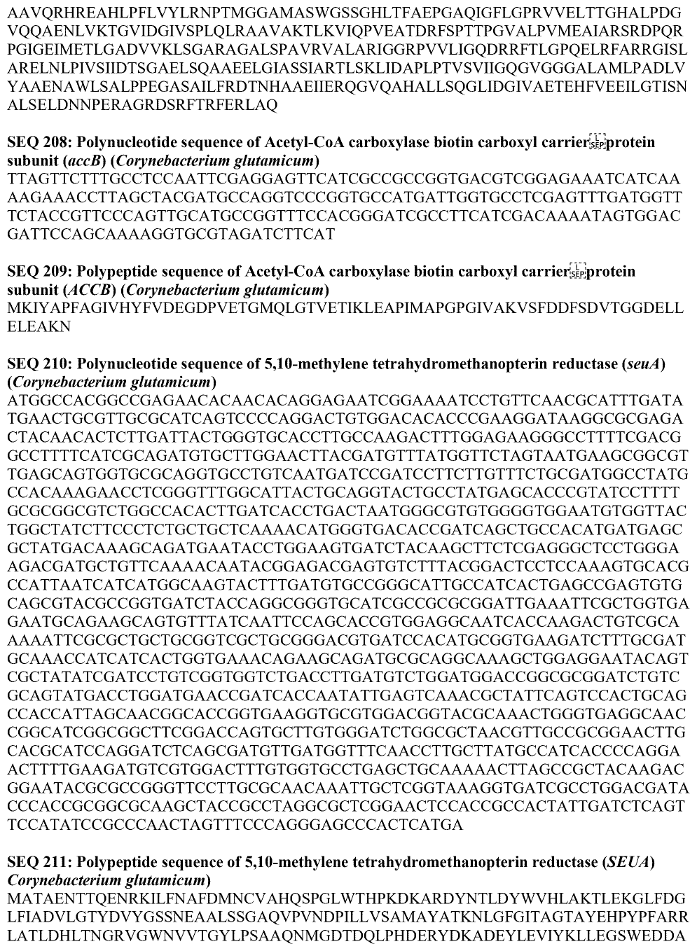

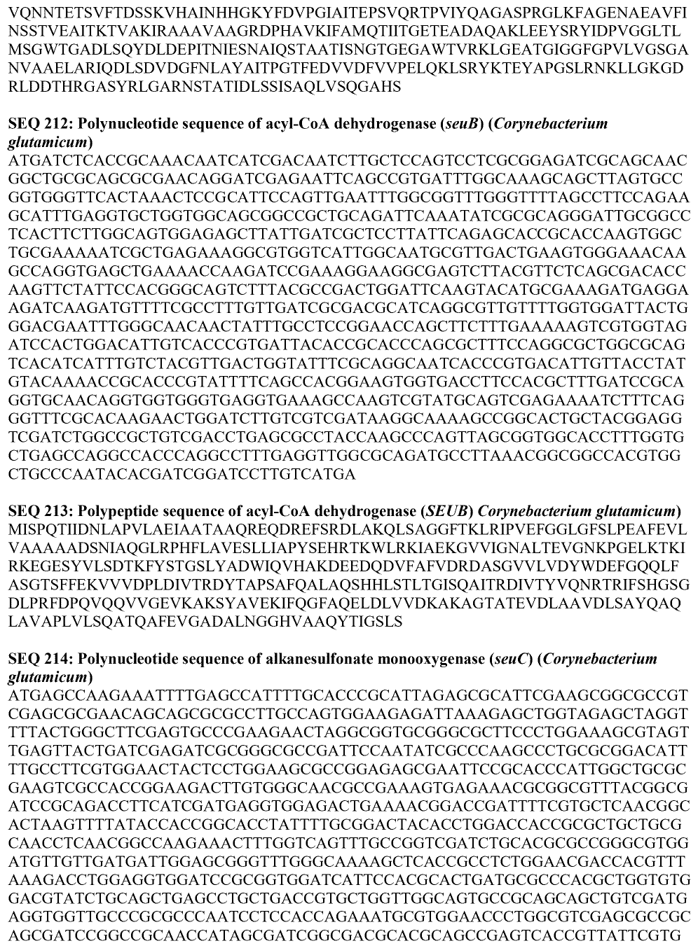

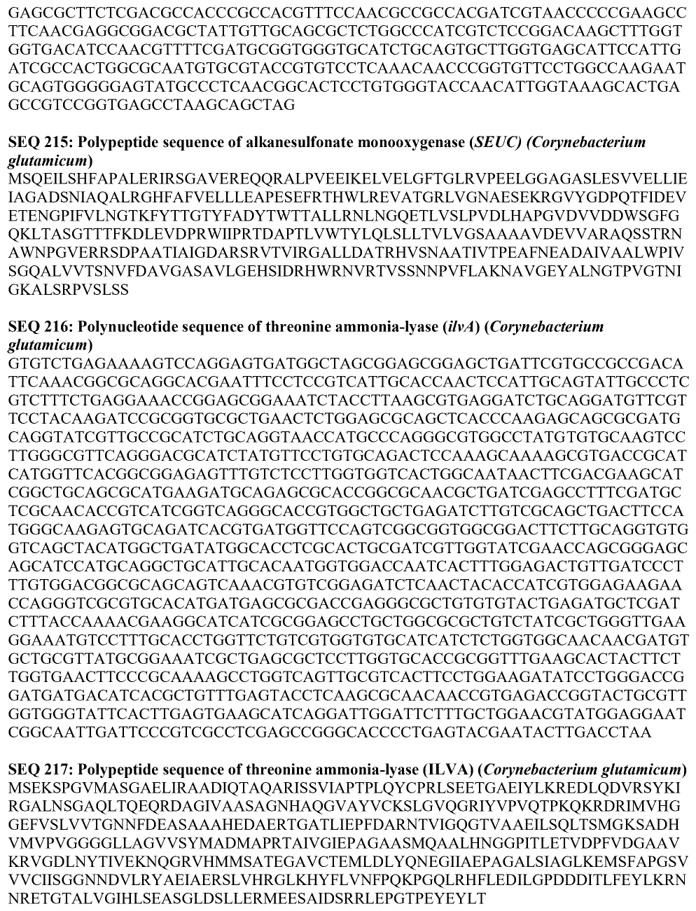

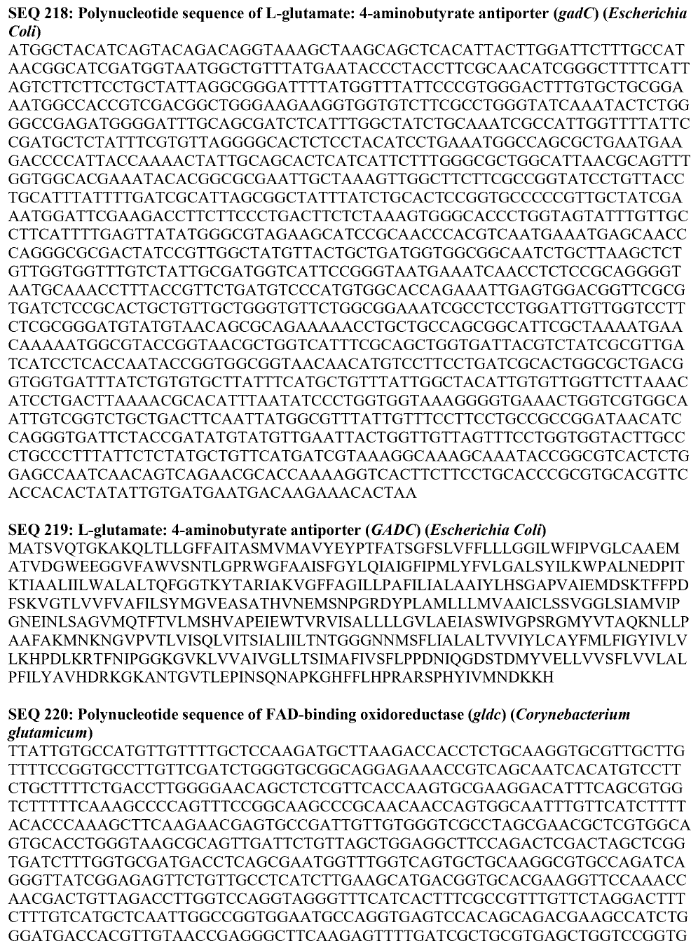

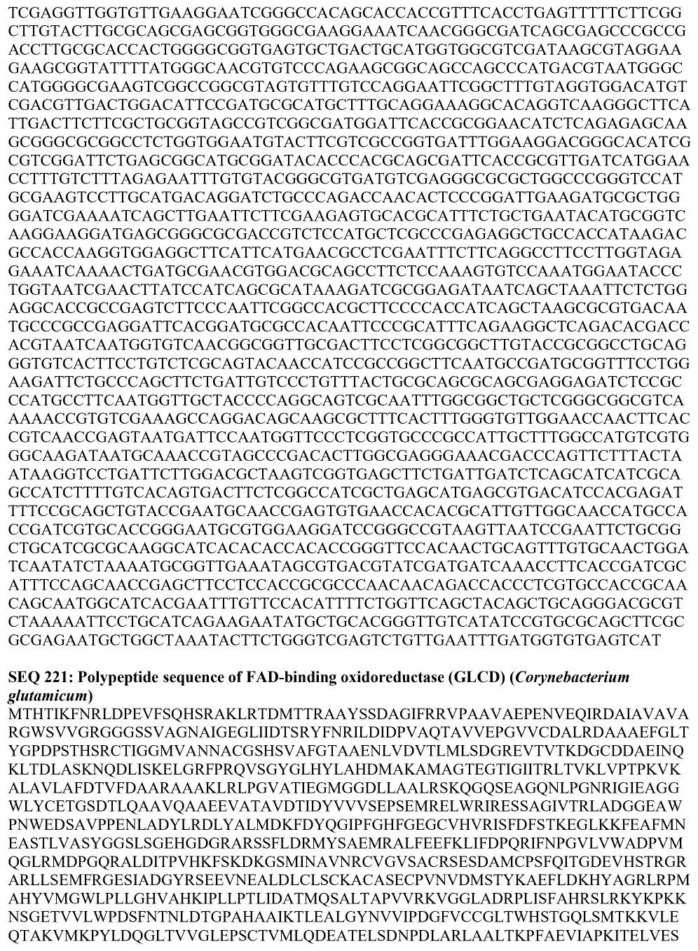

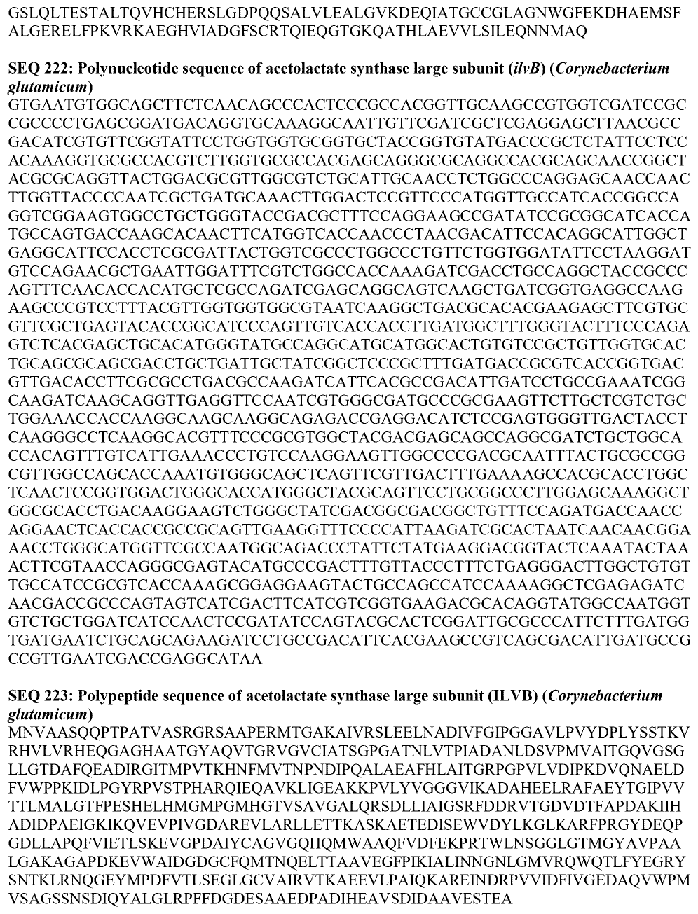

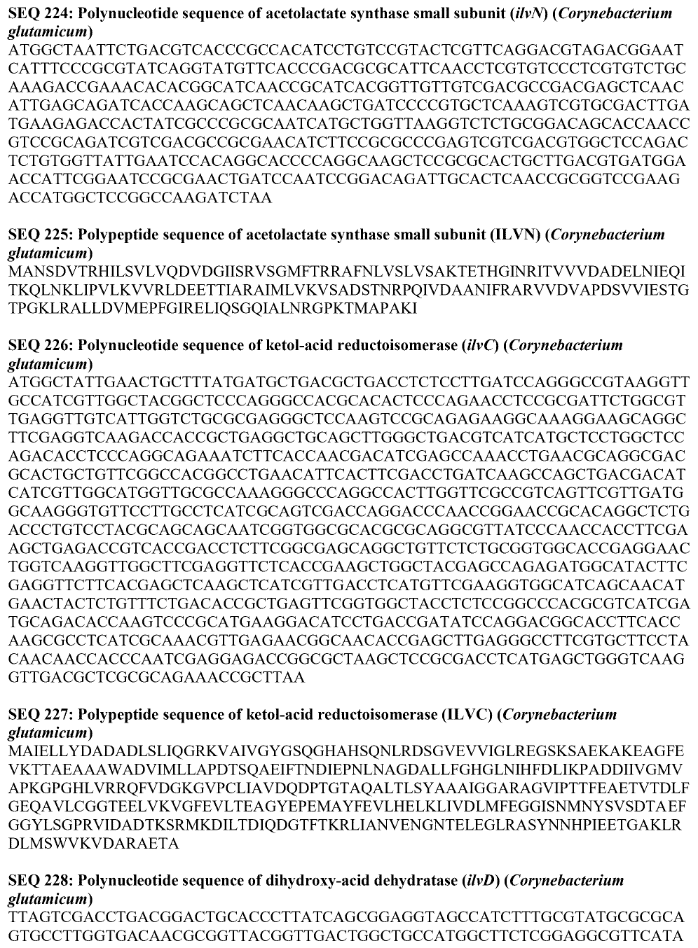

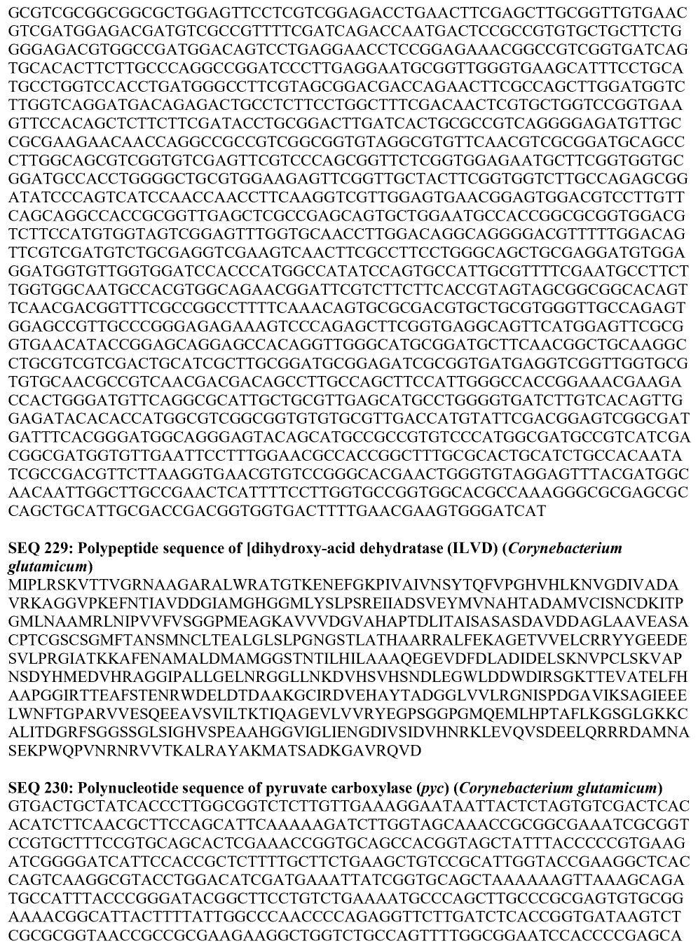

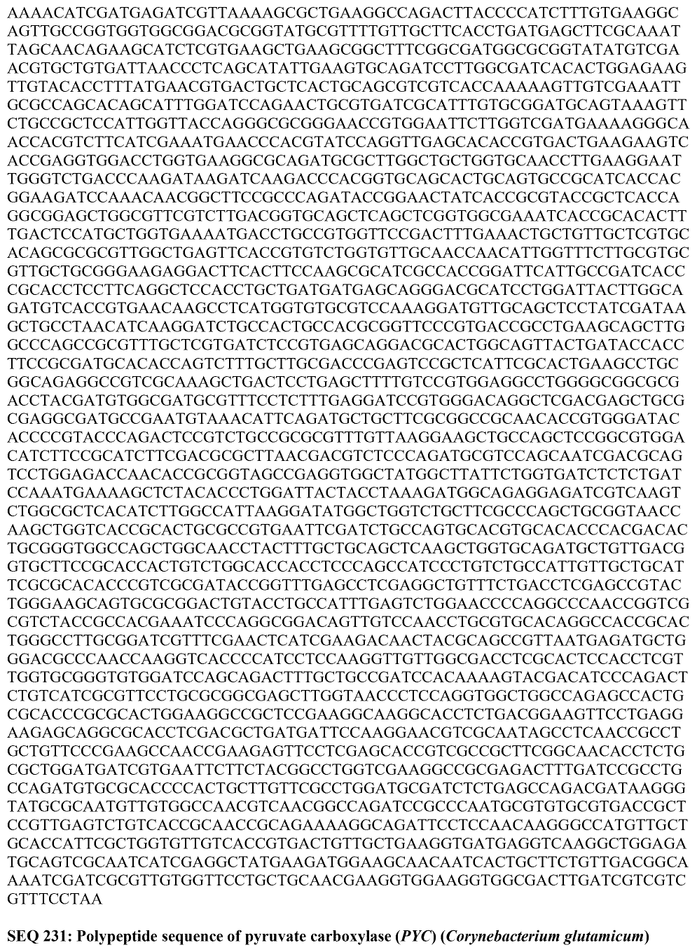

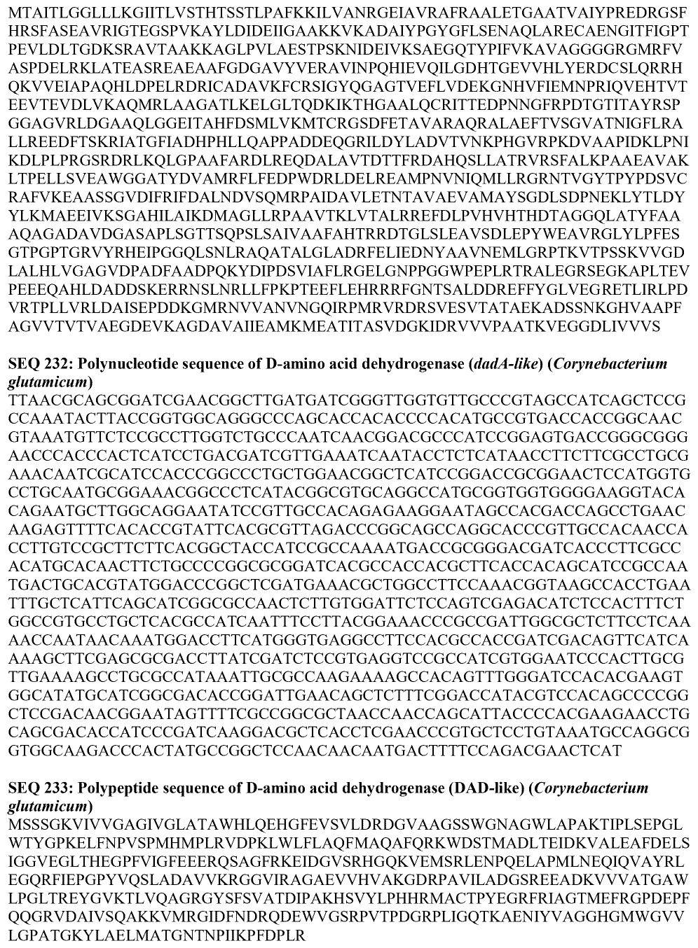

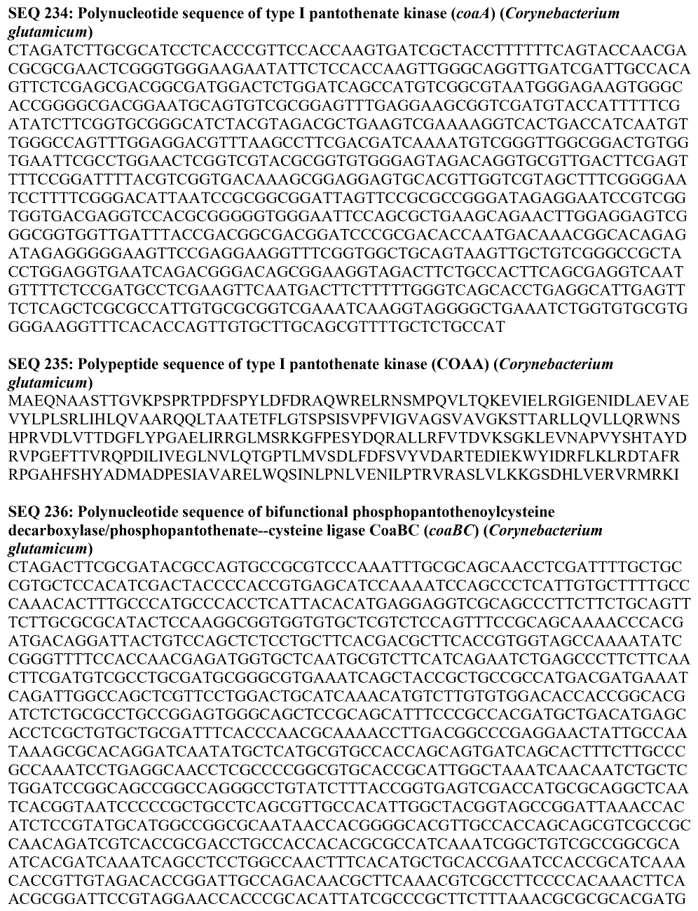

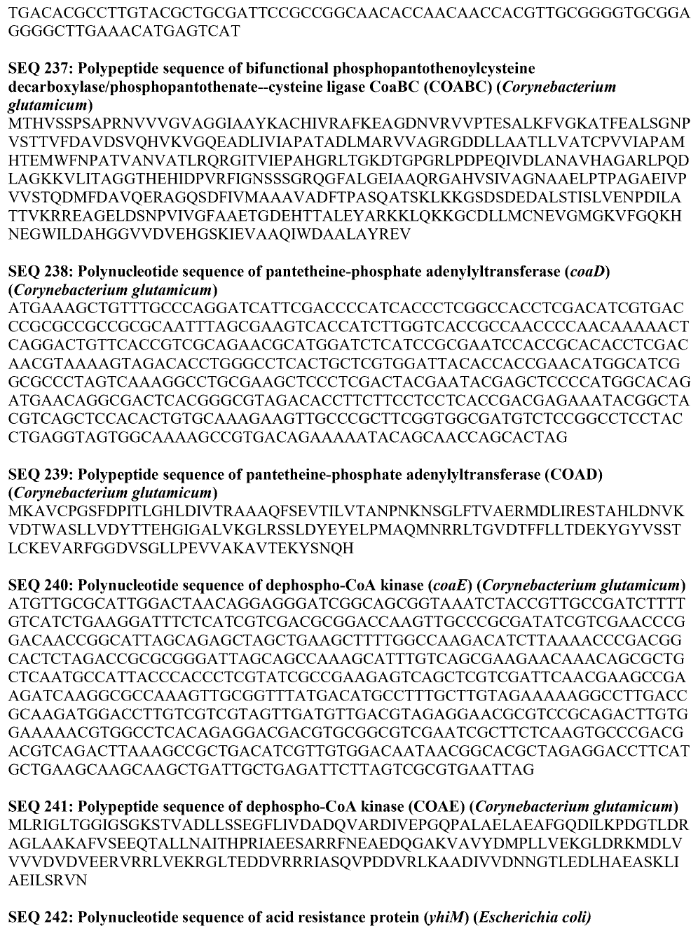

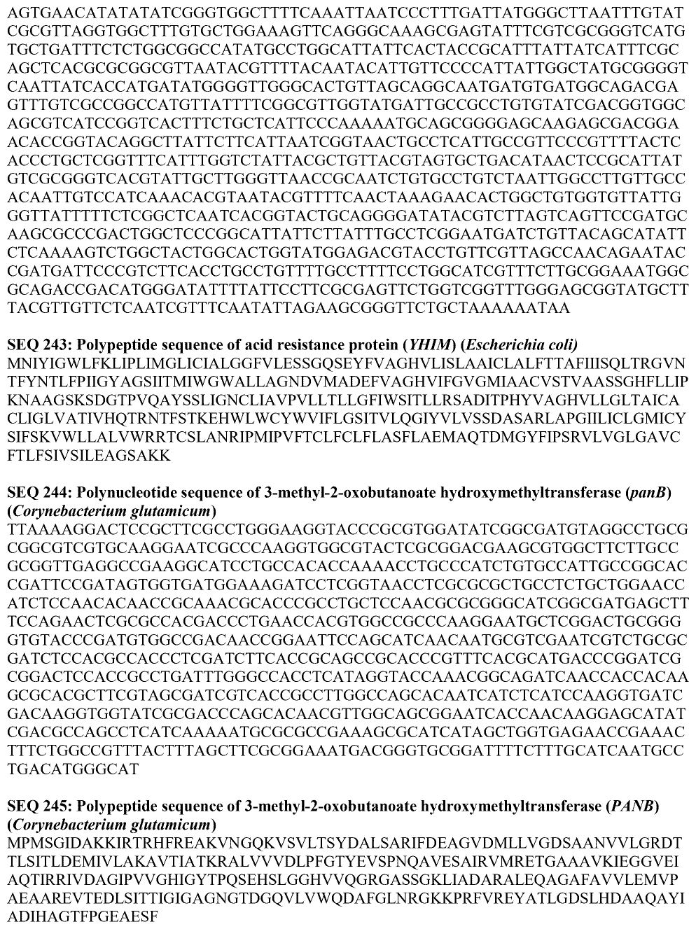

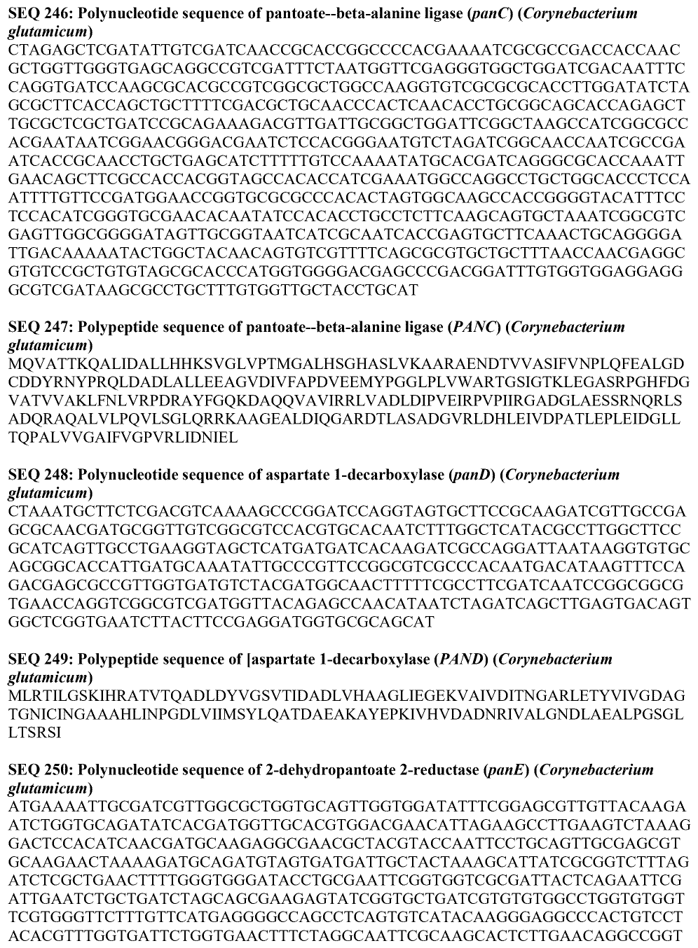

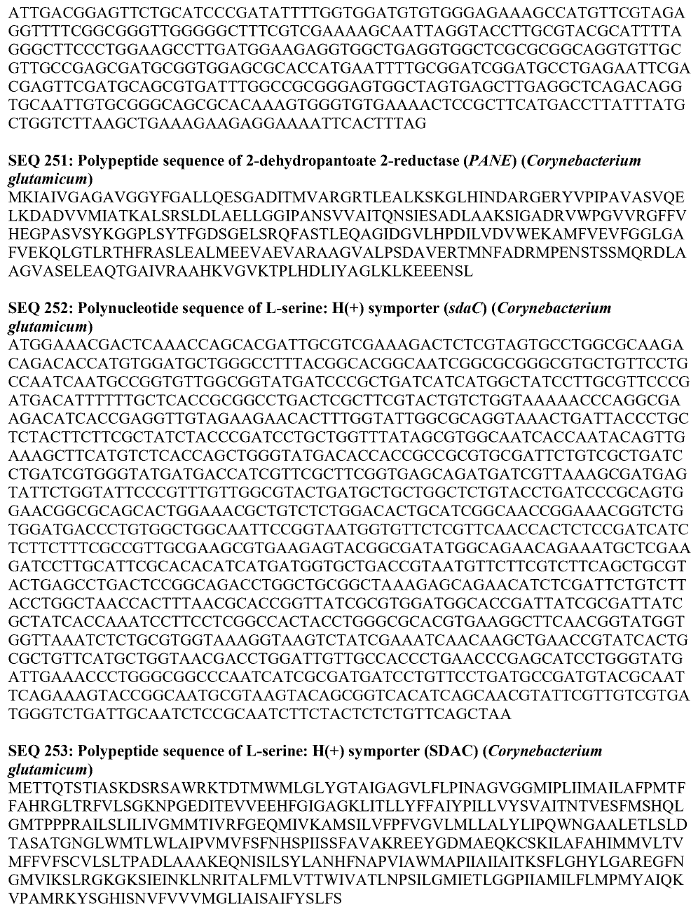

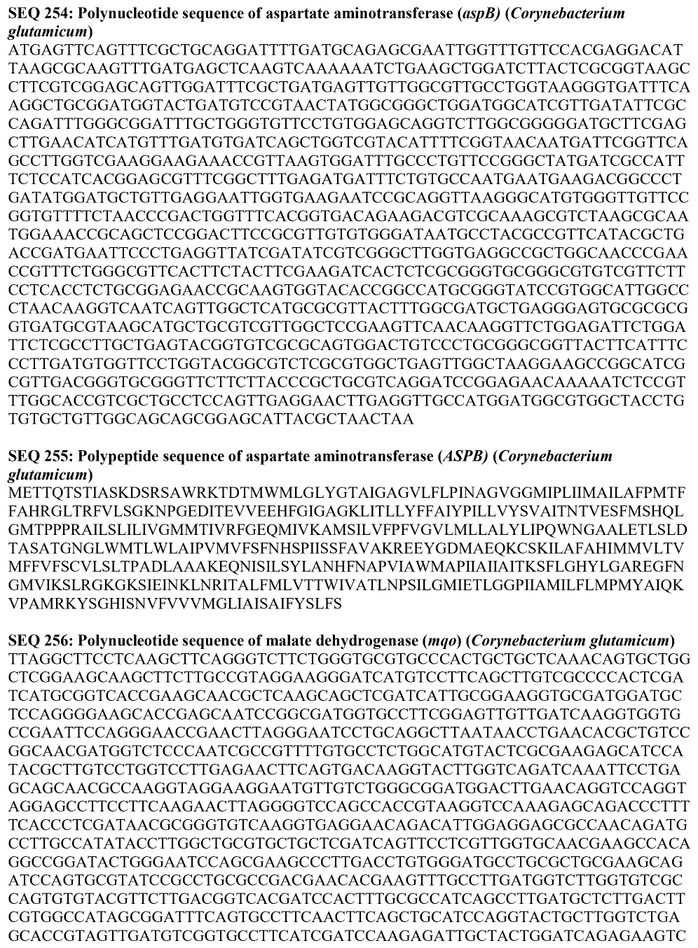

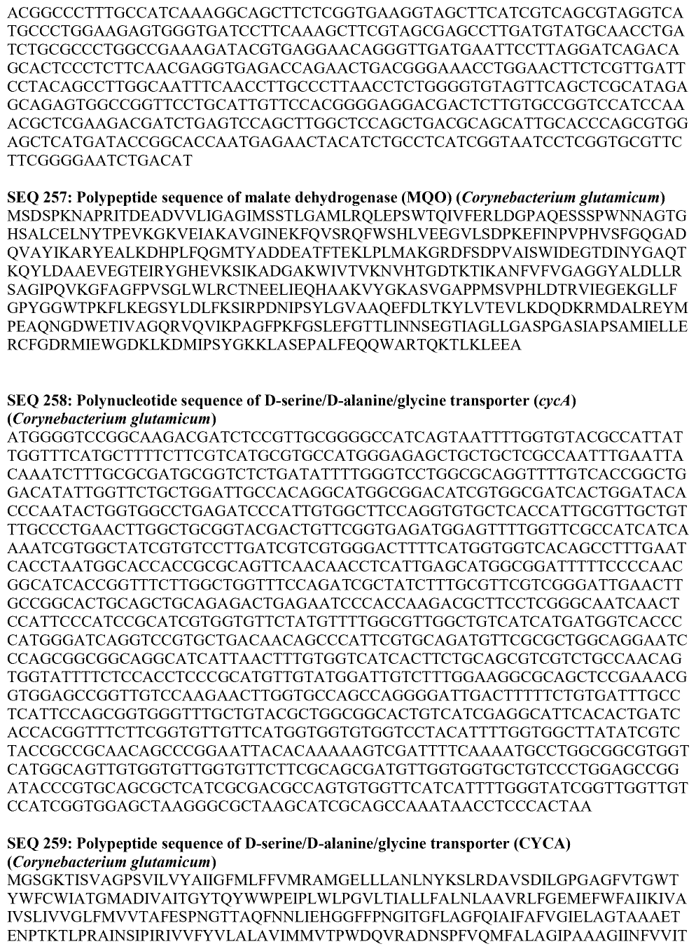

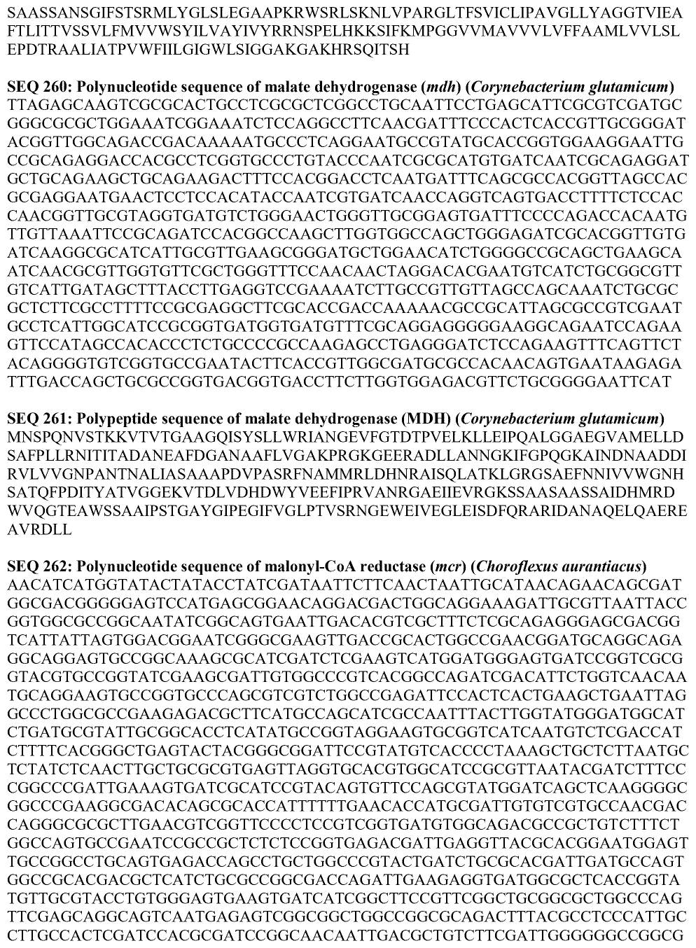

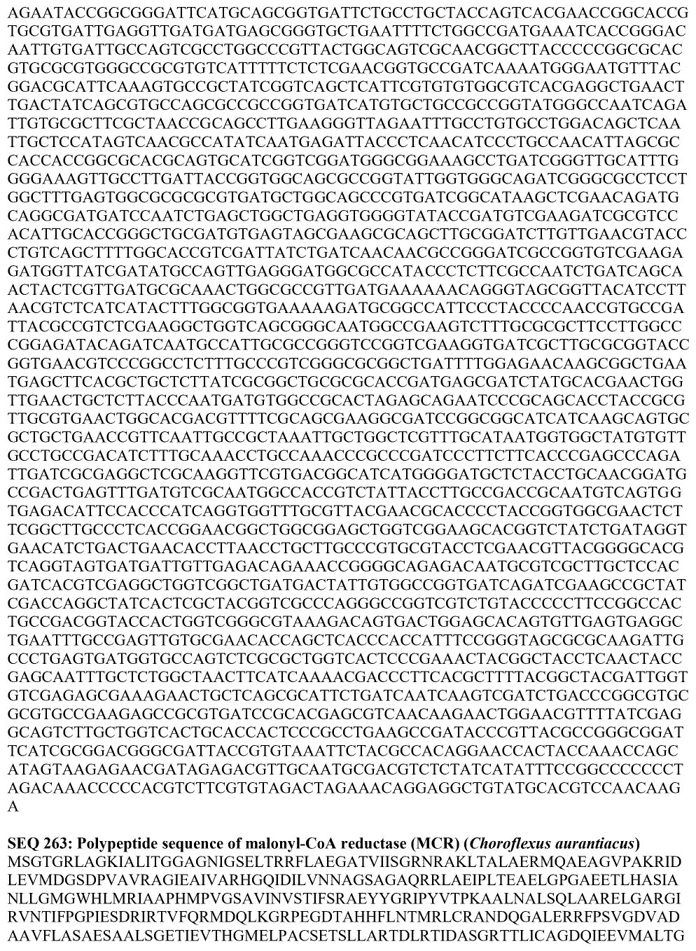

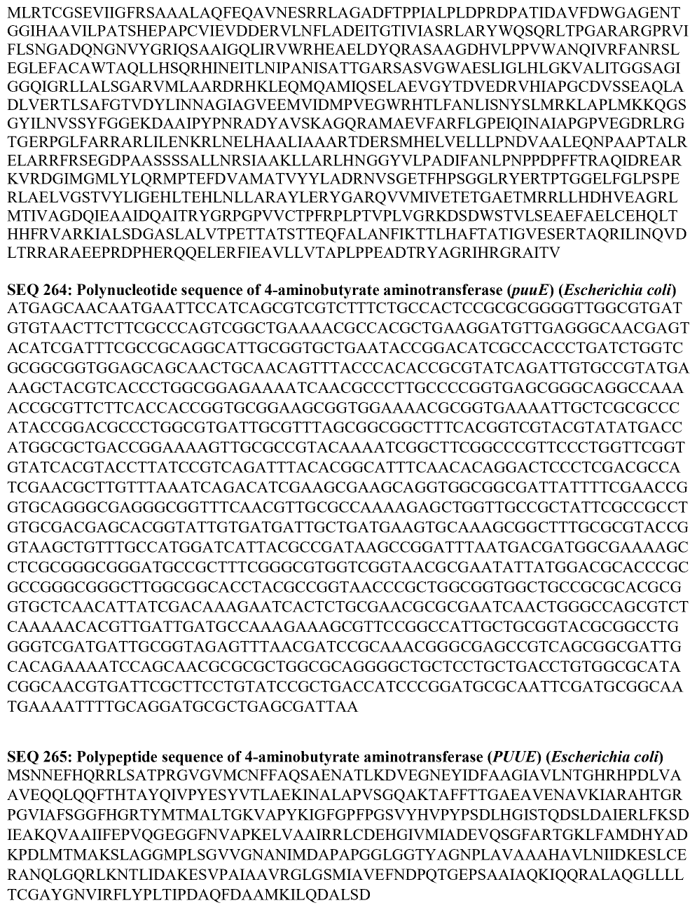

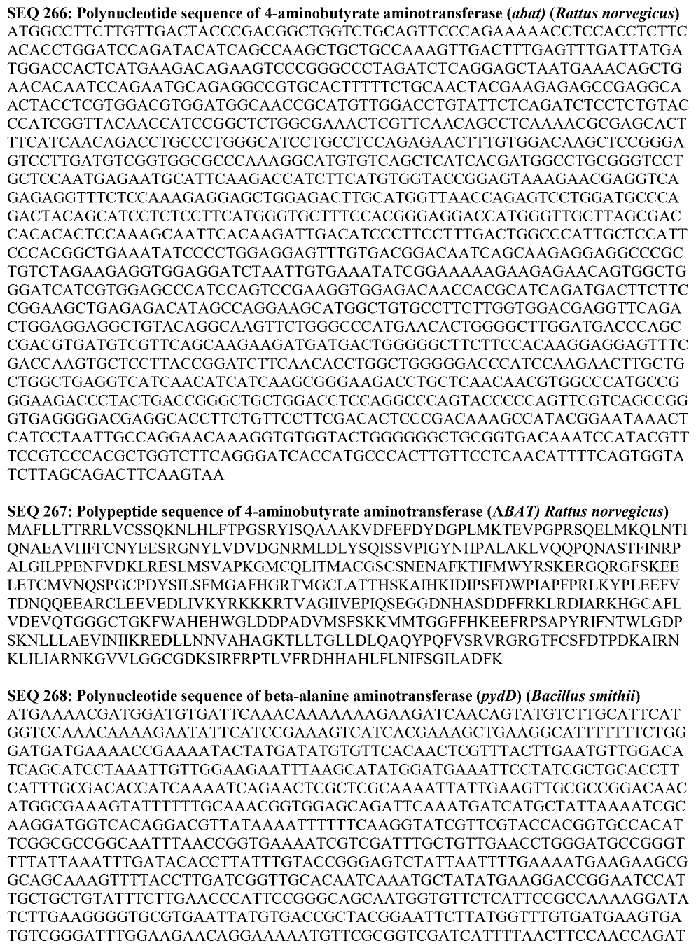

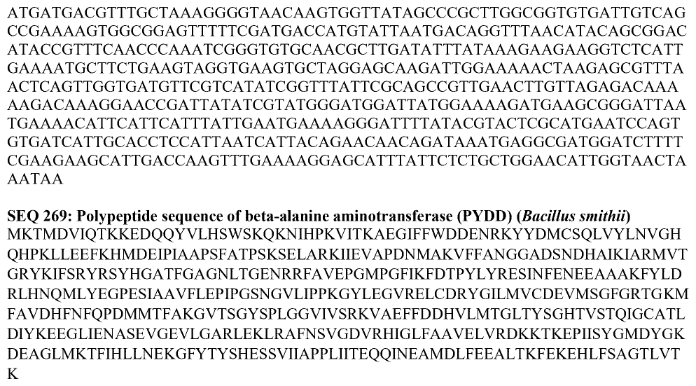

The embodiments of the present invention are generally related to genetic modifications for the optimized production of taurine in a novel prokaryotic organism. More particularly, according to one aspect of the present invention there is provided methods for the genetic modifications of bacterial organisms which further promote the organism to produce taurine through the introduction of eukaryotic and/or prokaryotic genes into the prokaryotic genome of a cell or the modification of already existing genes of the organism itself. As seen in FIG. 2, through the inclusion, alteration, or removal of polynucleotide sequences and therefore genes of a prokaryotic cell, herein are proposed modifications to both novel and endogenous metabolic pathways of the prokaryotic organism itself to promote the production of taurine by said organism.

According to a first aspect of the present invention, herein is provided genetic modifications to optimize an already established novel biosynthetic pathway from cysteamine to taurine which is engineered within a biological system and overcome the necessity for high energy intensive chemical processes.

According to a preferred method of the present invention, the prokaryotic cell being modified to promote the production of taurine is a bacterial cell. In a preferred method of the present invention, the prokaryotic cell is selected from the group consisting of the genera: Brevibacterium, Bacillus, Corynebacterium, Escherichia, Lactococcus, Pseudomonas, Rhodococcus, and Serratia. More preferably, the cell belongs to the genus Corynebacterium.

The person skilled in the art knows that organisms of a wide variety of species commonly express and utilize homologous genes and proteins, which contain insertions, substitutions and deletions in the polynucleotide and/or polypeptide sequences provided, and effectively provide a similar function. For example, the gene ssuR and its corresponding protein sequence from Corynebacterium glutamicum or Corynebacterium suranareeae or Brevilbacterium flavum or Corynebacterium glutamicum strain XV or Corynebacterium glutamicum strain SCgG1 or Corynebacterium glutamicum strain B414; or tauA and its protein sequence from Corynebacterium or Escherichia; or accB and its protein sequence from Corynebacterium or Phaeobacter inhibens or Clostridium tetani may differ to different degrees from the polynucleotide and/or polypeptide sequences seen between these organisms yet maintain similar or identical functions within the organism with respect to regulatory or catalytic function. Protein and gene sequences comprising such variations are included within the scope of the present invention and are considered substantially or sufficiently similar to the reference polynucleotide and polypeptide sequences provided. Although it is not intended that the present invention is limited by any theory by which it achieves its advantageous result, it is believed and supported by biochemical knowledge that the identity between polynucleotide and/or polypeptide sequences that is necessary to maintain proper functionality is related to maintaining the tertiary (3D) structure of the polypeptide. This maintenance of the tertiary structure is associated with the specific interactive/catalytic portions of the protein sequence and will therefore have the desired activity, and it is contemplated that a protein including these interactive sequences in the proper spatial context will have this activity.

According to a preferred method of the present invention, genes and promoter sequences were introduced into the genome or genetics of the organism using a genetic engineering method such as two-step allelic exchange or via introduction into the bacteria via an expression plasmid. Natural unmodified promoters and ribosomal binding sites from the bacterial expression strain or synthetic or modified promoters and ribosomal binding sites were attached to the genes upstream of the gene's start codon to drive gene expression within the bacterium. In another aspect of the present invention, there is provided a method to transfer these genes from cloning vectors into the organisms of interest. Preferably, these methods are modelled after the protocol seen for the knock in or knock out of genes in a different bacterium, Pseudomonas aeruginosa, using the aforementioned two step allelic exchange. However, the methods discussed herein are in no way meant to exclude the use of other methods, such as CRISPR cloning, from being used.

The person skilled in the art will recognize that genetic modifications may be carried out a multitude of ways, all with the purpose of knocking out, knocking in, decreasing or increasing the expression of a gene, or altering the functionality of a gene of interest. Gene and/or protein expression may be decreased through physically excising a part or all of a gene from the genome or transcriptome of the organism with the purpose being to prevent a functional or bioactive protein from being produced; using systems like CRSPR to degrade produced mRNA in the cell prior to protein production; introducing sequences into the organism that will produce inhibitory or antisense RNA, which can be designed to downregulate or prevent the translation of a protein from produced mRNA due to the interference of antisense or inhibitory RNA produced by the cell: by altering or eliminating the promoters and/or RBS's that drive the expression of a gene of interest; swapping a gene product of interest for a less efficient copy of the gene from another organism; or by altering key parts of the gene nucleotide sequence to decrease the activity of the resultant protein of interest. Gene and/or expression/transcription and/or protein translation may be increased by adding multiple copies of a gene into the genome; swapping natural promoters for stronger natural or synthetic promoters; altering key parts of the gene nucleotide sequence to increase the activity of the resultant protein of interest; or swapping a gene product of interest for a more efficient copy of the gene from another organism.

According to a preferred embodiment of the present invention, genes that are involved in the synthesis and/or transportation of taurine that may be integrated into prokaryotic organisms are provided in the appended sequence listings. The expression and production of these sequences within the cell are partially driven by the genetic polynucleotide promoter and ribosomal binding site sequences as provided in the sequence listings: SEQ 7 (P_{gWyA}), SEQ 8 (P_{SOD}), SEQ 9 (P_{pgk}), SEQ 10 (P_{tuf}), SEQ11 (P_{fbaA}), SEQ 12 (P_{lysc}), SEQ 13 (Pₜₖₜ), SEQ 14 (P_{ginA}), SEQ 15 (P_{pvc}), SEQ 16 (Pₕₒₘ), SEQ 17 (P_{gnd}), SEQ 18 (P_{lysA}), SEQ 19 (P_{aspB}), SEQ 20 (P_{adh}), SEQ 21 (P_{dapB}), SEQ 22 (P_{dapA}) and SEQ 23 (P_{tac}). The aforementioned sequences and associated taurine genes related to optimizing its production provided by way of example and are in no way limiting and/or comprehensive.

According to a preferred embodiment of the present invention, the cell has been genetically modified to add, remove, or inactivate at least one gene whose function is to alter positively the synthesis of taurine and alter positively or negatively the synthesis of other taurine-related precursors within the cell. These modifications could be achieved through several mechanisms, including but not limited to: the deletion of the offending gene product from the genome; the modification of the promoter sequence to reduce or prevent gene expression; the degradation of the messenger RNA produced by the polynucleotide sequence, which is used to produce the final polypeptide sequence; the introduction of sequences or genes that directly regulate gene/protein production, or are used to produce antisense RNA products to prevent and downregulate the production of a polypeptide through a process called antisense transcription; or a combination of several methods. Some ways not limited to those listed to upregulate or modify gene activity or transcription/translation can be brought about by expressing multiple copies of the same gene in the genome under the original promoter, replacing the promoter with another naturally occurring or synthetic promoter, making key mutations in the gene to artificially increase protein activity, and/or replacing the gene with another copy of the gene of interest from an alternate genetic source, or a combination of the above methods to upregulate or modify gene activity or transcription/translation.

According to a preferred embodiment of the present invention, the at least one gene to promote taurine production is selected from the group consisting of: transcriptional repressors controlling sulfur assimilation; transcriptional repressors controlling nitrogen assimilation; taurine dehydrogenases and FAD-dependent oxidoreductases; oxidoreductases; sulfitonases; sulfur compound transporter proteins; taurine dioxygenases; oxidoreductases; FMNH2-dependent sulfonatases; NAD(P)H-dependent FMN reductases; reductases that reduce taurine precursors to produce substrates for other metabolic pathways in the cell; taurine dioxygenases; sulfoacetaldehyde acetyltransferases; PLP dependent aminotransferases; branched-chain-amino-acid aminotransferases; taurine: 2-oxoglutarate aminotransferases; taurine-pyruvate aminotransferases; aminotransferase family proteins; regulatory proteins for organosulfur compound degradation; genes that divert precursor molecules away from taurine such as carboxylases or synthase/synthesis proteins; carboxyl transferases; malonyl-CoA reductases; aminotransferase family proteins; FMNH2-dependent monooxygenases; taurine-pyruvate aminotransferases; hydroxymethyltransferases; synthetases; synthases; reductases; decarboxylases; dehydratase proteins; and kinases or any combination thereof.

According to a preferred embodiment of the present invention, the genetically modified bacterial organism comprises the addition, deletion, repression, or modification of one or more genes that encode protein(s) which are responsible for the production of taurine. Such modifications can include at least one of the following polynucleotide sequences selected from the group consisting of: mebR (SEQ 184), amtr (SEQ 186), xsc-like (SEQ 188), ssuI (SEQ 190), ssuD1 (SEQ 192), ssuD2 (SEQ 194), ssuR (SEQ 196), accA (SEQ 206), accB (SEQ 208), seuA (SEQ 210), seuB (SEQ 212), seuC (SEQ 214), ilvA (SEQ 216), gldc (SEQ 220), ilvB (SEQ 222), ilvN (SEQ 224), ilvC (SEQ 226), ilvD (SEQ 228), pyc (SEQ 230), dadA-like (SEQ 232), coaA (SEQ 234), coaBC (SEQ 236), coaD (SEQ 238), coaE (SEQ 240), panB (SEQ 244), panC (SEQ 246), panD (SEQ 248), panE (SEQ 250), aspB (SEQ 254), mqo (SEQ 256), and mdh (SEQ 260), mer (SEQ 262), puuE (SEQ 264), abat (SEQ 266), and pydD (SEQ 268). These polynucleotide sequences upon transcription and translation may provide a polypeptide sequence selected from the group consisting of: MCBR (SEQ 185), AMTR (SEQ 187), XSC-like (SEQ 189), SSUI (SEQ 191), SSUD1 (SEQ 193). SSUD2 (SEQ 195), SSUR (SEQ 197), ACCA (SEQ 207), ACCB (SEQ 209), SEUA (SEQ 211), SEUB (SEQ 213), SEUC (SEQ 215), ILVA (SEQ 217), GLDC (SEQ 221), ILVB (SEQ 223), ILVN (SEQ 225), ILVC (SEQ 227), ILVD (SEQ 229), PYC (SEQ 231), DADA-like (SEQ 233), COAA (SEQ 235), COABC (SEQ 237), COAD (SEQ 239), COAE (SEQ 241), PANB (SEQ 245), PANC (SEQ 247), PAND (SEQ 249), PANE (SEQ 251), ASPB (SEQ 255), MQO (SEQ 257), and MD (SEQ 261), MCR (SEQ 263), PUUE (SEQ 265), ABAT (SEQ 267), and PYDD (SEQ 269). The sequences and related genes/proteins provided herein associated with the production of taurine are by way of example and are in no way limiting and/or comprehensive.

According to a preferred embodiment of the present invention, the genetically modified bacterial organism comprises the addition, deletion, repression, or modification of one or more genes that encode protein(s) which are responsible for the cellular transportation of taurine. Taurine import/export proteins that may be used in the present invention include but are not limited to sulfur compound transporter proteins, glutamatein/γ-aminobutyrate (GABA) electrogenic-like antiporters; amino acid transporters; polyamine transporters; and/or amino acid permease proteins.

According to a preferred embodiment of the present invention, the genetically modified bacterial organism is modified through the addition, alteration, and/or deletion of genes related to the cellular transportation of taurine. Such modifications can include at least one of the polynucleotide sequences selected from the group consisting of: ssuA (SEQ 198), ssuB (SEQ 200), ssuC (SEQ 202), tauE (SEQ 204), gadC (SEQ 218), yhiM (SEQ 242), sdaC (SEQ 252), and cycA (SEQ 258). These polynucleotide sequences upon transcription and translation may provide a polypeptide sequence selected from the group consisting of: SSUA (SEQ 199), SSUB (SEQ 201), SSUC (SEQ 203), TAUE (SEQ 205), GADC (SEQ 219), YHIM (SEQ 243), SDAC (SEQ 253), and CYCA (SEQ 259). The sequences and related genes/proteins provided herein associated with the transportation of taurine are by way of example and are in no way limiting and/or comprehensive.

The person skilled in the art will recognize that many different organisms will have functionally similar polynucleotide and polypeptide sequences (or homology between the sequences), however there may be differences between these sequences when compared to a reference sequence. As examples, suitable polynucleotides and their corresponding polypeptide sequences for the production of taurine can be seen below. Note that the following sequences by no means are meant to limit the scope of the invention. In fact, any substantially similar polynucleotide sequences or substantially similar produced polypeptide sequences for the ADO. VNN, and FMO1 genes with similar function or similarity to these genes in the taurine biosynthesis pathway.

The person skilled in the art will recognize that changes in the protein sequence, resulting from individual single or multi-nucleotide substitutions, deletions, or additions to a polynucleotide will lead to changes in the resulting translated polypeptide sequence. Small mutations, such as the change of an amino acid from one to another, or the addition or elimination of single amino acids, or a small to moderate percentage of amino acids from the encoded polypeptide sequence can be considered “sufficiently similar” when the alteration results in the substitutions of an amino acid with a chemically similar amino acid. Thus, any number of amino acid residues in a polypeptide chain, selected from a group of integers from 1-50, can be so altered. Thus, for example, 1, 2, 3, 5, 10, 12, 20, 32, 41, or even 50 alterations can be made. Conservatively modified variants typically provide similar biological activity as the unmodified polypeptide sequence from which they are derived. For example, modification of ADO, VNN, and FMO1 to yield functional proteins generally have, preferably, a sequence identity of at least 40%, 50%, 60%, 70%, 80%, or 90%, preferably a sequence identity of greater than 50%, of the native protein to allow processing of its native substrate. Tables of conserved substitution provide lists of functionally similar amino acids. Amino acids in polypeptide chains that are similar to one another include, but are not limited to, the following groups: (1) Serine (S), Threonine (T); (2) Aspartic acid (D), Glutamic acid (E); (3) Asparagine (N), Glutamine (Q); (4) Alanine (A), Leucine (L), and Isoleucine (I).

According to a preferred embodiment of the present invention, the utilized native or synthetic promoter sequence is followed by a polynucleotide sequence encoding for a vanin-1 (vnn-1) gene natively found in eukaryotic organisms. In some embodiments, vnn-/genes can be acquired from eukaryotic organisms such as Sus scrofa, Homo sapiens, Ursus maritimus, Lutra lutra, Nycticebus coucang, Mus musculus, Salvelinus alpinus, Phrynosoma platyrhinos, Vombatus ursinus, Bucco capensis, Notechis scutatus, Sinocyclocheilus anshuiensis, Salmo salar, Marmota monax, Clupea harengus, and Harpia harpyja although the listed organisms are only given as examples and are in no way meant to limit what organisms these genes can be acquired from. In a preferred embodiment of the present invention, the vanin-1 (vnn-1) polynucleotide sequence SEQ 3 isolated from the eukaryotic species Sus scrofa (pig) is utilized in the process described herein under the transcriptional control of a native or artificial promoter and a ribosomal binding site. However, in other embodiments of the present invention, polynucleotide sequences that are homologous and/or substantially similar to SEQ 3 may also be used. In another embodiment of the present invention, polynucleotide sequences that are homologous and/or substantially similar to SEQ 98 may also be used. Polynucleotide sequences for vanin-1 in these embodiments will have at least 70% sequence coverage, or more preferentially greater than 75%, 80%, 85%, 90%, 95%, 97%, or 98% sequence coverage, or most preferentially greater than 99% sequence coverage to SEQ 3 or SEQ 98, and the polynucleotide sequence of vanin-1 has at least 70% sequence identity, or more preferentially 80%, 85%, 90%, 95%, 97%, or 98% sequence identity, or most preferentially greater than 99% sequence identity to SEQ 3 or SEQ 98. These polynucleotide sequences may include, but by no means are limited to, the following sequences: SEQ 45, SEQ 46, SEQ 47, SEQ 48, SEQ 49, SEQ 50, SEQ 51, SEQ 52, SEQ 53, and SEQ 54.

According to a preferred embodiment of the present invention, the vanin-1 (VNN1) polypeptide SEQ 4 from the eukaryotic species Sus scrofa (pig), is utilized to produce taurine by the cell, whereby SEQ 4 is produced from the transcription and translation of the vanin-1 polynucleotide SEQ 3 or SEQ 98. However, in other embodiments of the invention, polypeptide sequences that are homologous and/or substantially similar to SEQ 4 may also be used in the present invention to produce taurine. Polypeptide sequences for vanin-1 in these embodiments will, preferably, have at least 70% sequence coverage, or more preferentially greater than 80%, 90%, 95%, 98%, or most preferentially greater than 99% sequence coverage of SEQ 4, and a sequence identity of at least 25% to SEQ 4, or more preferentially greater than 30%, 35%, 40%, 45%, 50%, 55%, 60%, 65%, 70% 75%, 80%, 85%, 90%, 95%, 97%, or most preferentially greater than 99% sequence identity to SEQ 4. These polypeptide sequences may include, but are by no means limited to, the following sequences: SEQ 55, SEQ 56, SEQ. 57, SEQ 58, SEQ 59, SEQ 60, SEQ 61, SEQ 62. SEQ 63, SEQ 64, SEQ 65, SEQ. 66, SEQ 67, SEQ 68, SEQ 69, and SEQ 70.

According to a preferred embodiment of the present invention, the utilized native or synthetic promoter sequence is followed by a polynucleotide sequence encoding for a vanin-2 (vnn-2) gene natively found in eukaryotic organisms. In some embodiments, vnn-2 genes can be acquired from eukaryotic organisms such as Bos taurus, Sus scrofa, Homo sapiens, Ursus maritimus, Lutra lutra, Nycticebus coucang, Mus musculus, Salvelinus alpinus, Phrynosoma platyrhinos, Vombatus ursinus, Bucco capensis, Notechis scutatus, Sinocyclocheilus anshuiensis, Salmo salar, Marmota monax, Clupea harengus, and Harpia harpyja although the listed organisms are only given as examples and are in no way meant to limit the list of organisms from which these genes can be acquired from. In a preferred embodiment of the present invention, the vanin-2 (vnn2) polynucleotide sequence (SEQ 100) isolated from the eukaryotic species Bos taurus (cattle), can be utilized within this invention in place of vanin-1 (vnn1) (such as SEQ 3 or SEQ 98). Said vnn2 polynucleotide sequence is utilized in the process described herein under the transcriptional control of a native or artificial promoter and a ribosomal binding site. However, in other embodiments of the present invention, polynucleotide sequences that are homologous and/or substantially similar to SEQ 100 may also be used. In another embodiment of the present invention, polynucleotide sequences for vanin-2 in these embodiments have at least 70% sequence coverage, or more preferentially greater than 80%. 85%, 90%, 95%, 96%, or 97% sequence coverage, or most preferentially greater than 99% sequence coverage of SEQ 100, and the polynucleotide sequence of vanin-2 has at least 70% sequence identity, or more preferentially 80%, 85%, 90%, 95%, or 96% sequence identity, or most preferentially greater than 99% sequence identity to SEQ 100. These polynucleotide sequences may include, but by no means are limited to, the following sequences: SEQ 101; SEQ 102; SEQ 103; SEQ 104; SEQ 105; SEQ 106; SEQ 107; SEQ 108; SEQ 109; SEQ 110; SEQ 111; SEQ 112; and SEQ 113.

According to a preferred embodiment of the present invention, the vanin-2 (VNN2) polypeptide sequence SEQ 114 isolated from the eukaryotic species Sus scrofa (pig) can be utilized within this invention, whereby SEQ 114 is produced from the transcription and translation of the vanin-2 polynucleotide SEQ 100. However, in other embodiments of the invention, polypeptide sequences that are homologous and/or substantially similar to SEQ 114 may also be used. Polypeptide sequences for vanin-2 in these embodiments will, preferably, have at least 70% sequence coverage, or more preferentially greater than 75%, 80%, 90%, 95%, or most preferentially greater than 99% sequence coverage of SEQ 114, and a sequence identity of at least 25% to SEQ 114, or more preferentially greater than 30%, 35%, 40%, 45%, 50%, 55%, 60%, 65%, 70% 75%, 80%, 85%, 90%, 95%, 98%, or most preferentially greater than 99% sequence identity to SEQ 114. These polypeptide sequences may include, but are by no means limited to, the following sequences: SEQ 115; SEQ 116; SEQ 117; SEQ 118; SEQ 119; SEQ 120; SEQ 121; SEQ 122; SEQ 123; SEQ 124; SEQ 125; SEQ 126; SEQ 127; SEQ 128; SEQ 129; SEQ 130; SEQ 131; SEQ 132; SEQ 133; SEQ 134; SEQ 135; SEQ 136; SEQ 137; SEQ 138; SEQ 139; and SEQ 140.

According to a preferred embodiment of the present invention, the utilized native or synthetic promoter sequence is followed by a polynucleotide sequence encoding for a vanin-3 (vnn-3) gene natively found in eukaryotic organisms. In some embodiments, vnn-3 genes can be acquired from eukaryotic organisms such as Bos taurus, Sus scrofa, Homo sapiens, Ursus maritimus, Lutra lutra, Nycticebus coucang, Mus musculus, Salvelinus alpinus, Phrynosoma platyrhinos, Vombatus ursinus, Bucco capensis, Notechis scutatus, Sinocyclocheilus anshuiensis, Salmo salar, Marmota monax, Clupea harengus, and Harpia harpyja although the listed organisms are only given as examples and are in no way meant to limit what organisms these genes can be acquired from. In a preferred embodiment of the present invention, the vanin-3 (vnn-3) polynucleotide sequence SEQ 141 isolated from the eukaryotic species Mus musculus (house mouse) can be utilized within this invention in place of vanin-1 (vnn-1) SEQ 3 or SEQ 98. Said vnn3 polynucleotide sequence is utilized in the process described herein under the transcriptional control of a native or artificial promoter and a ribosomal binding site. However, in other embodiments of the invention, polynucleotide sequences that are homologous and/or substantially similar to SEQ 141 may also be used. In another embodiment of the present invention, polynucleotide sequences for vanin-3 in these embodiments will have at least 70% sequence coverage, or more preferentially greater than 75%, 80%, 85%, 90%, 95%, 96%, or 97% sequence coverage, or most preferentially greater than 99% sequence coverage of SEQ 141, and the polynucleotide sequence of vanin-3 has at least 70% sequence identity, or more preferentially 75%, 80%, 90%, 95%, or 97% sequence identity, or most preferentially greater than 99% sequence identity to SEQ 141. These polynucleotide sequences may include, but by no means are limited to, the following sequences: SEQ 142; SEQ 143; SEQ 144; SEQ 145; SEQ 146; SEQ 147; SEQ 148; SEQ 149; SEQ 150; SEQ 151; SEQ 152; SEQ 153; SEQ 154; SEQ 155; SEQ 156; and SEQ 157.

According to a preferred embodiment of the present invention, the vanin-3 (VNN3) polypeptide SEQ 158 isolated from the eukaryotic species Mus musculus (house mouse) can be utilized within this invention, whereby SEQ 158 is produced from the transcription and translation of the vanin-3 polynucleotide SEQ 141. However, in other embodiments of the invention, polypeptide sequences that are homologous and/or substantially similar to SEQ 158 may also be used. Polypeptide sequences for vanin-3 in these embodiments will, preferably, have at least 70% sequence coverage, or more preferentially greater than 75%, 80%, 85%, 90%, 95%, 96%, 97%, 98%, or most preferentially greater than 99% sequence coverage of SEQ 158, and a sequence identity of at least 25% to SEQ 158, or more preferentially greater than 30%, 35%, 40%, 45%, 50%, 55%, 60%, 65%, 70% 75%, 80%, 85%, 90%, 95%, 98%, or most preferentially greater than 99% sequence identity to SEQ 158. These polypeptide sequences may include, but are by no means limited to, the following sequences: SEQ 159; SEQ 160; SEQ 161; SEQ 162; SEQ 163; SEQ 164; SEQ 165; SEQ 166; SEQ 167; SEQ 168; SEQ 169; SEQ 170; SEQ 171; SEQ 172; SEQ 173; SEQ 174; SEQ 175; SEQ 176; SEQ 177; SEQ 178; SEQ 179; SEQ 180; SEQ 181; SEQ 182; and SEQ 183.

According to a preferred embodiment of the present invention, the utilized native or synthetic promoter sequence is followed by a polynucleotide sequence, such as cysteamine dioxygenase (ado) natively found in eukaryotic organisms. In some embodiments, the ado genes can be acquired from eukaryotic organisms such as Sus scrofa, Homo sapiens, Ursus maritimus, Lutra lutra, Nycticebus coucang, Mus musculus, Salvelinus alpinus, Phrynosoma platyrhinos, Vombatus ursinus, Bucco capensis, Notechis scutatus, Sinocyclocheilus anshuiensis, Salmo salar, Marmota monax, Clupea harengus, and Harpia harpyja although the listed organisms are only given as examples and are in no way meant to limit what organisms these genes can be acquired from. In a preferred embodiment of the present invention, the cysteamine dioxygenase polynucleotide (ado) sequence SEQ 1 isolated from the eukaryotic species Sus scrofa (pig) is utilized in the process described herein under the transcriptional control of a native or artificial promoter and a ribosomal binding site. However, in other embodiments of the invention, polynucleotide sequences that are homologous and/or substantially similar to SEQ 1 may also be used. In another embodiment of the present invention, polynucleotide sequences for cysteamine dioxygenase in these embodiments will, preferably, have at least 70% sequence coverage, or more preferably greater than 80%, 90%, 95%, 98%, or most preferentially greater than 99% sequence coverage to SEQ 1, and sequence identities of at least 70%, or more preferentially greater than 80%, 90%, 95%, 97% sequence identity, and most preferentially 99% sequence identity to SEQ 1. These polynucleotide sequences may include, but by no means limited to, the following sequences: SEQ 24, SEQ 25, SEQ 26, SEQ 27, SEQ 28, and SEQ 29.

According to a preferred embodiment of the present invention, the cysteamine dioxygenase polypeptide (ADO) sequence SEQ 2 isolated from the eukaryotic species Sus scrofa (pig), is utilized within this invention, whereby SEQ 2 is produced from the transcription and translation of the cysteamine dioxygenase polynucleotide SEQ 1. However, in other embodiments of the invention, polypeptide sequences that are homologous and/or substantially similar to SEQ 2 may also be used in the present invention to produce taurine. Polypeptide sequences for cysteamine dioxygenase in these embodiments will, preferably, have at least 70% sequence coverage, or more preferentially greater than 80%, 90%, 95%, 98%, or most preferentially greater than 99% sequence coverage to SEQ 2, and a sequence identity of, preferably, at least 25% to SEQ 2, or more preferentially greater than 30%, 35%, 40%, 45%, 50%, 55%, 60%, 65%, 70% 75%, 80%, 85%, 90%, 95%, 97%, or most preferentially greater than 99% sequence identity to SEQ 2. These polypeptide sequences may include, but are not limited to, the following sequences: SEQ 30, SEQ 31. SEQ 32, SEQ 33, SEQ 34, SEQ 35, SEQ 36, SEQ 37, SEQ 38, SEQ 39, SEQ 40, SEQ 41, SEQ 42, SEQ 43, and SEQ 44.

According to a preferred embodiment of the present invention, the utilized native or synthetic promoter sequence is followed by a polynucleotide sequence encoding for a flavin-containing monooxygenase 1 (fmo1) natively found in eukaryotic organisms. In some embodiments, the fmo1 gene is acquired from eukaryotic organisms such as Sus scrofa, Capra hircus, Microtus fortis, Panthera pardus, Homo sapiens, Varanus komodoensis, Apodemus sylvaticus, Eublepharis macularius, Alca torda, Chordeiles acutipennis, Grantiella picta, Caloenas nicobarica, Regulus satrapa, and Lutra lutra, although the listed organisms are only given as examples and are in no way meant to limit what organisms these genes can be acquired from. In a preferred embodiment of the present invention, the fmo1 gene is sourced from Sus scrofa (pig). In a preferred embodiment of the present invention, the flavin-containing monooxygenase 1 (fmo1) polynucleotide sequence SEQ 5 isolated from the eukaryotic species Sus scrofa (pig), is utilized in the process described herein under the transcriptional control of a native or artificial promoter and a ribosomal binding site. However, in other embodiments of the invention, polynucleotide sequences that are homologous and/or substantially similar to SEQ 5 may also be used in the present invention to produce taurine. In another embodiment of the present invention, polynucleotide sequences that are homologous and/or substantially similar to SEQ 99 may also be used in a preferred embodiment of the present invention. Polynucleotide sequences for flavin-containing monooxygenase 1 in these embodiments will, preferably, have at least 70% sequence coverage, or more preferentially greater than 75%, 80%, 85%, 90%, 95%, 97%, 98%, or most preferentially greater than 99% sequence coverage to SEQ 5 or SEQ 99, and a sequence identity of at least 70%, or more preferentially greater than 75%, 80%, 85%. 90%, 95%, 97%, or most preferentially greater than 99% sequence identity to SEQ 5 or SEQ 99. These polynucleotide sequences may include, but are by no means limited to, the following sequences: SEQ 71, SEQ 72, SEQ 73, SEQ 74, SEQ 75, SEQ 76, SEQ 77, SEQ 78, SEQ 79, SEQ 80, and SEQ 81.

According to a preferred embodiment of the present invention, the flavin-containing monooxygenase 1 (FMO1) polypeptide sequence SEQ 6 from the eukaryotic species Sus scrofa (pig) is utilized within this invention, whereby SEQ 6 is produced from the transcription and translation of the flavin-containing monooxygenase 1 polynucleotide SEQ 5 or SEQ 99. However, in other embodiments of the invention, polypeptide sequences that are homologous and substantially similar to SEQ 6 may also be used in the present invention to produce taurine. Polypeptide sequences for flavin-containing monooxygenase 1 in these embodiments will, preferably, have at least 70% sequence coverage, or more preferentially greater than 75%, 80%, 85%, 90%, 95%, 97%, 98%, or most preferentially greater than 99% sequence coverage of SEQ 6, and a sequence identity of at least 50%, or more preferably greater than 55%. 60%, 65%, 70%, 75%, 80%, 85%, 90%, 95%, 97%, 98% sequence identity, or most preferentially greater than 99% sequence identity to SEQ 6. These polypeptide sequences may include, but are by no means limited to, the following sequences: SEQ 82. SEQ 83, SEQ 84, SEQ 85, SEQ 86, SEQ 86, SEQ 87, SEQ 88. SEQ 89, SEQ 90, SEQ 91, SEQ 92, SEQ 93, SEQ 94, SEQ 95, SEQ 96, and SEQ 97.

Embodiments of this invention are described herein, including the best mode known to the inventors for carrying out the invention. Variations of those embodiments may become apparent to those of ordinary skill in the art upon reading the foregoing description. The person skilled in the art is understood to have knowledge of such variations but such variations are understood to form part of the present invention. Accordingly, it is understood by the person skilled in the art that the present invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Within the context of the present invention all terms and technical parameters described fall within their commonly known meanings as known by individuals within the region of science that the proposed invention is associated with, unless otherwise stated. Furthermore, unless otherwise indicated, all techniques utilized within the context of the present invention are commonly conducted within the fields of molecular biology, cell biology, biochemistry, and microbiology.

The term taurine-related precursor is defined within the context of the present invention as any molecule that is associated with the production of taurine as a final product.

A polynucleotide within the context of the present invention is defined as the collection of individual nucleotides in any organization or size that relates to the DNA sequence.

A polypeptide within the context of the present invention is defined as the combination of multiple peptides of any organization or size that relates to the amino acid sequence. The term polypeptide and protein within the context of this invention can be used interchangeably.

A vector within the context of the present invention refers to the composition of a polynucleotide with the intended purpose of introducing nucleic acids into one or more organism types. Vectors are further defined based on their functional purpose and can be designated as expression vectors, cloning vectors, plasmids, or shuttle vectors.

The term “expression” within the context of the present invention refers to the generation of a polypeptide or protein sequence which is produced based on its polynucleotide sequence or gene.

An “expression vector” within the context of the present invention references a polynucleotide sequence containing a coding sequence or gene that enhances or promotes the generation of a polypeptide when introduced into an organism. An expression vector contains all the necessary polypeptide producing features such as a promoter and ribosomal binding site which allow for the production (or expression) of a desired gene due to transcription and translation processes.

A promoter within the context of the present invention is used to describe the nucleic acid sequence for the regulation and binding of polymerases for the purpose of transcribing a gene. This promoter can be native to an organism, or a non-endogenous promoter can be introduced into an organism to alter the regulation of gene expression.

The term gene refers to a DNA sequence that encodes for a specific polypeptide sequence. A gene can include both sequences between coding regions (introns) and the encoding sequence itself (exon).

The term recombinant within the context of the present invention refers to the modification or alteration of a sequence associated with either a polypeptide or polynucleotide sequence. Recombination can be utilized for altering expression and coding segments of a gene of interest that would produce a non-native or non-naturally occurring product.

The term exogenous refers to the addition of either polypeptide and/or polynucleotide molecules that are not normally found within the organism. This includes any un-altered or altered genes and/or proteins that are not found conventionally within an organism.

The term homology refers to the level of similarity between two or more polypeptide or polynucleotide sequences.

The terms transfection, transformation, or introduced refer to the addition of polynucleotide sequence(s) that would normally be considered exogenous to the organism. This can include the addition of a polynucleotide directly to the genome of an organism or the transfer of a plasmid and/or vector to be maintained within the organism.

Within the context of the present invention, the terms native or natural refers to polypeptide and/or polynucleotides present within the organism prior to any modification. These native or naturally occurring polypeptides and/or polynucleotides would be present or produced by the organism without any external alterations.

The term metabolic pathway refers to the subsequential biochemical reactions involved in the formation of a biologically relevant product within an organism.

Within the context of the present invention the terms “knock-in” and “knock-out” refer to the addition or removal of DNA sequences within an organism and can also be interchangeable with the terms insertion and deletion, respectively.

A coding sequence within the context of the present invention refers to a sequence of polynucleotides or DNA that facilitates the generation of a protein through transcription and translational processes (also known as transcribed and translated).

Genetic modification or related statements herein refer to the alteration of the genetic code of an organism which includes the insertion or deletion of DNA sequences within an organism. Within the context of the present invention, genetic modification could include insertion and maintenance of an expression vector into the organism, or the direct modification of the organisms genome by directly adding or deleting genes through processes like, but not limited to, 2 step allelic exchange or CRISPR cloning.

The term ribosomal binding site (RBS) refers to the region within a polynucleotide sequence that allows for the appropriate binding of a ribosome to a polynucleotide sequence to facilitate the translation of a polynucleotide sequence to produce a polypeptide sequence, which includes the terms protein, enzyme, and plasmid.

The term synthetic promoter refers to the addition or modification of a promoter sequence that would not or does not exist within the organism naturally. This can include the insertion or utilization of non-native promoters, or regions of non-native promoters utilized in the modification of protein synthesis.

The term biosynthetic in the context of the present invention refers to the generation of a biological compound by a living organism. This can include but is not limited to the formation of a biological compound that naturally occurs with the organism or the formation of a compound by an organism due to modifications to its genetic code.

The term transgenic, as used herein, refers to the combination of multiple organism polynucleotide sequences within a single organism. For example, if a polynucleotide sequence was sourced from an organism outside of the intended organism of interest within the invention, the organism of the invention's interest would be considered transgenic in nature.

The term cloning vector herein refers to a polynucleotide sequence or plasmid that can be replicated within a host organism for storage or amplification purposes. A cloning vector may contain all the necessary regulatory sequences needed to facilitate the transcription and translation of a protein.

The term unmodified promoter is defined as a promoter sequence which is unaltered and/or exists within the host organism itself.

The term two-step allelic exchange is referring to a process by which a gene of interest is either inserted or deleted from an organism through specific selective conditions. The insertion or deletion of a specific gene of interest is done so through the utilization of distinct polynucleotide sequences which allows for the exchange of genetic material between two sources.

The term CRISPR cloning is defined as a process by which the gene of interest is inserted or removed from an organism's genome using the CRISPR-CAS9 cloning system.

The terms upstream and downstream refer to regions of polynucleotides which are found prior to or after a specific gene of interest within a plasmid and/or genome of an organism.

The term enzyme within the context of the present invention defines a polypeptide sequence, specifically in the form of a protein, that can modify a biological molecule or take part within its generation through direct or indirect interactions. The process by which an enzyme influences the modification and/or production of a biological molecule and/or product is termed enzymatic activity.

The term “bioactive” refers to a substance within the cell that has a biological or chemical effect upon or within the cell. Bioactive substances are not limited to antibiotics, functional enzymes, or vitamins/critical nutrients.

The terms metabolic precursor or precursor within the context of the current invention are compounds that are either uptaken or produced by the cell which are used by the cell to produce the compound of interest.

The term open reading frame (ORF) refers to the collection of nucleotides which are found in between the start and stop codons of a polypeptide encoding DNA sequence.

The term codon(s) refers to 3 adjacent nucleotides in a polynucleotide sequence that are used by the cell to “decode” the polynucleotide sequence when the polynucleotide sequence is translated to make the polypeptide sequence and are responsible defining the order of protein residues in a polypeptide sequence based on this code. Based on a 3-letter code, and 4 different nucleotide bases, these codons include 64 different combinations that are able to be used by the cell, which with some redundancy codes for 22 possible protein residues, as well as 1 start and 3 stop codons.

A start and stop codon refers to nucleotide codon sequences comprised of three specific nucleotides in succession of each other, which allows for the identification of the initiation (start) and termination (stop) for the translation of a polypeptide sequence by the cell.

A unicellular organism refers to any organism of which complete organismal composition consists of a single cell.

The term transcriptional repressor herein refers to a protein that binds to DNA in order to reduce the transcription of a polynucleotide sequence of a specific individual protein and/or enzyme or set of proteins and/or enzymes.

The term central dogma of molecular biology states that genetic material flows in a single direction to produce protein. This dogma states that DNA is transcribed to produce messenger RNA, which in turn is translated to produce the final protein/polypeptide sequence. Simply put: DNA→messenger RNA→Protein.

The term messenger RNA or mRNA refers to a transitory molecule that is found between the polypeptide sequence and the DNA polynucleotide sequence. Simply, the messenger RNA is transcribed from the polynucleotide sequence and the messenger RNA is translated to produce the final protein.

The term antisense RNA refers to a transitory molecule that is used to downregulate the process of converting messenger RNA to the polypeptide sequence, either by causing the degradation of the messenger RNA or by blocking or inhibiting the process to produce the polypeptide sequence from the messenger RNA molecule.

The term antisense transcription refers to the process by which antisense RNA inhibits the production of the final polypeptide product for a gene by interfering with or signalling the degradation of the corresponding messenger RNA molecule.

The term metabolic engineering herein refers to the alteration of an organism's metabolic pathway potential. This can include both the deactivation and/or altering of pre-existing metabolic pathways of an organism or the inclusion of additional metabolic processes.

The term “Sequence alignment” herein refers to a bioinformatic technique by which two polynucleotide sequences or two polypeptide sequences are arranged or aligned in such a way as to identify regions of similarity between a reference sequence (the sequence that is known) and the quarry sequence (the sequence to be compared to the reference sequence). The person skilled in the art understands that alignment algorithms such as, but by no means limited to, the BLAST, ALIGN, or CLUSTAL algorithms can be used to obtain this information for polynucleotide or polypeptide sequences, respectively.

The term “Percentage sequence identity” herein refers to the similarity between 2 sequences that have been processed through a sequence alignment, to provide insight into how similar aligned sequences are at either the nucleotide or peptide level for polynucleotide or polypeptide sequences, respectively. The percentage identity is used to determine the similarity of a query sequence to a reference sequence.

The term “Percentage sequence coverage” refers to the number of aligned nucleotides or peptides in a query sequence relative to the length of the reference sequence. The percentage coverage provides an indication of how much of the reference polynucleotide or polypeptide sequence is covered by the query sequence, allowing for instance the lengths of the found genes or proteins to be compared.

The BLASTN algorithm was used herein as one method to determine the percentage identity and percentage coverage between one or even multiple different polynucleotide sequences with respect to an inputted reference sequence, allowing for the determination of the percentage identity and percentage coverage of one or many query sequences to said reference sequence. The person skilled in the art will recognize that search results from a BLASTN search will be influenced by the search parameters used in the search. Therefore, for all BLASTN searches done with respect to this invention to identify other sequences which have been catalogued in the NCBI polynucleotide databases relative to a reference include the following parameters:
Search set parameters are comprising of “standard databases (nr ect)”, with the specific database used being the “Nucleotide collection (nr/nt)”, and no exclusions or limitations were placed on the search (all default parameters)
Program selection algorithm parameters includes the highly similar sequences (known as the megablast algorithm) (the default parameter)
Algorithm parameters altered include the Max target sequences, which was set at 5000, otherwise all default parameters are used for relevant searches (other parameters in “General parameters”, and all parameters in “Scoring parameters” and “Filters and masking” are default parameters)

The BLASTP algorithm was used herein as one method to determine the percentage identity and percentage coverage between one or even multiple different polypeptide sequences with respect to an inputted reference sequence, allowing for the determination of the percentage identity and percentage coverage of one or many query sequences to said reference sequence. The person skilled in the art will recognize that search results from a BLASTP search will be influenced by the search parameters used in the search. Therefore, for all BLASTP searches done with respect to this invention to identify other sequences which have been catalogued in the NCBI polypeptide databases relative to a reference include the following parameters:
Search set parameters are comprising of “standard databases (nr ect)”, with the specific database used being the “Non-redundant protein sequences (nr)”, and no exclusions or limitations were placed on the search (all default parameters)
Program selection algorithm parameters includes the BLASTP (known as the “protein-protein” BLAST algorithm) (the default parameter)
Algorithm parameters altered include the Max target sequences, which was set at 5000. Otherwise all default parameters are used for relevant searches (other parameters in “General parameters”, and all parameters in “Scoring parameters” and “Filters and masking” are default parameters). Notable default parameters include an “Expect Threshold and word size of 0.05 and 5, respectively in the general parameters, the usage of the BLOSUM62 matrix with gap costs of Existence:11 and Extension:1 for the Scoring parameters, and no filter or masking components selected.

The phrases “substantially similar” or “substantially identical” in the context of at least 2 nucleic acid sequences or at least 2 polypeptide sequences typically means that a polynucleotide, polypeptide, or region or domain of a polypeptide has, preferably, a percentage coverage of at least 70%, 75%, 80%, 85%, 90%, 95%, 97%, 99%, or even 99.5%, and at least 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% percentage identity to the reference sequence. Some polynucleotide or polypeptide sequences that fall in this category are sequences that share genetic or protein homology to the reference sequence.

The terms “genetic homology” and “protein homology”, or “homologous sequences” refer to polynucleotide sequences or translated polypeptide sequences that have a similar or identical function in the cell. For example, 2 different proteins share a similar or identical function even though they were isolated from 2 different organisms. Polynucleotide sequences with homology are generally understood to have similar or identical biochemical functionality.

In scientific literature, genes/proteins are often renamed as more about the gene is determined, often leaving several different associated names for each gene. The FMO1 protein is known as flavin-containing monooxygenase 1. The ADO protein is known as cysteamine dioxygenase and 2-aminoethanethiol dioxygenase. The VNN1 protein is known as vanin-1 and pantetheinase. The VNN2 protein is known as vanin-2 and as pantetheinase. The VNN3 protein is known as vanin-3 and as pantetheinase.

Those of ordinary skill in the art know that many different amino acids contain similar properties between each other and can serve similar functions in the final polypeptide sequence. Thus, when one amino acid is changed with another amino acid from this group, such as a non-polar amino acid, an uncharged polar amino acid, a charged polar acidic amino acid, or a charged polar basic amino acid, some polypeptide functionality is generally maintained. For example, it is known that the uncharged polar amino acid serine may be substituted for the uncharged polar amino acid threonine in a polypeptide without substantially altering the protein structure and functionality. Whether a given substitution will affect the functionality of the enzyme may be determined without undue experimentation using synthetic techniques and screening assays known to a person of ordinary skill in the art.

1. A genetically modified prokaryotic cell comprising:
at least one of the following:
i. an addition, deletion and/or alteration of at least one gene to promote to taurine production; and
ii. an addition, deletion and/or alteration of at least one gene related to taurine cellular transportation; and
at least one of the following polynucleotide sequences:
i. a vanin (vnn) polynucleotide sequence selected from the group consisting of:
vanin-1 (vnn1), wherein said vnn1 polynucleotide sequence has at least 70% sequence coverage to SEQ 3 or SEQ 98, and at least 70% sequence identity to SEQ 3 or SEQ 98;
vanin-2 (vnn2), wherein said vnn2 polynucleotide sequence has at least 70% sequence coverage to SEQ 100, and at least 70% sequence identity to SEQ 100; and
vanin-3 (vnn3), wherein said vnn3 polynucleotide sequence has at least 70% sequence coverage to SEQ 141, and at least 70% sequence identity to SEQ 141;
ii. a cysteamine dioxygenase (ado) polynucleotide sequence which has at least 70% sequence coverage to SEQ 1, and at least 70% sequence identity to SEQ 1; and
iii. a flavin-containing monooxygenase 1 (fmo1) polynucleotide sequence which has at least 70% sequence coverage to SEQ 5 or SEQ 99, and at least 70% of sequence identity to SEQ 5 or SEQ 99.

2. The prokaryotic cell according to claim 1 wherein:
at least one of the following
i. an addition, deletion and/or alteration of at least one gene to promote to taurine production; and
ii. an addition, deletion and/or alteration of at least one gene related to taurine cellular transportation; and
at least one of the following polynucleotide sequences:
i. a vanin (vnn) polynucleotide sequence selected from the group consisting of:
a vanin 1 (vnn1) polynucleotide sequence, wherein said vnn1 polynucleotide sequence is selected from the group consisting of: SEQ 3; SEQ 45, SEQ 46, SEQ, 47, SEQ 48, SEQ 49, SEQ 50, SEQ 51, SEQ 52, SEQ 53, SEQ 54, and SEQ 98;
vanin 2 (vnn2) polynucleotide sequence, wherein said vnn2 polynucleotide sequence is selected from the group consisting of: SEQ 100; SEQ 101; SEQ 102; SEQ 103; SEQ 104; SEQ 105; SEQ 106; SEQ 107; SEQ 108; SEQ 109; SEQ 110; SEQ 111; SEQ 112; and SEQ 113; and
vanin 3 (vnn3) polynucleotide sequence, wherein said vnn3 polynucleotide sequence is selected from the group consisting of: SEQ 141; SEQ 142; SEQ 143; SEQ 144; SEQ 145; SEQ 146; SEQ 147; SEQ 148; SEQ 149; SEQ 150; SEQ 151; SEQ 152; SEQ 153; SEQ 154; SEQ 155; SEQ 156; and SEQ 157;
ii. a cysteamine dioxygenase (ado) polynucleotide sequence selected from the group consisting of: SEQ 1; SEQ 24; SEQ 25; SEQ 26; SEQ 27; SEQ 28; and SEQ 29; and
iii. a flavin-containing monooxygenase 1 (fmo1) polynucleotide sequence selected from the group consisting of: SEQ 5; SEQ 71; SEQ 72; SEQ 73; SEQ 74; SEQ 75; SEQ 76; SEQ 77; SEQ 78; SEQ 79; SEQ 80; SEQ 81; and SEQ 99.

3. The prokaryotic cell according to claim 1 wherein, upon transcription and translation under the control of a native or synthetic promoter and Ribosomal binding site (RBS):
said vanin (vnn) polynucleotide sequence provides a vanin (VNN) polypeptide sequence selected from the group consisting of:
i. a vanin-1 (VNN1) polypeptide sequence, wherein said VNN1 polypeptide sequence has at least 70% sequence coverage to SEQ 4, and at least 25% sequence identity to SEQ 4;
ii. a vanin-2 (VNN2) polypeptide sequence, wherein said VNN2 polypeptide sequence has at least 70% sequence coverage to SEQ 114, and at least 25% sequence identity to SEQ 114; and
iii. a vanin-3 (VNN3) polypeptide sequence, wherein said VNN3 polypeptide sequence has at least 70% sequence coverage to SEQ 158, and at least 25% sequence identity to SEQ 158; and
said cysteamine dioxygenase (ado) polynucleotide sequences provides a cysteamine dioxygenase (ADO) polypeptide sequence which has at least 70% sequence coverage to SEQ 2, and at least 25% sequence identity to SEQ 2; and
said flavin-containing monooxygenase 1 (fmo1) polynucleotide sequence provides a flavin-containing monooxygenase 1 (FMO1) polypeptide sequence which has at least 70% sequence coverage to SEQ 6, and at least 50% of sequence identity to SEQ 6.

4. The prokaryotic cell according to claim 3 wherein:
said vanin-1 (VNN1) polypeptide sequence is selected from the group consisting of: SEQ 4; SEQ 55, SEQ 56, SEQ, 57, SEQ 58, SEQ 59, SEQ 60, SEQ 61, SEQ 62, SEQ 63, SEQ 64, SEQ 65, SEQ, 66, SEQ 67, SEQ 68, SEQ 69, and SEQ 70;
said vanin-2 (VNN2) polypeptide sequence is selected from the group consisting of: SEQ 114; SEQ 115; SEQ 116; SEQ 117; SEQ 118; SEQ 119; SEQ 120; SEQ 121; SEQ 122; SEQ 123; SEQ 124; SEQ 125; SEQ 126; SEQ 127; SEQ 128; SEQ 129; SEQ 130; SEQ 131; SEQ 132; SEQ 133; SEQ 134; SEQ 135; SEQ 136; SEQ 137; SEQ 138; SEQ 139; and SEQ 140;
said vanin-3 (VNN3) polypeptide sequence is selected from the group consisting of SEQ 158; SEQ 159; SEQ 160; SEQ 161; SEQ 162; SEQ 163; SEQ 164; SEQ 165; SEQ 166; SEQ 167; SEQ 168; SEQ 169; SEQ 170; SEQ 171; SEQ 172; SEQ 173; SEQ 174; SEQ 175; SEQ 176; SEQ 177; SEQ 178; SEQ 179: SEQ 180; SEQ 181; SEQ 182; and SEQ 183;
said cysteamine dioxygenase (ADO) polypeptide sequence is selected from the group consisting of: SEQ 2; SEQ 30; SEQ 31; SEQ 32; SEQ 33; SEQ 34; SEQ 35; SEQ 36; SEQ 37; SEQ 38; SEQ 39; SEQ 40; SEQ 41; SEQ 42; SEQ 43; and SEQ 44; and
said flavin-containing monooxygenase 1 (FMO1) polypeptide sequence is selected from the group consisting of SEQ 6; SEQ 82; SEQ 83; SEQ 84; SEQ 85; SEQ 86; SEQ 87; SEQ 88; SEQ 89; SEQ 90; SEQ 91; SEQ 92; SEQ 93; SEQ 94; SEQ 95; SEQ 96; and SEQ 97.

5. The prokaryotic cell according to claim 1 wherein said at least one gene to promote taurine production is selected from the group consisting of: mcbR (SEQ 184), amtr (SEQ 186), xsc-like (SEQ 188), ssuI (SEQ 190), ssuD1 (SEQ 192), ssuD2 (SEQ 194), ssuR (SEQ 196), accA (SEQ 206), accB (SEQ 208), seuA (SEQ 210), seuB (SEQ 212), seuC (SEQ 214), ilvA (SEQ 216), gldc (SEQ 220), ilvB (SEQ 222), ilvN (SEQ 224), ilvC (SEQ 226), ilvD (SEQ 228), pyc (SEQ 230), dadA-like (SEQ 232), coaA (SEQ 234), coaBC (SEQ 236), coaD (SEQ 238), coaE (SEQ 240), panB (SEQ 244), panC (SEQ 246), panD (SEQ 248), panE (SEQ 250), aspB (SEQ 254), mqo (SEQ 256), mdh (SEQ 260), mcr (SEQ 262), puuE (SEQ 264), abat (SEQ 266), and pydD (SEQ 268).

6. The prokaryotic cell according to claim 5 wherein, upon transcription and translation under the control of a native or synthetic promoter and Ribosomal binding site (RBS), said at least one gene to promote taurine production generates a polypeptide sequence selected from the group consisting of: SEQ 185, SEQ 187, SEQ 189, SEQ 191, SEQ 193, SEQ 195, SEQ 197, SEQ 207, SEQ 209, SEQ 211, SEQ 213, SEQ 215, SEQ 217, SEQ 221, SEQ 223, SEQ 225, SEQ 227, SEQ 229, SEQ 231, SEQ 233, SEQ 235, SEQ 237, SEQ 239, SEQ 241, SEQ 245, SEQ 247, SEQ 249, SEQ 251, SEQ 255, SEQ 257, SEQ 261, SEQ 263, SEQ 265, SEQ 267, and SEQ 269.

7. The prokaryotic cell according to claim 1 wherein said at least one gene related to the cellular transport of taurine is selected from the group consisting of: ssuA (SEQ 198), ssuB (SEQ 200), ssuC (SEQ 202), tauE (SEQ 204), gadC (SEQ 218), yhiM (SEQ 242), sdaC (SEQ 252), and cycA (SEQ 258).

8. The prokaryotic cell according to claim 7 wherein, upon transcription and translation under the control of a native or synthetic promoter and Ribosomal binding site (RBS), said at least one gene involved in the transport of taurine generates a polypeptide sequence selected from the group consisting of: SEQ 199, SEQ 201, SEQ 203, SEQ 205, SEQ 219, SEQ 243, SEQ 253, and SEQ 259.

9. The prokaryotic cell according to claim 1 further comprising a promoter and Ribosomal Binding Site (RBS) sequence which drives gene expression, wherein the genetic sequence for the promoter/RBS generates a polypeptide sequence selected from the group consisting of: SEQ 7; SEQ 8; SEQ 9: SEQ 10; SEQ 11; SEQ 12; SEQ 13; SEQ 14; SEQ 15; SEQ 16; SEQ 17; SEQ 18; SEQ 19; SEQ 20; SEQ 21; SEQ 22; and SEQ 23.

10. The prokaryotic cell according to claim 1, where the cell is a bacterial cell.

11. The prokaryotic cell according to claim 1, where the cell is selected from the group consisting of the genera: Brevibacterium, Bacillus, Corynebacterium, Escherichia, Lactococcus, Pseudomonas, Rhodococcus, and Serratia.

12. The prokaryotic cell according to claim 1, where the cell belongs to the genus Corynebacterium.

13. A genetically modified prokaryotic cell comprising:
at least one of the following:
i. an addition, deletion and/or alteration of at least one gene to promote to taurine production; and
ii. an addition, deletion and/or alteration of at least one gene related to taurine cellular transportation; and
at least one of the following polynucleotide sequences:
i. a vanin (vnn) polynucleotide sequence selected from the group consisting of:
vanin-1 (vnn1), wherein said vnn1 polynucleotide sequence has at least 70% sequence coverage to SEQ 3 or SEQ 98, and at least 70% sequence identity to SEQ 3 or SEQ 98;
vanin-2 (vnn2), wherein said vnn2 polynucleotide sequence has at least 70% sequence coverage to SEQ 100, and at least 70% sequence identity to SEQ 100; and
vanin-3 (vnn3), wherein said vnn3 polynucleotide sequence has at least 70% sequence coverage to SEQ 141, and at least 70% sequence identity to SEQ 141;
ii. a cysteamine dioxygenase (ado) polynucleotide sequence which has at least 70% sequence coverage to SEQ 1, and at least 70% sequence identity to SEQ 1; and
iii. a flavin-containing monooxygenase 1 (fmo1) polynucleotide sequence which has at least 70% sequence coverage to SEQ 5 or SEQ 99, and at least 70% of sequence identity to SEQ 5 or SEQ 99.
